(19) United States
(12) Patent Application Publication
(10) Pub. No.: US 2010/0068806 A1
(43) Pub. Date: Mar. 18, 2010

(54) NOVEL METHODS AND REAGENTS DIRECTED TO PRODUCTION OF CELLS

(75) Inventors: Jarmo Laine, Helsinki (FI); Tero Satomaa, Helsinki (FI); Jari Natunen, Vantaa (FI); Annamari Heiskanen, Helsinki (FI); Maria Blomqvist, Itäsalmi (FI); Anne Olonen, Lahti (FI); Juhani Saarinen, Helsinki (FI); Sari Tiitiene, Vantaa (FI); Ulla Impola, Helsinki (FI); Milla Mikkola, Helsinki (FI); Leena Valmu, Helsinki (FI); Minna Tiittanen, Espoo (FI)

Correspondence Address: BIRCH STEWART KOLASCH & BIRCH, PO BOX 747, FALLS CHURCH, VA, 22040-0747, US

(73) Assignees: SUOMEN PUNAINEN RISTI, VERIPALVELU, Helsinki (FI); Glykos Finland Ltd., Helsinki (FI)

(21) Appl. No.: 12/522,801

(22) PCT Filed: Jan. 18, 2008
(86) PCT No.: PCT/FI2008/050016

§ 371 Date: Nov. 10, 2009

(30) Foreign Application Priority Data
Jan. 18, 2007 (FI) 20075033
Jan. 18, 2007 (FI) 20075034

(51) Int. Cl. C12N 5/0735; C12N 5/0775; C07H 21/04; C07K 14/42

(52) U.S. Cl. 435/366; 536/23.6; 530/396

(57) ABSTRACT

The present invention provides methods and materials to modulate and grow stem cells by contacting stem cells with a binder recognizing terminal glycan structures of stem cells. The modulation can be morphological change, change in differentiation status, biological status or adherence. The materials provided in the present invention are also useful to screen such a binding agents and binders.

### FIELD OF THE INVENTION

The invention describes reagents and methods for specific binders to glycan structures of stem cells and the use of these in context of cultivation of cells. Furthermore the invention is directed to screening of additional binding reagents against specific glycan epitopes on the surfaces of the stem cells. The preferred binders of the glycans structures includes proteins such as enzymes, lectins and antibodies.

### BACKGROUND OF THE INVENTION

### Stem Cells

Stem cells are undifferentiated cells which can give rise to a succession of mature functional cells. For example, a hematopoietic stem cell may give rise to any of the different types of terminally differentiated blood cells. Embryonic stem (ES) cells are derived from the embryo and are pluripotent, thus possessing the capability of developing into any organ or tissue type or, at least potentially, into a complete embryo.

The first evidence for the existence of stem cells came from studies of embryonic carcinoma (EC) cells, the undifferentiated stem cells of teratocarcinomas, which are tumors derived from germ cells. These cells were found to be pluripotent and immortal, but possess limited developmental potential and abnormal karyotypes (Rossant and Papaioannou, Cell Differ 15,155-161, 1984). ES cells, on the other hand, are thought to retain greater developmental potential because they are derived from normal embryonic cells, without the selective pressures of the teratocarcinoma environment.

Pluripotent embryonic stem cells have traditionally been derived principally from two embryonic sources. One type can be isolated in culture from cells of the inner cell mass of a pre-implantation embryo and are termed embryonic stem (ES) cells (Evans and Kaufman, Nature 292,154-156, 1981; U.S. Pat. No. 6,200,806). A second type of pluripotent stem cell can be isolated from primordial germ cells (PGCS) in the mesenteric or genital ridges of embryos and has been termed embryonic germ cell (EG) (U.S. Pat. No. 5,453,357, U.S. Pat. No. 6,245,566). Both human ES and EG cells are pluripotent. This has been shown by differentiating cells in vitro and by injecting human cells into immunocompromised (SCUM) mice and analyzing resulting teratomas (U.S. Pat. No. 6,200,806). The term “stem cell” as used herein means stem cells including embryonic stem cells or embryonic type stem cells and stem cells differentiated thereof to more tissue specific stem cells, adults stem cells including mesenchymal stem cells and blood stem cells such as stem cells obtained from bone marrow or cord blood.

The present invention provides novel markers and target structures and binders to these for especially embryonic and adult stem cells, when these cells are not hematopoietic stem cells. From hematopoietic CD34+ cells certain terminal structures such as terminal sialylated type two N-acetyllactosamines such as NeuNAcα3Galβ4GlcNAc (Magnani J. U.S. Pat. No. 6,362,010) has been suggested and there is indications for low expression of Slex type structures NeuNAcα3Galβ4(Fucα3)GlcNAc (Xia L et al Blood (2004) 104 (10) 3091-6). The invention is also directed to the NeuNAcα3Galβ4GlcNAc non-polylactosamine variants separately from specific characteristic O-glycans and N-glycans. The invention further provides novel markers for CD133+ cells and novel hematopoietic stem cell markers according to the invention, especially when the structures does not include NeuNAcα3Galβ4(Fucα3)₀₋₁GlcNAc. Preferably the hematopoietic stem cell structures are non-sialylated, fucosylated structures Galβ1-3-structures according to the invention and even more preferably type 1 N-acetyllactosamine structures Galβ3GlcNAc or separately preferred Galβ3GalNAc based structures.

Human ES, EG and EC cells, as well as primate ES cells, express alkaline phosphatase, the stage-specific embryonic antigens SSEA-3 and SSEA-4, and surface proteoglycans that are recognized by the TRA-1-60; and TRA-1-81 antibodies. All these markers typically stain these cells, but are not entirely specific to stem cells, and thus cannot be used to isolate stem cells from organs or peripheral blood.

The SSEA-3 and SSEA-4 structures are known as galactosylgloboside and sialylgalactosylgloboside, which are among the few suggested structures on embryonal stem cells, though the nature of the structures in not ambiguous. An antibody called K21 has been suggested to bind a sulfated polysaccharide on embryonal carcinoma cells (Badcock G et al Cancer Res (1999) 4715-19. Due to cell type, species, tissue and other specificity aspects of glycosylation (Furukawa, K., and Kobata, A. (1992) Curr. Opin. Struct. Biol. 3, 554-559, Gagneux, and Varki, A. (1999) Glycobiology 9, 747-755; Gawlitzek, M. et al. (1995), J. Biotechnol. 42, 117-131; Goelz, S., Kumar, R., Potvin, B., Sundaram, S., Brickelmaier, M., and Stanley, P. (1994) J. Biol. Chem. 269, 1033-1040; Kobata, A (1992) Eur. J. Biochem. 209 (2) 483-501.) This result does not indicate the presence of the structure on native embryonal stem cells. The present invention is directed to human stem cells.

Some low specificity plant lectin reagents have been reported in binding of embryonal stem cell like materials. Venable et al 2005, (Dev. Biol. 5:15) measured lectins the binding of SSEA-4 antibod positive subpopulation of embryonal stem cells. This approach suffers obvious problems. It does not tell the expression of the structures in antive non-selected embryonal stem cells. The SSEA-4 was chosen select especially pluripotent stem cells. The scientists of the same Bresagen company have further revealed that actual role of SSEA-4 with the specific stem cell lines is not relevant for the pluripotency.

The work does not reveal: 1) The actual amount of molecules binding to the lectins or 2) presence of any molecules due to defects caused by the cell sorting and experimental problems such as trypsination of the cells. It is really alerting that the cells were trypsinized, which removes protein and then enriched by possible glycolipid binding SSEA4 antibody and secondary antimouse antibody, fixed with paraformaldehyde without removing the antibodies, and labelled by simultaneous with lectin and the same antibody and then the observed glycan profile is the similar as revealed by lectin analysis by same scientist for antibody glycosylation (M. Pierce US2005) or 3) the actual structures, which are bound by the lectins. To reveal the possible residual binding to the cells would require analysis of the glycosylations of the antibodies used (sources and lots not revealed). The purity of the SSEA-4 positive cells was reported to be 98-99%, which is unusually high. The quantitation of the binding is not clear as FIG. 3 shows about 10% binding by lectins LTL and DBA, which are not bound to hESC-cells 3^{rd} page, column 2, paragraph 2 and by immunocytochemistry 4 the page last line.

It appears that skilled artisan would consider the results of Venable et al such convenient colocalization of SSEA-4 and the lectin binding by binding of the lectins to the anti-SSEA-4 antibody. It appears that the more rare binding would reflect lower proportion of the terminal epitope per antibody molecule leading to lower density of the labellable antibodies. It is also realized that the non-controlled cell culture process with animal derived material would lead to contamination of the cells by N-glycolyl-neuraminic acid, which may be recognized by anti-mouse antibodies used as secondary antibody (not defined what kind of anti-mouse) used in purification and analysis of purity, which could lead to conveniently high cell purity. The work is directed only to the “pluripotent” embryonal stem cells associated with SSEA-4 labelling and not to differentiated variants thereof as the present invention. The results indicated possible binding (likely on the antibodies) to certain potential monosaccharide epitopes (6^{th} page, Table 1, and column 2) such Gal and Galactosamine for RCA (ricin, inhitable by Gal or lactose), GlcNAc for TL (tomato lectin), Man or Glc for ConA, Sialic acid/Sialic acid α6GalNAc for SNA, Manα for HHL; lectins with partial binding not correlating with SSEA-4: GalNAc/GalNAcβ4Gal (in text) WFA, Gal for PNA, and Sialic acid/Sialic acid α6GalNAc for SNA; and lectins associated by part of SSEA-4 cells were indicated to bind Gal by PHA-L and PHA-E, GalNAc by VVA and Fuc by UEA, and Gal by MAA (inhibited by lactose). UEA binding was discussed with reference as endothelial marker and O-linked fucose which is directly bound to Ser (Thr) on protein. The background has indicated a H type 2 specificity for the endothelial UEA receptor. The specifities of the lectins are somawhat unusual, but the product codes or isolectin numbers/names of the lectins were not indicated (except for PHA-E and PHA-L) and it is known that plants contain numerous isolectins with varying specificities.

Wearne K A et al Glycobiology (2006) 16 (10) 981-990 studied also staining of embryonic stem cells by plant lectins. The data using the low specificity reagents doe not reveal exact glycan structures and specifically not the elongated structure on specific glycan core structures as described by the present invention for human embryonic stem cells nor useful antibody reagent specificities for specific recognition of terminal epitopes. The authors guess some binding/non-binding structures based on the lectin bindings, which appear to be at least partially different from ones revealed by the invention indicating possible technical problems. This work does not imply any other type of usefulness of the lectins in other cell/cell materials directed methods and it does not indicate anything with regard to mesenchymal or other cell types according to the present invention.

The present invention revealed specific structures by mass spectrometric profiling, NMR spectrometry and binding reagents including glycan modifying enzymes. The lectins are in general low specificity molecules. The present invention revealed binding epitopes larger than the previously described monosaccharide epitopes. The larger epitopes allowed us to design more specific binding substances with typical binding specificities of at least disaccharides. The invention also revealed lectin reagents with specified with useful specificities for analysis of native embryonal stem cells without selection against an uncontrolled marker and/or coating with an antibody or two from different species. Clearly the binding to native embryonal stem cells is different as the binding with MAA was clear to most of cells, there was differences between cell line so that RCA, LTA and UEA was clearly binding a HESC cell line but not another.

Methods for separation and use of stem cells are known in the art.

Characterizations and isolation of hematopoietic stem cells are reported in U.S. Pat. No. 5,061,620. The hematopoietic CD34 marker is the most common marker known to identify specifically blood stem cells, and CD34 antibodies are used to isolate stem cells from blood for transplantation purposes. However, CD34+ cells can differentiate only or mainly to blood cells and differ from embryonic stem cells which have the capability of developing into different body cells. Moreover, expansion of CD34+ cells is limited as compared to embryonic stem cells which are immortal. U.S. Pat. No. 5,677,136 discloses a method for obtaining human hematopoietic stem cells by enrichment for stem cells using an antibody which is specific for the CD59 stem cell marker. The CD59 epitope is highly accessible on stem cells and less accessible or absent on mature cells. U.S. Pat. No. 6,127,135 provides an antibody specific for a unique cell marker (EM10) that is expressed on stem cells, and methods of determining hematopoietic stem cell content in a sample of hematopoietic cells. These disclosures are specific for hematopoietic cells and the markers used for selection are not absolutely absent on more mature cells.

There have been great efforts toward isolating pluripotent or multipotent stem cells, in earlier differentiation stages than hematopoietic stem cells, in substantially pure or pure form for diagnosis, replacement treatment and gene therapy purposes. Stem cells are important targets for gene therapy, where the inserted genes are intended to promote the health of the individual into whom the stem cells are transplanted. In addition, the ability to isolate stem cells may serve in the treatment of lymphomas and leukemias, as well as other neoplastic conditions where the stem cells are purified from tumor cells in the bone marrow or peripheral blood, and reinfused into a patient after myelosuppressive or myeloablative chemotherapy.

Multiple adult stem cell populations have been discovered from various adult tissues. In addition to hematopoietic stem cells, neural stem cells were identified in adult mammalian central nervous system (Ourednik et al. Clin. Genet. 56, 267, 1999). Adult stem cells have also been identified from epithelial and adipose tissues (Zuk et al. Tissue Engineering 7, 211, 2001). Mesenchymal stem cells (MSCs) have been cultured from many sources, including liver and pancreas (Hu et al. J. Lab Clin Med. 141, 342-349, 2003). Recent studies have demonstrated that certain somatic stem cells appear to have the ability to differentiate into cells of a completely different lineage (Pfendler K C and Kawase E, Obstet Gynecol Surv 58, 197-208, 2003). Monocyte derived (Zhao et al. Proc. Natl. Acad. Sci. USA 100, 2426-2431, 2003) and mesodermal derived (Schwartz et al. J. Clin. Invest 109, 1291-1301, 2002) cells that possess some multipotent characteristics were identified. The presence of multipotent “embryonic-like” progenitor cells in blood was suggested also by in-vivo experiments following bone marrow transplantations (Zhao et al. Brain Res Protoc 11, 38-45, 2003). However, such multipotent “embryonic-like” stem cells cannot be identified and isolated using the known markers.

The possibility of recovering fetal cells from the maternal circulation has generated interest as a possible means, non-invasive to the fetus, of diagnosing fetal anomalies (Simpson and Elias, J. Am. Med. Assoc. 270, 2357-2361, 1993). Prenatal diagnosis is carried out widely in hospitals throughout the world. Existing procedures such as fetal, hepatic or chorionic biopsy for diagnosis of chromosomal disorders including Down's syndrome, as well as single gene defects including cystic fibrosis are very invasive and carry a considerable risk to the fetus. Amniocentesis, for example, involves a needle being inserted into the womb to collect cells from the embryonic tissue or amniotic fluid. The test, which can detect Down's syndrome and other chromosomal abnormalities, carries a miscarriage risk estimated at 1%. Fetal therapy is in its very early stages and the possibility of early tests for a wide range of disorders would undoubtedly greatly increase the pace of research in this area. Thus, relatively non-invasive methods of prenatal diagnosis are an attractive alternative to the very invasive existing procedures. A method based on maternal blood should make earlier and easier diagnosis more widely available in the first trimester, increasing options to parents and obstetricians and allowing for the eventual development of specific fetal therapy.

The present invention provides methods of identifying, characterizing and separating stem cells having characteristics of embryonic stem (ES) cells for diagnostic, therapy and tissue engineering. In particular, the present invention provides methods of identifying, selecting and separating embryonic stem cells or fetal cells from maternal blood and to reagents for use in prenatal diagnosis and tissue engineering methods. The present invention provides for the first time a specific marker/binder/binding agent that can be used for identification, separation and characterization of valuable stem cells from tissues and organs, overcoming the ethical and logistical difficulties in the currently available methods for obtaining embryonic stem cells.

The present invention overcomes the limitations of known binders/markers for identification and separation of embryonic or fetal stem cells by disclosing a very specific type of marker/binder, which does not react with differentiated somatic maternal cell types. In other aspect of the invention, a specific binder/marker/binding agent is provided which does not react, i.e. is not expressed on feeder cells, thus enabling positive selection of feeder cells and negative selection of stem cells.

By way of exemplification, the binder to Formula (I) are now disclosed as useful for identifying, selecting and isolating pluripotent or multipotent stem cells including embryonic stem cells, which have the capability of differentiating into varied cell lineages.

According to one aspect of the present invention a novel method for identifying pluripotent or multipotent stem cells in peripheral blood and other organs is disclosed. According to this aspect an embryonic stem cell binder/marker is selected based on its selective expression in stem cells and/or germ stem cells and its absence in differentiated somatic cells and/or feeder cells. Thus, glycan structures expressed in stem cells are used according to the present invention as selective binders/markers for isolation of pluripotent or multipotent stem cells from blood, tissue and organs. Preferably the blood cells and tissue samples are of mammalian origin, more preferably human origin.

According to a specific embodiment the present invention provides a method for identifying a selective embryonic stem cell binder/marker comprising the steps of:

A method for identifying a selective stem cell binder to a glycan structure of Formula (I) which comprises:

i. selecting a glycan structure exhibiting specific expression in/on stem cells and absence of expression in/on feeder cells and/or differentiated somatic cells; ii. and confirming the binding of binder to the glycan structure in/on stem cells.

By way of a non-limiting example, adult, mesenchymal, embryonal type, or hematopoietic stem cells selected using the binder may be used in regenerating the hematopoietic or ther tissue system of a host deficient in any class of stem cells. A host that is diseased can be treated by removal of bone marrow, isolation of stem cells and treatment with drugs or irradiation prior to re-engraftment of stem cells. The novel markers of the present invention may be used for identifying and isolating various stem cells; detecting and evaluating growth factors relevant to stem cell self-regeneration; the development of stem cell lineages; and assaying for factors associated with stem cell development.

UEA has been indicated in context of erythroid progenitors related matter WO9425571, the present invention is directed to production of also non-erythrocyte celle and stem cells and novel effective reagents and conjugates. Certain lectins (PSA, PNA) have been indicated for negative cell selection for nerve stem cell preparation JP2003189847 (Kainosu Muramatsu et al.): and (PHA-E, WGA, LACA and AA1 have been idicated for liver stem cell preparation JP2004344031 (Takara Bio, Hidemoto et al). Due to cell type and species specificity of glycosylation these are not relevant with regard to present invention.

Con A/Pha E have been implicated for animal mesenchymal stem cell culture, especially for ossification or chondrification, due to species specificity and cell type specificity of glycosylation data is not relevant with regard to present invention. Furthermore the lectins recognize different structures than the most preferred to terminals tructures according to the invention and the present conjugates were not disclosed. JP20040377953; JP2006204200; Exp Cell Res (2004) 295 (1) 119-27. The methods including use of lectins Con A and Pha-E has been reported for specific animal cells including mesenchymal cells of rabbit and mouse. It is realized that the glycosylation is species specific and therefore the data is not relevant for human. This is also demonstrated by the same invention Figure wherein the only human cell line was activated much more weakly than the animal cells.

The invention further showed that two other lectins WGA and were devoid of activity. Therefore
a. There is no teaching what lectin if any should be used in context of human stem cells
b. Preferred terminal glycan epitopes of present invention where not indicated, but the active lectin ConA recognizes quite unspecifically N-glycan core structures especially mannose comprising N-glycans including complex and other type structures and Pha-E recognized specifically bisecting GlcNAc branch in the middle of N-glycan core structure. The specificities of the inactive lectins WGA includes GlcNAc comprising structures in the middle of various glycans and non-specific recognition of sialic acids
c. The effect of the lectin was reduction of the growth of the cells
d. The immobilization of the lectin and specific preferably covalent immobilizations were not indicated.

When considering the species and cell/tissue type specificity of the glycosylations and glycan recognition, the speculation from the animal mesenchymal stem cells can not be generalized to any human cells and even less to different cell type such as blood derived stem cells.

The invention revealed that it would be useful to cultivate hematopoietic stem cell in the presence of binder recognizing terminal epitopes glycans of the cells. The preferred terminal epitopes include terminal reducing end epitopes and non-reducing end epitopes of the glycans. The terminal epitopes are especially preferred because availability of the structures for the recognition.

Certain galactose binding lectins have been implicated for removal of lymphocytes from bone marrow transplants WO8000058, EP0015′6790 (Sharon N Reisner Y), this is negative selection and use is especially for bone marrow cells, which differs from the preferred cord blood cells of the invention.

Lectins named as FRIL and related materials have been reported to have some kind(s) of mannose binding activity and have stem cell maintenance related activities or other contextes: WO2007066352 (Dolichos lab lab; garlic lectin (GL), Musa paradise (BL), Arthrocarpus integrifolia (AL); Wo9825457, US2003049339, WO0149851: Phaseolus vulgaris Pha-E, D. lab lab, Sphellostylis stenocarpa. The present invention reveals new lectin when many lectins appears to have been screened, and novel preferred optimal specificity for mannose binding lectins, the invention is further directed to novel material can conjugates.

### BRIEF DESCRIPTION OF THE DRAWINGS

FIG. 1. FACS analysis of seven cord blood mononuclear cell samples (parallel columns) by FITC-labelled lectins. The percentages refer to proportion of cells binding to lectin. For abbreviations of FITC-labelled lectins see text.

FIG. 2. Lectin staining of hESC colonies grown on mouse feeder cell layers, with (A) Maackia amuriensis agglutinin (MAA) that recognizes α2,3-sialylated glycans, and with (B) Pisum sativum agglutinin (PSA) that recognizes α-mannosylated glycans. Lectin binding to hESC was inhibited by α3′-sialyllactose and D-mannose for MAA and PSA, respectively, and PSA recognized hESC only after cell permeabilization (data not shown). Mouse fibroblasts had complementary staining patterns with both lectins, indicating that their surface glycans differed from hESC. C. The results indicate that mannosylated N-glycans are localized in the intracellular compartments in hESC, whereas α2,3-sialylated glycans occur on the cell surface.

FIG. 3. Implications of hESC fucosyltransferase gene expression profile. A. hESC express three fucosyltransferase genes: FUT1, FUT4, and FUT8. B. The expression levels of FUT1 and FUT4 are increased in hESC compared to EB, which potentially leads to more complex fucosylation in hESC. Known fucosyltransferase glycan products are shown. Arrows indicate sites of glycan chain elongation. Asn indicates linkage to glycoprotein.

FIG. 4. Portrait of the hESC N-glycome. MALDI-TOF mass spectrometric profiling of the most abundant 50 neutral N-glycans (A.) and 50 sialylated N-glycans (B.) of the four hESC lines FES 21, 22, 29, and 30 (black columns), four EB samples (gray columns), and four st.3 differentiated cell samples (white columns) derived from the four hESC lines, respectively. The columns indicate the mean abundance of each glycan signal (% of the total glycan signals). The observed m/z values for either [M+Na]+ or [M-H]− ions for the neutral and sialylated N-glycan fractions, respectively, are indicated on the x-axis.

FIG. 5. Detection of hESC glycans by structure-specific reagents. To study the localization of the detected glycan components in hESC, stem cell colonies grown on mouse feeder cell layers were labeled by fluoresceinated glycan-specific reagents selected based on the analysis results. A. The hESC surfaces were stained by Maackia amurensis agglutinin (MAA), indicating that α2,3-sialylated glycans are abundant on hESC but not on feeder cells (MEF, mouse feeder cells). B. In contrast, the hESC cell surfaces were not stained by Pisum sativum agglutinin (PSA) that recognized mouse feeder cells, indicating that α-mannosylated glycans are not abundant on hESC surfaces but are present on mouse feeder cells. C. Addition of 3′-sialyllactose blocks MAA binding, and D. addition of D-mannose blocks PSA binding.

FIG. 6. hESC-associated glycan signals selected from the 50 most abundant sialylated N-glycan signals of the analyzed hESC, EB, and st.3 samples (data taken from FIG. 4.B).

FIG. 7. Differentiated cell associated glycan signals selected from the 50 most abundant sialylated N-glycan signals of the analyzed hESC, EB, and st.3 samples (data taken from FIG. 4.B).

FIG. 8. Schematic representation of the N-glycan change during differentiation (details do not necessarily refer to actual structures). According to characterization of the Finnish hESC lines FES 21, 22, 29, and 30, hESC differentiation leads to a major change in hESC surface molecules. St.3 means differentiation stage after EB stage.

FIG. 9. Stem cell nomenclature used to describe the present invention.

FIG. 10. MALDI-TOF mass spectrometric profile of isolated human stem cell neutral glycosphingolipid glycans. x-axis: approximate m/z values of [M+Na]⁺ ions as described in Table. y-axis: relative molar abundance of each glycan component in the profile. hESC, BM MSC, CB MSC, CB MNC: stem cell samples as described in the text.

FIG. 11. MALDI-TOF mass spectrometric profile of isolated human stem cell acidic glycosphingolipid glycans. x-axis: approximate m/z values of [M-H]⁻ ions as described in Table. y-axis: relative molar abundance of each glycan component in the profile. hESC, BM MSC, CB MSC, CB MNC: stem cell samples as described in the text.

FIG. 12. Mass spectrometric profiling of human embryonic stem cell and differentiated cell N-glycans. a Neutral N-glycans and b 50 most abundant acidic N-glycans of the four hESC lines (white columns), embryoid bodies derived from FES 29 and FES 30 hESC lines (EB, light columns), and stage 3 differentiated cells derived from FES 29 (st.3, black columns). The columns indicate the mean abundance of each glycan signal (% of the total detected glycan signals). Error bars indicate the range of detected signal intensities. Proposed monosaccharide compositions are indicated on the x-axis. H: hexose, N: N-acetylhexosamine, F: deoxyhexose, S: N-acetylneuraminic acid, G: N-glycolylneuraminic acid, P: sulphate/phosphate ester.

FIG. 13. A) Baboon polyclonal anti-Galα3Gal antibody staining of mouse fibroblast feeder cells (left) showing absence of staining in hESC colony (right). B) UEA (Ulex Europaeus) lectin staining of stage 3 human embryonic stem cells. FES 30 line.

FIG. 14. A) UEA lectin staining of FES22 human embryonic stem cells (pluripotent, undifferentiated). B) UEA staining of FES30 human embryonic stem cells (pluripotent, undifferentiated).

FIG. 15. A) RCA lectin staining of FES22 human embryonic stem cells (pluripotent, undifferentiated). B) WFA lectin staining of FES30 human embryonic stem cells (pluripotent, undifferentiated).

FIG. 16. A) PWA lectin staining of FES30 human embryonic stem cells (pluripotent, undifferentiated). B) PNA lectin staining of FES30 human embryonic stem cells (pluripotent, undifferentiated).

FIG. 17. A) GF 284 immunostaining of FES30 human embryonic stem cell line. Immunostaining is seen in the edges of colonies in cells of early differentiation (10× magnification). Mouse feeder cells do not stain. B) Detail of GF284 as seen in 40× magnification. This antibody is suitable for detecting a subset of hESC lineage.

FIG. 18. A) GF 287 immunostaining of FES30 human embryonic stem cell line. Immunostaining is seen throughout the colonies (10× magnification). Mouse feeder cells do not stain. B) Detail of GF287 as seen in 40× magnification. This antibody is suitable for detecting undifferentiated, pluripotent stem cells.

FIG. 19. A) GF 288 immunostaining of FES30 human embryonic stem cells. Immunostaining is seen mostly in the edges of colonies in cells of early differentiation (10× magnification). Mouse feeder cells do not stain. B) Detail of GF288 as seen in 40× magnification. This antibody is suitable for detecting a subset of hESC lineage.

FIG. 20. Immunostaining of CA15-3 in MSC and osteogenically differentiated cells (sialylated carbohydrate epitope in MUC-1, =GF275). Punctate staining is seen in MSC and more cell membrane localized staining pattern in osteogenically differentiated cells (6 weeks of differentiation, confluent culture). The FACS analysis shows the percentage of MSCs expressing GF275 immunostaining. Majority (more than 80-90%) of osteogenically differentiated cells express GF275

FIG. 21. Immunostaining of MSC and osteogenically differentiated cells. Blood group H1(0) antigen, Lewis d (BG4=GF303). No clear staining is seen in MSC whereas osteogenically differentiated cells show clear immunostaining in more than 70-90% of cells.

FIG. 22. H type 2 blood group antigen (=GF302) immunostaining of MSC and osteogenically differentiated MSCs. The immunostaining in MSCs is seen in approx. 20-75% of both cell types.

FIG. 23. Lewis x (SSEA-1=GF305) immunostaining of MSC and osteogenically differentiated MSCs. Very few cells, less than 10% stain with GF305 in MSCs. Osteogenically differentiated cells do not show or show very little of immunostaining. Sialyl Lewis x (=GF307) immunostaining of MSC and osteogenically differentiated MSCs. Sialyl Lewis x immunostaining decreases when MSC differentiate into osteogenic direction.

FIG. 24. CD77 (globotriose (GB3), pk-blood group=GF298) immunostaining of MSC and osteogenically differentiated MSCs. (Subpopulations of) MSCs and osteogenic direction differentiated MSCs express CD77. Globoside GB4 (=GF297) immunostaining of MSC and osteogenically differentiated MSCs. More punctuate staining of GB4 can be seen in MSCs than in osteogenically differentiated cells.

FIG. 25. SSEA-3 (=GF353) and SSEA-4 (=GF354) immunostaining of MSC and osteogenically differentiated MSCs. SSEA-3 immunostaining decreases when MSC differentiate into osteogenic direction. SSEA-4 (=GF354) immunostaining decreases when MSC differentiate into osteogenic direction.

FIG. 26. Tn (CD175=GF278) immunostaining of MSC and osteogenically differentiated MSCs. Few (5-45%) MSCs express CD175 compared to MSCs differentiated into osteogenic direction.

FIG. 27. sialyl Tn (sCD175=GF277) immunostaining of MSC and osteogenically differentiated MSCs. Few MSCs express sialyl Tn, 5-45%. Osteogenically differentiated cells express more or mainly the epitope.

FIG. 28. Oncofetal antigen (TAG-72=GF276) immunostaining of MSC and osteogenically differentiated MSCs. TAG-72 immunostaining increases or is upregulated when MSC differentiate into osteogenic direction.

FIG. 29. Morphologically cells growing on PSA coating differed from the others by their way of forming a netlike monolayer. Cells on MAA and PSA were also more tightly attached to the surface and their detachment with trypsin was not possible, those cells needed to be scratched off mechanically.

FIG. 30. hESC grown in ECA and matrigel coating. The embryonic stem cells grew more evenly on ECA-coated than on Matrigel™-coated plates with no apparent batch-to-batch variation in growing density. They formed small colonies, which was different from Matrigel. The colonies were smaller than those formed by hESC grown on feeder cells.

FIG. 31. Stem cell and differentiation markers for hESC grown on ECA and Matrigel. The figure shows that stem cell marker Oct-4 is upregulated on mouse feeder cells but not on ECA coated plates after 2 and 4 passages. Among differentiation markers Goosecoid shows brief upregulation after passage 2 but is decreased at the same level as or lower level than hESC grown on matrigel by passage 4. Other differentiation marker Sox7 does not show changes when hESC are grown on ECA coated plates.

FIG. 32. A. Passages P4 and P6. B, After 4 passages FACS analysis Tra-1-60 32% and SSEA3 83%. Matrigel 49% and 79%. C, passages p5. D, FACS analysis of markers and hESC (FES29 p36) for culturing on ECA. E, FACS analysis of Matrigel p4 vs. Matrigel p2+ECA.

FIG. 33. A, FES29 p38, Matrigel p3, and lectin p1. FACS: Tra-1-60 70% and SSEA3 89%. B, passage 4 images of cells grown on lectins. UEA, DSA and galectin.

FIG. 34. MSC cells grown on different lectins. PSA lectin, cells are CD105 pos, CD73 pos, CD 45 neg, and HLA-DR is 21.6%. MSCs on HHA show CD105 pos, CD73 pos, CD 45 neg, and HLA-DR is 27.4%.

FIG. 35. MSC cells grown on different lectins. LcHA lectin, cells are CD105 pos, CD73 pos, CD 45 neg and HLA-DR is 27.3%.

ECA lectin, cells are CD 105 pos, CD73 pos, CD 45 neg, and HLA-DR is 26%.

FIG. 36. MSC cells grown on different lectins. ConA lectin, cells are CD105 pos, CD73 pos, CD 45 neg, and HLA-DR is 19.6%.

MAA lectin, cells are CD105 pos, CD73 pos, CD 45 neg, and HLA-DR is R 28.2%.

FIG. 37. MSC cells grown on different lectins. SNA lectin, cells are CD105 pos, CD73 pos, CD 45 neg, and HLA-DR is 18.3%. Galectin-1 lectin, cells are CD105 pos, CD73 pos, CD 45 neg, and HLA-DR is 23.8%. On plastic HLA-DR is 56.5%.

FIG. 38. A synthetic gene (Gene seq. No 899) coding for partial amino acid sequence of Erythrina cristagalli lectin. See Example 24.

FIG. 39. A synthetic gene (Gene seq. No 900) coding for non-glycosylated partial amino acid sequence of Erythrina cristagalli lectin, containing point mutations at nucleotide positions 368 (A>C) and 370 (C>A) in comparison to sequence No 899. See Example 24.

FIG. 40. SDS-PAGE analysis of non-glycosylated ngECA purification steps. Lane 1: Unbound material (flowthrough) of Lac-agarose step. Lane 2: Eluated material during washing. Lane 3: Affinity-purified and dialysed ngECA (c. 30 kDa based on MW standards on the first lane from the left), showing no significant impurities. See Example 24.

### SUMMARY OF THE INVENTION

In an aspect of the invention, a method for the modulation of the status of stem cells is provided by contacting at least one stem cell with a binder which recognizes terminal glycan structures of stem cells.

In another embodiment a method for supporting of the undifferentiated status of stem cells is provided by contacting at least one stem cell with a binder which recognizes terminal glycan structures of stem cells.

In another embodiment a method for change of biological status including but not limited to morphologic status and differentiation related status of cells is provided by contacting at least one stem cell with a binder which recognizes terminal glycan structures of stem cells.

In another embodiment a method for change of the adherence status is provided by contacting at least one stem cell or stem cells with binder which recognizes terminal glycan structures of stem cells.

In another embodiment a method for changing growth speed of stem cells is provided by contacting at least one stem cell or stem cells with binder which recognizes terminal glycan structures of stem cells.

In one embodiment of the methods the surface has attached thereto a binder, wherein said binder modulates biological status of stem cell. In related embodiments the surface may be biocompatible, natural or synthetic, or comprise a polymer. In certain embodiments, the polymer is selected from polystyrene, polyesters, polyethers, polyanhydrides, polyalkylcyanoacrylates, polyacrylamides, polyorthoesters, polyphosphazenes, polyvinylacetates, block copolymers, polypropylene, polytetrafluoroethylene (PTFE), or polyurethanes. In yet other embodiments, the polymer may comprise lactic acid or a copolymer. While in still yet other embodiments, the polymer may be a copolymer. Such copolymers can be a variety of known copolymers and may include lactic acid and/or glycolic acid (PLGA).

With respect to biocompatible surfaces, such surfaces may be biodegradable or non-biodegradable. In related embodiments, while not limited thereto, the non-biodegradable surfaces may comprise poly(dimethylsiloxane) and/or poly(ethylene-vinyl acetate). Further, the biocompatible surface, while not limited thereto, may include collagen, metal, hydroxyapatite, glass, aluminate, bioceramic materials, hyaluronic acid polymers, alginate, acrylic ester polymer, lactic acid polymer, glycolic acid polymer, lactic acid/glycolic acid polymer, purified proteins, purified peptides, and/or extracellular matrix compositions.

In still yet further embodiments, the biocompatible surface is associated with an implantable device. The implantable device may be any that is desired to be used and may include a stent, a catheter, a fiber, a hollow fiber, a patch, or a suture. In related embodiments the surface may be glass, silica, silicon, collagen, hydroxyapatite, hydrogels, PTFE, polypropylene, polystyrene, nylon, or polyacrylamide. Yet additional embodiments include wherein the surface comprises a lipid, a plate, a bag, a rod, a pellet, a fiber, or a mesh. Other embodiments include wherein the surface is a particle and additionally wherein the particle comprises a bead, a microsphere, a nanoparticle, or a colloidal particle. Particle and bead sizes may also be chosen and may have a variety of sizes including wherein the bead is about 5 nanometers to about 500 microns in diameter.

In a preferred embodiment the binder is lectin. In another preferred embodiment the binder is an antibody. In another preferred embodiment the binder is a glycosidase, which may have been mutated in active site.

The stem cell can be, for example, a mesenchymal stem cell, or a fetal stem cell. The stem cells can be derived from an umbilical cord, such as, for example, from umbilical cord blood. The stem cells can be derived from an umbilical cord that expresses a CD34+ cell marker. The umbilical cord stem cells can be derived, for example, from a mammal, such as a human. The growth medium can also contain, if desired, a growth factor, combinations of growth factors, or substantial nutrient content allowing for increased viability of the stem cells.

In some embodiments of the invention, a method for the expansion or growth of stem cells is provided, by contacting at least one stem cell or stem cells with a binder. The stem cell can be a) A totipotent cell such as an embryonic stem cell, an extra-embryonic stem cell, a cloned stem cell, a parthenogenesis derived cell; b) A pluripotent cell such as a hematopoietic stem cell, an adipose derived stem cell, a mesenchymal stem cell, a cord blood stem cell, a placentally derived stem cell, an exfoliated tooth derived stem cells, a hair follicle stem cell or a neural stem cell; or c) A tissue specific progenitor cell such as a precursor cell for the neuronal, hepatic, adipogenic, osteoblastic, osteoclastic, cardiac, intestinal, or endothelial lineage.

Another embodiment of the invention is contacting stem cells with a binder wherein said binder stimulates proliferation of pluripotent stem cells such as mesenchymal stem cells characterized by markers such as LFA-3, ICAM-1, PECAM-1, P-selectin, L-selectin, CD49b/CD29, CD49c/CD29, CD49d/CD29, CD61, CD18, CD29, 6-19, thrombomodulin, telomerase, CD10, CD13, STRO-1, STRO-2, VCAM-1, CD146, THY-1. The binder can be used as a stimulator of proliferation alone, e g immobilized in a surface, or as an additive to media known to be useful for culturing said cells.

In some embodiments of the invention, a method for the expansion or growth of stem cells without substantially inducing differentiation is provided by contacting at least one stem cell with binder, which recognizes terminal glycan structures of stem cells. The at least one stem cell can be, for example, totipotent, capable of differentiating into cells of all histological types of the body. The totipotent stem cell can be selected, for example, from an embryonic stem cell, an extra-embryonic stem cell, a cloned stem cell, a parthenogenesis derived cell. The embryonic stem cell can express, for example, one or more of the following markers: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), or human telomerase reverse transcriptase (hTERT). The hematopoietic stem cells can express, for example, one or more of the following markers: CD34, c-kit, and the multidrug resistance transport protein (ABCG2). The adipose-derived stem cells can express, for example, one or more of the following markers: CD13, CD29, CD44, CD63, CD73, CD90, CD166, Aldehyde dehydrogenase (ALDH), and ABCG2. The mesenchymal stem cells can express, for example, one or more of the following markers: STRO-1, CD105, CD54, CD106, HLA-I markers, vimentin, ASMA, collagen-1, and fibronectin, but not HLA-DR, CD117, and hemopoietic cell markers. The cord blood stem cells can express, for example, one or more of the following markers: CD34, c-kit, and CXCR-4. The placental stem cells can express, for example, one or more of the following markers: Oct-4, Rex-1, CD9, CD13, CD29, CD44, CD166, CD90, CD105, SH-3, SH-4, TRA-1-60, TRA-1-81, SSEA-4 and Sox-2. The neural stem cell can be characterized, for example, by expression of RC-2, 3CB2, BLB, Sox-2hh, GLAST, Pax 6, nesting, Muashi-1, and prominin. The at least one stem cell can be pluripotent, capable of differentiating into numerous cells of the body, but not all. The pluripotent stem cell can be selected from hematopoietic stem cells, adipose stem cells, mesenchymal stem cells, cord blood stem cells, placental stem cells or neural stem cells. The at least one stem cell can be a progenitor cell, capable of differentiating into a restricted tissue type. The progenitor stem cell can be selected from, for example, neuronal, hepatic, adipogenic, osteoblastic, osteoclastic, alveolar, cardiac, intestinal, endothelial progenitor cells.

In some embodiments of the present invention, a method for the expansion or growth of stem cells without substantially inducing differentiation is provided, by contacting at least one stem cell with binder which recognizes terminal glycan structures of stem cells. The cell culture media can be supplemented, for example, with a single or a plurality of growth factors. The growth factors can be selected from, for example, a WNT signaling agonist, TGF-b, bFGF, IL-6, SCF, BMP-2, thrombopoietin, EPO, IGF-1, IL-11, IL-5, Flt-3/Flk-2 ligand, fibronectin, LIF, HGF, NFG, angiopoietin-like 2 and 3, G-CSF, GM-CSF, Tpo, Shh, Wnt-3a, Kirre, or a mixture thereof. The media can be selected, for example, from Roswell Park Memorial Institute (RPMI-1640), Dublecco's Modified Essential Media (DMEM), Eagle's Modified Essential Media (EMEM), Optimem, and Iscove's Media. The source of serum can be added to the media. The concentration of serum in the media can be approximately between 0.1% to 25%. The concentration of serum in the media can be approximately 10%. The serum can be selected from adult human serum, fetal human serum, fetal calf serum and umbilical cord blood serum.

In an additional embodiment of the present invention, a stem cell with the preserved ability to proliferate, but having a block in differentiation state is provided, which can be induced by culturing stem cells in contact with binder.

The stem cell can be selected, for example, from a totipotent stem cell, a pluripotent stem cell, and a progenitor stem cell. The stem cell can be maintained in contact with the binder, for example, for a period of 10, 20, 30, 40, 50, 60, 70, 80, 90, or 100 passages. The stem cell can be initially cultured in contact with the binder for a period of time, subsequently to which it can be cultured in a second culture with a different binder and an identical or variable mix of cytokines or growth factors. The stem cell can be initially cultured for e.g. 20 passages contacted with a binder and a growth factor. The stem cell can be maintained in a cell culture media that can be supplemented with at least one growth factor selected from the group consisting of WNT signaling agonist, TGF-b, bFGF, IL-6, SCF, BMP-2, thrombopoietin, EPO, IGF-1, IL-11, IL-5, Flt-3/Flk-2 ligand, fibronectin, LIF, HGF, NFG, angiopoietin-like 2 and 3, G-CSF, GM-CSF, Tpo, Shh, Wnt-3a, Kirre, and a mixture thereof. The stem cell can be maintained in a growth media with the following growth factors also in DMEM media: IL-3 (about 20 ng/ml), IL-6 (about 250 ng/ml), SCF (about 10 ng/ml), TPO (about 250 ng/ml), flt-3L (about 100 ng/ml). The stem cell can be maintained in the presence of an agent selected from one or more of the following: an inhibitor of GSK-3, an inhibitor of histone deacetylase activity, and inhibitor of DNA methyltransferase activity.

An embodiment of the present disclosure is directed to a purified preparation of pluripotent human ES cells, wherein the cells comprise: (i) the ability to differentiate to derivatives of endoderm, mesoderm, and ectoderm tissues, (ii) a normal karyotype, (iii) the ability to propagate in an in vitro culture for at least about 10 passages, and (iv) obtained from contacting said cells with a binder of the present invention.

In a preferred embodiment binder is lectin, antibody or glycosidase.

The term “purified preparation of pluripotent human ES cells” as used herein means that substantially all of the human ES cells in the purified preparation have the recited characteristics. Therefore, a purified preparation of pluripotent human ES cells may comprise cells wherein at least about 50%, 55%, 60%, 65%, 70%, 75%, 80%, 85%, 90%, 95%, or 99% have the characteristics of the general population of the human ES cells in the preparation, such as, for example, the ability to differentiate to derivatives of endoderm, mesoderm, and ectoderm tissues, a normal karyotype, and the ability to propagate in an in vitro culture for at least about 10 or 20 passages.

The term “agent” or “binder”, or “binding agent”, as used herein, refers to a molecule that binds and/or recognizes terminal glycan structures on stem cells. The binder may bind any cell surface moiety or cell surface moiety bearing terminal glycan structures, such as a receptor, an antigenic determinant, or other binding site present on the target cell population. The binder may be a protein, peptide, antibody and antibody fragments thereof, lectin, glycosidase, glycosyl transferrin enzyme or the like. Within the specification and in the context of stem cell modulation, lectins and antibodies are used as a prototypical example of such a binder.

A “surface”, as used herein, refers to any surface capable of having an agent attached thereto and includes, without limitation, metals, glass, plastics, co-polymers, colloids, lipids, cell surfaces, and the like. Essentially any surface that is capable of retaining an agent bound or attached thereto.

For example, the human ES cells of the present disclosure (1) may proliferate in an in vitro culture for 10, 20, 40 or more than 60 passages; (2) are inhibited from differentiating when cultured in the presence of a binder, e.g. lectin, antibody or glycosidase; (3) are positive for the SSEA-3 and SSEA-4 markers; (4) are positive for the TRA-1-60, and TRA-1-81 markers; (5) are positive for the Oct-4 markers; or (6) are capable of forming embryoid bodies when placed in suspension culture or transplanted in an immunocompromised animal, preferably into a mouse. Preferably, the preparations of pluripotent human ES cells of the present disclosure have not been exposed to animal generated antibodies and sera.

In preferred embodiments, the preparation remains substantially undifferentiated after about 10 passages in culture, more preferably after about 20 passages in culture, even more preferably after about 40 passages in culture, even more preferably after about 60 passages in culture and most preferably after about 100 passages in culture. Although colonies of undifferentiated ES cells within the preparation may be adjacent to neighboring cells that are differentiated, the preparation will nevertheless remain substantially undifferentiated when the preparation is cultured or passaged under appropriate conditions in the presence of a binder, and individual undifferentiated ES cells constitute a substantial proportion of the cell population. Preparations that are substantially undifferentiated contain at least about 20% undifferentiated ES cells, and may contain at least about 40%, 50%, 60%, 70%, 80%, or 90% ES cells.

The present invention is directed to analysis of broad glycan mixtures from stem cell samples by specific binder (binding) molecules.

The present invention is specifically directed to glycomes of stem cells according to the invention comprising glycan material with monosaccharide composition for each of glycan mass components according to the Formula I:

R₁Hexβz{R₃}ₙ₁HexNAcXyR₂ (I),

wherein
X is nothing or a glycosidically linked disaccharide epitope β4(Fucα6)ₙGN, wherein
n is 0 or 1;

### Hex is Gal or Man or GlcA;

### HexNAc is GlcNAc or GalNAc;

y is anomeric linkage structure α and/or β or a linkage from a derivatized anomeric carbon,
z is linkage position 3 or 4, with the provision that when z is 4, then HexNAc is GlcNAc and Hex is Man or Hex is Gal or Hex is GlcA, and
when z is 3, then Hex is GlcA or Gal and HexNAc is GlcNAc or GalNAc;
R₁ indicates 1-4 natural type carbohydrate substituents linked to the core structures,
R₂ is reducing end hydroxyl, a chemical reducing end derivative or a natural asparagine linked N-glycoside derivative including asparagines, N-glycoside aminoacids and/or peptides derived from proteins, or a natural serine or threonine linked O-glycoside derivative including asparagines, N-glycoside aminoacids and/or peptides derived from proteins;
R3 is nothing or a branching structure representing GlcNAcβ6 or an oligosaccharide with GlcNAcβ6 at its reducing end linked to GalNAc, when HexNAc is GalNAc, or R3 is nothing or Fucα4, when Hex is Gal, HexNAc is GlcNAc, and z is 3, or R3 is nothing or Fucα3, when z is 4.

Typical glycomes comprise of subgroups of glycans, including N-glycans, O-glycans, glycolipid glycans, and neutral and acidic subglycomes.

The invention is directed to diagnosis of clinical state of stem cell samples, based on analysis of glycans present in the samples. The invention is especially directed to diagnosing cancer and the clinical state of cancer, preferentially to differentiation between stem cells and cancerous cells and detection of cancerous changes in stem cell lines and preparations.

The invention is further directed to structural analysis of glycan mixtures present in stem cell samples.

### DESCRIPTION OF THE INVENTION

The invention present invention is directed to a method for the modulation of the status of stem cells wherein at least one stem cell is contacted with a glycan binding protein, which alternatively referred here as a binder. In a preferred embodiment the binder is capable of binding to at least one glycan structure on the surface of the stem cell. More preferably the binder recognizes terminal glycan structures of stem cells.

The invention is directed to modulating of or culturing of non-hematopoietic stem cells, comprising: (i) providing at least one stem cell or stem cell population; and (ii) contacting said at least one stem cell or stem cell population with one or more binders, which bind glycan structures. The invention is further directed to the method comprising step (iii) incubating said cells for a period of time sufficient to achieve desired stimulation, status change or growth or iii) culturing the stem cells when growth of stem cells occurs without substantially differentiation.

In an aspect of the invention, a method for the modulation of the status of stem cells is provided by contacting at least one stem cell with a binder. The binder preferably recognizes terminal glycan structures of stem cells.

In an aspect of the invention the binder is a conjugate of a glycan binding protein, preferably polyvalent conjugate. In a preferred embodiment the invention is directed to methods of modulating stem cells in presence of a binder when the binder is immobilized. The preferred immobilization is immobilization by non-covalent interactions and covalent immobilization.

In another embodiment a method for supporting of the undifferentiated status of stem cells is provided by contacting at least one stem cell with a binder which recognizes terminal glycan structures of stem cells. The invention is directed to culturing stem cells, wherein growth of stem cells occurs without substantially inducing differentiation.

The invention is in a preferred embodiment directed to non-hematopoietic stem cells according to the invention, most preferably embryonic or mesenchymal stem cells.

The invention is further directed to method for selecting a binder for modulating of or culturing of hematopoietic stem cells, comprising: (i) providing at least one stem cell or stem cell population; and (ii) contacting said at least one stem cell or stem cell population with one or more binders, which bind glycan structures and wherein the binder is not Manα binding lectin FRIL-group lectin or lectin with similar specificity, or other lectin used for culture of hematopoietic stem cells or the binder is covalently attached to a surface.

The preferred binder for the culture of hematopoietic stem cells has specificity for binding to glycans of hematopoietic stem cells as revealed by the invention.

The invention is further directed to modulation of stem cells including hematopoietic stem cells wherein the modulation involves differentiation of the cells.

In another embodiment a method for change of biological status including but not limited to morphologic status and differentiation related status of cells is provided by contacting at least one stem cell with a binder which recognizes terminal glycan structures of stem cells.

In another embodiment a method for change of the adherence status is provided by contacting at least one stem cell with a binder which recognizes terminal glycan structures of stem cells.

In another embodiment a method for changing growth speed of stem cells is provided by contacting at least one stem cell with a binder which recognizes terminal glycan structures of stem cells.

In a preferred embodiment the binder is lectin. The most preferred lectin for human embryonic stem cells is ECA (E. cristacalli). In a preferred embodiment hESC are grown on an ECA coated surface and essentially feeder cell free. Preferably, ECA coated surfaces maintain hESC substantially in undifferentiated state. In another preferred embodiment hESC culture media comprises a conditioned media, preferably with mEF or hEF conditioned. Preferably, hESC are grown on mouse feeder cells and transferred to grow on ECA coated plates. In a more preferred embodiment hESC are obtained from a blastocyst and directly coated on ECA coated surfaces. hESCs can be propagated using collagenase treatment. Preferably, hESC can be propagated/passaged using phosphate buffered saline (PBS), which would decrease the possible cellular damage caused by repeated exposure to proteases.

In another preferred embodiment the binder is a glycosidase, which may have been mutated in active site.

The present invention provides a method for supporting of the undifferentiated status of stem cells by contacting at least one stem cell with binder which recognizes terminal glycan structures of stem cells. Preferably, the method involves contacting stem cell with a binder that has been immobilized on a surface. Preferably the surface is the bottom of a culture plate or a Petri dish.

Furthermore, there is a need for agents which, in addition to increasing the rate of stem cell proliferation, also maintain the stem cells in an undifferentiated state. Further, there is a need for agents which decrease the rate of stem cell proliferation and/or maintain the stem cells in an undifferentiated state. Further, there is a need for agents which change of the adherence status, morphology, growth speed and/or differentiation status of stem cells.

This becomes particularly apparent when one considers that, in general, stem cells reside in unique physiological niches, and while growing cells within mimics of such niches has been performed, the mimics of the stem cell niche are often unusable in clinical situations. An example of this is the fact that optimal growth of embryonic stem cells is still primarily achieved using murine feeders. The current invention teaches methods and compositions for recreating conditions essentially without feeder cells and potential sources of contamination.

Accordingly, whether a stem cell population is derived from adult or embryonic sources, the stem cells can be grown in a culture medium to increase the population of a heterogeneous mixture of cells, or a purified cell population. The cell growth can be slow, however, and the cells can differentiate to unwanted cell types during the culture period. Thus, methods of improving the growth rate of stem cells, in general, and defined stem cell populations in particular, will be useful for advancing the clinical use of stem cells. Accordingly, what is needed is novel methods of increasing the rate of expansion or growth of the stem cells when grown in culture. Further, what is needed is novel methods of modifying the biological characteristics, for example, adherence status, morphology, growth speed and/or differentiation status or growth of the stem cells when grown in culture.

Contacting a cell population with a binder (e.g., a lectin) that binds to a cell surface moiety can stimulate/modulate the cell population. The binder may be in solution but also may be attached to a surface. Binding of the binder on cell surface moieties/glycan structures may generally induce activation of signaling pathways.

The invention revealed specific binding structures, binders, recognizing terminal glycan structures of stem cells. The invention is specifically directed to use of the binders for the modulation of stem cells. Furthermore present invention is especially directed to novel conjugates of the stem cell binding molecules. The conjugated stem cell binding molecules are especially preferred for the modulation of the stem cells in polyvalent form, especially in immobilized form. The binding molecules are preferably immobilized on a surface.

Glycomes—Novel Glycan Mixtures from Stem Cells

The present invention revealed novel glycans of different sizes from stem cells. The stem cells contain glycans ranging from small oligosaccharides to large complex structures. The analysis reveals compositions with substantial amounts of numerous components and structural types. Previously the total glycomes from these rare materials has not been available and nature of the releasable glycan mixtures, the glycomes, of stem cells has been unknown.

The invention revealed that the glycan structures on cell surfaces vary between the various populations of the early human cells, the preferred target cell populations according to the invention. It was revealed that the cell populations contained specifically increased “reporter structures”.

The glycan structures on cell surfaces in general have been known to have numerous biological roles. Thus the knowledge about exact glycan mixtures from cell surfaces is important for knowledge about the status of cells. The invention revealed that multiple conditions affect the cells and cause changes in their glycomes. The present invention revealed novel glycome components and structures from human stem cells. The invention revealed especially specific terminal Glycan epitopes, which can be analyzed by specific binder molecules.

Preferred terminal epitopes has been represented in Formulas according to the invention in the structure tables, derived from the extensive structural data of the examples. The invention revealed novel elongated binder target epitopes which are preferably recognized by a binder, preferably by a high specifificity binder not recognizing effectively the same terminal structure on other carrier structures. The invention is especially directed to the use of specific binder for enrichment and/or cultivation of mesenchymal or embryonal stem cells, The invention is further directed to the recognition of terminal epitomes wherein the terminal N-glycan epitopes are β2-linked to mannose, O-glycan N-acetyllactosamine based epitopes are β6-linked to GalNAc and glycolipid N-acetyllactosamine besed epitopes are β3-linked to Gal.

### Fucosylated Structures

### i) α3-Fucosylated Structures,

Preferred α3-fucosylated structures includes especially Lewis x and more preferably sialyl-Lewis x. The invention is in a preferred embodiment directed to stem cell populations enriched by binding to α3-fucosylated structures on the cell surfaces by specific binder reagents.

The invention is further directed to complex of α3-fucose specific binder reagent and stem cells, especially for the use of cell cultivation.

Specific sialyl-Lewis x structures were revealed to be effectively mesenchymal or embryonic stem cell specific and useful for binding and manipulation of the cells.

The preferred binding reagent for sLex includes GF 526, and GF307.

In a preferred embodiment the sialyl_Lewis x specific reagent bind especially core II sLex [SAα3Galβ4(Fucα3)GlcNAcβ6(R1Galβ3)GalNAcαSer/Thr, wherein R1 ie sialic acid (SAα3) or nothing.] as the antibody GF526. The invention is especially directed to the selection of sLex and core II sLEx positive cells byt specific binder reagens from material comprising stem cells and especially for the culture of stem cells. In a preferred embodiment the cell sorting system is FACS or solid phase comprising the binders.

### Preferred Lectin Reagents for Growing Stem Cells

The present invention revealed novel lectin reagents useful in the context of growing stem cells.

### Recombinant Lectins

A preferred type of lectin is recombinant protein produced in non-mammalian, preferably in non-animal cell culture. It is realized that such protein have especially low risk of contamination. Preferred production hosts include bacteria, insect, yeast, fungal or plant cells, yeast or fungi are preferred due to lowest level of potentially harmful component in comparison to allergenic plant materials or potential endotoxin containing bacterial production. The example 24 shows a novel recombinant lectins especially useful for the culture of hESC cells.

### Glycan Remodeled Lectins

In a preferred embodiment the invention is directed to use of a naturally glycosylated lectin, which is remodeled to reduce bioactive glycosylation. It is realized that animal glycosylation and even non-animal glycosylation includes bioactive, antigenic or immunogenic structures, which would be harmful if would be transferred to patient with a therapeutic stem cell preparation or cause misleading studies in animal models or cause alterations in cultivated cells through natural glycan binding receptors.

The glycan is preferably remodeled by
a) removal of the glycan/glycosylation site or
b) inactivating the glycan

### Non-Glycosylated Lectins

It is further realized that the non-glycosylated forms of naturally glycosylated lectins such as plant lectins would be useful for biotechnical processes because homogeneity of the protein in comparison to glycosylated protein carrying multiple glycoforms. Non-glycosylated lectin may be produced in prokaryotic system such as by E. coli, e.g ECA lectin has been produced in bacteria. Due to bacterial endotoxins and potential bacterial lectins or glycosidases reactive with sugar affinity column yeast of fungal expression are preferred.

### Glycan Inactived Lectin

The invention is further directed to modifying the glycan of the lectin to inactive form. In a preferred embodiment the glycan is modified by oxidation, preferably by perjodate oxidation and further derived to inactive form or conjugated from to glycan to solid phase so that the glycan is not sterically available for the recognition by the cells.

### Glycan Conjugated Lectin

It further realized that the glycan conjugated forms of glycan inactivated lectins have other benefits in comparison to the passively or non-specifically chemically solid phase adhered lectins, because these methods would at least partially hinder the binding sites of the lectin. Furthermore the glycan conjugated lectins can be attached uniformly to surfaces. The invention revealed that regular conjugation means such as biotinylation to protein would reduce the biological activity of a protein. In the example

### Glycosylation Site Mutated Recombinant ECA Lectin

The invention is in a preferred embodiment directed to a recombinant aglycosylated ECA protein wherein N glycosylation site of said protein is mutated.

A preferred mutated form of the ECA lectin comprises mutation of amino acid residue at position 113 N to Q changing the glycosylation site NNS to form QNS. The Q residue is preferred as closest mimic of the natural aminoacid residue. It is realized that the asparagine residue can be altered to several other residues and it would be possible to maintain the activity of the lectin. It is further realized that the NNS glycosylation site may be mutated to inactive form by altering other residues such as the serine residue, e.g. to alanine or introducing bulky or praline residue between N and S, with such approach the properties of the protein can be partially changed.

The invention is further directed to the recombinant aglycosylated ECA protein conjugated to a surface. It is realized that the protein may be passively adsorbed to a surface or cloned comprise conjugatable amino acid residue or conjugated from naturally available residue specifically or non-specifically maintaining the carbohydrate binding activity of the lectin. The invention revealed that the recombinant form of ECA was equally or even more effective in the cell culture than the ECA preparations on average.

The invention is directed to an amino acid sequence encoding the recombinant aglycosylated N-glycosylation site mutated ECA protein or functional fragment thereof.

It is further realized that there are homologous variants of mutated ECA lectin, which are functionally equivalent with only difference of a few amino acid residues. The invention is directed to lectins practically identical to ECA lectin with difference of 1-6, more preferably 1-4 amino acid residues, or with over 97% homology or even more preferably 98% and most preferably 99% of homology. The invention is directed to homologous lectins wherein the protein sequence is at least 50%, more preferably 65%, even more preferably 75%, even more preferably 85% and most preferably 95% homologous and the lectin bind effectively N-acetyllactosamine and has similar oligosaccharide specificity as ECA.

The invention is further directed to a nucleic acid sequence encoding the aglycosylated ECA protein or a functional homolog or a functional fragment thereof. The invention is further directed to a host cell comprising the nucleic.

### Embryonic Stem Cells Grown on Lectins

The invention reveled that it is possible to grow HESC cells on various lectins. The invention provides method to produce embryonal stem cells effectively and on controlled conditions. It is realized that current heterogenous and animal derived materials such as fibroblast feeder cells or matrigel include severe problems with regard to reproducibility, possible contamination with animal derived contamination with harmful molecules such as antigenic structures e.g. N-glycolylneuraminic acid (NeuGc) and risk of viruses, prions and other infections agents. The lectin proteins are available from acceptable animal sources such as The present invention provides matrixes comprising single pure protein coated of the cell culture vessels and supporting the cells.

There was changes in levels of stem cell marker expression and morphology during the cultivation of cells of lectins. However these appear to be reversible during the culture or change to tradiotiona, when the cell are transferred to Matrigel from the lectins.

The lectins support the attachment and growth of the cells. The growing cells have unusual morphology of small cell clusters and shape of cells when compared to stem cell colonies formed on matrigel or together traditional supports. The cells grow on the matrix with temporarily alteration of characteristics.

### Passaging

The novel method of growing stem cells on the lectins revealed additional benefit. It would be possible to detach the cells by gentle shaking type movement without use of enzymes or scraping which could be harmful to the cells.

### Control of Cell Release by Inhibition of Lectin Activity

The inventors further realized that it would be possible to use inhibitors lectins in order to detach the cells from cell culture vessel or container.

### Preferred Lectin Types

The invention revealed that human embryonic stem cells are especially effectively cultivated in contact with (Fucα2)ₙGalβ4GlcNAc, wherein n is 0 or 1, recognizing lectins, preferably selected from the group ECA, galectin, DSA and UEA-1. The Galβ4GlcNAc specific such as lectins ECA, galectin, DSA are preferred because better initial adherence and growth, while Fucα2Galβ4GlcNAc is preferred for substantiallater stage cell yield. ECA type lectins are more preferred than galectin or DSA type lectins because of better preservation of stem cell markers, see example 27.

Release of Binders from the Cells by Carbohydrate Inhibition

The invention is in a preferred embodiment directed to the release of glycans from binders.

This is preferred for several methods including:
a) release of cells from soluble binders after enrichment or isolation of cells by a method invlogin a binder
b) release from solid phase bound binders after enrichment or isolation of cells or during cell cultivation e.g. for passaging of the cells

The inhibitin carbohydrate is selected to correspond to the binding epitope of the lectin or part(s) thereof. The preferred carbohydrates includes oligosaccharides, monosaccharides and conjugates thereof. The preferred concentrations of carbohydrates includes contrations tolerable by the cells from 1 mM to 500 mM, more preferably 10 mM to 250 mM and even more preferably 10-100 mM, higher concentrations are preferred for monosaccharides and method involving solid phase bound binders. Preferred oligosaccharide sequences including oligosaccharides and reducing end conjugates includes Galβ4Glc, Galβ4GlcNAc, Galβ3GlcNAc, Galβ3GalNAc, and sialylated and fucosylated variants of these as described in TABLE 15 and formulas according to the invention,

The preferred reducing enstructure in conjugates is AR, wherein A is anomeric structure preferably beta for Galβ4Glc, Galβ4GlcNAc, Galβ3GlcNAc, and alfa for Galβ3GalNAc and R is organic residue linked glycosidically to the saccharide, and preferably alkyl such as method, ethyl or propyl or ring structure such as a cyclohexyl or aromatic ring structure optionally modified with further functional group. Preferred monosaccharides includes terminal or two or three terminal monosaccharides of the binding epitope such as Fuc, Gal, GalNAc, GlcNAc, Man, preferably as anomeric conjugates: as FucαR, GalβR, GalNAcβR, GalNAcαR GlcNAcβR, ManαR. For example PNA lectin is preferably inhibited by Galβ3GalNAc or lactose or Gal, STA is inhibited by Galβ4Glc, Galβ4GlcNAc or oligomers or poly-LacNAc epitopes derived thereof and LTA is inhibited by fucosylalactose Galβ4(Fucα3)Glc, Galβ4(Fucα3)GlcNAc or Fuc or FucαR. Examples of monovalent inhibition condition are shown in Venable A. et al. (2005) BMC Developmental biology, for inhibition when the cells are bound to polyvalently to solid phase larger epitopes and/or concentrations or multi/polyvalent conjugates are preferred.

The invention is further directed to methods of release of binders by protease digestion similarily as known for release of cells from CD34+ magnetic beads.

### Immobilized Binders Preferably Binder Proteins Protein

The present invention is directed to the use of the specific binder for or in context of cultivation of the stem cells wherein the binder is immobilized.

The immobilization includes non-covalent immobilization and covalent bond including immobilization method and further site specific immobilization and unspecific immobilization.

A preferred non-covalent immobilization methods includes passive adsorption methods. In a preferred method a surface such as plastic surface of a cell culture dish or well is passively absorbed with the binder. The preferred method includes absorption of the binder protein in a solvent or humid condition to the surface, preferably evenly on the surface. The preferred even distribution is produced using slight shaking during the absorption period preferably form 10 min to 3 days, more preferably from 1 hour to 1 day, and most preferably over night for about 8 to 20 hours. The washing steps of the immobilization are preferably performed gently with slow liquid flow to avoid detachment of the lectin.

### Specific Immobilization

The specific immobilization aims for immobilization from protein regions which does not disturb the binding of the binding site of the binder to its ligand glycand such as the specific cell surface glycans of stem cells according to the invention.

Preferred specific immobilization methods includes chemical conjugation from specific aminoacid residues from the surface of the binder protein/peptide. In a preferred method specific amino acid residue such as cysteine is cloned to the site of immobilization and the conjugation is performed from the cysteine, in another preferred method N-terminal cytsteine is oxidized by periodic acid and conjugated to aldehyde reactive reagents such as amino-oxy-methyl hydroxylamine or hydrazine structures, further preferred chemistries includes “click” chemistry marketed by Invitrogen and aminoacid specific coupling reagents marketed by Pierce and Molecular probes.

A preferred specific immobilization occurs from protein linked carbohydrate such as O- or N-glycan of the binder, preferably when the glycan is not close to the binding site or longer specar is used.

### Glycan Immobilized Binder Protein

Preferred glycan immobilization occurs through a reactive chemoselective ligation group R1 of the glycans, wherein the chemical group can be specifically conjugated to second chemoselective ligation group R2 without major or binding destructive changes to the protein part of the binder. Chemoselective groups reacting with aldehydes and ketones includes as amino-oxy-methyl hydroxylamine or hydrazine structures. A preferred R1-group is a carbonyl such as an aldehyde or a ketone chemically synthesized on the surface of the protein. Other preferred chemoselective groups includes maleimide and thiol; and “Click”-reagents including azide and reactive group to it.

Preferred synthesis steps includes
a) chemical oxidation by carbohydrate selectively oxidizing chemical, preferably by periodic acid or
b) enzymatic oxidation by non-reducing end terminal monosaccharide oxidizing enzyme such as galactose oxidase or by transferring a modified monosaccharide residue to the terminal monosaccharide of the glycan.

Use of oxidative enzymes or periodic acid are known in the art has been described in patent application directed conjugating HES-polysaccharide to recombinant protein by Kabi-Frensenius (WO2005EP02637, WO2004EP08821, WO2004EP08820, WO2003EP08829, WO2003EP08858, WO2005092391, WO2005014024 included fully as reference) and a German research institute.

Preferred methods for the transferring the terminal monosaccharide reside includes use of mutant galactosyltransferase as described in patent application by part of the inventors US2005014718 (included fully as reference) or by Qasba and Ramakrishman and colleagues US2007258986 (included fully as reference) or by using method described in glycopegylation patenting of Neose (US2004132640, included fully as reference).

### Conjugates Including High Specificity Chemical Tag

In a preferred embodiment the binder is, specifically or non-specifically conjugated to a tag, referred as T, specifically recognizable by a ligand L, examples of tag includes such as biotin biding ligand (strept)avidin or a fluorocarbonyl binding to another fluorocarbonyl or peptide/antigen and specific antibody for the peptide/antigen

### Preferred Conjugate Structures

### Tag-Conjugate Structures

The preferred conjugate structures are according to the

B-(G-)ₘR1-R2-(S1-)ₙT-, Formula CONJ

Wherein

B is the binder, G is glycan (when the binder is glycan conjugated),
R1 and R2 are chemoselective ligation groups, T is tag, preferably biotin, L is specifically binding ligand for the tag; S1 is an optional spacer group, preferably C₁-C₁₀ alkyls,
m and n are integers being either 0 or 1, independently.

Methods to chemically attach spacer structures ligation groups or ligand such as (strept)avidin to solid phases is known in the art.

### Complex Structure

The preferred conjugate structures are according to the

B-(G-)ₘR1-R2-(S1-)ₙ(T-)ₚ(L-)ᵣ-(S2)ₛ-SOL, Formula COMP

Wherein

B is the binder, SOL is solid phase or matrix or surface, G is glycan (when the binder is glycan conjugated),
R1 and R2 are chemoselective ligation groups, T is tag, preferably biotin, L is specifically binding ligand for the tag; S1 and S2 are optional spacer groups, preferably C₁-C₁₀ alkyls,
m, n, p, r and s are integers being either 0 or 1, independently.

Methods to chemically attach spacer structures to solid phase are known in the art,

The invention is in a preferred embodiment directed to

1, Testing and selection of specific binder structures recognizing stem cells and/or associated cells for the culture of stem cells
2. Use of the specific binder for selection of cells during or before culture of stem cells, especially mesenchymal or embryonic stem cells, preferably in two types of methods:
a) selection of cells by soluble binder molecules, preferably by physical methods recognizing labeled cells such as FACS and/or
b) selection of cells by solid phase blound binder molecules, such as binders
b1) bound to cell cultivation vessels such as plates or containers and/or
b2) binder bound to a polymeric material such as a macromolecules or gel forming material useful for cell culture
b3) binder bound to microparticles, including beads especially magnetic beads 3, Use of specific binder structures recognizing stem cells or a contaminating/associated cell population in soluble or surface bound form during the cell culture.
   Recognition of Structures from Glycome Materials and on Cell Surfaces by Binding Methods

The present invention revealed that beside the physicochemical analysis by NMR and/or mass spectrometry several methods are useful for the analysis of the structures. The invention is especially directed to a method:
i) Recognition by molecules binding glycans referred as the binders
These molecules bind glycans and include property allowing observation of the binding such as a label linked to the binder. The preferred binders include
   a) Proteins such as antibodies, lectins and enzymes
   b) Peptides such as binding domains and sites of proteins, and synthetic library derived analogs such as phage display peptides
   c) Other polymers or organic scaffold molecules mimicking the peptide materials

The peptides and proteins are preferably recombinant proteins or corresponding carbohydrate recognition domains derived thereof, when the proteins are selected from the group of monoclonal antibody, glycosidase, glycosyl transferring enzyme, plant lectin, animal lectin or a peptide mimetic thereof, and wherein the binder may include a detectable label structure.

The genus of enzymes in carbohydrate recognition is continuous to the genus of lectins (carbohydrate binding proteins without enzymatic activity).

a) Native glycosyltransferases (Rauvala et al. (1983) PNAS (USA) 3991-3995) and glycosidases (Rauvala and Hakomori (1981) J. Cell Biol. 88, 149-159) have lectin activities.
b) The carbohydrate binding enzymes can be modified to lectins by mutating the catalytic amino acid residues (see WO9842864; Aalto J. et al. Glycoconjugate J. (2001, 18(10); 751-8; Mega and Hase (1994) BBA 1200 (3) 331-3).
c) Natural lectins, which are structurally homologous to glycosidases are also known indicating the continuity of the genus enzymes and lectins (Sun, Y-J. et al. J. Biol. Chem. (2001) 276 (20) 17507-14).

The genus of the antibodies as carbohydrate binding proteins without enzymatic activity is also very close to the concept of lectins, but antibodies are usually not classified as lectins.

Obviousness of the Peptide Concept and Continuity with the Carbohydrate Binding Protein Concept

It is further realized that proteins consist of peptide chains and thus the recognition of carbohydrates by peptides is obvious. E.g. it is known in the art that peptides derived from active sites of carbohydrate binding proteins can recognize carbohydrates (e.g. Geng J-G. et al (1992) J. Biol. Chem. 19846-53).

As described above antibody fragment are included in description and genetically engineered variants of the binding proteins. The obvious genetically engineered variants would included truncated or fragment peptides of the enzymes, antibodies and lectins.

### Revealing Cell or Differentiation and Individual Specific Terminal Variants of Structures

The invention is directed use the glycomics profiling methods for the revealing structural features with on-off changes as markers of specific differentiation stage or quantitative difference based on quantitative comparison of glycomes. The individual specific variants are based on genetic variations of glycosyltransferases and/or other components of the glycosylation machinery preventing or causing synthesis of individual specific structure.

### Terminal Structural Epitopes

We have previously revealed glycome compositions of human glycomes, here we provide structural terminal epitopes useful for the characterization of stem cell glycomes, especially by specific binders.

The examples of characteristic altering terminal structures includes expression of competing terminal epitopes created as modification of key homologous core Galβ-epitopes, with either the same monosaccharides with difference in linkage position Galβ3GlcNAc, and analogue with either the same monosaccharides with difference in linkage position Galβ4GlcNAc; or the with the same linkage but 4-position epimeric backbone Galβ3GalNAc. These can be presented by specific core structures modifying the biological recognition and function of the structures. Another common feature is that the similar Galβ-structures are expressed both as protein linked (O- and N-glycan) and lipid linked (glycolipid structures). As an alternative for α2-fucosylation the terminal Gal may comprise NAc group on the same 2 position as the fucose. This leads to homologous epitopes GalNAcβ4GlcNAc and yet related GalNAcβ3Gal-structure on characteristic special glycolipid according to the invention.

The invention is directed to novel terminal disaccharide and derivative epitopes from human stem cells, preferably from human embryonal stem cells or adult stem cells, when these are not hematopoietic stem cells, which are preferably mesenchymal stem cells. It should realized that glycosylations are species, cell and tissue specific and results from cancer cells usually differ dramatically from normal cells, thus the vast and varying glycosylation data obtained from human embryonal carcinomas are not actually relevant or obvious to human embryonal stem cells (unless accidentally appeared similar). Additionally the exact differentiation level of teratocarcinomas cannot be known, so comparison of terminal epitope under specific modification machinery cannot be known. The terminal structures by specific binding molecules including glycosidases and antibodies and chemical analysis of the structures.

The present invention reveals group of terminal Gal(NAc)β1-3/4Hex(NAc) structures, which carry similar modifications by specific fucosylation/NAc-modification, and sialylation on corresponding positions of the terminal disaccharide epitopes. It is realized that the terminal structures are regulated by genetically controlled homologous family of fucosyltransferases and sialyltransferases. The regulation creates a characteristic structural patterns for communication between cells and recognition by other specific binder to be used for analysis of the cells. The key epitopes are presented in the TABLE 28. The data reveals characteristic patterns of the terminal epitopes for each types of cells, such as for example expression on hESC-cells generally much Fucα-structures such as Fucα2-structures on type 1 lactosamine (Galβ3GlcNAc), similarily β3-linked core I Galβ3GlcNAcα, and type 4 structure which is present on specific type of glycolipids and expression of α3-fucosylated structures, while α6-sialic on type II N-acetyllactosamine appear on N-glycans of embryoid bodies and st3 embryonal stem cells. E.g. terminal type lactosamine and poly-lactosamines differentiate mesenchymal stem cells from other types. The terminal Galb-information is preferably combined with information about

The invention is directed especially to high specificity binding molecules such as monoclonal antibodies for the recognition of the structures.

The structures can be presented by Formula T1. the formula describes first monosaccharide residue on left, which is a β-D-galactopyranosyl structure linked to either 3 or 4-position of the α- or β-D-(2-deoxy-2-acetamido)galactopyranosyl structure, when R5 is OH, or β-D-(2-deoxy-2-acetamido)glucopyranosyl, when R4 comprises O-. The unspecified stereochemistry of the reducing end in formulas T1 and T2 is indicated additionally (in claims) with curved line. The sialic acid residues can be linked to 3 or 6-position of Gal or 6-position of GlcNAc and fucose residues to position 2 of Gal or 3- or 4-position of GlcNAc or position 3 of Glc.

The invention is directed to Galactosyl-globoside type structures comprising terminal Fucα2-revealed as novel terminal epitope Fucα2Galβ3GalNAcβ or Galβ3GalNAcβGalα3-comprising isoglobotructures revealed from the embryonal type cells.

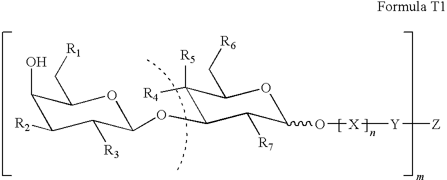

wherein
X is linkage position
R₁, R₂, and R₆ are OH or glycosidically linked monosaccharide residue Sialic acid, preferably Neu5Acα2 or Neu5Gc α2, most preferably Neu5Acα2 or
R₃, is OH or glycosidically linked monosaccharide residue Fucα1 (L-fucose) or N-acetyl (N-acetamido, NCOCH₃);
R₄, is H, OH or glycosidically linked monosaccharide residue Fucα1 (L-fucose),
R₅ is OH, when R4 is H, and R5 is H, when R4 is not H;

### R₇ is N-acetyl or OH

X is natural oligosaccharide backbone structure from the cells, preferably N-glycan, O-glycan or glycolipid structure; or X is nothing, when n is 0,
Y is linker group preferably oxygen for O-glycans and O-linked terminal oligosaccharides and glycolipids and N for N-glycans or nothing when n is 0;
Z is the carrier structure, preferably natural carrier produced by the cells, such as protein or lipid, which is preferably a ceramide or branched glycan core structure on the carrier or H;
The arch indicates that the linkage from the galactopyranosyl is either to position 3 or to position 4 of the residue on the left and that the R4 structure is in the other position 4 or 3;
n is an integer 0 or 1, and m is an integer from 1 to 1000, preferably 1 to 100, and most preferably 1 to 10 (the number of the glycans on the carrier),
With the provisions that one of R2 and R3 is OH or R3 is N-acetyl,
R₆ is OH, when the first residue on left is linked to position 4 of the residue on right:
X is not Galα4Galβ4Glc, (the core structure of SSEA-3 or 4) or R3 is Fucosyl
R7 is preferably N-acetyl, when the first residue on left is linked to position 3 of the residue on right:

Preferred terminal β3-linked subgroup is represented by Formula T2 indicating the situation, when the first residue on the left is linked to the 3 position with backbone structures Gal(NAc)β3Gal/GlcNAc.

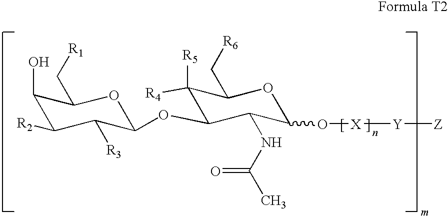

Wherein the variables including R₁ to R₇

are as described for T1

Preferred terminal β4-linked subgroup is represented by the Formula 3

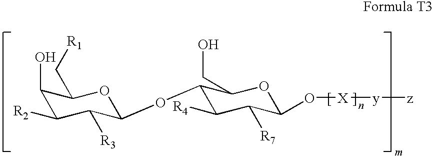

Wherein the variables including R₁ to R₄ and R7

are as described for T1 with the provision that
R₄, is OH or glycosidically linked monosaccharide residue Fucα1 (L-fucose),

Alternatively the epitope of the terminal structure can be represented by Formulas T4 and T5

Galβ1-xHex(NAc)ₚ, Core Galβ-epitopes formula T4

x is linkage position 3 or 4,

### and Hex is Gal or Glc

with provision
p is 0 or 1
when x is linkage position 3, p is 1 and HexNAc is GlcNAc or GalNAc,
and when x is linkage position 4, Hex is Glc.

The core Galβ1-3/4 epitope is optionally substituted to hydroxyl

by one or two structures SAα or Fucα, preferably selected from the group
Gal linked SAα3 or SAα6 or Fucα2, and
Glc linked Fucα3 or GlcNAc linked Fucα3/4.

[Mα]ₘGalβ1-x[Nα]ₙHex(NAc)ₚ, Formula T5

wherein
m, n and p are integers 0, or 1, independently

### Hex is Gal or Glc,

X is linkage position
M and N are monosaccharide residues being
independently nothing (free hydroxyl groups at the positions)
and/or
SA which is Sialic acid linked to 3-position of Gal or/and 6-position of HexNAc and/or
Fuc (L-fucose) residue linked to 2-position of Gal
and/or 3 or 4 position of HexNAc, when Gal is linked to the other position (4 or 3),
and HexNAc is GlcNAc, or 3-position of Glc when Gal is linked to the other position (3), with the provision that sum of m and n is 2
preferably m and n are 0 or 1, independently.

The exact structural details are essential for optimal recognition by specific binding molecules designed for the analysis and/or manipulation of the cells.

The terminal key Galβ-epitopes are modified by the same modification

monosaccharides NeuX (X is 5 position modification Ac or Gc of sialic acid) or Fuc, with the same linkage type alfa(modifying the same hydroxyl-positions in both structures.

NeuXα3, Fucα2 on the terminal Galβ of all the epitopes and

NeuXα6 modifying the terminal Galβ of Galβ4GlcNAc, or HexNAc, when linkage is 6 competing
or Fucα modifying the free axial primary hydroxyl left in GlcNAc (there is no free axial hydroxyl in GalNAc-residue).

The preferred structures can be divided to preferred Galβ1-3 structures analogously to T2,

[Mα]ₘGalβ1-3[Nα]ₙHexNAc, Formula T6

Wherein the variables are as described for T5.

The preferred structures can be divided to preferred Galβ1-4 structures analogously to T4,

[Mα]ₘGalβ1-4[Nα]ₙGlc(NAc)ₚ, Formula T7

Wherein the variables are as described for T5.

These are preferred type II N-acetyllactosamine structures and related lactosylderivatives, in a preferred embodiment p is 1 and the structures includes only type 2 N-acetyllactosamines.

The invention revealed that the these are very useful for recognition of specific subtypes of stem cells, preferably mesenchymal stem cells, or embryonal type stem cells or differentiated variants thereof (tissue type specifically differentiated mesenchymal stem cells or various stages of embryonal stem cells). It is notable that various fucosyl- and or sialic acid modification created characteristic pattern for the stem cell type.

### Preferred Type I and Type II N-Acetyllactosamine Structures

The preferred structures can be divided to preferred type one (I) and type two (II) N-acetyllactosamine structures comprising oligosaccharide core sequence Galβ1-3/4 GlcNAc structures analogously to T4,

[Mα]ₘGalβ1-3/4[Nα]ₙGlcNAc, Formula T8

Wherein the variables are as described for T5.

The preferred structures can be divided to preferred Galβ1-3 structures analogously to T8,

[Mα]ₘGalβ1-3[Nα]ₙGlcNAc Formula T9

Wherein the variables are as described for T5.

These are preferred type I N-acetyllactosamine structures. The invention revealed that the these are very useful for recognition of specific subtypes of stem cells, preferably mesenchymal stem cells, or embryonal type stem cells or differentiated variants thereof (tissue type specifically differentiated mesenchymal stem cells or various stages of embryonal stem cells). It is notable that various fucosyl- and or sialic acid modification created characteristic pattern for the stem cell type.

The preferred structures can be divided to preferred Galβ1-4GlcNAc core sequence comprising structures analogously to T8,

[Mα]ₘGalβ1-4[Nα]ₙGlcNAc Formula T10

Wherein the variables are as described for T5.

These are preferred type II N-acetyllactosamine structures. The invention revealed that the these are very useful for recognition of specific subtypes of stem cells, preferably mesenchymal stem cells, or embryonal type stem cells or differentiated variants thereof (tissue type specifically differentiated mesenchymal stem cells or various stages of embryonal stem cells).

It is notable that various fucosyl- and or sialic acid modificationally N-acetyllactosamine structures create especially characteristic pattern for the stem cell type. The invention is further directed to use of combinations binder reagents recognizing at least two different type I and type II acetyllactosamines including at least one fucosylated or sialylated variant and more preferably at least two fucosylated variants or two sialylated variants

### Preferred Structures Comprising Terminal Fucα2/3/4-Structures

The invention is further directed to use of combinations binder reagents recognizing:
a) type I and type II acetyllactosamines and their fucosylated variants, and in a preferred embodiment
b) non-sialylated fucosylated and even more preferably
c) fucosylated type I and type II N-acetyllactosamine structures preferably comprising Fucα2-terminal and/or Fucα3/4-branch structure and even more preferably
d) fucosylated type I and type II N-acetyllactosamine structures preferably comprising Fucα2-terminal
for the methods according to the invention of various stem cells especially embryonal type and mesenchymal stem cells and differentiated variants thereof.

Preferred subgroups of Fucα2-structures includes monofucosylated H type and H type II structures, and difucosylated Lewis b and Lewis y structures.

Preferred subgroups of Fucα3/4-structures includes monofucosylated Lewis a and Lewis x structures, sialylated sialyl-Lewis a and sialyl-Lewis x-structures and difucosylated Lewis b and Lewis y structures.

Preferred type II N-acetyllactosamine subgroups of Fucα3-structures includes monofucosylated Lewis x structures, and sialyl-Lewis x-structures and Lewis y structures.

Preferred type I N-acetyllactosamine subgroups of Fucα-4-structures includes monofucosylated Lewis a sialyl-Lewis a and difucosylated Lewis b structures.

The invention is further directed to use of at least two differently fucosylated type one and or and two N-acetyllactosamine structures preferably selected from the group monofucosylated or at least two difucosylated, or at least one monofucosylated and one difucosylated structures.

The invention is further directed to use of combinations binder reagents recognizing fucosylated type I and type II N-acetyllactosamine structures together with binders recognizing other terminal structures comprising Fucα2/3/4-comprising structures, preferably Fucα2-terminal structures, preferably comprising Fucα2Galβ3GalNAc-terminal, more preferably Fucα2Galβ3GalNAcα/β and in especially preferred embodiment antibodies recognizing Fucα2Galβ3GalNAcβ- preferably in terminal structure of Globo- or isoglobotype structures.

### Preferred Globo- and Ganglio Core Type-Structures

The invention is further directed to general formula comprising globo and gangliotype Glycan core structures according to formula

[M]ₘGalβ1-x[Nα]ₙHex(NAc)ₚ, Formula T11

wherein
m, n and p are integers 0, or 1, independently
Hex is Gal or Glc, X is linkage position;
M and N are monosaccharide residues being
independently nothing (free hydroxyl groups at the positions)
and/or
SAα which is Sialic acid linked to 3-position of Gal or/and 6-position of HexNAc
Galα linked to 3 or 4-position of Gal, or
GalNAcβ linked to 4-position of Gal and/or
Fuc (L-fucose) residue linked to 2-position of Gal
and/or 3 or 4 position of HexNAc, when Gal is linked to the other position (4 or 3),
and HexNAc is GlcNAc, or 3-position of Glc when Gal is linked to the other position (3), with the provision that sum of m and n is 2
preferably m and n are 0 or 1, independently, and
with the provision that when M is Galα then there is no sialic acid linked to Galβ1, and
n is 0 and preferably x is 4.
with the provision that when M is GalNAcβ, then there is no sialic acid α6-linked to Galβ1, and n is 0 and x is 4.

The invention is further directed to general formula comprising globo and gangliotype Glycan core structures according to formula

[M][SAα3]ₙGalβ1-4Glc(NAc)ₚ, Formula T12

wherein
n and p are integers 0, or 1, independently
M is Galα linked to 3 or 4-position of Gal, or GalNAcβ linked to 4-position of Gal
and/or SAα is Sialic acid branch linked to 3-position of Gal
with the provision that when M is Galα then there is no sialic acid linked to Galβ1 (n is 0).

The invention is further directed to general formula comprising globo and gangliotype Glycan core structures according to formula

[M][SAα]ₙGalβ1-4Glc, Formula T13

wherein
n and p are integer 0, or 1, independently
M is Galα linked to 3 or 4-position of Gal, or
GalNAcβ linked to 4-position of Gal
and/or
SAα which is Sialic acid linked to 3-position of Gal
with the provision that when M is Galα then there is no sialic acid linked to Galβ1 (n is 0).

The invention is further directed to general formula comprising globo type Glycan core structures according to formula

Galα3/4Galβ1-4Glc. Formula T14

The preferred Globo-type structures includes Galα3/4Galβ1-4Glc, GalNAcβ3Galα3/4Galβ4Glc, Galα4Galβ4Glc (globotriose, Gb3), Galα3Galβ4Glc (isoglobotriose), GalNAcβ3Galα4Galβ4Glc (globotetraose, Gb4 (or G14)), and Fucα2Galβ3GalNAcβ3Galα3/4Galβ4Glc. or

when the binder is not used in context of non-differentiated emrbyonal or mesenchymal stem cells or the binder is used together with another preferred binder according to the invention, preferably an other globo-type binder the preferred binder targets further includes Galβ3GalNAcβ3Galα4Galβ4Glc (SSEA-3 antigen) and/or NeuAcα3Galβ3GalNAcβ3Galα4Galβ4Glc (SSEA-4 antigen) or terminal non-reducing end di or trisaccharide epitopes thereof.

The preferred globotetraosylceramide antibodies does not recognize non-reducing end elongated variants of GalNAcβ3Galα4Galβ4Glc. The antibody in the examples has such specificity as

The invention is further directed to binders for specific epitopes of the longer oligosaccharide sequences including preferably NeuAcα3Galβ3GalNAc, NeuAcα3Galβ3GalNAcβ, NeuAcα3Galβ3GalNAcβ3Galα4Gal when these are not linked to glycolipids and novel fucosylated target structures:

### Fucα2Galβ3GalNAcβ3Galα3/4Gal, Fucα2Galβ3GalNAcβ3Galα, Fucα2Galβ3GalNAcβ3Gal, Fucα2Galβ3GalNAcβ3, and Fucα2Galβ3GalNAc.

The invention is further directed to general formula comprising globo and gangliotype Glycan core structures according to formula

[GalNAcβ4][SAα]ₙGalβ1-4Glc, Formula T15

wherein n and p are integer 0, or 1, independently GalNAcα linked to 4-position of Gal and/or SAα which is Sialic acid branch linked to 3-position of Gal.

The preferred Ganglio-type structures includes GalNAcβ4Galβ1-4Glc, GalNAcβ4[SAα3]Galβ1-4Glc, and Galβ3GalNAcβ4[SAα3]Galβ1-4Glc.

The preferred binder target structures further include glycolipid and possible glycoprotein conjugates of the preferred oligosaccharide sequences. The preferred binders preferably specifically recognizes at least di- or trisaccharide epitope

### GalNAcα-Structures

The invention is further directed to recognition of peptide/protein linked GalNAcα-structures according to the Formula T16:[SAα6]ₘGalNAcα[Ser/Thr]ₙ-[Peptide]ₚ, wherein m, n and p are integers 0 or 1, independently,

wherein SA is sialic acid preferably NeuAc,Ser/Thr indicates linking serine or threonine residues, Peptide indicates part of peptide sequence close to linking residue,
with the provisio that either m or n is 1.

Ser/Thr and/or Peptide are optionally at least partially necessary for recognition for the binding by the binder. It is realized that when Peptide is included in the specificity, the antibody have high specificity involving part of a protein structure. The preferred antigen sequences of sialyl-Tn: SAα6GalNAcα, SAα6GalNAcαSer/Thr, and SAα6GalNAcαSer/Thr-Peptide and Tn-antigen: GalNAcαSer/Thr, and GalNAcαSer/Thr-Peptide. The invention is further directed to the use of combinations of the GalNAcα-structures and combination of at least one GalNAcα-structure with other preferred structures.

### Combinations of Preferred Binder Groups

The present invention is especially directed to combined use of at least a) fucosylated, preferably α2/3/4-fucosylated structures and/or b) globo-type structures and/or c) GalNAcα-type structures. It is realized that using a combination of binders recognizing structures involving different biosynthesis and thus having characteristic binding profile with a stem cell population. More preferably at least one binder for a fucosylated structure and globostructures, or fucosylated structure and GalNAcα-type structure is used, most preferably fucosylated structure and globostructure are used.

### Fucosylated and Non-Modified Structures

The invention is further directed to the core disaccharide epitope structures when the structures are not modified by sialic acid (none of the R-groups according to the Formulas T1-T3 or M or N in formulas T4-T7 is not sialic acid.

The invention is in a preferred embodiment directed to structures, which comprise at least one fucose residue according to the invention. These structures are novel specific fucosylated terminal epitopes, useful for the analysis of stem cells according to the invention. Preferably native stem cells are analyzed.

The preferred fucosylated structures include novel α3/4fucosylated markers of human stem cells such as (SAα3)₀ₒᵣ₁Galβ3/4(Fucα4/3)GlcNAc including Lewis x and sialylated variants thereof.

Among the structures comprising terminal Fucα1-2 the invention revealed especially useful novel marker structures comprising Fucα2Galβ3GalNAcα/β and Fucα2Galβ3(Fucα4)₀ₒᵣ₁GlcNAcβ, these were found useful studying embryonal stem cells. A especially preferred antibody/binder group among this group is antibodies specific for Fucα2Galβ3GlcNAcβ, preferred for high stem cell specificity. Another preferred structural group includes Fucα2Gal comprising glycolipids revealed to form specific structural group, especially interesting structure is globo-H-type structure and glycolipids with terminal Fucα2Galβ3GalNAcβ, preferred with interesting biosynthetic context to earlier speculated stem cell markers.

Among the antibodies recognizing Fucα2Galβ4GlcNAcβ substantial variation in binding was revealed likely based on the carrier structures, the invention is especially directed to antibodies recognizing this type of structures, when the specificity of the antibody is similar to the ones binding to the embryonal stem cells as shown in Example 14 with fucose recognizing antibodies. The invention is preferably directed to antibodies recognizing Fucα2Galβ4GlcNAcβ on N-glycans, revealed as common structural type in terminal epitope Table 28. In a separate embodiment the antibody of the non-binding clone is directed to the recognition of the feeder cells.

The preferred non-modified structures includes Galβ4Glc, Galβ3GlcNAc, Galβ3GalNAc, Galβ4GlcNAc, Galβ3GlcNAcβ, Galβ3GalNAcβ/α, and Galβ4GlcNAcβ. These are preferred novel core markers characteristics for the various stem cells. The structure Galβ3GlcNAc is especially preferred as novel marker observable in hESC cells. Preferably the structure is carried by a glycolipid core structure according to the invention or it is present on an O-glycan. The non-modified markers are preferred for the use in combination with at least one fucosylated or/and sialylated structure for analysis of cell status.

Additional preferred non-modified structures includes GalNAcβ-structures includes terminal LacdiNAc, GalNAcβ4GlcNAc, preferred on N-glycans and GalNAcβ3Gal GalNAcβ3Gal present in globoseries glycolipids as terminal of globotetraose structures.

Among these characteristic subgroup of Gal(NAc)β3-comprising Galβ3GlcNAc, Galβ3GalNAc, Galβ3GlcNAcβ, Galβ3GalNAcβ/α, and GalNAcβ3Gal GalNAcβ3Gal and the characteristic subgroup of Gal(NAc)β4-comprising Galβ4Glc, Galβ4GlcNAc, and Galβ4GlcNAc are separately preferred.

### Preferred Sialylated Structures

The preferred sialylated structures includes characteristic SAα3Galβ-structures SAα3Galβ4Glc, SAα3Galβ3GlcNAc, SAα3Galβ3GalNAc, SAα3Galβ4GlcNAc, SAα3Galβ3GlcNAcβ, SAα3Galβ3GalNAcβ/α, and SAα3Galβ4GlcNAcβ; and biosynthetically partially competing SAα6Galβ-structures SAα6Galβ4Glc, SAα6Galβ4Glcβ; SAα6Galβ4GlcNAc and SAα6Galβ4GlcNAcβ; and disialo structures SAα3Galβ3(SAα6)GalNAcβ/α,

The invention is preferably directed to specific subgroup of Gal(NAc)β3-comprising SAα3Galβ3GlcNAc, SAα3Galβ3GalNAc, SAα3Galβ4GlcNAc, SAα3Galβ3GlcNAcβ, SAα3Galβ3GalNAcβ/α and SAα3Galβ3(SAα6)GalNAcβ/α, and

Gal(NAc)β4-comprising sialylated structures. SAα3Galβ4Glc, and SAα3Galβ4GlcNAcβ; and SAα6Galβ4Glc, SAα6Galβ4Glcβ; SAα6Galβ4GlcNAc and SAα6Galβ4GlcNAcβ

These are preferred novel regulated markers characteristics for the various stem cells.

Use Together with a Terminal ManαMan-Structure

The terminal non-modified or modified epitopes are in preferred embodiment used together with at least one ManαMan-structure. This is preferred because the structure is in different N-glycan or glycan subgroup than the other epitopes.

### Preferred Structural Groups for Hematopoietic Stem Cells.

The present invention provides novel markers and target structures and binders to these for especially embryonic and adult stem cells, when these cells are not heamtopoietic stem cells. From hematopoietic CD34+ cells certain terminal structures such as terminal sialylated type two N-acetyllactosamines such as NeuNAcα3Galβ4GlcNAc (Magnani J. U.S. Pat. No. 6,362,010) has been suggested and there is indications for low expression of Slex type structures NeuNAcα3Galβ4(Fucα3)GlcNAc (Xia L et al Blood (2004) 104 (10) 3091-6). The invention is also directed to the NeuNAcα3Galβ4GlcNAc non-polylactosamine variants separately from specific characteristic O-glycans and N-glycans. The invention further provides novel markers for CD133+ cells and novel hematopoietic stem cell markers according to the invention, especially when the structures does not include NeuNAcα3Galβ4(Fucα3)₀₋₁GlcNAc. Preferably the hematopoietic stem cell structures are non-sialylated, fucosylated structures Galβ1-3-structures according to the invention and even more preferably type 1 N-acetyllactosamine structures Galβ3GlcNAc or separately preferred Galβ3GalNAc based structures.

### Core Structures of the Terminal Epitopes

It is realized that the target epitope structures are most effectively recognized on specific N-glycans, O-glycan, or on glycolipid core structures.

Elongated epitopes—Next monosaccharide/structure on the reducing end of the epitope The invention is especially directed to optimized binders and production thereof, when the binding epitope of the binder includes the next linkage structure and even more preferably at least part of the next structure (monosaccharide or aminoacid for O-glycans or ceramide for glycolipid) on the reducing side of the target epitope. The invention has revealed the core structures for the terminal epitopes as shown in the Examples and ones summarized in Table 28.

It is realized that antibodies with longer binding epitopes have higher specificity and thus will recognize that desired cells or cell derived components more effectively. In a preferred embodiment the antibodies for elongated epitopes are selected for effective analysis of embryonal type stem cells.

The invention is especially directed to the methods of antibody selection and optionally further purification of novel antibodies or other binders using the elongated epitopes according to the invention. The preferred selection is performed by contacting the glycan structure (synthetic or isolated natural glycan with the specific sequence) with a serum or an antibody or an antibody library, such as a phage display library. Data about these methods are well known in the art and available from internet for example by searching pubmed-medical literature database (www.ncbi.nlm.nih.gov/entrez) or patents e.g. in espacenet (fi.espacenet.com).

The specific antibodies are especially preferred for the use of the optimized recognition of the glycan type specific terminal structures as shown in the Examples and ones summarized in the Table 28.

It is further realized that part of the antibodies according to the invention and shown in the examples have specificity for the elongated epitopes. The inventors found out that for example Lewis x epiotpe can be recognized on N-glycan by certain terminal Lewis x specific antibodies, but not so effectively or at all by antibodies recognizing Lewis xβ1-3Gal present on poly-N-acetyllactosamines or neolactoseries glycolipids.

### N-Glycans

The invention is especially directed to recognition of terminal N-glycan epitopes on biantennary N-glycans. The preferred non-reducing end monosaccharide epitope for N-glycans comprise β2Man

and its reducing end further elongated variants

### β2Man, β2Manα, β2Manα3, and β2Manα6

The invention is especially directed to recognition of lewis x on N-glycan by N-glycan Lewis x specific antibody described by Ajit Varki and colleagues Glycobiology (2006) Abstracts of Glycobiology society meeting 2006 Los Angeles, with possible implication for neuronal cells, which are not directed (but disclaimed) with this type of antibody by the present invention. Invention is further directed to antibodies with specificity of type 2 N-acetyllactosamineβ2Man recognizing biantennary N-glycan directed antibody as described in Ozawa H et al (1997) Arch Biochem Biophys 342, 48-57.

### O-Glycans, Reducing End Elongated Epitopes

The invention is especially directed to recognition of terminal O-glycan epitopes as terminal core I epitopes and as elongated variants of core I and core II O-glycans.

The preferred non-reducing end monosaccharide epitope for O-glycans comprise:

a) Core I epitopes linked to αSer/Thr-[Peptide]₀₋₁,
wherein Peptide indicates peptide which is either present or absent. The invention is preferable
b) Preferred core II-type epitopes
R1β6[R2β3Galβ3]ₙGalNAcαSer/Thr, wherein n is = or 1 indicating possible branch in the structure and R1 and R2 are preferred positions of the terminal epitopes, R1 is more preferred
c) Elongated Core I epitope
β3Gal and its reducing end further elongated variants β3Galβ3GalNAcα, β3Galβ3GalNAcαSer/Thr

O-glycan core I specific and ganglio/globotype core reducing end epitopes have been described in (Saito S et al. J Biol Chem (1994) 269, 5644-52), the invention is preferably directed to similar specific recognition of the epitopes according to the invention. O-glycan core II sialyl-Lewis x specific antibody has been described in Walcheck B et al. Blood (2002) 99, 4063-69.

Peptide specificity including antibodies for recognition of O-glycans includes mucin specific antibodies further recognizing GalNAcalfa (Tn) or Galb3GalNAcalfa (T/TF) structures (Hanisch F-G et al (1995) cancer Res. 55, 4036-40; Karsten U et al. Glycobiology (2004) 14, 681-92;

### Glycolipid Core Structures

The invention is furthermore directed to the recognition of the structures on lipid structures.

The preferred lipid core structures include:
a) βCer (ceramide) for Galβ4Glc and its fucosyl or sialyl derivatives
b) β3/6Gal for type I and type II N-acetyllactosamines on lactosyl Cer-glycolipids, preferred elongated variants includes β3/6[Rβ6/3]ₙGalβ, β3/6[Rβ6/3]ₙGalβ4 and β3/6[Rβ6/3]ₙGalβ4Glc, which may be further banched by another lactosamine residue which may be partially recognized as larger epitope and n is 0 or 1 indicating the branch, and R1 and R2 are preferred positions of the terminal epitopes. Preferred linear (non-branched) common structures include β3Gal, β3Galβ, β3Galβ4 and β3Galβ4Glc
c) α3/4Gal, for globoseries epitopes, and elongated variants α3/4Galβ, α3/4Galβ4Glc preferred globoepitopes have elongated epitopes α4Gal, α4Galβ, α4Galβ4Glc, and preferred isogloboepitopes have elongated epitopes α3Gal, α3Galβ, α3Galβ4Glc
d) β4Gal for ganglio-series epitopes comprising, and preferred elongated variants include β4Galβ, and β4Galβ4Glc
   O-glycan core specific and ganglio/globotype core reducing end epitopes have been described in (Saito S et al. J Biol Chem (1994) 269, 5644-52), the invention is preferably directed to similar specific recognition of the epitopes according to the invention.

Poly-N-Acetyllactosamines

Poly-N-acetyllactosamine backbone structures on O-glycans, N-glycans, or glycolipids comprise characteristic structures similar to lactosyl(cer) core structures on type I (lactoseries) and type II (neolacto) glycolipids, but terminal epitopes are linked to another type I or type II N-acetyllactosamine, which may from a branched structure. Preferred elongated epitopes include:

β3/6Gal for type I and type II N-acetyllactosamines epitope, preferred elongated variants includes R1β3/6[R2β6/3]Galβ, R1β3/6[R2β6/3]ₙGalβ3/4 and
R1β3/6[R2β6/3]ₙGalβ3/4GlcNAc, which may be further banched by another lactosamine residue which may be partially recognized as larger epitope and n is 0 or 1 indicating the branch, and R1 and R2 are preferred positions of the terminal epitopes. Preferred linear (non-branched) common structures include β3Gal, β3Galβ, β3Galβ4 and β3Galβ4GlcNAc.

Numerous antibodies are known for linear (I-antigen) and branched poly-N-acetyllactosamines (I-antigen), the invention is further directed to the use of the lectin PWA for recognition of I-antigens. The inventors revealed that poly-N-acetyllactosamines are characteristic structures for specific types of human stem cells. Another preferred binding regent, enzyme endo-beta-galactosidase was used for characterization poly-N-acetyllactosamines on glycolipids and on glycoprotein of the stem cells. The enzyme revealed characteristic expression of both linear and branched poly-N-acetyllactosamine, which further comprised specific terminal modifications such as fucosylation and/or sialylation according to the invention on specific types of stem cells.

### Combinations of Elongated Core Epitopes

It is realized that stronger labeling may be obtained if the same terminal epitope is recognized by antibody binding to target structure present on two or three of the major carrier types O-glycans, N-glycans and glycolipids. It is further realized that in context of such use the terminal epitope must be specific enough in comparison to the epitopes present on possible contaminating cells or cell materials. It is further realized that there is highly terminally specific antibodies, which allow binding to on several elongation structures.

The invention revealed each elongated binder type useful in context of stem cells. Thus the invention is directed to the binders recognizing the terminal structure on one or several of the elongating structures according to the invention

### Preferred Group of Monosaccharide Elongation Structures

The invention is directed to use of binders with elongated specificity, when the binders recognize or is able to bind at least one reducing end elongation monosaccharide epitope according to the formula

AxHex(NAc)ₙ, wherein A is anomeric structure alfa or beta, X is linkage position 2, 3, 4, or 6 And Hex is hexopyranosyl residue Gal, or Man, and n is integer being 0 or 1, with the provisions that when n is 1 then AxHexNAc is β4GalNAc or β6GalNAc, when Hex is Man, then AxHex is β2Man, and when Hex is Gal, then AxHex is β3Gal or β6Gal.

Beside the monosaccharide elongation structures αSer/Thr are preferred reducing end elongation structures for reducing end GalNAc-comprising O-glycans and βCer is preferred for lactosyl comprising glycolipid epitopes.

The preferred subgroups of the elongation structures includes i) similar structural epitopes present on O-glycans, polylactosamine and glycolipid cores: β3/6Gal or β6GalNAc; with preferred further subgroups ia) β6GalNAc/β6Gal and ib) β3Gal; ii) N-glycan type epitope β2Man; and iii) globoseries epitopes α3Gal or α4Gal. The groups are preferred for structural similarity on possible cross reactivity within the groups, which can be used for increasing labeling intensity when background materials are controlled to be devoid of the elongated structure types.

Useful binder specifities including lectin and elongated antibody epitopes is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and L is, Halina) Kluwer Academic publishers Dordrecht, The Netherlands and internet databases such as pubmed/espacenet or antibody databases such as www.glyco.is.ritsumei.ac.ip/epitope/, which list monoclonal antibody glycan specificities).

### Preferred Binder Molecules

The present invention revealed various types of binder molecules useful for characterization of cells according to the invention and more specifically the preferred cell groups and cell types according to the invention. The preferred binder molecules are classified based on the binding specificity with regard to specific structures or structural features on carbohydrates of cell surface. The preferred binders recognize specifically more than single monosaccharide residue.

It is realized that most of the current binder molecules such as all or most of the plant lectins are not optimal in their specificity and usually recognize roughly one or several monosaccharides with various linkages. Furthermore the specificities of the lectins are usually not well characterized with several glycans of human types.

The preferred high specificity binders recognize
A) at least one monosaccharide residue and a specific bond structure between those to another monosaccharides next monosaccharide residue referred as MS1B1-binder,
B) more preferably recognizing at least part of the second monosaccharide residue referred as MS2B1-binder,
C) even more preferably recognizing second bond structure and or at least part of third mono saccharide residue, referred as MS3B2-binder, preferably the MS3B2 recognizes a specific complete trisaccharide structure.
D) most preferably the binding structure recognizes at least partially a tetrasaccharide with three bond structures, referred as MS4B3-binder, preferably the binder recognizes complete tetrasaccharide sequences.

The preferred binders includes natural human and or animal, or other proteins developed for specific recognition of glycans. The preferred high specificity binder proteins are specific antibodies preferably monoclonal antibodies; lectins, preferably mammalian or animal lectins; or specific glycosyltransferring enzymes more preferably glycosidase type enzymes, glycosyltransferases or transglycosylating enzymes.

### Modulation of Cells by the Binders

The invention revealed that the specific binders directed to a cell type can be used to modulate cells. In a preferred embodiment the (stem) cells are modulated with regard to carbohydrate mediated interactions. The invention revealed specific binders, which change the glycan structures and thus the receptor structure and function for the glycan, these are especially glycosidases and glycosyltransferring enzymes such as glycosyltransferases and/or transglycosylating enzymes. It is further realized that the binding of a non-enzymatic binder as such select and/or manipulate the cells. The manipulation typically depend on clustering of glycan receptors or affect of the interactions of the glycan receptors with counter receptors such as lectins present in a biological system or model in context of the cells. The invention further reveled that the modulation by the binder in context of cell culture has effect about the growth velocity of the cells.

### Modulation of Stem Cells

The preferred modulation of the stem cells includes following

1) modulation of the status of the cells including one or several of the following modulation types
1.1. support of the undifferentiated status of the stem cells
1.2. change of biological status of the stem cells including
   1.2.1. morphologic status
   and/or
   1.2.2. differentiation related status of cells
1.3. change of the adherence status of cells including
   1.3.1. change of adherence of homogenous cell population
   or
   1.3.2. change of adherence status of heterogenous cell population

The modulation is useful to maintain the undifferentiated status of the stem cells, when the aim is to increase the amount of the stem cells.

The change of biological cell status is useful for production of useful stem cell derived cell preparations and proving novel cell population for studies of stem cells and optimisation of stem cell populations.

The present method is especially useful for affecting the morphological status of stem cells. The invention provides novel specific binding molecules affecting the cell surfaces and thus useful for changing morphological cell status. The invention especially provides polyvalently represented binder molecules useful for the changing of the morphology, as the cell surface molecules and their extracellular contacts regulates the morphology. It is realized that the various morphological statuses of the stem cell reflect potential change of differentiation status. It is therefore useful to produce stem cell preparations of various morphologic status to search for various useful differentiated forms of stem cells.

It is realized that it is further more useful to change the adherence status of the stem cells. The change of adherence status of homogenous cell population is useful and is in preferred mode of invention used for affecting the morphology cells by polyvalent conjugates especially on solid surface. The increased adherence is also useful for anchoring cells for growing these as a layer.

The change of adherence status of heterogenous cell population is useful for separating adherent and non-adherent cell population. The cell cultivation are in a preferred methods directed to support the adherent and/or non-adherent cell population, more preferably the cell culture conditions are selected to support the adherent cell population.

2) changing the growth speed of the stem cells
2.1. Increasing the growth speed of the stem cells
or
2.2. Decreasing the growth speed of the stem cells.

It is realized that the increasing the growth of cells would allow production of more cells within a certain time frame. This would make the process more cost effective and allow saving reagents and energy.

The method for decreasing the growth speed is useful for maintaining alive cells ready for a specific biological and/or scientific use. In a preferred mode of invention the maintaining is further directed to maintaining or changing the biological or adherence status of the cells.

It is realized that the modulation may include both 1) modulation the status of the cells and 2) changing the growth speed of the cells to obtain preferred cell populations.

The present invention revealed lectins and binders are especially useful for cultivation of stem cells.

Target structure specificities of the lectins share common epitopes, it is realized that the lectins may also bind different structures, but there is homologous general structural theme in the specificities

The invention revealed that binders recognizing terminal Gal, GalNAc, Fuc, GlcNAc Man, preferably binders recognizing terminal Galβ, GalNAcβ, GlcNAcβ, or

1) β-linked D-hexopyranosides according to Formula Hex(NAc)ₙ, wherein n is 0 or 1 and Hex is Gal or Glc, with provisio that n is 1, when Hex is Glc: comprising terminals Galβ, GalNAcβ, GlcNAcβ, and
2) α-linked pyranoside residues Manα Fucα, op sialic acidα, preferably Neu5Ac or Neu5Gc, Manα, and Fucα-comprising glycan structures are useful for modulation of the growth of stem cells.

Target structure specificities of the lectins share common structural features related to type II, N-acetyllactosamine structures comprising core epitope

Glc/Gal(NAc)_{0 or 1}β4GlcNAc, wherein reducing end GlcNAc can be derivatised by Fuc-residue and non-reducing end residue can be further elongated preferably sialic acid or N-glycan core oligosaccharides

The invention is specifically directed to binder recognizing at least one structure according to the Formula CC0

[SA]ₚHex(NAc)ₙβ4[FucαX]ₘGlcNAcβR,

wherein
n, m, and p are 0 or 1, independently
X is linkage position being either 3 or 6,

### Hex is Gal or Glc

SA is elongating mono- or oligosaccharide structure,
preferably sialic acid, which is preferably SAα3, or SAα6 and preferred sialic acid type is Neu5Ac or Neu5Gc
   or N-glycan core structure Manα3[Manα6]Manβ4, wherein the Manα-residues can be further elongated by one or several complex type terminal structures such as GlcNAcβ2 or LacNAcβ2,
   R is optional elongating monosaccharide residue structure, preferably 3/6Gal(NAc) of N-acetyllactosamine/of glycolipid such as lactosyl-ceramide/of O-glycan/or 2Man of N-glycan, or Asn-(Peptide)_{0 or 1}, indicating potential linkage core protein/peptide when Hex(NAc) is GlcNAc
   with the provision that
   when m is 1 and X is 6, then n is 1, and Hex is Glc and SA is N-glycan core structure Manα3[Manα6]Manβ or its elongated variant,
   when n is 1 and Hex is Gal then p is 0.

The preferred target structures are

### Galβ4GlcNAc, Neu5Acα3Galβ4GlcNAc, Neu5Acα6Galβ4GlcNAc, Fucα2Galβ4GlcNAc GalNAcβ4GlcNAc, and GlcNAcβ4(Fucα6)GlcNAc

The most preferred binder lectins recognizing the target structures are ECA, PWA, and WFA (weaker binding) recognizing Galβ4GlcNAc, MAA recognizing especially Neu5Acα3Galβ4GlcNAc, SNA recognizing Neu5Acα6Galβ4GlcNAc, WFA recognizing GalNAcβ4GlcNAc, UEA recognizing Fucα2Galβ4GlcNAc, LTA recognizing Galβ4(Fucα3)GlcNAc and PSA recognizing GlcNAcβ4(Fucα6)GlcNAc-structures.

The invention is further directed to the plant lectin group recognizing truncated terminal epitopes GlcNAcβ or Manα, preferably GSAII or NPA, or other lectins with similar specificity.

The invention is specifically directed to binder recognizing at least one structure according to the Formula CC1

[SA]ₚHex(NAc)ₙβ4[Fucα6]ₘGlcNAcβR,

wherein
n, m, and p are 0 or 1, independently

### Hex is Gal or Glc

SA is elongating mono- or oligosaccharide structure,
preferably sialic acid, which is preferably SAα3 and preferred sialic acid type is

### Neu5Ac or Neu5Gc

or N-glycan core structure Manα3[Manα6]Manβ4, wherein the Manα-residues can be further elongated by one or several complex type terminal structures such as GlcNAcβ2 or LacNAcβ2,
R is optional elongating monosaccharide residue structure, preferably 3/6Gal(NAc) of N-acetyllactosamine/of glycolipid such as lactosyl-ceramide/of O-glycan/or 2Man of N-glycan, or Asn-(Peptide)_{0 or 1}, indicating potential linkage core protein/peptide when Hex(NAc) is GlcNAc
with the provision that
when m is 1, then n is 1 and Hex is Glc and SA is N-glycan core structure
Manα3[Manα6]Manβ or its elongated variant,
when n is 1 and Hex is Gal then p is 0.

The preferred target structures are

### Galβ4GlcNAc, Neu5Acα3Galβ4GlcNAc, GalNAcβ4GlcNAc, and GlcNAcβ4(Fucα6)GlcNAc

The most preferred binder lectins recognizing the target structures are ECA, PWA, and WFA(weaker binding) recognizing Galβ4GlcNAc, MAA recognizing especially Neu5Acα3Galβ4GlcNAc, WFA recognizing GalNAcβ4GlcNAc, and PSA recognizing GlcNAcβ4(Fucα6)GlcNAc-structures.

The preferred target structure subgroups include:

Structures according to the formula CC2

[SA]ₚGal(NAc)ₙβ4GlcNAcβR,

wherein remain
p and n are 0 or 1, independently
SA is sialic acid SAα3 and preferred sialic acid type is Neu5Ac or Neu5Gc, more preferably Neu5Ac,
when n is 1 and Hex is Gal then p is 0.

Preferred target structure epitopes according to CC2 includes: Galβ4GlcNAc, Neu5Acα3Galβ4GlcNAc, and GalNAcβ4GlcNAc.

The preferred target structure subgroups include:

structures according to the formula CC3

Manα3[Manα6]Manβ4GlcNAcβ4(Fucα6)GlcNAcβR

wherein the Manα-residues can be further elongated by one or several complex type terminal structures such as GlcNAcβ2 or LacNAcβ2 or terminally sialylated variant of LacNAc, which is preferably Galβ4GlcNAc
and R is optionally Asn-(Peptide)_{0 or 1}, indicating potential linkage core protein/peptide.

Preferred target structure epitopes according to CC3 includes:

### GlcNAcβ4(Fucα6)GlcNAc, GlcNAcβ4(Fucα6)GlcNAcβ GlcNAcβ4(Fucα6)GlcNAcβAsn,

### Manβ4GlcNAcβ4(Fucα6)GlcNAc, Manβ4GlcNAcβ4(Fucα6)GlcNAcβR Manβ4GlcNAcβ4(Fucα6)GlcNAcβAsn

### Manα3[Manα6]Manβ4GlcNAcβ4(Fucα6)GlcNAcβ

### Manα3[GlcNAcβ2Manα6]Manβ4GlcNAcβ4(Fucα6)GlcNAcβ

### GlcNAcβ2Manα3[Manα6]Manβ4GlcNAcβ4(Fucα6)GlcNAcβ

### GlcNAcβ2Manα3[GlcNAcβ2Manα6]Manβ4GlcNAcβ4(Fucα6)GlcNAcβ

### Preferred Effects of Proliferation Rates of Stem Cells Especially Mesenchymal Stem Cells

Table 24 shows proliferation rates of mesenchymal stem cells on various binders with different carbohydrate specificities. The data reveals that it is possible to cultivate several the cells on various types of lectins and the proteins modulate the growth rate of the cells in comparison to the plastic surface of the experiment. The lectin RCA in passively immobilized form may show some toxicity to the cells, the invention is especially directed to non-toxic variant or covalently conjugated form of cytotoxic lectins such as ricin. The invention is directed to modulation of the growth rate under various conditions, in a preferred embodiment under the shorter cultivation period, such as in two weeks as in the example.

### Lectins for Preferably Increasing Proliferation

The highest proliferation rate was obtained with GSAII-lectin, which is especially specific for terminal N-acetylglucosamine residues. In a preferred embodiment the invention is directed to cultivation of stem cells in presence of lectin with similar specificity as GSAII. The cultivation method is especially directed for changing growth speed of the cells. Preferably the stem cell preparation to be grown with GSAII comprises glycans binding to GSAII, more preferably terminal GlcNAc comprising glycans, even more preferably terminal GlcNAcβ-comprising glycans.

Relatively high proliferation rate was obtained with ECA-lectin, which is especially specific for terminal N-acetyllactosamine residues. In a preferred embodiment the invention is directed to cultivation of stem cells in presence of lectin with similar specificity as ECA. The cultivation method is especially directed for changing growth speed of the cells. Preferably the stem cell preparation to be grown with ECA comprises glycans binding to ECA, more preferably terminal N-acetyllactosamine comprising glycans, even more preferably terminal N-acetyllactosamineβ-comprising glycans.

Increased proliferation rate was obtained with PWA-lectin, which is especially specific for terminal N-acetyllactosamine residues. In a preferred embodiment the invention is directed to cultivation of stem cells in presence of lectin with similar specificity as PWA. The cultivation method is especially directed for changing growth speed of the cells. Preferably the stem cell preparation to be grown with PWA comprises glycans binding to PWA, more preferably terminal N-acetyllactosamine comprising glycans, even more preferably terminal N-acetyllactosamineβ-comprising glycans.

Some increase of proliferation rate was also obtained with LTA-lectin, which is especially specific for fucose, preferably in terminal Lewis x structure. In a preferred embodiment the invention is directed to cultivation of stem cells in presence of lectin with similar specificity as LTA. The cultivation method is especially directed for changing growth speed of the cells. Preferably the stem cell preparation to be grown with LTA comprises glycans binding to LTA, more preferably fucose residues comprising glycans, even more preferably fucose of terminal Lewis x comprising glycans.

Some increase of proliferation rate was also obtained with PSA-lectin, which is especially specific for core fucose and/or mannose residues, preferably core fucose of complex type N-glycans. In a preferred embodiment the invention is directed to cultivation of stem cells in presence of lectin with similar specificity as PSA. The cultivation method is especially directed for changing growth speed of the cells. Preferably the stem cell preparation to be grown with PSA comprises glycans binding to PSA, more preferably core fucose and/or mannose residues, comprising glycans, even more preferably fucose of complex type N-glycans comprising glycans.

### Lectins for Preferably Retaining or Decreasing of Initial Proliferation

The invention revealed also lectin surfaces with similar or a little reduced proliferation activity with lectin SNA-lectin, which is especially specific α6-linked sialic acids, and lectin MAA, specific for specific α3-linked sialic acids residues. In a preferred embodiment the invention is directed to cultivation of stem cells in presence of lectin with similar specificity as SNA or MAA. The cultivation method is especially directed for changing growth speed of the cells and/or other preferred properties according to the invention. Preferably the stem cell preparation to be grown with SNA or MAA comprises glycans binding to SNA or MAA, respectively, more preferably α3-linked sialic acids for lectin MAA, and α6-linked sialic acids for SNA. In preferred embodiment stem cells comprising specific N-glycan, O-glycan or Glycolipid structures as described by the invention comprising the terminal target glycan epitopes are selected. The preferred common specificity is according to the formula SAα3/6Galβ4GlcNAc, wherein SA is sialic acid preferably Neu5Ac either α3 or α6-linked to the N-acetyllactosamine

The invention further revealed, that mannose specific lectin NPA supports proliferation of cells with somewhat reduced growth rate. The NPA lectin is especially specific for α-linked Man, preferably Manα3/6 structures. In a preferred embodiment the invention is directed to cultivation of stem cells in presence of lectin with similar specificity as NPA. The cultivation method is especially directed for changing growth speed of the cells. Preferably the stem cell preparation to be grown with NPA comprises glycans binding to NPA, more preferably Manα, even more preferably Manα3/6 comprising glycans. In preferred embodiment stem cells comprising specific N-glycan, structures as described by the invention comprising the terminal target glycan epitopes are selected for cultivation with NPA.

It is realized that it is also useful to slow down proliferation of stem cells during the culture in order to preserve stem cell characteristics of a preparation. Preferred lectin for reducing the proliferation rate includes WFA, binding GalNAc-structures, especially lacdiNac GalNAcβ4GlcNAc, and N-acetyllactosamine structure; STA, which bind N-acetyllactosmines, especially linear poly-N-acetyllactosamines and UEA, which bind fucosylated structures, especially, Fucα2Gal-type structures, such as Fucα2Galβ4GlcNAc. In a preferred embodiment the invention is directed to cultivation of stem cells in presence of lectin with similar specificity as WFA, STA or UEA. The cultivation method is especially directed for changing, preferably reducing growth speed of the cells and/or other preferred properties according to the invention. Preferably the stem cell preparation to be grown with the lectins comprises one or several of target glycans of the lectins preferably as indicated above. In preferred embodiment stem cells comprising specific N-glycan, O-glycan or Glycolipid structures as described by the invention comprising the terminal target glycan epitopes are selected.

The invention revealed a specific target structure group of the lectins with this specificity including reducing end elongated poly-Nacetyllactosamines (like STA) or 2-modified Gal comprising structures of LacdiNAc and Fucα2Gal- for WFA and UEA, respectively. The invention is directed to the group of lectins with these N-acetyllactosamine type specificities for modulation of the growth of stem cells. The preferred common specificity is according to the formula [R2]ₙGalβ4GlcNAc[β3Galβ]ₘ, wherein n and m are 0 or 1 and R2 is N-acetyl group (NAc) replacing hydroxyl on position 2 of galactopyranosyl or glycosidically linked Fucα-residue on position2.

### Other Modulation Effects to Cells

The example 10 describes further effects of cell culture in a longer cultivation experiment. Cells proliferated perhaps most efficiently on MAA and ECA when compared to plastic or other types of surfaces. All wells reached confluency within a week. Cells cultivated on WFA and PWA seemed to loose their proliferation capacity during 5 weeks period and on WFA coating there were some morphologically different cells. The lectins MAA and ECA are especially preferred for the longer term proliferation effects. The lectin WFA is preferred for affecting cellular morphology.

Cell morphology and attachment effects. The invention is especially directed to alterations of cell morphology and/or attachment strength by the binder such as lectins. Morphologically cells growing on PSA coating differed from the others by their way of forming a netlike monolayer. Cells on MAA and PSA were also more tightly attached to the surface and their detachment with trypsin was not possible, those cells needed to be scratched off mechanically. The PSA lectin and lectins with similar specificity especially with regard to fucose and/or mannose structures are preferred due to its activity in affecting morphology of the cells and/or causing increased binding preferably a protease resistant binding. The MAA lectin and lectins with similar specificity especially with regard to α3-linked sialic acid structures are preferred due to its activity in causing increased binding preferably a protease resistant binding.

### Preferred Combinations of the Binders

The invention revealed useful combination of specific terminal structures for the analysis of status of a cells. In a preferred embodiment the invention is directed to measuring the level of two different terminal structures according to the invention, preferably by specific binding molecules, preferably at least by two different binders. In a preferred embodiment the binder molecules are directed to structures indicating modification of a terminal receptor glycan structures, preferably the structures represent sequential (substrate structure and modification thereof, such as terminal Gal-structure and corresponding sialylated structure) or competing biosynthetic steps (such as fucosylation and sialylation of terminal Galβ or terminal Galβ3GlcNAc and Galβ4GlcNAc). In another embodiment the binders are directed to three different structures representing sequential and competing steps such as such as terminal Gal-structure and corresponding sialylated structure and corresponding sialylated structure.

The invention is further directed to recognition of at least two different structures according to the invention selected from the groups of non-modified (non-sialylated or non-fucosylated) Gal(NAc)β3/4-core structures according to the invention, preferred fucosylated structures and preferred sialylated structures according to the invention. It is realized that it is useful to recognize even 3, and more preferably 4 and even more preferably five different structures, preferably within a preferred structure group.

### Target Structures for Specific Binders and Examples of the Binding Molecules

Combination of Terminal Structures with Specific Glycan Core Structures

It is realized that part of the structural elements are specifically associated with specific glycan core structure. The recognition of terminal structures linked to specific core structures are especially preferred, such high specificity reagents have capacity of recognition almost complete individual glycans to the level of physicochemical characterization according to the invention. For example many specific mannose structures according to the invention are in general quite characteristic for N-glycan glycomes according to the invention. The present invention is especially directed to recognition terminal epitopes.

### Common Terminal Structures on Several Glycan Core Structures

The present invention revealed that there are certain common structural features on several glycan types and that it is possible to recognize certain common epitopes on different glycan structures by specific reagents when specificity of the reagent is limited to the terminal without specificity for the core structure. The invention especially revealed characteristic terminal features for specific cell types according to the invention. The invention realized that the common epitopes increase the effect of the recognition. The common terminal structures are especially useful for recognition in the context with possible other cell types or material, which do not contain the common terminal structure in substantial amount. The invention revealed the presence of the terminal structures on specific core structures such as N-glycan, O-glycan and/or glycolipids. The invention is preferably directed to the selection of specific binders for the structures including recognition of specific glycan core types.

The invention is further directed to glycome compositions of protein linked glycomes such as N-glycans and O-glycans and glycolipids each composition comprising specific amounts of glycan subgroups. The invention is further directed to the compositions when these comprise specific amount of Defined terminal structures.

### Specific Preferred Structural Groups

The present invention is directed to recognition of oligosaccharide sequences comprising specific terminal monosaccharide types, optionally further including a specific core structure. The preferred oligosaccharide sequences are in a preferred embodiment classified based on the terminal monosaccharide structures.

The invention further revealed a family of terminal (non-reducing end terminal) disaccharide epitopes based on β-linked galactopyranosylstructures, which may be further modified by fucose and/or sialic acid residues or by N-acetylgroup, changing the terminal Gal residue to GalNAc. Such structures are present in N-glycan, O-glycan and glycolipid subglycomes. Furthermore the invention is directed to terminal disaccharide epitopes of N-glycans comprising terminal ManαMan.

The structures were derived by mass spectrometric and optionally NMR analysis and by high specificity binders according to the invention, for the analysis of glycolipid structures permethylation and fragmentation mass spectrometry was used. Biosynthetic analysis including known biosynthetic routes to N-glycans, O-glycans and glycolipids was additionally used for the analysis of the glycan compositions and additional support, though not direct evidence due to various regulation levels after mRNA, for it was obtained from gene expression profiling data of Skottman, H. et al. (2005) Stem cells and similar data obtained from the mRNA profiling for cord blood cells and used to support the biosynthetic analysis using the data of Jaatinen T et al. Stem Cells (2006) 24 (3) 631-41.

Structures with Terminal Mannose Monosaccharide

Preferred mannose-type target structures have been specifically classified by the invention. These include various types of high and low-mannose structures and hybrid type structures according to the invention.

### The Preferred Terminal Manα-Target Structure Epitopes

The invention revealed the presence of Manα on low mannose N-glycans and high mannose N-glycans. Based on the biosynthetic knowledge and supporting this view by analysis of mRNAs of biosynthetic enzymes and by NMR-analysis the structures and terminal epitopes could be revealed:

Manα2Man, Manα3Man, Manα6Man and Manα3(Manα6)Man, wherein the reducing end Man is preferably either α- or β-linked glycoside and α-linked glycoside in case of Manα2Man:

The general structure of terminal Manα-structures is

Manαx(Manαy)_{z}Manα/β

Wherein

x is linkage position 2, 3 or 6, and y is linkage position 3 or 6,
z is integer 0 or 1, indicating the presence or the absence of the branch,
with the provision that x and y are not the same position and
when x is 2, the z is 0 and reducing end Man is preferably α-linked;

The low_mannose structures includes preferably non-reducing end terminal epitopes with structures with α3- and/or α6-mannose linked to another mannose residue

Manαx(Manαy)_{z}Manα/β
wherein x and y are linkage positions being either 3 or 6,
z is integer 0 or 1, indicating the presence or the absence of the branch,

The high mannose structure includes terminal α2-linked Mannose:

Manα2Man(α) and optionally on or several of the terminal α3- and/or α6-mannose-structures as above.

The presence of terminal Manα-structures is regulated in stem cells and the proportion of the high-Man-structures with terminal Manα2-structures in relation to the low Man structures with Manα3/6- and/or to complex type N-glycans with Gal-backbone epitopes varies cell type specifically.

The data indicated that binder revealing specific terminal Manα2Man and/or Manα3/6Man is very useful in characterization of stem cells. The prior science has not characterized the epitopes as specific signals of cell types or status.

The invention is especially directed to the measuring the levels of both low-Man and high-Man structures, preferably by quantifying two structure type the Manα2Man-structures and the Manα3/6Man-structures from the same sample.

The invention is especially directed to high specificity binders such as enzymes or monoclonal antibodies for the recognition of the terminal Manα-structures from the preferred stem cells according to the invention, more preferably from differentiated embryonal type cells, more preferably differentiated beyond embryoid bodies such as stage 3 differentiated cells, most preferably the structures are recognized from stage 3 differentiated cells. The invention is especially preferably directed to detection of the structures from adult stem cells more preferably mesenchymal stem cells, especially from the surface of mesenchymal stem cells and in separate embodiment from blood derived stem cells, with separately preferred groups of cord blood and bone marrow stem cells. In a preferred embodiment the cord blood and/or peripheral blood stem cell is not hematopoietic stem cell.

### Low or Uncharacterised Specificity Binders

preferred for recognition of terminal mannose structures includes mannose-monosaccharide binding plant lectins. The invention is in preferred embodiment directed to the recognition of stem cells such as embryonal type stem cells by a Manα-recognizing lectin such as lectin PSA. In a preferred embodiment the recognition is directed to the intracellular glycans in permeabilized cells. In another embodiment the Manα-binding lectin is used for intact non-permeabilized cells to recognize terminal Manα-from contaminating cell population such as fibroblast type cells or feeder cells as shown in corresponding Example 3.

### Preferred High Specific High Specificity Binders

include
i) Specific mannose residue releasing enzymes such as linkage specific mannosidases, more preferably an α-mannosidase or β-mannosidase.

Preferred α-mannosidases includes linkage specific α-mannosidases such as α-Mannosidases cleaving preferably non-reducing end terminal, an example of preferred mannosidases is jack bean α-mannosidase (Canavalia ensiformis; Sigma, USA) and homologous α-mannosidases α2-linked mannose residues specifically or more effectively than other linkages, more preferably cleaving specifically Manα2-structures; or

α3-linked mannose residues specifically or more effectively than other linkages, more preferably cleaving specifically Manα3-structures; or
α6-linked mannose residues specifically or more effectively than other linkages, more preferably cleaving specifically Manα6-structures;

Preferred β-mannosidases includes fl-mannosidases capable of cleaving β4-linked mannose from non-reducing end terminal of N-glycan core Manβ4GlcNAc-structure without cleaving other β-linked monosaccharides in the glycomes.

ii) Specific binding proteins recognizing preferred mannose structures according to the invention. The preferred reagents include antibodies and binding domains of antibodies (Fab-fragments and like), and other engineered carbohydrate binding proteins. The invention is directed to antibodies recognizing MS2B1 and more preferably MS3B2-structures.

Mannosidase analyses of neutral N-glycans Examples of detection of mannosylated by α-mannosidase binder and mass spectrometric profiling of the glycans cord blood and peripheral blood mesenchymal cells in Example 1; for cord blood cells in example 15, hESC EB and stage 3 cells in Example 7, in Example 17 and 2 for embryonal stem cells and differentiated cells; and, and indicates presence of all types of Manβ4, Manα3/6 terminal structures of Man₁₋₄GlcNAcβ4(Fucα6)₀₋₁GlcNAc- comprising low Mannose glycans as described by the invention.

### Lectin Binding

α-linked mannose was demonstrated in Example 4 for human mesenchymal cell by lectins Hippeastrum hybrid (HHA) and Pisum sativum (PSA) lectins suggests that they express mannose, more specifically α-linked mannose residues on their surface glycoconjugates such as N-glycans. Possible α-mannose linkages include α1→2, α1→3, and α1→6. The lower binding of Galanthus nivalis (GNA) lectin suggests that some α-mannose linkages on the cell surface are more prevalent than others. The combination of the terminal Manα-recognizing low affinity reagents appears to be useful and correspond to results obtained by mannosidase screening; NMR and mass spectrometric results. Lectin binding of cord blood cells is in example 5. PSA has specificity for complex type N-glycans with core Fucα6-epitopes.

Mannose-binding lectin labelling. Labelling of the mesenchymal cells in Example 4 was also detected with human serum mannose-binding lectin (MBL) coupled to fluorescein label. This indicate that ligands for this innate immunity system component may be expressed on in vitro cultured BM MSC cell surface.

The present invention is especially directed to analysis of terminal Manα-on cell surfaces as the structure is ligand for MBL and other lectins of innate immunity. It is further realized that terminal Manα-structures would direct cells in blood circulation to mannose receptor comprising tissues such as Kupfer cells of liver. The invention is especially directed to control of the amount of the structure by binding with a binder recognizing terminal Manα-structure.

In a preferred embodiment the present invention is directed to the testing of presence of ligands of lectins present in human, such as lectins of innate immunity and/or lectins of tissues or leukocytes, on stem cells by testing of the binding of the lectin (purified or preferably a recombinant form of the lectin, preferably in labeled form) to the stem cells. It is realized that such lectins includes especially lectins binding Manα and Galβ/GalNAcβ-structures (terminal non-reducing end or even α6-sialylated forms according to the invention.

### Mannose Binding Antibodies

A high-mannose binding antibody has been described for example in Wang L X et al (2004) 11 (1) 127-34. Specific antibodies for short mannosylated structures such as the trimannosyl core structure have been also published.

Structures with Terminal Gal-Monosaccharide

Preferred galactose-type target structures have been specifically classified by the invention. These include various types of N-acetyllactosamine structures according to the invention.

### Low or Uncharacterised Specificity Binders for Terminal Gal

Preferred for recognition of terminal galactose structures includes plant lectins such as ricin lectin (ricinus communis agglutinin RCA), and peanut lectin(/agglutinin PNA). The low resolution binders have different and broad specificities.

### Preferred High Specific High Specificity Binders Include

i) Specific galactose residue releasing enzymes such as linkage specific galactosidases, more preferably α-galactosidase or β-galactosidase.

Preferred α-galactosidases include linkage galactosidases capable of cleaving Galα3Gal-structures revealed from specific cell preparations

Preferred β-galactosidases includes β-galactosidases capable of cleaving

β4-linked galactose from non-reducing end terminal Galβ4GlcNAc-structure without cleaving other β-linked monosaccharides in the glycomes and
β3-linked galactose from non-reducing end terminal Galβ3GlcNAc-structure without cleaving other β-linked monosaccharides in the glycomes
ii) Specific binding proteins recognizing preferred galactose structures according to the invention. The preferred reagents include antibodies and binding domains of antibodies (Fab-fragments and like), and other engineered carbohydrate binding proteins and animal lectins such as galectins.

### Specific Binder Experiments and Examples for Galβ-Structures

Specific exoglycosidase and glycosyltransferase analysis for the structures are included in Example 17 and 2 for embryonal stem cells and differentiated cells; Example 1 mesenchymal cells, for cord blood cells in example 15 and in example 16 on cell surface and including glycosyltransferases, for glycolipids in Example 11. Sialylation level analysis related to terminal Galβ and Sialic acid expression is in Example 6.

Preferred enzyme binders for the binding of the Galβ-epitopes according to the invention includes β1,4-galactosidase e.g from S. pneumoniae (rec. in E. coli, Calbiochem, USA), β1,3-galactosidase (e.g rec. in E. coli, Calbiochem); glycosyltransferases: α2,3-(N)-sialyltransferase (rat, recombinant in S. frugiperda, Calbiochem), α1,3-fucosyltransferase VI (human, recombinant in S. frugiperda, Calbiochem), which are known to recognize specific N-acetyllactosamine epitopes, Fuc-TVI especially Galβ4GlcNAc.

Plant low specificity lectin, such as RCA, PNA, ECA, STA, and

PWA, data is in Example 3 for hESC, Example 4 for MSCs, Example 5 for cord blood, effects of the lectin binders for the cell proliferation is in Example 10, cord blood cell selection is in Example 12.

Human lectin analysis by various galectin expression is Example 13 from cord blood and embryonal cells,

In example 14 there is antibody labeling of especially fucosylated and galactosylated structures.

Poly-N-acetyllactosamine sequences. Labelling of the cells by pokeweed (PWA) and less intense labelling by Solanum tuberosum (STA) lectins suggests that the cells express poly-N-acetyllactosamine sequences on their surface glycoconjugates such as N- and/or O-glycans and/or glycolipids. The results further suggest that cell surface poly-N-acetyllactosamine chains contain both linear and branched sequences.

Structures with Terminal GalNAc-Monosaccharide

Preferred GalNAc-type target structures have been specifically revealed by the invention. These include especially LacdiNAc, GalNAcβGlcNAc-type structures according to the invention.

### Low or Uncharacterised Specificity Binders for Terminal GalNAc

Several plant lectins has been reported for recognition of terminal GalNAc. It is realized that some GalNAc-recognizing lectins may be selected for low specificity recognition of the preferred LacdiNAc-structures.

β-linked N-acetylgalactosamine. Abundant labelling of hESC by Wisteria floribunda lectin (WFA) suggests that hESC express β-linked non-reducing terminal N-acetylgalactosamine residues on their surface glycoconjugates such as N- and/or O-glycans. The absence of specific binding of WFA to mEF suggests that the lectin ligand epitopes are less abundant in mEF.

The low specificity binder plant lectins such as Wisteria floribunda agglutinin and Lotus tetragonolobus agglutinin bind to oligosaccharide sequences Srivatsan J. et al. Glycobiology (1992) 2 (5) 445-52: Do, K Y et al. Glycobiology (1997) 7 (2) 183-94; Yan, L., et al (1997) Glycoconjugate J. 14 (1) 45-55. The article also shows that the lectins are useful for recognition of the structures, when the cells are verified not to contain other structures recognized by the lectins.

In a preferred embodiment a low specificity lectin reagent is used in combination with another reagent verifying the binding.

### Preferred High Specific High Specificity Binders Include

i) The invention revealed that β-linked GalNAc can be recognized by specific β-N-acetylhexosaminidase enzyme in combination with β-N-acetylhexosaminidase enzyme. This combination indicates the terminal monosaccharide and at least part of the linkage structure.

Preferred β-N-acetylhexosaminidase, includes enzyme capable of cleaving β-linked GalNAc from non-reducing end terminal GalNAcβ4/3-structures without cleaving α-linked HexNAc in the glycomes; preferred N-acetylglucosaminidases include enzyme capable of cleaving β-linked GlcNAc but not GalNAc.

Specific binding proteins recognizing preferred GalNAcβ4, more preferably GalNAcβ4GlcNAc, structures according to the invention. The preferred reagents include antibodies and binding domains of antibodies (Fab-fragments and like), and other engineered carbohydrate binding proteins.

Examples antibodies recognizing LacdiNAc-structures includes publications of Nyame A. K. et al. (1999) Glycobiology 9 (10) 1029-35; van Remoortere A. et al (2000) Glycobiology 10 (6) 601-609; and van Remoortere A. et al (2001) Infect. Immun 69 (4) 2396-2401. The antibodies were characterized in context of parasite (Schistosoma) infection of mice and humans, but according to the present invention these antibodies can also be used in screening stem cells. The present invention is especially directed to selection of specific clones of LacdiNac recognizing antibodies specific for the subglycomes and glycan structures present in N-glycomes of the invention.

The articles disclose antibody binding specificities similar to the invention and methods for producing such antibodies, therefore the antibody binders are obvious for person skilled in the art. The immunogenicity of certain LacdiNAc-structures are demonstrated in human and mice.

The use of glycosidase in recognition of the structures in known in the prior art similarily as in the present invention for example in Srivatsan J. et al. (1992) 2 (5) 445-52.

Structures with Terminal GlcNAc-Monosaccharide

Preferred GlcNAc-type target structures have been specifically revealed by the invention. These include especially GlcNAcβ-type structures according to the invention.

### Low or Uncharacterised Specificity Binders for Terminal GlcNAc

Several plant lectins has been reported for recognition of terminal GlcNAc. It is realized that some GlcNAc-recognizing lectins may be selected for low specificity recognition of the preferred GlcNAc-structures.

### Preferred High Specific High Specificity Binders Include

i) The invention revealed that β-linked GlcNAc can be recognized by specific β-N-acetylglucosaminidase enzyme.

Preferred β-N-acetylglucosaminidase includes enzyme capable of cleaving β-linked GlcNAc from non-reducing end terminal GlcNAcβ2/3/6-structures without cleaving β-linked GalNAc or α-linked HexNAc in the glycomes;

ii) Specific binding proteins recognizing preferred GlcNAcβ2/3/6, more preferably GlcNAcβ2Manα, structures according to the invention. The preferred reagents include antibodies and binding domains of antibodies (Fab-fragments and like), and other engineered carbohydrate binding proteins.

### Specific Binder Experiments and Examples for Terminal HexNAc(GalNAc/GlcNAc and GlcNAc Structures

Specific exoglycosidase analysis for the structures are included in Example 17 and 2 for embryonal stem cells and differentiated cells; Example 1 for mesenchymal cells, for cord blood cells in example 15 and for glycolipids in Example 11.

Plant low specificity lectin, such as WFA and GNAII, and data is in Example 3 for hESC, Example 4 for MSCs, Example 5 for cord blood, effects of the lectin binders for the cell proliferation is in Example 10, cord blood cell selection is in Example 12.

Preferred enzymes for the recognition of the structures includes general hexosaminidase β-hexosaminidase from Jack beans (C. ensiformis, Sigma, USA) and specific N-acetylglucosaminidases or N-acetylgalactosaminidases such as β-glucosaminidase from S. pneumoniae (rec. in E. coli, Calbiochem, USA). Combination of these allows determination of LacdiNAc.

The invention is further directed to analysis of the structures by specific monoclonal antibodies recognizing terminal GlcNAcβ-structures such as described in Holmes and Greene (1991) 288 (1) 87-96, with specificity for several terminal GlcNAc structures.

The invention is specifically directed to the use of the terminal structures according to the invention for selection and production of antibodies for the structures.

Verification of the target structures includes mass spectrometry and

permethylation/fragmentation analysis for glycolipid structures
Structures with Terminal Fucose-Monosaccharide

Preferred fucose-type target structures have been specifically classified by the invention. These include various types of N-acetyllactosamine structures according to the invention. The invention is further more directed to recognition and other methods according to the invention for lactosamine similar α6-fucosylated epitope of N-glycan core, GlcNAcβ4(Fucα6)GlcNAc. The invention revealed such structures recognizable by the lectin PSA (Kornfeld (1981) J Biol Chem 256, 6633-6640; Cummings and Kornfeld (1982) J Biol Chem 257, 11235-40) are present e.g. in embryonal stem cells and mesenchymal stem cells.

### Low or Uncharacterised Specificity Binders for Terminal Fuc

Preferred for recognition of terminal fucose structures includes fucose monosaccharide binding plant lectins. Lectins of Ulex europeaus and Lotus tetragonolobus has been reported to recognize for example terminal Fucoses with some specificity binding for α2-linked structures, and branching α3-fucose, respectively. Data is in Example 3 for hESC, Example 4 for MSCs, Example 5 for cord blood, effects of the lectin binders for the cell proliferation is in Example 10, cord blood cell selection is in Example 12.

### Preferred High Specific High Specificity Binders Include

i) Specific fucose residue releasing enzymes such as linkage fucosidases, more preferably α-fucosidase.

Preferred α-fucosidases include linkage fucosidases capable of cleaving Fucα2Gal-, and Galβ4/3(Fucα3/4)GlcNAc-structures revealed from specific cell preparations.

Specific exoglycosidase and for the structures are included in Example 17 and 2 for embryonal stem cells and differentiated cells; Example 1 for mesenchymal cells, for cord blood cells in example 15 and in example 16 on cell surface for glycolipids in Example 11. Preferred fucosidases includes α1,3/4-fucosidase e.g. α1,3/4-fucosidase from Xanthomonas sp. (Calbiochem, USA), and α1,2-fucosidase e.g α1,2-fucosidase from X. manihotis (Glyko),

ii) Specific binding proteins recognizing preferred fucose structures according to the invention. The preferred reagents include antibodies and binding domains of antibodies (Fab-fragments and like), and other engineered carbohydrate binding proteins and animal lectins such as selectins recognizing especially Lewis type structures such as Lewis x, Galβ4(Fucα3)GlcNAc, and sialyl-Lewis x, SAα3Galβ4(Fucα3)GlcNAc.

The preferred antibodies includes antibodies recognizing specifically Lewis type structures such as Lewis x, and sialyl-Lewis x. More preferably the Lewis x-antibody is not classic SSEA-1 antibody, but the antibody recognizes specific protein linked Lewis x structures such as Galβ4(Fucα3)GlcNAcβ2Manα-linked to N-glycan core.

iii) the invention is further directed to recognition of ab-fucosylated epitope of N-glycan core, GlcNAcβ4(Fucα6)GlcNAc. The invention directed to recognition of such structures by structures by the lectin PSA or lentil lectin (Kornfeld (1981) J Biol Chem 256, 6633-6640) or by specific monoclonal antibodies (e.g. Srikrishna G. et al (1997) J Biol Chem 272, 25743-52). The invention is further directed to methods of isolation of cellular glycan components comprising the glycan epitope and isolation stem cell N-glycans, which are not bound to the lectin as control fraction for further characterization.
Structures with Terminal Sialic Acid-Monosaccharide

Preferred sialic acid-type target structures have been specifically classified by the invention.

### Low or Uncharacterised Specificity Binders for Terminal Sialic Acid

Preferred for recognition of terminal sialic acid structures includes sialic acid monosaccharide binding plant lectins.

### Preferred High Specific High Specificity Binders Include

i) Specific sialic acid residue releasing enzymes such as linkage sialidases, more preferably α-sialidases.

Preferred α-sialidases include linkage sialidases capable of cleaving SAα3Gal- and SAα6Gal-structures revealed from specific cell preparations by the invention.

Preferred low specificity lectins, with linkage specificity include the lectins, that are specific for SAα3Gal-structures, preferably being Maackia amurensis lectin and/or lectins specific for SAα6Gal-structures, preferably being Sambucus nigra agglutinin.

ii) Specific binding proteins recognizing preferred sialic acid oligosaccharide sequence structures according to the invention. The preferred reagents include antibodies and binding domains of antibodies (Fab-fragments and like), and other engineered carbohydrate binding proteins and animal lectins such as selectins recognizing especially Lewis type structures such as sialyl-Lewis x, SAα3Galβ4(Fucα3)GlcNAc or sialic acid recognizing Siglec-proteins. The preferred antibodies includes antibodies recognizing specifically sialyl-N-acetyllactosamines, and sialyl-Lewis x.

Preferred antibodies for NeuGc-structures includes antibodies recognizes a structure NeuGcα3Galβ4Glc(NAc)_{0 or 1} and/or GalNAcβ4[NeuGcα3]Galβ4Glc(NAc)_{0 or 1}, wherein [ ] indicates branch in the structure and ( )_{0 or 1} a structure being either present or absent. In a preferred embodiment the invention is directed recognition of the N-glycolyl-Neuraminic acid structures by antibody, preferably by a monoclonal antibody or human/humanized monoclonal antibody. A preferred antibody contains the variable domains of P3-antibody.

### Specific Binder Experiments and Examples for α3/6 Sialylated Structures

Specific exoglycosidase analysis for the structures are included in Example 17 and 2 for embryonal stem cells and differentiated cells; Example 1 for mesenchymal cells, for cord blood cells in example 15 and in example 16 on cell surface and including glycosyltransferases, for glycolipids in Example 11. Sialylation level analysis related to terminal Galβ and Sialic acid expression is in Example 6.

Preferred enzyme binders for the binding of the Sialic acid epitopes according to the invention includes: sialidases such as general sialidase α2,3/6/8/9-sialidase from A. ureafaciens (Glyko), and α2,3-Sialidases such as: α2,3-sialidase from S. pneumoniae (Calbiochem, USA). Other useful sialidases are known from E. coli, and Vibrio cholerae.

α1,3-fucosyltransferase VI (human, recombinant in S. frugiperda, Calbiochem), which are known to recognize specific N-acetyllactosamine epitopes, Fuc-TVI especially including SAα3Galβ4GlcNAc.

Plant low specificity lectin, such as MAA and SNA, and data is in Example 3 for hESC, Example 4 for MSCs, Example 5 for cord blood, effects of the lectin binders for the cell proliferation is in Example 10, cord blood cell selection is in Example 12.

In example 14 there is antibody labeling of sialylstructures.
Preferred Uses for Stem Cell Type Specific Galectins and/or Galectin Ligands

As described in the Examples, the inventors also found that different stem cells have distinct galectin expression profiles and also distinct galectin (glycan) ligand expression profiles. The present invention is further directed to using galactose-binding reagents, preferentially galactose-binding lectins, more preferentially specific galectins; in a stem cell type specific fashion to modulate or bind to certain stem cells as described in the present invention to the uses described. In a further preferred embodiment, the present invention is directed to using galectin ligand structures, derivatives thereof, or ligand-mimicking reagents to uses described in the present invention in stem cell type specific fashion. The preferred galectins are listed in Example 13.

The invention is in a preferred embodiment directed to the recognition of terminal N-acetyllactosamines from cells by galectins as described above for recognition of Galβ4GlcNAc and Galβ3GlcNAc structures: The results indicate that both CB CD34+/CD133+ stem cell populations and hESC have an interesting and distinct galectin expression profiles, leading to different galectin ligand affinity profiles (Hirabayashi et al., 2002). The results further correlate with the glycan analysis results showing abundant galectin ligand expression in these stem cells, especially non-reducing terminal β-Gal and type II LacNAc, poly-LacNAc, β1,6-branched poly-LacNAc, and complex-type N-glycan expression.

### Specific Technical Aspects of Stem Cell Glycome Analysis

### Isolation of Glycans and Glycan Fractions

Glycans of the present invention can be isolated by the methods known in the art. A preferred glycan preparation process consists of the following steps:

1^{o} isolating a glycan-containing fraction from the sample,
2^{o} . . . . Optionally purification the fraction to useful purity for glycome analysis

The preferred isolation method is chosen according to the desired glycan fraction to be analyzed. The isolation method may be either one or a combination of the following methods, or other fractionation methods that yield fractions of the original sample:

1^{o} extraction with water or other hydrophilic solvent, yielding water-soluble glycans or glycoconjugates such as free oligosaccharides or glycopeptides,
2^{o} extraction with hydrophobic solvent, yielding hydrophilic glycoconjugates such as glycolipids,
3^{o} N-glycosidase treatment, especially Flavobacterium meningosepticum N-glycosidase F treatment, yielding N-glycans,
4^{o} alkaline treatment, such as mild (e.g. 0.1 M) sodium hydroxide or concentrated ammonia treatment, either with or without a reductive agent such as borohydride, in the former case in the presence of a protecting agent such as carbonate, yielding β-elimination products such as O-glycans and/or other elimination products such as N-glycans,
5^{o} endoglycosidase treatment, such as endo-3-galactosidase treatment, especially Escherichia freundii endo-β-galactosidase treatment, yielding fragments from poly-N-acetyllactosamine glycan chains, or similar products according to the enzyme specificity, and/or
6^{o} protease treatment, such as broad-range or specific protease treatment, especially trypsin treatment, yielding proteolytic fragments such as glycopeptides.

The released glycans are optionally divided into sialylated and non-sialylated subfractions and analyzed separately. According to the present invention, this is preferred for improved detection of neutral glycan components, especially when they are rare in the sample to be analyzed, and/or the amount or quality of the sample is low. Preferably, this glycan fractionation is accomplished by graphite chromatography.

According to the present invention, sialylated glycans are optionally modified in such manner that they are isolated together with the non-sialylated glycan fraction in the non-sialylated glycan specific isolation procedure described above, resulting in improved detection simultaneously to both non-sialylated and sialylated glycan components. Preferably, the modification is done before the non-sialylated glycan specific isolation procedure. Preferred modification processes include neuraminidase treatment and derivatization of the sialic acid carboxyl group, while preferred derivatization processes include amidation and esterification of the carboxyl group.

### Glycan Release Methods

The preferred glycan release methods include, but are not limited to, the following methods:

Free glycans—extraction of free glycans with for example water or suitable water-solvent mixtures.
Protein-linked glycans including O- and N-linked glycans—alkaline elimination of protein-linked glycans, optionally with subsequent reduction of the liberated glycans.
Mucin-type and other Ser/Thr O-linked glycans—alkaline β-elimination of glycans, optionally with subsequent reduction of the liberated glycans.
N-glycans—enzymatic liberation, optionally with N-glycosidase enzymes including for example N-glycosidase F from C. meningosepticum, Endoglycosidase H from Streptomyces, or N-glycosidase A from almonds.
Lipid-linked glycans including glycosphingolipids—enzymatic liberation with endoglycoceramidase enzyme; chemical liberation; ozonolytic liberation.
Glycosaminoglycans—treatment with endo-glycosidase cleaving glycosaminoglycans such as chondroinases, chondroitin lyases, hyalurondases, heparanases, heparatinases, or keratanases/endo-beta-galactosidases; or use of O-glycan release methods for β-glycosidic Glycosaminoglycans; or N-glycan release methods for N-glycosidic glycosaminoglycans or use of enzymes cleaving specific glycosaminoglycan core structures; or specific chemical nitrous acid cleavage methods especially for amine/N-sulphate comprising glycosaminoglycans
Glycan fragments—specific exo- or endoglycosidase enzymes including for example keratanase, endo-β-galactosidase, hyaluronidase, sialidase, or other exo- and endoglycosidase enzyme; chemical cleavage methods; physical methods

### Preferred Target Cell Populations and Types for Analysis According to the Invention

### Early Human Cell Populations

### Human Stem Cells and Multipotent Cells

Under broadest embodiment the present invention is directed to all types of human stem cells, meaning fresh and cultured human stem cells. The stem cells according to the invention do not include traditional cancer cell lines, which may differentiate to resemble natural cells, but represent non-natural development, which is typically due to chromosomal alteration or viral transfection. Stem cells include all types of non-malignant multipotent cells capable of differentiating to other cell types. The stem cells have special capacity stay as stem cells after cell division, the self-reneval capacity.

Under the broadest embodiment for the human stem cells, the present invention describes novel special glycan profiles and novel analytics, reagents and other methods directed to the glycan profiles. The invention shows special differences in cell populations with regard to the novel glycan profiles of human stem cells.

The present invention is further directed to the novel structures and related inventions with regard to the preferred cell populations according to the invention. The present invention is further directed to specific glycan structures, especially terminal epitopes, with regard to specific preferred cell population for which the structures are new.

### Preferred Types of Early Human Cells

The invention is directed to specific types of early human cells based on the tissue origin of the cells and/or their differentiation status.

The present invention is specifically directed to early human cell populations meaning multipotent cells and cell populations derived thereof based on origins of the cells including the age of donor individual and tissue type from which the cells are derived, including preferred cord blood as well as bone marrow from older individuals or adults. Preferred differentiation status based classification includes preferably “solid tissue progenitor” cells, more preferably “mesenchymal-stem cells”, or cells differentiating to solid tissues or capable of differentiating to cells of either ectodermal, mesodermal, or endodermal, more preferentially to mesenchymal stem cells.

The invention is further directed to classification of the early human cells based on the status with regard to cell culture and to two major types of cell material. The present invention is preferably directed to two major cell material types of early human cells including fresh, frozen and cultured cells.

### Cord Blood Cells, Embryonal-Type Cells and Bone Marrow Cells

The present invention is specifically directed to early human cell populations meaning multipotent cells and cell populations derived thereof based on the origin of the cells including the age of donor individual and tissue type from which the cells are derived.
a) from early age-cells such 1) as neonatal human, directed preferably to cord blood and related material, and 2) embryonal cell-type material
b) from stem and progenitor cells from older individuals (non-neonatal, preferably adult), preferably derived from human “blood related tissues” comprising, preferably bone marrow cells.

### Cells Differentiating to Solid Tissues, Preferably to Mesenchymal Stem Cells

The invention is specifically under a preferred embodiment directed to cells, which are capable of differentiating to non-hematopoietic tissues, referred as “solid tissue progenitors”, meaning to cells differentiating to cells other than blood cells. More preferably the cell population produced for differentiation to solid tissue are “mesenchymal-type cells”, which are multipotent cells capable of effectively differentiating to cells of mesodermal origin, more preferably mesenchymal stem cells.

Most of the prior art is directed to hematopoietic cells with characteristics quite different from the mesenchymal-type cells and mesenchymal stem cells according to the invention.

Preferred solid tissue progenitors according to the invention includes selected multipotent cell populations of cord blood, mesenchymal stem cells cultured from cord blood, mesenchymal stem cells cultured/obtained from bone marrow and embryonal-type cells. In a more specific embodiment the preferred solid tissue progenitor cells are mesenchymal stem cells, more preferably “blood related mesenchymal cells”, even more preferably mesenchymal stem cells derived from bone marrow or cord blood.

Under a specific embodiment CD34+ cells as a more hematopoietic stem cell type of cord blood or CD34+ cells in general are excluded from the solid tissue progenitor cells.

### Early Blood Cell Populations and Corresponding Mesenchymal Stem Cells

### Cord Blood

The early blood cell populations include blood cell materials enriched with multipotent cells. The preferred early blood cell populations include peripheral blood cells enriched with regard to multipotent cells, bone marrow blood cells, and cord blood cells. In a preferred embodiment the present invention is directed to mesenchymal stem cells derived from early blood or early blood derived cell populations, preferably to the analysis of the cell populations.

### Bone Marrow

Another separately preferred group of early blood cells is bone marrow blood cells. These cell do also comprise multipotent cells. In a preferred embodiment the present invention is directed to directed to mesenchymal stem cells derived from bone marrow cell populations, preferably to the analysis of the cell populations.

### Preferred Subpopulations of Early Human Blood Cells

The present invention is specifically directed to subpopulations of early human cells. In a preferred embodiment the subpopulations are produced by selection by an antibody and in another embodiment by cell culture favouring a specific cell type. In a preferred embodiment the cells are produced by an antibody selection method preferably from early blood cells. Preferably the early human blood cells are cord blood cells.

The CD34 positive cell population is relatively large and heterogenous. It is not optimal for several applications aiming to produce specific cell products. The present invention is preferably directed to specifically selected non-CD34 populations meaning cells not selected for binding to the CD34-marker, called homogenous cell populations. The homogenous cell populations may be of smaller size mononuclear cell populations for example with size corresponding to CD133+ cell populations and being smaller than specifically selected CD34+ cell populations. It is further realized that preferred homogenous subpopulations of early human cells may be larger than CD34+ cell populations.

The homogenous cell population may a subpopulation of CD34+ cell population, in preferred embodiment it is specifically a CD133+ cell population or CD133-type cell population. The “CD133-type cell populations” according to the invention are similar to the CD133+ cell populations, but preferably selected with regard to another marker than CD133. The marker is preferably a CD133-coexpressed marker. In a preferred embodiment the invention is directed to CD133+ cell population or CD133+ subpopulation as CD133-type cell populations. It is realized that the preferred homogeneous cell populations further includes other cell populations than which can be defined as special CD133-type cells.

Preferably the homogenous cell populations are selected by binding a specific binder to a cell surface marker of the cell population. In a preferred embodiment the homogenous cells are selected by a cell surface marker having lower correlation with CD34-marker and higher correlation with CD133 on cell surfaces. Preferred cell surface markers include α3-sialylated structures according to the present invention enriched in CD133-type cells. Pure, preferably complete, CD133+ cell population are preferred for the analysis according to the present invention.

The present invention is directed to essential mRNA-expression markers, which would allow analysis or recognition of the cell populations from pure cord blood derived material. The present invention is specifically directed to markers specifically expressed on early human cord blood cells.

The present invention is in a preferred embodiment directed to native cells, meaning non-genetically modified cells. Genetic modifications are known to alter cells and background from modified cells. The present invention further directed in a preferred embodiment to fresh non-cultivated cells.

The invention is directed to use of the markers for analysis of cells of special differentiation capacity, the cells being preferably human blood cells or more preferably human cord blood cells.

Preferred Purity of Reproducibly Highly Purified Mononuclear Complete Cell Populations from Human Cord Blood

The present invention is specifically directed to production of purified cell populations from human cord blood. As described above, production of highly purified complete cell preparations from human cord blood has been a problem in the field. In the broadest embodiment the invention is directed to biological equivalents of human cord blood according to the invention, when these would comprise similar markers and which would yield similar cell populations when separated similarly as the CD133+ cell population and equivalents according to the invention or when cells equivalent to the cord blood is contained in a sample further comprising other cell types. It is realized that characteristics similar to the cord blood can be at least partially present before the birth of a human. The inventors found out that it is possible to produce highly purified cell populations from early human cells with purity useful for exact analysis of sialylated glycans and related markers.

### Preferred Bone Marrow Cells

The present invention is directed to multipotent cell populations or early human blood cells from human bone marrow. Most preferred are bone marrow derived mesenchymal stem cells. In a preferred embodiment the invention is directed to mesenchymal stem cells differentiating to cells of structural support function such as bone and/or cartilage.

A variety of factors previously mentioned influence ability of stem cells to survive, replicate, and differentiate. For example, in terms of nutrients the amino acid taurine under certain conditions preferentially inhibits murine bone marrow cells from forming osteoclasts (Koide, et al., 1999, Arch Oral Biol 44:711-719), the amino acid L-arginine stimulates erythrocyte differentiation and proliferation of erythroid progenitors (Shima, et al., 2006, Blood 107:1352-1356), extracellular ATP acting through P2Y receptors mediates a wide variety of changes to both hematopoietic and non-hematopoietic stem cells (Lee, et al., 2003, Genes Dev 17:1592-1604), arginine-glycine-aspartic acid attached to porous polymer scaffolds increase differentiation and survival of osteoblast progenitors (Hu, et al., 2003, J Biomed Mater Res A 64:583-590), each of which is incorporated by reference herein in its entirety. Accordingly, one skilled in the art would know to use various types of nutrients for inducing differentiation, or maintaining viability, of certain types of stem cells and/or progeny thereof.

### Embryonal-Type Cell Populations

The present invention is specifically directed to methods directed to embryonal-type cell populations, preferably when the use does not involve commercial or industrial use of human embryos nor involve destruction of human embryos. The invention is under a specific embodiment directed to use of embryonal cells and embryo derived materials such as embryonal stem cells, whenever or wherever it is legally acceptable. It is realized that the legislation varies between countries and regions.

The present invention is further directed to use of embryonal-related, discarded or spontaneously damaged material, which would not be viable as human embryo and cannot be considered as a human embryo. In yet another embodiment the present invention is directed to use of accidentally damaged embryonal material, which would not be viable as human embryo and cannot be considered as human embryo.

It is further realized that early human blood derived from human cord or placenta after birth and removal of the cord during normal delivery process is ethically uncontroversial discarded material, forming no part of human being.

The invention is further directed to cell materials equivalent to the cell materials according to the invention. It is further realized that functionally and even biologically similar cells may be obtained by artificial methods including cloning technologies.

### Mesenchymal Multipotent Cells

The present invention is further directed to mesenchymal stem cells or multipotent cells as preferred cell population according to the invention. The preferred mesenchymal stem cells include cells derived from early human cells, preferably human cord blood or from human bone marrow. In a preferred embodiment the invention is directed to mesenchymal stem cells differentiating to cells of structural support function such as bone and/or cartilage, or to cells forming soft tissues such as adipose tissue.

### Control of Cell Status and Potential Contaminations by Glycosylation Analysis

### Control of Cell Status

### Control of Raw Material Cell Population

The present invention is directed to control of glycosylation of cell populations to be used in therapy.

The present invention is specifically directed to control of glycosylation of cell materials, preferably when
1) there is difference between the origin of the cell material and the potential recipient of transplanted material. In a preferred embodiment there are potential inter-individual specific differences between the donor of cell material and the recipient of the cell material. In a preferred embodiment the invention is directed to animal or human, more preferably human specific, individual person specific glycosylation differences. The individual specific differences are preferably present in mononuclear cell populations of early human cells, early human blood cells and embryonal type cells. The invention is preferably not directed to observation of known individual specific differences such as blood group antigens changes on erythrocytes.
2) There is possibility in variation due to disease specific variation in the materials. The present invention is specifically directed to search of glycosylation differences in the early cell populations according to the present invention associated with infectious disease, inflammatory disease, or malignant disease. Part of the inventors have analysed numerous cancers and tumors and observed similar types glycosylations as certain glycosylation types in the early cells.
3) There is for a possibility of specific inter-individual biological differences in the animals, preferably humans, from which the cell are derived for example in relation to species, strain, population, isolated population, or race specific differences in the cell materials.
4) When it has been established that a certain cell population can be used for a cell therapy application, glycan analysis can be used to control that the cell population has the same characteristics as a cell population known to be useful in a clinical setting.

### Time Dependent Changes During Cultivation of Cells

Furthermore during long term cultivation of cells spontaneous mutations may be caused in cultivated cell materials. It is noted that mutations in cultivated cell lines often cause harmful defects on glycosylation level.

It is further noticed that cultivation of cells may cause changes in glycosylation. It is realized that minor changes in any parameter of cell cultivation including quality and concentrations of various biological, organic and inorganic molecules, any physical condition such as temperature, cell density, or level of mixing may cause difference in cell materials and glycosylation. The present invention is directed to monitoring glycosylation changes according to the present invention in order to observe change of cell status caused by any cell culture parameter affecting the cells.

The present invention is in a preferred embodiment directed to analysis of glycosylation changes when the density of cells is altered. The inventors noticed that this has a major impact of the glycosylation during cell culture.

It is further realized that if there is limitations in genetic or differentiation stability of cells, these would increase probability for changes in glycan structures. Cell populations in early stage of differentiation have potential to produce different cell populations. The present inventors were able to discover glycosylation changes in early human cell populations.

### Differentiation of Cell Lines

The present invention is specifically directed to observe glycosylation changes according to the present invention when differentiation of a cell line is observed. In a preferred embodiment the invention is directed to methods for observation of differentiation from early human cell or another preferred cell type according to the present invention to mesodermal types of stem cell

In case there is heterogeneity in cell material this may cause observable changes or harmful effects in glycosylation.

Furthermore, the changes in carbohydrate structures, even non-harmful or functionally unknown, can be used to obtain information about the exact genetic status of the cells.

The present invention is specifically directed to the analysis of changes of glycosylation, preferably changes in glycan profiles, individual glycan signals, and/or relative abundancies of individual glycans or glycan groups according to the present invention in order to observe changes of cell status during cell cultivation.

### Analysis of Supporting/Feeder Cell Lines

The present invention is specifically directed to observe glycosylation differences according to the present invention, on supporting/feeder cells used in cultivation of stem cells and early human cells or other preferred cell type. It is known in the art that some cells have superior activities to act as a support/feeder cells than other cells. In a preferred embodiment the invention is directed to methods for observation of differences on glycosylation on these supporting/feeder cells. This information can be used in design of novel reagents to support the growth of the stem cells and early human cells or other preferred cell type.

### Contaminations or Alterations in Cells Due to Process Conditions

### Conditions and Reagents Inducing Harmful Glycosylation or Harmful Glycosylation Related Effects to Cells During Cell Handling

The inventors further revealed conditions and reagents inducing harmful glycans to be expressed by cells with same associated problems as the contaminating glycans. The inventors found out that several reagents used in a regular cell purification processes caused changes in early human cell materials.

It is realized, that the materials during cell handling may affect the glycosylation of cell materials. This may be based on the adhesion, adsorption, or metabolic accumulation of the structure in cells under processing.

In a preferred embodiment the cell handling reagents are tested with regard to the presence glycan component being antigenic or harmful structure such as cell surface NeuGc, Neu-O-Ac or mannose structure. The testing is especially preferred for human early cell populations and preferred subpopulations thereof.

The inventors note effects of various effector molecules in cell culture on the glycans expressed by the cells if absorption or metabolic transfer of the carbohydrate structures have not been performed. The effectors typically mediate a signal to cell for example through binding a cell surface receptor.

The effector molecules include various cytokines, growth factors, and their signalling molecules and co-receptors. The effector molecules may be also carbohydrates or carbohydrate binding proteins such as lectins.

Controlled Cell Isolation/Purification and Culture Conditions to Avoid Contaminations with Harmful Glycans or Other Alteration in Glycome Level

### Stress Caused by Cell Handling

It is realized that cell handling including isolation/purification, and handling in context of cell storage and cell culture processes are not natural conditions for cells and cause physical and chemical stress for cells. The present invention allows control of potential changes caused by the stress. The control may be combined by regular methods may be combined with regular checking of cell viability or the intactness of cell structures by other means.

Examples of Physical and/or Chemical Stress in Cell Handling Step

Washing and centrifuging cells cause physical stress which may break or harm cell membrane structures. Cell purifications and separations or analysis under non-physiological flow conditions also expose cells to certain non-physiological stress. Cell storage processes and cell preservation and handling at lower temperatures affects the membrane structure. All handling steps involving change of composition of media or other solution, especially washing solutions around the cells affect the cells for example by altered water and salt balance or by altering concentrations of other molecules effecting biochemical and physiological control of cells.

### Observation and Control of Glycome Changes by Stress in Cell Handling Processes

The inventors revealed that the method according to the invention is useful for observing changes in cell membranes which usually effectively alter at least part of the glycome observed according to the invention. It is realized that this related to exact organization and intact structures cell membranes and specific glycan structures being part of the organization.

The present invention is specifically directed to observation of total glycome and/or cell surface glycomes, these methods are further aimed for the use in the analysis of intactness of cells especially in context of stressful condition for the cells, especially when the cells are exposed to physical and/or chemical stress. It is realized that each new cell handling step and/or new condition for a cell handling step is useful to be controlled by the methods according to the invention. It is further realized that the analysis of glycome is useful for search of most effectively altering glycan structures for analysis by other methods such as binding by specific carbohydrate binding agents including especially carbohydrate binding proteins (lectins, antibodies, enzymes and engineered proteins with carbohydrate binding activity).

Controlled Cell Preparation (Isolation or Purification) with Regard to Reagents

The inventors analysed process steps of common cell preparation methods. Multiple sources of potential contamination by animal materials were discovered.

The present invention is specifically directed to carbohydrate analysis methods to control of cell preparation processes. The present invention is specifically directed to the process of controlling the potential contaminations with animal type glycans, preferably N-glycolylneuraminic acid at various steps of the process.

The invention is further directed to specific glycan controlled reagents to be used in cell isolation

The glycan-controlled reagents may be controlled on three levels:
1. Reagents controlled not to contain observable levels of harmful glycan structure, preferably N-glycolylneuraminic acid or structures related to it
2. Reagents controlled not to contain observable levels of glycan structures similar to the ones in the cell preparation
3. Reagent controlled not to contain observable levels of any glycan structures.

The control levels 2 and 3 are useful especially when cell status is controlled by glycan analysis and/or profiling methods. In case reagents in cell preparation would contain the indicated glycan structures this would make the control more difficult or prevent it. It is further noticed that glycan structures may represent biological activity modifying the cell status.

### Cell Preparation Methods Including Glycan-Controlled Reagents

The present invention is further directed to specific cell purification methods including glycan-controlled reagents.

### Preferred Controlled Cell Purification Process

When the binders are used for cell purification or other process after which cells are used in method where the glycans of the binder may have biological effect the binders are preferably glycan controlled or glycan neutralized proteins.

The present invention is especially directed to controlled production of human early cells containing one or several following steps. It was realized that on each step using regular reagents in following process there is risk of contamination by extragenous glycan material. The process is directed to the use of controlled reagents and materials according to the invention in the steps of the process.

Preferred purification of cells includes at least one of the steps including the use of controlled reagent, more preferably at least two steps are included, more preferably at least 3 steps and most preferably at least steps 1, 2, 3, 4, and 6.
1. Washing cell material with controlled reagent.
2. When antibody based process is used cell material is in a preferred embodiment blocked with controlled Fc-receptor blocking reagent. It is further realized that part of glycosylation may be needed in a antibody preparation, in a preferred embodiment a terminally depleted glycan is used.
3. Contacting cells with immobilized cell binder material including controlled blocking material and controlled cell binder material. In a more preferred the cell binder material comprises magnetic beads and controlled gelatin material according the invention. In a preferred embodiment the cell binder material is controlled, preferably a cell binder antibody material is controlled. Otherwise the cell binder antibodies may contain even N-glycolylneuraminic acid, especially when the antibody is produced by a cell line producing N-glycolylneuraminic acid and contaminate the product.
4. Washing immobilized cells with controlled protein preparation or non-protein preparation.

In a preferred process magnetic beads are washed with controlled protein preparation, more preferably with controlled albumin preparation.
5. Optional release of cells from immobilization.
6. Washing purified cells with controlled protein preparation or non-protein preparation.

In a preferred embodiment the preferred process is a method using immunomagnetic beads for purification of early human cells, preferably purification of cord blood cells.

The present invention is further directed to cell purification kit, preferably an immunomagnetic cell purification kit comprising at least one controlled reagent, more preferably at least two controlled reagents, even more preferably three controlled reagents, even preferably four reagents and most preferably the preferred controlled reagents are selected from the group: albumin, gelatin, antibody for cell purification and Fc-receptor blocking reagent, which may be an antibody.

Contaminations with Harmful Glycans Such as Antigenic Animal Type Glycans

Several glycans structures contaminating cell products may weaken the biological activity of the product.

The harmful glycans can affect the viability during handling of cells, or viability and/or desired bioactivity and/or safety in therapeutic use of cells.

The harmful glycan structures may reduce the in vitro or in vivo viability of the cells by causing or increasing binding of destructive lectins or antibodies to the cells. Such protein material may be included e.g. in protein preparations used in cell handling materials. Carbohydrate targeting lectins are also present on human tissues and cells, especially in blood and endothelial surfaces. Carbohydrate binding antibodies in human blood can activate complement and cause other immune responses in vivo. Furthermore immune defense lectins in blood or leukocytes may direct immune defense against unusual glycan structures.

Additionally harmful glycans may cause harmful aggregation of cells in vivo or in vitro. The glycans may cause unwanted changes in developmental status of cells by aggregation and/or changes in cell surface lectin mediated biological regulation.

Additional problems include allergenic nature of harmful glycans and misdirected targeting of cells by endothelial/cellular carbohydrate receptors in vivo.

### Common Structural Features of all Glycomes and Preferred Common Subfeatures

The present invention reveals useful glycan markers for stem cells and combinations thereof and glycome compositions comprising specific amounts of key glycan structures. The invention is furthermore directed to specific terminal and core structures and to the combinations thereof.

The preferred glycome glycan structure(s) and/or glycomes from cells according to the invention comprise structure(s) according to

the formula C0:

R₁Hexβz{R₃}ₙ₁Hex(NAc)ₙ₂XyR₂,

Wherein

X is glycosidically linked disaccharide epitope β4(Fucα6)ₙGN, wherein n is 0 or 1, or X is nothing and

### Hex is Gal or Man or GlcA,

### HexNAc is GlcNAc or GalNAc,

y is anomeric linkage structure α and/or β or linkage from derivatized anomeric carbon,
z is linkage position 3 or 4, with the provision that when z is 4 then HexNAc is GlcNAc and then Hex is Man or Hex is Gal or Hex is GlcA, and
when z is 3 then Hex is GlcA or Gal and HexNAc is GlcNAc or GalNAc;
n1 is 0 or 1 indicating presence or absence of R3;
n2 is 0 or 1, indicating the presence or absence of NAc, with the proviso that n2 can be 0 only when Hexβz is Galβ4, and n2 is preferably 0, n2 structures are preferably derived from glycolipids;
R₁ indicates 1-4, preferably 1-3, natural type carbohydrate substituents linked to the core structures or nothing;
R₂ is reducing end hydroxyl, chemical reducing end derivative or natural asparagine N-glycoside derivative such as asparagine N-glycosides including asparagine N-glycoside aminoacids and/or peptides derived from protein, or natural serine or threonine linked O-glycoside derivative such as serine or threonine linked O-glycosides including asparagine N-glycoside aminoacids and/or peptides derived from protein, or when n2 is 1 R2 is nothing or a ceramide structure or a derivative of a ceramide structure, such as lysolipid and amide derivatives thereof;
R3 is nothing or a branching structure representing a GlcNAcβ6 or an oligosaccharide with GlcNAcβ6 at its reducing end linked to GalNAc (when HexNAc is GalNAc); or when Hex is Gal and HexNAc is GlcNAc, and when z is 3 then R3 is Fucα4 or nothing, and when z is 4 R3 is Fucα3 or nothing.

The preferred disaccharide epitopes in the glycan structures and glycomes according to the invention include structures Galβ4GlcNAc, Manβ4GlcNAc, GlcAβ4GlcNAc, Galβ3GlcNAc, Galβ3GalNAc, GlcAβ3GlcNAc, GlcAβ3GalNAc, and Galβ4Glc, which may be further derivatized from reducing end carbon atom and non-reducing monosaccharide residues and is in a separate embodiment branched from the reducing end residue. Preferred branched epitopes include Galβ4(Fucα3)GlcNAc, Galβ3(Fucα4)GlcNAc, and Galβ3(GlcNAcβ6)GalNAc, which may be further derivatized from reducing end carbon atom and non-reducing monosaccharide residues.

### Preferred Epitopes for Methods According to the Invention

### N-Acetyllactosamine Galβ3/4GlcNAc Terminal Epitopes

The two N-acetyllactosamine epitopes Galβ4GlcNAc and/or Galβ3GlcNAc represent preferred terminal epitopes present on stem cells or backbone structures of the preferred terminal epitopes for example further comprising sialic acid or fucose derivatisations according to the invention. In a preferred embodiment the invention is directed to fucosylated and/or non-substituted glycan non-reducing end forms of the terminal epitopes, more preferably to fucosylated and non-substituted forms. The invention is especially directed to non-reducing end terminal (non-substituted) natural Galβ4GlcNAc and/or Galβ3GlcNAc-structures from human stem cell glycomes. The invention is in a specific embodiment directed to non-reducing end terminal fucosylated natural Galβ4GlcNAc and/or Galβ3GlcNAc-structures from human stem cell glycomes.

### Preferred Fucosylated N-Acetyllactosamines

The preferred fucosylated epitopes are according to the Formula TF:

(Fucα2)ₙ₁Galβ3/4(Fucα4/3)ₙ₂GlcNAcβ-R

Wherein

n1 is 0 or 1 indicating presence or absence of Fucα2;
n2 is 0 or 1, indicating the presence or absence of Fucα4/3 (branch), and
R is the reducing end core structure of N-glycan, O-glycan and/or glycolipid.

The preferred structures thus include type 1 lactosamines (Galβ3GlcNAc based):

Galβ3(Fucα4)GlcNAc (Lewis a), Fucα2Galβ3GlcNAc H-type 1, structure and,

### Fucα2Galβ3(Fucα4)GlcNAc (Lewis b) and

type 2 lactosamines (Galβ4GlcNAc based):
Galβ4(Fucα3)GlcNAc (Lewis x), Fucα2Galβ4GlcNAc H-type 2, structure and,

### Fucα2Galβ4(Fucα3)GlcNAc (Lewis y).

The type 2 lactosamines (fucosylated and/or terminal non-substituted) form an especially preferred group in context of adult stem cells and differentiated cells derived directly from these. Type 1 lactosamines (Galβ3GlcNAc-structures) are especially preferred in context of embryonal-type stem cells.

### Lactosamines Galβ3/4GlcNAc and Glycolipid Structures Comprising Lactose Structures (Galβ4Glc)

The lactosamines form a preferred structure group with lactose-based glycolipids. The structures share similar features as products of β3/4Gal-transferases. The β3/4 galactose based structures were observed to produce characteristic features of protein linked and glycolipid glycomes.

The invention revealed that furthermore Galβ3/4GlcNAc-structures are a key feature of differentiation related structures on glycolipids of various stem cell types. Such glycolipids comprise two preferred structural epitopes according to the invention. The most preferred glycolipid types include thus lactosylceramide based glycosphingolipids and especially lacto-(Galβ3GlcNAc), such as

lactotetraosylceramide Galβ3GlcNAcβ3Galβ4GlcβCer, preferred structures further including its non-reducing terminal structures selected from the group: Galβ3(Fucα4)GlcNAc (Lewis a), Fucα2Galβ3GlcNAc (H-type 1), structure and, Fucα2Galβ3(Fucα4)GlcNAc (Lewis b) or sialylated structure SAα3Galβ3GlcNAc or SAα3Galβ3(Fucα4)GlcNAc, wherein SA is a sialic acid, preferably Neu5Ac preferably replacing Galβ3GlcNAc of lactotetraosylceramide and its fucosylated and/or elongated variants such as preferably according to the Formula:

(Sacα3)ₙ₅(Fucα2)ₙ₁Galβ3(Fucα4)ₙ₃GlcNAcβ3[Galβ3/4(Fucα4/3)ₙ₂GlcNAcβ3]ₙ₄Galβ4GlcβCer

wherein
n1 is 0 or 1, indicating presence or absence of Fucα2;
n2 is 0 or 1, indicating the presence or absence of Fucα4/3 (branch),
n3 is 0 or 1, indicating the presence or absence of Fucα4 (branch)
n4 is 0 or 1, indicating the presence or absence of (fucosylated) N-acetyllactosamine elongation;
n5 is 0 or 1, indicating the presence or absence of Sacα3 elongation;
Sac is terminal structure, preferably sialic acid, with α3-linkage, with the proviso that when Sac is present, n5 is 1, then n1 is 0
and
neolacto (Galβ4GlcNAc)-comprising glycolipids such as
neolactotetraosylceramide Galβ4GlcNAcβ3Galβ4GlcβCer, preferred structures further including its non-reducing terminal Galβ4(Fucα3)GlcNAc (Lewis x), Fucα2Galβ4GlcNAc H-type 2, structure and, Fucα2Galβ4(Fucα3)GlcNAc (Lewis y)
and
its fucosylated and/or elongated variants such as preferably

(Sacα3/6)ₙ₅(Fucα2)ₙ₁Galβ4(Fucα3)ₙ₃GlcNAcβ3[Galβ4(Fucα3)ₙ₂GlcNAcβ3]ₙ₄Galβ4GlcβCer

n1 is 0 or 1 indicating presence or absence of Fucα2;
n2 is 0 or 1, indicating the presence or absence of Fucα3 (branch),
n3 is 0 or 1, indicating the presence or absence of Fucα3 (branch)
n4 is 0 or 1, indicating the presence or absence of (fucosylated) N-acetyllactosamine elongation,
n5 is 0 or 1, indicating the presence or absence of Sacα3/6 elongation;
Sac is terminal structure, preferably sialic acid (SA) with α3-linkage, or sialic acid with α6-linkage, with the proviso that when Sac is present, n5 is 1, then n1 is 0, and when sialic acid is bound by α6-linkage preferably also n3 is 0.

### Preferred Stem Cell Glycosphingolipid Glycan Profiles, Compositions, and Marker Structures

The inventors were able to describe stem cell glycolipid glycomes by mass spectrometric profiling of liberated free glycans, revealing about 80 glycan signals from different stem cell types. The proposed monosaccharide compositions of the neutral glycans were composed of 2-7 Hex, 0-5 HexNAc, and 0-4 dHex. The proposed monosaccharide compositions of the acidic glycan signals were composed of 0-2 NeuAc, 2-9 Hex, 0-6 HexNAc, 0-3 dHex, and/or 0-1 sulphate or phosphate esters. The present invention is especially directed to analysis and targeting of such stem cell glycan profiles and/or structures for the uses described in the present invention with respect to stem cells.

The present invention is further specifically directed to glycosphingolipid glycan signals specific tostem cell types as described in the Examples. In a preferred embodiment, glycan signals typical to hESC, preferentially including 876 and 892 are used in their analysis, more preferentially FucHexHexNAcLac, wherein α1,2-Fuc is preferential to α1,3/4-Fuc, and Hex₂HexNAc₁Lac, and more preferentially to Galβ3[Hex₁HexNAc₁]Lac. In another preferred embodiment, glycan signals typical to MSC, especially CB MSC, preferentially including 1460 and 1298, as well as large neutral glycolipids, especially Hex₂₋₃HexNAc₃Lac, more preferentially poly-N-acetyllactosamine chains, even more preferentially β1,6-branched, and preferentially terminated with type II LacNAc epitopes as described above, are used in context of MSC according to the uses described in the present invention.

Terminal glycan epitopes that were demonstrated in the present experiments in stem cell glycosphingolipid glycans are useful in recognizing stem cells or specifically binding to the stem cells via glycans, and other uses according to the present invention, including terminal epitopes: Gal, Galβ4Glc (Lac), Galβ4GlcNAc (LacNAc type 2), Galβ3, Non-reducing terminal HexNAc, Fuc, α1,2-Fuc, α1,3-Fuc, Fucα2Gal, Fucα2Galβ4GlcNAc (H type 2), Fucα2Galβ4Glc (2′-fucosyllactose), Fucα3GlcNAc, Galβ4(Fucα3)GlcNAc (Lex), Fucα3Glc, Galβ4(Fucα3)Glc (3-fucosyllactose), Neu5Ac, Neu5Acα2,3, and Neu5Acα2,6. The present invention is further directed to the total terminal epitope profiles within the total stem cell glycosphingolipid glycomes and/or glycomes.

The inventors were further able to characterize in hESC the corresponding glycan signals to SSEA-3 and SSEA-4 developmental related antigens, as well as their molar proportions within the stem cell glycome. The invention is further directed to quantitative analysis of such stem cell epitopes within the total glycomes or subglycomes, which is useful as a more efficient alternative with respect to antibodies that recognize only surface antigens. In a further embodiment, the present invention is directed to finding and characterizing the expression of cryptic developmental and/or stem cell antigens within the total glycome profiles by studying total glycan profiles, as demonstrated in the Examples for α1,2-fucosylated antigen expression in hESC in contrast to SSEA-1 expression in mouse ES cells.

The present invention revealed characteristic variations (increased or decreased expression in comparison to similar control cell or a contaminating cell or like) of both structure types in various cell materials according to the invention. The structures were revealed with characteristic and varying expression in three different glycome types: N-glycans, O-glycans, and glycolipids. The invention revealed that the glycan structures are a characteristic feature of stem cells and are useful for various analysis methods according to the invention. Amounts of these and relative amounts of the epitopes and/or derivatives varies between cell lines or between cells exposed to different conditions during growing, storage, or induction with effector molecules such as cytokines and/or hormones.

### Preferred Binder Molecules for Cell Culture

Preferred binder molecules for the cell culture methods includes lectins, antibodies and glycan modifying enzymes.

### Lectins

It is realized that specific lectin molecules are a preferred group of molecules for maintaining the cell under cell culture. More preferred groups lectins includes plant lectins and animal lectins directed to the terminal glycan epitopes according to the invention.
a) Plant lectins. Plant lectins are especially preferred when these are derived non-mammalian cell cultures or biological materials.
b) Preferred animal lectins includes galectins and selectins.
   Lectins are especially preferred when these are derived from non-animal sources such as plants or non-mammalian or non-animal cell cultures. Preferred cell cultures includes microbial cell cultures, such as bacterial or fungal or yeast cell cultures or plant cell cultures.

Lectins are proteins or glycoproteins, commonly derived from plants or marine animals (lectins from bacteria, viruses, and mammals are also well-known) that have binding specificity for a particular sugar or sugars, usually a mono- or disaccharide structure. For example, Concanvalin A (Con A) binds .alpha.-D-Glc and .alpha.-D-Man. Lectin binding, like antibody binding to antigen, is noncovalent and reversible (typically by a sufficient concentration of the saccharide ligand. Thus, for example, a solution of glucose or mannose (or .alpha.-methylmannoside-) will release Con A that has bound to cells or to an immobilized glycoprotein. For thorough description of plant lectins, see, for example, EJM Van Damme et al., Handbook of Plant Lectins: Properties and Biomedical Applications John Wiley & Sons, New York, 1998; see also the web site http://www.plab.ku.dk/tcbh/ and http://www.vectorlabs.com/Lecti-ns/Lindex.html for commercially available lectins. Other useful reviews include Goldstein, I J et al., 1978, Adv. Carbohydr. Chem. Biochem. 35:127-340; D. Mirelman (ed.), Microbial Lectins and Agglutinins: Properties and Biological Activity, Wiley, N.Y. (1986); Goldstein I J, Indian J Biochem Biophys, 1990, 27:368-369.

Lectins can be immobilized directly on the surface (passively), or, as with antibodies, can be used in a sandwich fashion where a first lectin binding protein has binding specificity and affinity for the lectin (such as an anti-lectin antibody or streptavidin when the lectin is biotinylated) and the lectin serves as a binder and is bound noncovalently to the first lectin binding protein. The lectin acts as the capture agent to bind its specific target preferably a cell that displays a particular glycan structure on a cell surface. Typically, such glycan structures are in the form of carbohydrate chains on glycoproteins or glycolipids.

Table A below lists a number of useful lectins and their sugar-binding specificities.

Also included in the present invention as an lectin is a covalently coupled lectin-antibody or lectin-antigen conjugate (see, e.g., Chu, U.S. Pat. No. 4,493,793).

Yet another class of binder in the present invention is a basic molecules that has affinity for the lipid bilayer of the cell membrane, for example, protamine and the membrane binding portion of the bee venom peptide, mellitin. While these target structures may not formally be considered “ligands” the concept is the same—affinity capture of cells which bind to this binder when it is immobilized to a solid surface.

TABLE A
Lectins and their Binding Specificity

Lectin (agglutinin) Abbrev Carbohydrate Specificity
Allium sativum (garlic bulb) ASA .alpha.(1,3)-linked Man units
Arachis hypogaea (peanut) PNA Gal(.beta.1,3)-GalNAc
Bauhinia purpurea BPA GalNAc, Gal
Bendeirea simplicifolia BSA .alpha.-Gal
Canavalia ensorformis (jackbean) Con A .alpha.-Man, .alpha.-Glc
Crocus vernus (Crocus bulb) terminal Man(.alpha.1,3)Man
Dolichos biflorus (horse gram) DBA GalNAc
Erythrina cristagalli (coral tree) ECA Gal(.beta.1,4)GlcNAc
Glycine max (soybean) SBA Gal, GalNAc
Griffonia simplicifolia-1 GS-1 N-linked glycans from murine IgD
Griffonia simplicifolia-1-B4 GS-1-B4 Gal (.alpha.1,3)Gal
Griffonia simplicifolia 1-A4 GS I-A4 terminal .alpha.GalNAc
Helix pomatia HPA GalNAc
Lens culinaris (lentil) LcH .alpha.-Man, .alpha.-Glc
Limulus polyhemus (horseshoe LPA Sialic Acid (“NeuAc5”) crab)
Lotus tetragonolobus Lotus A .alpha.-L-Fucose
Marasmius oreades (mushroom) MOA Gal(.alpha. 1,3)Gal
Musa acuminata (banana) BanLec .alpha.-Man; .alpha.-Glc (internal .alpha. 1,3-linked Glc in
certain linear polysaccharides, .beta. 1,3-linked glucosyl oligosaccharides and .beta. 1,6-linked
glucosyl end groups)
Narcissus pseudonarcissus, NPA Man/Glc type structures
Phaseolus limensis LBA I .alpha.-D-GalNAc
Phaseolus lunatus (lima bean) LBL, GalNAc(.alpha.1,3)Fuc(.alpha.1,2)Gal(.beta.1,R).
Phaseolus vulgaris (red kidney bean) PHA-L GalNAc PHA-H GalNAc PHA-E
Oligosaccharide Pisum sativum (pea) PEA .alpha.-D-Man, .alpha.-D-Glc.
Phytolacca americana (pokeweed) PWM (GlcNAc).sub.3
Polysporus squamosus (mushroom) PSL NeuAc5(.alpha.2,6)Gal(.beta.1,-4)Glc/GlcNAc (of
N-linked oligosacch
Ricinus communis (castor bean) RCA I .beta.-D-Gal RCA II .beta.-D-Gal, D-GalNAc
Sambucus nigra (elderberry bark) SNA NeuAc5(.alpha.2,6)Gal/GalNAc (does not
discriminate between O-linked and N-linked oligosaccharides
Sophora japonica (pagoda tree) SJA .alpha.GalNAc
Triticum vulgaris (wheat germ) WGA (GlcNAc).sub.2; NeuAc5
Ulex Europaeus (Furze gorse) UEA I .alpha.-L-Fucose UEA II (GlcNAc).sub.2
Wisteria Floribunda (Japanese Wister) WFA GalNAc

Contacting the Stem Cells with the Binder

The prepared stem cells can be contacted with a binder. This can be done, for example, by simply mixing the binder with the culture of stem cell preparations. Mixing can be performed in a plethora of suitable vessels capable of maintaining viability of the stem cells. Said vessels can include but are not limited to tissue culture flasks, conical tubes, culture bags, bioreactors, or cultures that are continuously mixed. The stem cell/LPCM mixture can then be allowed to grow as desired.

The prepared stem cells can be contacted with a binder on a surface. This can be done, for example, by coating the binder on the culture plate. Coating can be performed in a plethora of suitable vessels capable of maintaining viability of the stem cells. Said vessels can include but are not limited to tissue culture flasks, conical tubes, culture bags, bioreactors, or cultures. The stem cell population can then be allowed to grow as desired. An example of stem cell growth and contacted with a binder is shown in Examples 10 and 22. A coating process of a cell culture well is shown in Example 10.

Methods of coating culture plates and vessels suitable of maintaining viability of the stem cells are known for the skilled artisan. Typically, an agent, for example, a binder of the present invention is applied on the surface of the culture plate in a buffer, allowed to adhere overnight, washed and stem cells are plated onto the wells and grown. Skilled artisan can consult e.g. Pierce Instruction Book (www.piercenet.com/) for further protocols for coating and covalently linking binders of the present invention on surfaces and for use of stem cell cultures.

As indicated above, the methods of the present invention preferably use binders bound to a surface. The surface may be any surface capable of having a binder bound thereto or integrated into and that is biocompatible, that is, substantially non-toxic to the target cells to be stimulated. The biocompatible surface may be biodegradable or non-biodegradable. The surface may be natural or synthetic, and a synthetic surface may be a polymer. Other polymers may include polyesters, polyethers, polyanhydrides, polyalkylcyanoacrylates, polyacrylamides, polyorthoesters, polyphosphazenes, polyvinylacetates, block copolymers, polypropylene, polytetrafluorethylene (PTFE), or polyurethanes. The polymer may be lactic acid or a copolymer. A copolymer may comprise lactic acid and glycolic acid (PLGA). Non-biodegradable surfaces may include polymers, such as poly(dimethylsiloxane) and poly(ethylene-vinyl acetate). Biocompatible surfaces include for example, glass (e.g., bioglass), collagen, metal, hydroxyapatite, aluminate, bioceramic materials, hyaluronic acid polymers, alginate, acrylic ester polymers, lactic acid polymer, glycolic acid polymer, lactic acid/glycolic acid polymer, purified proteins, purified peptides, or extracellular matrix compositions. Other polymers comprising a surface may include glass, silica, silicon, hydroxyapatite, hydrogels, collagen, acrolein, polyacrylamide, polypropylene, polystyrene, nylon, or any number of plastics or synthetic organic polymers, or the like. The surface may comprise a biological structure, such as a liposome. The surface may be in the form of a lipid, a plate, bag, pellet, fiber, mesh, or particle. A particle may include, a colloidal particle, a microsphere, nanoparticle, a bead, or the like. In the various embodiments, commercially available surfaces, such as beads or other particles, are useful (e.g., Miltenyi Particles, Miltenyi Biotec, Germany; Sepharose beads, Pharmacia Fine Chemicals, Sweden; DYNABEADS™, Dynal Inc., New York; PURABEADS™, Prometic Biosciences).

When beads are used, the bead may be of any size that effectuates target cell stimulation. In one embodiment, beads are preferably from about 5 nanometers to about 500 um in size. Accordingly, the choice of bead size depends on the particular use the bead will serve. For example, when separation of beads by filtration is desired, bead sizes of no less than 50 .mu.m are typically used. Further, when using paramagnetic beads, the beads typically range in size from about 2.8 .mu.m to about 500 .mu.m and more preferably from about 2.8 .mu.m to about 50 .mu.m. Lastly, one may choose to use super-paramagnetic nanoparticles which can be as small as about 10 nm. Accordingly, as is readily apparent from the discussion above, virtually any particle size may be utilized.

A binder may be attached or coupled to, or integrated into a surface by a variety of methods known and available in the art. The attachment may be covalent or noncovalent, electrostatic, or hydrophobic and may be accomplished by a variety of attachment means, including for example, chemical, mechanical, enzymatic, or other means whereby a binder is capable of stimulating/modulating the cells. For example, the antibody first may be attached to a surface, or avidin or streptavidin may be attached to the surface for binding to a biotinylated binder/antibody. The antibody may be attached to the surface via an anti-idiotype antibody. Another example includes using protein A or protein G, or other non-specific antibody binding molecules, attached to surfaces to bind an antibody. Alternatively, the binder may be attached to the surface by chemical means, such as cross-linking to the surface, using commercially available cross-linking reagents (Pierce, Rockford, Ill.) or other means. In certain embodiments, the binders are covalently bound to the surface. Further, in one embodiment, commercially available tosyl-activated DYNABEADS™ or DYNABEADS™ with epoxy-surface reactive groups are incubated with the polypeptide binder of interest according to the manufacturer's instructions. Briefly, such conditions typically involve incubation in a phosphate buffer from pH 4 to pH 9.5 at temperatures ranging from 4 to 37 degrees C.

### Covalent Coupling

Surfaces coated with binder are described above and in the Examples. Coating with binders, e.g. lectins and antibodies, can be performed by series of chemical coupling reactions involving creation of two reactive aldehyde groups the methods of which are knows for skilled artisan. For example and not bound to any particular theory, when a aldehyde moiety (RCHO) reacts with a primary amine moiety (R′NH.sub.2), an equilibrium is established with the reaction product, which is a relatively unstable imine moiety (R′N CHR). Coupling reaction can be carried out under the same conditions as for the oxidation, which are designed to protect the glycoprotein from damage. To stabilize the linkage between the glycoprotein and the biomaterial surface, subsequent reductive alkylation of the imine moiety is carried out using reducing agents (i.e., stabilizing agents) such as, for example, sodium borohydride, sodium cyanoborohydride, and amine boranes, to form a secondary amine (R′NH—CH.sub.2R). This reaction can also be carried out under the same conditions as for the oxidation. Typically, however, the coupling and stabilizing reactions are carried out in a neutral or slightly basic solution and at a temperature of about 0-50.degree. C. Preferably, the pH is about 6-10, and the temperature is about 4-37.degree. C., for the coupling and stabilizing reactions. These reactions (coupling and stabilizing) can be allowed to proceed for just a few minutes or for many hours. Commonly, the reactions are complete (i.e., coupled and stabilized) within 24 hours.

In one aspect, the binder, such as certain lectins may be of singular origin or multiple origins and may be antibodies or fragments thereof. These binders are coupled to the surface by any of the different attachment means discussed above.

The lectin ECA molecule to be coupled to the surface may be isolated e.g. from a plant cell expressing it. Fragments, mutants, or variants of the ECA lectin molecule that retain the capability to bind and maintain hESC in undifferentiated state can also be used. Furthermore, one of ordinary skill in the art will recognize that any binder useful in the activation/modulation of proliferation/adherence/morphology/growth status of a subset of stem cells may also be immobilized on beads or culture vessel surfaces or any surface. In addition, while covalent binding of the binder to the surface is one preferred methodology, adsorption or capture by a secondary monoclonal antibody may also be used. The amount of a particular binder attached to a surface may be readily determined by flow cytometry (FACS) analysis if the surface is that of beads or determined by enzyme-linked immunosorbant assay (ELISA) if the surface is a tissue culture dish, mesh, fibers, bags, for example.

In some situations it will be desirable to use a combination culture system in which cells are first grown in contact with a binder and then subsequently in another culture condition, e.g. when differentiating cells. For example, stem cells can be passaged in contact with a binder and subsequently cytokines and/or growth factors are added to differentiate and/or modulate biological characteristics of the stem cells

Cytokine can be IL-3, IL-6, SCF, TPO, and flt-3L.

The concentration of a binder, for example, immobilized on a surface can be determined by one of skill in the art.

The binder concentration can vary, for example, depending on temperature, incubation time, number of stem cells, the desired activity sought in the stem cells, the type of stem cells, the purity of stem cells, and the like. The stem cells can be isolated from their original source, grown in the presence of feeder layer and contacted with the binder, or the stem cells can be isolated from their source and contacted with the binder. Preferably, hESC are obtained from blastocysts and cultured on binder coated culture plates.

The present invention is directed to stem cell growth promoting and/or modulating coating densities of surfaces with lectin, i.e. coating densities which promote growth and/or modulation of stem cells, preferably human embryonic stem cells. It is realized that the exact efficient densities are dependant on surface geometry and texture. As described in Examples, the inventors were able to obtain efficient coating of growth-supporting surface with lectin. An abundance of coating molecule may be needed to obtain a suitable coating density of lectin protein/surface area, and a skilled artisan is able to obtain a preferred coating efficiency according to the present invention, preferably 1 ng-1000 ng protein/cm² surface area, more preferably 10 ng-1000 ng/cm², even more preferably 100 ng-900 ng/cm², or most preferably 200 ng-800 ng/cm². Efficient coating densities based on surface geometry are known to a skilled artisan and described in the literature, for example, in Nunc Bulletin No. 6 “Principles in adsorption to polystyrene” available from the manufacturer of Nunc microtiter well plates.

Furthermore, conditions promoting certain type of cellular proliferation or differentiation can be used during the culture. These conditions include but are not limited to, alteration in temperature, alternation in oxygen/carbon dioxide content, alternations in turbidity of said media, or exposure to small molecules modifiers of cell cultures such as nutrients, inhibitors of certain enzymes, stimulators of certain enzymes, inhibitors of histone deacetylase activity such as valproic acid (Bug, et al., 2005, Cancer Res 65:2537-2541), trichostatin-A (Young, et al., 2004, Cytotherapy 6:328-336), trapoxin A (Kijima, et al., 1993, J Biol Chem 268:22429-22435), or Depsipeptide (Gagnon, et al., 2003, Anticancer Drugs 14:193-202; Fujieda, et al., 2005, Int J Oncol 27:743-748), each of which is incorporated by reference herein in its entirety, inhibitors of DNA methyltransferase activity such as 5-azacytidine, inhibitors of the enzyme GSK-3 (Trowbridge, et al., 2006, Nat Med 12:89-98, which is incorporated by reference herein in its entirety), and the like.

A variety of factors previously mentioned influence ability of stem cells to survive, replicate, and differentiate. For example, in terms of nutrients the amino acid taurine under certain conditions preferentially inhibits murine bone marrow cells from forming osteoclasts (Koide, et al., 1999, Arch Oral Biol 44:711-719), the amino acid L-arginine stimulates erythrocyte differentiation and proliferation of erythroid progenitors (Shima, et al., 2006, Blood 107:1352-1356), extracellular ATP acting through P2Y receptors mediates a wide variety of changes to both hematopoietic and non-hematopoietic stem cells (Lee, et al., 2003, Genes Dev 17:1592-1604), arginine-glycine-aspartic acid attached to porous polymer scaffolds increase differentiation and survival of osteoblast progenitors (Hu, et al., 2003, J Biomed Mater Res A 64:583-590), each of which is incorporated by reference herein in its entirety. Accordingly, one skilled in the art would know to use various types of nutrients for inducing differentiation, or maintaining viability, of certain types of stem cells and/or progeny thereof.

### Stimulation of a Cell Population

The methods of the present invention relates to the stimulation of a stem cell by contacting a binder that binds to a terminal glycan structure. Binding of the binder to the cell may trigger a signaling pathway that in turn activates particular phenotypic or biological changes in the cell. The activation of the cell may enhance normal cellular functions or initiate normal cell functions in an abnormal cell.

Stimulation of a cell may be enhanced or a particular cellular event may be stimulated by introducing a binder. This method may be applied to any stem cell for which ligation of a cell surface terminal glycan structure leads to a signaling event. The invention further provides means for selection or culturing the stimulated/modulated stem cells.

The prototypic example described is stimulation of mesenchymal stem cells (see Examples, but one of ordinary skill in the art will readily appreciate that the method may be applied to other stem cell types. By way of example, cell types that may be stimulated and selected include hematopoietic stem cells and hematopoietic progenitor cells (CD34+ cells), pluripotent stem cells, and multi-potent stem cells, etc. Accordingly, the present invention also provides populations of cells resulting from this methodology as well as cell populations having distinct phenotypical characteristics, including mesenchymal stem cells with specific phenotypic characteristics.

Two examples are given below that illustrate how such a binding of cell surface glycan structures could be of practical benefit.

In one example, normal mesenchymal stem cell activation by binder (se lectins in Examples) results in morphological changes and changes in adherence, for example. Using man-made approaches, such as those described herein, in the absence of “normal” in-vivo activation, one could accelerate, improve, or otherwise affect the functions described above, in particular through the accelerated, controlled, and spatially oriented ligation of glycan bearing proteins. Benefits could be improved cell expansion in vitro resulting in higher numbers of infuseable and more robust cells for therapeutic applications. Other benefits could be improved cell adherence to surfaces.

Prior to expansion, a source of stem cells is obtained from a subject. The term “subject” is intended to include living organisms in which an immune response can be elicited (e.g., mammals). Examples of subjects include humans, dogs, cats, mice, rats, and transgenic species thereof.

Using methodologies of the present invention it may be advantageous to maintain long-term stimulation/modulation of a population of stem cells. Of particular preference is human embryonic stem cells which can be maintained in an undifferentiated state for several passages and which maintain their phenotypic characteristics.

In a preferred embodiment, a surface of a culture flask is coated with a binder, e.g. ECA lectin (with or without an intermediate layer) and a population of human embryonic stem cells is added to the surface and allowed to adhere.

The surface of the present invention can be prepared with the binder distributed in any pattern or array, such as a microarray pattern of dots arranged in preselected patterns on the, polymer, surface. Thus, for example, microarrays of one or more different types of binders, for example lectins or antibodies, may be immobilized to a surface as described herein. In addition to growth or binding or modulation or intact cells, the coated surfaces described herein, for example in the form of an antibody and/or lectin microarray, are used to detect or quantitate or modulate the growth, adherence, or morphology of stem cells, or any of a number of corresponding antigens or epitopes on stem cells, stem cell lysate or other subcellular preparation. Thus, the present invention provides a method for producing a device comprising a high density array of binders of the present invention, such as antibodies or lectins for stem cell modulation and/or analysis. Such a device may is useful in a method for quantitating expression levels of specific glycan structures in a stem cell population, for example, cells treated in vitro in a selected manner to induce differentiation or another cellular activity. These devices and methods can be readily adapted to high throughput analysis of stem cells treated (or not treated) with a test agent such as a drug or induced to differentiate. For example, stem cells can be contacted with binder, preferably grown on a binder coated array, and treated with various drugs followed by lysing and taking lysates, or culture supernatants can be taken, and analysed.

hESCs

The pluripotent ES cells of the present disclosure are lineage uncommitted (i.e., they are not committed to a particular germ lineage such as ectoderm, mesoderm and endoderm). Pluripotent human ES cells may also have a high self-renewal capacity and possess differentiation potential, both in vitro and in vivo, or can remain dormant or quiescent within a cell, tissue, or organ. The isolated blastocyst from which human ES cells are isolated may be produced by a number of methods well known to those skilled in the art, such as in vitro fertilization, intracytoplasmic sperm injection, and ooplasm transfer. In certain embodiments, the isolated human ES cells are grown on embryonic fibroblast cells including, but not limited to, mouse embryonic fibroblasts, human embryonic fibroblasts or fibroblast-like cells derived from adult human tissues. In a preferred embodiments, the human ES cells are grown in the presence of a binder.

A population of human ES cells derived from blastocysts, as described in the preferred embodiments, express specific markers of ES cells, including but not limited to, Oct-4, Nanog, Rex1, Sox-2, FGF4, Utf1, Thy1, Criptol, ABCG2, Dppa5, hTERT, Connexin-43, Connexin-45. Human ES cells do not express markers characteristic of differentiated cells, such as Keratin 5, Keratin 15, Keratin 18, Sox-1, NFH (ectoderm); brachyury, Msx1, MyoD, HAND1, cardiac actin (mesoderm); GATA4, AFP, HNF-4-a, HNF-30, albumin, and PDX 1 (endoderm). The human ES cells also express cell surface markers such as stage specific embryonic antigen 3 (SSEA-3), SSEA-4, tumor-recognition antigen 1-60 (TRA-1-60), TRA-1-81, Oct-4, E-cadherin, Connexin-43, and alkaline phosphatase. Expression levels may be detected by immunocytochemistry. The extensive molecular characterization of the human ES cell lines of the present disclosure may provide invaluable insight into early embryonic development.

In certain embodiments of the present disclosure, isolated human ES cells are cultured in a nutrient medium, preferably which comprises growth factors, and maintained by manual passaging. As used herein the term “growth factor” refers to proteins that bind to cell surface receptors with the primary result of activating cellular proliferation and differentiation through the activation of signaling pathways. The majority of growth factors/supplements are quite versatile and capable of stimulating cellular division in numerous different cell types, while the specificity of some growth factors is restricted to certain cell types. Growth factors may be used that are specific to pluripotent ES cells and their induction to differentiate into various lineages such as neurons, hepatocytes, cardiomyocytes, beta-islets, chondrocytes, osteoblast, myocytes, and the like. An example of ES cell media contains 80% DMEM/F-12, 15% ES-tested FBS, 5% Serum replacement, 1% nonessential amino acid solution, 1 mM glutamine (GIBCO), 0.1% beta mercaptoethanol, 4 ng/ml human bFGF and 10 ng/ml human Leukemia inhibitory factor (LIF). The method of manually passaging the cells is advantageous over the commonly used method of passaging by enzymatic treatment, because it helps to maintain the genetic stability of the cell line. Maintenance of the normal karyotype of a cell line is important for its use in therapeutic purposes.

### Preferred Epitopes and Antibody Binders Especially for Analysis of Embryonal Stem Cells

The antibody labelling experiment Table 19 with embryonal stem cells revealed specific of type 1 N-acetyllactosamine antigen recognizing antibodies recognizing non-modified disaccharide Galβ3GlcNAc (Le c, Lewis c), and fucosylated derivatives H type and Lewis b. The antibodies were effective in recognizing hESC cell populations in comparison to mouse feeder cells mEF used for cultivation of the stem cells.

Specific different H type 2 recognizing antibodies were revealed to recognize different subpopulations of embryonal stem cells and thus usefulness for defining subpopulations of the cells. The invention further revealed a specific Lewis x and sialyl-Lewis x structures on the embryonal stem cells (see Figures of the present invention).

Preferred Epitopes and Lectin Binders for hESC (See Figures of the Present Invention)

Other preferred binders and/or lectins comprise of binders which bind to the same epitope than ECA (Erythrina cristacalli). In a preferred embodiment, the lectin binds to XXXX epitope. A more preferred lectin comprises of the lectin ECA. This epitope is useful for growth of stem cells or modulation of the status of stem cells or subset of stem cells. In a more preferred embodiment stem cells comprise human embryonic stem cells. The ECA coated surface(s), preferably culture plates, is a preferred embodiment of the present invention. In a preferred embodiment hESC are grown on an ECA coated surface and essentially feeder cell free. Preferably, ECA coated surfaces maintain hESC substantially in undifferentiated state. In a preferred embodiment, hESC are obtained directly from blastocysts without the exposure to mouse feeder cells. In another preferred embodiment hESC culture media comprises a conditioned media, preferably with mEF or hEF conditioned. Preferably, hESC are grown on mouse feeder cells and transferred to grow on ECA coated plates. In a more preferred embodiment hESC are obtained from a blastocyst and grown on ECA coated surfaces.

Other preferred binders and/or antibodies comprise of binders which bind to the same epitope than GF 287 (H type 1). In a preferred embodiment, an antibody binds to Fucα2Galβ3GlcNAc epitope. A more preferred antibody comprises of the antibody of clone 17-206 (ab3355) by Abcam. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells. The binder(s) and epitope recognized by it is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other preferred binders and/or antibodies comprise of binders which bind to the same epitope than GF 279 (Lewis c, Galβ3GlcNAc). In a preferred embodiment, an antibody binds to Galβ3GlcNAc epitope in glycoconjugates, more preferably in glycoproteins and glycolipids such as lactotetraosylceramide. A more preferred antibody comprises of the antibody of clone K21 (ab3352) by Abcam. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells. The binder(s) and epitope recognized by it is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other preferred binders and/or antibodies comprise of binders which bind to the same epitope than GF 288 (Globo H). In a preferred embodiment, an antibody binds to Fucα2Galβ3GalNAcβ epitope, more preferably Fucα2Galβ3GalNAcβ3GalαLacCer epitope. A more preferred antibody comprises of the antibody of clone A69-A/E8 (MAB-S206) by Glycotope. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells. The binder(s) and epitope recognized by it is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other preferred binders and/or antibodies comprise of binders which bind to the same epitope than GF 284 (H type 2). In a preferred embodiment, an antibody binds to Fucα2Galβ4GlcNAc epitope. A more preferred antibody comprises of the antibody of clone B393 (DM3015) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells. The binder(s) and epitope recognized by it is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other preferred binders and/or antibodies comprise of binders which bind to the same epitope than GF 283 (Lewis b). In a preferred embodiment, an antibody binds to Fucα2Galβ3(Fucα4)GlcNAc epitope. A more preferred antibody comprises of the antibody of clone 2-25LE (DM3122) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells. The binder(s) and epitope recognized by it is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other preferred binders and/or antibodies comprise of binders which bind to the same epitope than GF 286 (H type 2). In a preferred embodiment, an antibody binds to Fucα2Galβ4GlcNAc epitope. A more preferred antibody comprises of the antibody of clone B393 (BM258P) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells. The binder(s) and epitope recognized by it is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other preferred binders and/or antibodies comprise of binders which bind to the same epitope than GF 290 (H type 2). In a preferred embodiment, an antibody binds to Fucα2Galβ4GlcNAc epitope. A more preferred antibody comprises of the antibody of clone A51-B/A6 (MAB-S204) by Glycotope. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells. The binder(s) and epitope recognized by it is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other binders binding to feeder cells, preferably mouse feeder cells, comprise of binders which bind to the same epitope than GF 285 (H type 2). In a preferred embodiment, an antibody binds to Fucα2Galβ4GlcNAc, Fucα2Galβ3(Fucα4)GlcNAc, Fucα2Galβ4(Fucα3)GlcNAc epitope. A more preferred antibody comprises of the antibody of clone B389 (DM3014) by Acris. This epitope is suitable and can be used to detect, isolate and evaluate of feeder cells, preferably mouse feeder cells in culture with human embryonic stem cells. The detection can be performed in vitro, for FACS purposes and/or for cell lineage specific purposes. This antibody can be used to positively isolate and/or separate and/or enrich feeder cells (negatively select stem cells), preferably mouse embryonic feeder cells from a mixture of cells comprising feeder and stem cells.

Other binders binding to stem cells, preferably human stem cells, comprise of binders which bind to the same epitope than GF 289 (Lewis y). In a preferred embodiment, an antibody binds to Fucα2Galβ4(Fucα3)GlcNAc epitope. A more preferred antibody comprises of the antibody of clone A70-C/C8 (MAB-S201) by Glycotope. This epitope is suitable and can be used to detect, isolate and evaluate of stem cells, preferably human stem cells in culture with feeder cells. The detection can be performed in vitro, for FACS purposes and/or for cell lineage specific purposes. This antibody can be used to positively isolate and/or separate and/or enrich stem cells (negatively select feeder cells), preferably human stem cells from a mixture of cells comprising feeder and stem cells. The binder(s) and epitope recognized by it is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

The staining intensity and cell number of stained stem cells, i.e. glycan structures of the present invention on stem cells indicates suitability and usefulness of the binder for isolation and differentiation marker. For example, low relative number of a glycan structure expressing cells may indicate lineage specificity and usefulness for selection of a subset and when selected/isolated from the colonies and cultured. Low number of expression is less than 5%, less than 10%, less than 15%, less than 20%, less than 30% or less than 40%. Further, low number of expression is contemplated when the expression levels are between 1-10%, 10%-20%, 15-25%, 20-40%, 25-35% or 35-50%. Typically, FACS analysis can be performed to enrich, isolate and/or select subsets of cells expressing a glycan structure(s).

High number of glycan expressing cells may indicate usefulness in pluripotency/multipotency marker and that the binder is useful in identifying, characterizing, selecting or isolating pluripotent or multipotent stem cells in a population of mammalian cells. High number of expression is more than 50%, more preferably more than 60%, even more preferably more than 70%, and most preferably more than 80%, 90 or 95%. Further, high number of expression is contemplated when the expression levels are between 50-60, 55%-65%, 60-70%, 70-80, 80-90%, 90-100 or 95-100%. Typically, FACS analysis can be performed to enrich, isolate and/or select subsets of cells expressing a glycan structure(s).

The epitopes recognized by the binders GF 279, GF 287, and GF 289 and the binders are particularly useful in characterizing pluripotency and multipotency of stem cells in a culture. The epitopes recognized by the binders GF 283, GF 284, GF 286, GF 288, and GF 290 and the binders are particularly useful for selecting or isolating subsets of stem cells. These subset or subpopulations can be further propagated and studied in vitro for their potency to differentiate and for differentiated cells or cell committed to a certain differentiation path.

The percentage as used herein means ratio of how many cells express a glycan structure to all the cells subjected to an analysis or an experiment. For example, 20% stem cells expressing a glycan structure in a stem cell colony means that a binder, eg an antibody staining can be observed in about 20% of cells when assessed visually.

In colonies a glycan structure bearing cells can be distributed in a particular regions or they can be scattered in small patch like colonies. Patch like observed stem cells are useful for cell lineage specific studies, isolation and separation. Patch like characteristics were observed with GF 283, GF 284, GF 286, GF 288, and GF 290.

For positive selection of feeder cells, preferably mouse feeder cells, most preferably embryonic fibroblasts, GF 285 is useful. This antibody has lower specificity and may have binding to e.g. Lewis y, which has been observed also in mEF cells. It stains almost all feeder cells whereas very little if at all staining is found in stem cells. The antibody was however under optimized condition revealed to bind to thin surface of embryonal bodies, this was in complementary to Lewis y antibody to the core of embryoid body. For all percentages of expression, see Table 19.

### Mesenchymal Stem Cells and Differentiated Tissue Type Stem Cells Derived Thereof.

### Antibodies Useful for Evaluation of Differentiation Status of Mesenchymal Stem Cells

Example 14 and Table 19 (lower part) shows labelling of mesenchymal stem cells and differentiated mesenchymal stem cells

Invention revealed that structures recognized by antibody GF303, preferably Fucα2Galβ3GlcNAc, and GF276 appear during the differentiation of mesenchymal stem cells to osteogenically differentiated stem cells. It was further revealed, that the GalNAcα-group structures GF278, corresponding to Tn-antigen, and GF277, sialyl-Tn increase simultaneously.

The invention is further directed to the preferred uses according to the invention for binders to several target structures, which are characteristic to both mesenchymal stem cells (especially bone marrow derived) and the osteogenically differentiated mesenchymal stem cells. The preferred target structures include one GalNAcα-group structure recognizable by the antibody GF275, the antigen of the antibody is preferably sialylated O-glycan glycopeptide epitope as known for the antibody. The epitopes expressed in both mesenchymal and the osteonically differentiated stem cells further includes two characteristic globo-type antigen structures: the antigen of GF298, which binding correspond to globotriose(Gb3)-type antigens, and the antigen of GF297, which correspond to globotetraose(Gb4) type antigens. The invention has further revealed that terminal type two lactosamine epitopes are especially expressed in both types of mesenchymal stem cells and this was exemplified by staining both cell by antibody recognizing H type II antigen in Example 14 Table 19.

The invention is further directed to the preferred uses according to the invention for binders to several target structures which are substantially reduced or practically diminished/reduced to non-observable level when mesenchymal stem cells (especially bone marrow derived) differentiates to more differentiated, preferably osteogenic mesenchymal stem cells. These target structures include two globoseries structures, which are preferably Galactosyl-globoside type structure, recognized as antigen SSEA-3, and sialyl-galactosylgloboside type structure, recognized as antigen SSEA-4. The preferred reducing target structures further include two type two N-acetyllactosamine target structures Lewis x and sialyl-Lewis x. Globoside-type glycosphingolipid structures were detected by the inventors in MSC in minor but significant amounts compared to hESC in direct structural analysis, more specifically glycan signals corresponding to SSEA-3 and SSEA-4 glycan antigen monosaccharide compositions. These antigens were also detected by monoclonal antibodies in MSC. The present invention is therefore specifically directed to these globoside structures in context of MSC and cells derived from them in uses described in the invention.

In a preferred embodiment of the present invention, the antibodies or binders which bind to the same epitope than GF275, GF277, GF278, GF297, GF298, GF302, GF305, GF307, GF353, or GF354 are useful to detect/recognize, preferably bone marrow derived, mesenchymal stem cells (corresponding epitopes recognized by the antibodies are listed in Example 314). These epitopes are suitable and can be used to detect, isolate and evaluate of (mesenchymal) stem cells, preferably bone marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow from mixture of cells comprising other, bone marrow derived, cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other binders binding to stem cells, preferably human stem cells, comprise of binders which bind to the same epitope than GF275 (sialylated carbohydrate epitope of the MUC-1 glycoprotein). A more preferred antibody comprises of the antibody of clone BM3359 by Acris. This epitope is suitable and can be used to detect, isolate and evaluate of (mesenchymal) stem cells, preferably bone marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow, or differentiated in osteogenic direction from mixture of cells comprising other, bone marrow derived, cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other binders binding to stem cells, preferably human stem cells, comprise of binders which bind to the same epitope than GF305 (lewis x). A more preferred antibody comprises of the antibody of clone CBL144 by Chemicon. This epitope is suitable and can be used to detect, isolate and evaluate of (mesenchymal) stem cells, preferably bone marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow from mixture of cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other binders binding to stem cells, preferably human stem cells, comprise of binders which bind to the same epitope than GF307 (sialyl lewis x). A more preferred antibody comprises of the antibody of clone MAB2096 by Chemicon. This epitope is suitable and can be used to detect, isolate and evaluate of (mesenchymal) stem cells, preferably bone marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow from mixture of cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

In a preferred embodiment, the antibodies or binders which bind to the same epitope than GF305, GF307, GF353 or GF354 are useful for positive selection and/or enrichment of mesenchymal stem cells (corresponding epitopes recognized by the antibodies are listed in Example 14).

In another preferred embodiment of the present invention, antibodies or binders which bind to the same epitope than GF275, GF276, GF277, GF278, GF297, GF298, GF302, GF303, GF307 or GF353 are useful to detect/recognize differentiated, preferably bone marrow derived, mesenchymal stem cells and/or differentiated in osteogenic direction (corresponding epitopes recognized by the antibodies are listed in Example 14). These epitopes are suitable and can be used to detect, isolate and evaluate of (mesenchymal) stem cells, preferably bone marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow from mixture of cells comprising other, bone marrow derived, cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other binders binding to stem cells, preferably human stem cells, comprise of binders which bind to the same epitope than GF297 (globoside GL4). A more preferred antibody comprises of the antibody of clone ab23949 by Abcam. This epitope is suitable and can be used to detect, isolate and evaluate of undifferentiated (mesenchymal) stem cells, preferably bone marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other binders binding to stem cells, preferably human stem cells, comprise of binders which bind to the same epitope than GF298 (human CD77; GB3). A more preferred antibody comprises of the antibody of clone SM1160 by Acris. This epitope is suitable and can be used to detect, isolate and evaluate of undifferentiated (mesenchymal) stem cells, preferably bone marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other binders binding to stem cells, preferably human stem cells, comprise of binders which bind to the same epitope than GF302 (H type 2 blood antigen). In a preferred embodiment, an antibody binds to Fucα2Galβ4GlcNAc epitope. A more preferred antibody comprises of the antibody of clone DM3015 by Acris. This epitope is suitable and can be used to detect, isolate and evaluate of undifferentiated (mesenchymal) stem cells, preferably bone marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

In a preferred embodiment of the present invention, antibodies or binders which bind to the same epitope than GF276, GF277, GF278, GF303, GF305, GF307, GF353, or GF354 are useful to detect/recognize, preferably bone marrow derived, mesenchymal stem cells and differentiated in osteogenic direction (corresponding epitopes recognized by the antibodies are listed in Example 14). These epitopes are suitable and can be used to detect, isolate and evaluate of (mesenchymal) stem cells, preferably bone marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow, or differentiated in osteogenic direction from mixture of cells comprising other, bone marrow derived, cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Further, the binders which bind to the same epitope than GF276 or GF303, or antibodies GF276 and/or GF303 are particularly useful to detect, isolate and evaluate of osteogenically differentiated stem cells, in culture or in vivo (corresponding epitopes recognized by the antibodies are listed in Example 14).

Other binders binding to stem cells, preferably human stem cells, comprise of binders which bind to the same epitope than GF276 (oncofetal antigen). A more preferred antibody comprises of the antibody of clone DM288 by Acris. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other binders binding to stem cells, preferably human stem cells, comprise of binders which bind to the same epitope than GF277 (human sialosyl-Tn antigen; STn, sCD175). A more preferred antibody comprises of the antibody of clone DM3197 by Acris. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other binders binding to stem cells, preferably human stem cells, comprise of binders which bind to the same epitope than GF278 (human sialosyl-Tn antigen; STn, sCD175 B1.1). A more preferred antibody comprises of the antibody of clone DM3218 by Acris. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Other binders binding to stem cells, preferably human stem cells, comprise of binders which bind to the same epitope than GF303 (blood group H1 antigen, BG4). In a preferred embodiment, an antibody binds to Fucα2Galβ3GlcNAc epitope. A more preferred antibody comprises of the antibody of clone ab3355 by Abcam. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells. The binder(s) and epitope recognized by it/them is also useful in growth of stem cells, modulation of the status of stem cells or subset of stem cells, change of the adherence status, differentiation related status, changing growth speed, is provided by contacting stem cells a binder which recognizes terminal glycan structures of stem cells. The binders are also useful in screening binders for growth of stem cells on a surface, modulation of the status of stem cells or subset of stem cells, change of the adherence status, differentiation related status, changing growth speed, is provided by contacting stem cells a binder which recognizes terminal glycan structures of stem cells.

Further, the antibodies or binders are useful to isolate and enrich stem cells for osteogenic lineage. This can be performed with positive selection, for example, with antibodies GF276, GF277, GF278, and GF303 (corresponding epitopes recognized by the antibodies are listed in Example 314). For negative depletion, a preferred epitope is the same as recognized with the antibodies GF305, GF307, GF353, or GF354. For negative depletion, a preferred epitope is the same as recognized with the antibody GF354 (SSEA-4) or GF307 (Sialyl Lewis x).

### Comparision Between Different Stem Cell Types

The present data revealed that comparison of a group of type 1 and type two N-acetyllactosamines is useful method for characterization stem cells such as mesenchymal stem cells and embryonal stem cells and or separating the cells from contaminating cell populations such as fibroblasts like feeder cells. The non-differentiated mesenchymal cell were devoid of type I N-acetyllactosamine antigens revealed from the hESC cells, while both cell types and potential contaminating fibroblast have variable labelling with type II N-acetyllactosamine recognizing antibodies.

The term “mainly” indicates preferably at least 60%, more preferably at least 75% and most preferably at least 90%. In the context of stem cells, the term “mainly” indicates preferably at least 60%, more preferably at least 75% and most preferably at least 90% of cells expressing a glycan structure and useful for identifying, characterizing, selecting or isolating pluripotent or multipotent stem cells in a population of mammalian cells.

### Uses of the Binders for Isolation of Cellular Components and Mixtures Thereof.

The invention revealed novel binding reagents are in a preferred embodiment used for isolation of cellular components from stem cells comprising the novel target/marker structures. The isolated cellular are preferably free glycans or glycans conjugated to proteins or lipids or fragment thereof.

The invention is especially directed to isolation of the cellular components comprising the structures when the structures comprises one or several types glycan materials sele
a) Free glycans released from the stem cell materials and/or
b) Glycan conjugate material such as
   b1) glycoamino acid materials including
      b1a) glycoproteins
      b1b) glycopeptides including glyco-oligopeptides and glycopolypeptides
   and/or
   b2) lipid linked materials comprising the preferred carbohydrate structures revealed by the invention.

### General Method for Isolation Cellular Components Comprising the Target Structures

The isolation of cellular components according to the invention means production of a molecular fraction comprising increased (or enriched) amount of the glycans comprising the target structures according to the invention in method comprising the step of binding of the binder molecule according to the invention to the corresponding target structures, which are glycan structures bound by the specific binder.

The process of isolation the fraction involving the contacting the binder molecule according to the invention with the corresponding target structures derived from stem cells and isolating the enriched target structure composition.

The preferred method to isolate cellular component includes following steps

1) Providing a stem cell sample.
2) Contacting the binder molecule according to the invention with the corresponding target structures.
3) Isolating the complex of the binder and target structure at least from part of cellular materials.

It is realized that the components are in general enriched in specific fractions of cellular structures such as cellular membrane fractions including plasma membrane and organelle fractions and soluble glycan comprising fractions such as soluble protein, lipid or free glycans fractions. It is realized that the binder can be used to total cellular fractions. In a preferred embodiment the target structures are enriched within a fraction of cellular proteins such as cell surface proteins releasable by protease or detergent soluble membrane proteins.

The preferred target structure composition comprise glycoproteins or glycopeptides comprising glycan structure corresponding to the binder structure and peptide or protein epitopes specifically expressed in stem cells or in proportions characteristic to stem cells.

More preferably the invention is directed to purification of the target structure fraction in the isolation step. The purification is in a preferred mode of invention is at least partial purification. Preferably the target glycan containing material is purified at least two fold, preferably among the components of cell fraction wherein it is expressed. More preferred purification levels includes 5-fold and 10 fold purification, more preferably 100, and even more preferably 1000-fold purification. Preferably the purified fraction comprises at least 10% of the target glycan comprising molecules, even more preferably at least 30%, even more preferably at least 50%, even more preferably at least 70% pure and most preferably at least 90% pure. Preferably the % value is mole percent in comparison to other non-target glycan comprising glycaconjugate molecules, more preferably the material is essentially devoid of other major organic contaminating molecules.

### Preferred Purified Target Glycan Compositions and Target Glycan-Binder Complexes

The invention is also directed to isolated or purified target glycan-binder complexes and isolated target glycan molecule compositions, wherein the target glycans are enriched with a specific target structures according to the invention.

Preferably the purified target glycan-binder complex compositions comprises at least 10% of the target glycan comprising molecules in complex with binder, even more preferably at least 30%, even more preferably at least 50%, even more preferably at least 70% pure and most preferably at least 90% pure target glycan comprising molecules in complex with binder.

Preferably the purified target glycan composition comprises at least 10% of the target glycan comprising molecules, even more preferably at least 30%, even more preferably at least 50%, even more preferably at least 70% pure and most preferably at least 90% pure target glycan comprising molecules.

The invention is further directed to the enriched target glycan composition produced by the process of isolation the fraction involving the steps of the contacting the binder molecule according to the invention with the corresponding target structures derived from stem cell and isolating the enriched target structure.

### Binder Technology for Purification of Target Glycans

The methods for affinity purification of cellular glycoproteins, glycopeptides, free oligosaccharides and other glycan conjugates are well-known in the art. The preferred methods include solid phase involving binder technologies such as affinity chromatography, precipitation such as immunoprecipitation, binder-magnetic methods such as immunomegnetic bead methods. Affinity chromatographies has been described for purification of glycopeptides by using lectins (Wang Y et al (2006) Glycobiology 16 (6) 514-23) or by antibodies or purification of glycoproteins/peptides by using antibodies (e.g. Prat M et al cancer Res (1989) 49, 1415-21; Kim Y D et al et al Cancer Res (1989) 49, 2379) and/or lectins (e.g. Cumming and Kornfeld (1982) J Biol Chem 257, 11235-40; Yae E et al. (1991) 1078 (3) 369-76; Shibuya N et al (1988) 267 (2) 676-80; Gonchoroff D G et al. 1989, 35, 29-32; Hentges and Bause (1997) Biol Chem 378 (9) 1031-8). Specific methods have been developed for weakly binding antibodies even for recognition of free oligosaccharides as described e.g. in (Ohlson S et al. J Chromatogr A (1997) 758 (2) 199-208), Ohlson S et al. Anal Biochem (1988) 169 (1) 204-8). The methods may involve multiple steps by binders of different specificities as shown e.g. in (Cummings and Kornfeld (1982) J Biol Chem 257, 11235-40). Antibody or protein (lectin) binder affinity chromatography for oligosaccharide mixtures has been also described e.g. in (Kitagawa H et al. (1991) J Biochem 110 (49 598-604; Kitagawa H et al. (1989) Biochemistry 28 (22) 8891-7; Dakour J et al Arch Biochem Biophys (1988) 264, 203-13) and for glycolipids e.g. in (Bouhours D et al (1990) Arch Biochem Biophys 282 (1) 141-6). Further information of glycan directed affinity chromatography and/or useful lectin and antibody specificities is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and L is, Halina) Kluwer Academic publishers Dordrecht, The Netherlands).

The methods includes normal pressure or in HPLC chromatographies and may include additional steps using traditional chromatographic methods or other protein and peptide purification methods, a preferred additional isolation methods is gel filtration (size exclusion) chromatography for isolation of especially lower Mw glycans and conjugates, preferably glycopeptides.

It is further known that isolated proteins and peptides can be recognized by mass spectrometric methods e.g. (Wang Y et al (2006) Glycobiology 16 (6) 514-23). The invention is specifically directed to use of the binders according to the invention for purification of glycans and/or their conjugates and recognition of the isolated component by methods such as mass spectrometry, peptide sequencing, chemical analysis, array analysis or other methods known in the art.

### Revealing Presence Trypsin Sensitive Forms of Glycan Targets

The invention reveals in example 20 that part of the target structures of present glycan binders, especially monoclonal antibodies are trypsin sensitive. The antigen structures are essentially not observed or these are observed in reduced amount in FACS analysis of cell surface antigens when cells are treated (released from cultivation) by trypsin but observable after Versene treatment (0.02% EDTA in PBS). This was observed for example for labelling of mesenchymal stem cells by the antibody GF354, which has been indicated to bind SSEA-4 antigen. This target antigen structure has been traditionally considered to be sialyl-galactosylgloboside glycolipid, but obviously the antibody recognizes only an epitope at the non-reducing end of glycan sequence. The present invention is now especially directed to methods of isolation and characterization of mesenchymal stem cell glycopeptide bound glycan structure(s), which can be bound and enriched by the SSEA-4 antibodies, and to characterization of corresponding glycopeptides and glycoproteins. The invention is further directed to analysis of trypsin insensitive glycan materials from stem cell especially mesenchymal stem cells and embryonal stem cells.

The invention revealed also that major part of the sialyl-mucin type target of ab GF 275 is trypsin sensitive and minor part is not trypsin sensitive. The invention is directed to isolation of both trypsin sensitive and trypsin insensitive glycan fractions, preferably glycoprotein(s) and glycopeptides, by methods according to the invention. The invention is further directed to isolation and characterization of protein degrading enzyme (protease) sensitive likely glycopeptides and glycoproteins bound by antibody GF 302, preferably when the materials are isolated from mesenchymal stem cells.

As used herein, “binder”, “binding agent” and “marker” are used interchangeably.

### ‘Antibodies

Information about useful lectin and antibody specificities useful according to the invention and for reducing end elongated antibody epitopes is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and L is, Halina) Kluwer Academic publishers Dordrecht, The Netherlands and internet databases such as pubmed/espacenet or antibody databases such as www.glyco.is.ritsumei.ac.jp/epitope/, which list monoclonal antibody specificities).

Various procedures known in the art may be used for the production of polyclonal antibodies to peptide motifs and regions or fragments thereof. For the production of antibodies, any suitable host animal (including but not limited to rabbits, mice, rats, or hamsters) are immunized by injection with a peptide (immunogenic fragment). Various adjuvants may be used to increase the immunological response, depending on the host species, including but not limited to Freund's (complete and incomplete) adjuvant, mineral gels such as aluminum hydroxide, surface active substances such as lysolecithin, pluronic polyols, polyanions, oil emulsions, keyhole limpet hemocyanins, dinitrophenol, and potentially useful human adjuvants such as BCG {Bacille Calmette-Guerin) and Corynebacterium parvum.

A monoclonal antibody to a peptide motif(s) may be prepared by using any technique which provides for the production of antibody molecules by continuous cell lines in culture. These include but are not limited to the hybridoma technique originally described by kδhler et al., (Nature, 256: 495-497, 1975), and the more recent human B-cell hybridoma technique (Kosbor et al., Immunology Today, 4: 72, 1983) and the EBV-hybridoma technique (Cole et al., Monoclonal Antibodies and Cancer Therapy, Alan R Liss, Inc., pp. 77-96, 1985), all specifically incorporated herein by reference. Antibodies also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 41, Sp210-Ag14, FO, NSO/U, MPC-I1, MPC11-X45-GTG 1.7 and S194/5XX0 BuI; for rats, one may use R210.RCY3, Y3-Ag 1.2.3, IR983F and 4B210; and U-266, GM1500-GRG2, LICR-LON-HMy2 and UC729-6 all may be useful in connection with cell fusions.

In addition to the production of monoclonal antibodies, techniques developed for the production of “chimeric antibodies”, the splicing of mouse antibody genes to human antibody genes to obtain a molecule with appropriate antigen specificity and biological activity, can be used (Morrison et al, Proc Natl Acad Sd 81: 6851-6855, 1984; Neuberger et al, Nature 312: 604-608, 1984; Takeda et al, Nature 314: 452-454; 1985). Alternatively, techniques described for the production of single-chain antibodies (U.S. Pat. No. 4,946,778) can be adapted to produce influenza-specific single chain antibodies.

Antibody fragments that contain the idiotype of the molecule may be generated by known techniques. For example, such fragments include, but are not limited to, the F(ab′)₂ fragment which may be produced by pepsin digestion of the antibody molecule; the Fab′ fragments which may be generated by reducing the disulfide bridges of the F(ab′)₂ fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a complementarity determining region (CDR), of the antibody is derived from a non-human species. The human light chain constant region may be from either a kappa or lambda light chain, while the human heavy chain constant region may be from either an IgM, an IgG (IgG1, IgG2, IgG3, or IgG4) an IgD, an IgA, or an IgE immunoglobulin.

Methods for humanizing non-human antibodies are well known in the art (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. {Nature 321: 522-525, 1986), Riechmann et al, {Nature, 332: 323-327, 1988) and Verhoeyen et al. Science 239:1534-1536, 1988), by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165, 1994. Further changes can then be introduced into the antibody framework to modulate affinity or immunogenicity.

Likewise, using techniques known in the art to isolate CDRs, compositions comprising CDRs are generated. Complementarity determining regions are characterized by six polypeptide loops, three loops for each of the heavy or light chain variable regions. The amino acid position in a CDR and framework region is set out by Kabat et al., “Sequences of Proteins of Immunological Interest,” U.S. Department of Health and Human Services, (1983), which is incorporated herein by reference. For example, hypervariable regions of human antibodies are roughly defined to be found at residues 28 to 35, from residues 49-59 and from residues 92-103 of the heavy and light chain variable regions (Janeway and Travers, Immunobiology, 2nd Edition, Garland Publishing, New York, 1996). The CDR regions in any given antibody may be found within several amino acids of these approximated residues set forth above. An immunoglobulin variable region also consists of “framework” regions surrounding the CDRs. The sequences of the framework regions of different light or heavy chains are highly conserved within a species, and are also conserved between human and murine sequences.

Compositions comprising one, two, and/or three CDRs of a heavy chain variable region or a light chain variable region of a monoclonal antibody are generated. Polypeptide compositions comprising one, two, three, four, five and/or six complementarity determining regions of a monoclonal antibody secreted by a hybridoma are also contemplated. Using the conserved framework sequences surrounding the CDRs, PCR primers complementary to these consensus sequences are generated to amplify a CDR sequence located between the primer regions. Techniques for cloning and expressing nucleotide and polypeptide sequences are well-established in the art [see e.g., Sambrook et al., Molecular Cloning: A Laboratory Manual, 2nd Edition, Cold Spring Harbor, N.Y. (1989)]. The amplified CDR sequences are ligated into an appropriate plasmid. The plasmid comprising one, two, three, four, five and/or six cloned CDRs optionally contains additional polypeptide encoding regions linked to the CDR.

Preferably, the antibody is any antibody specific for a glycan structure of Formula (I) or a fragment thereof. The antibody used in the present invention encompasses any antibody or fragment thereof, either native or recombinant, synthetic or naturally-derived, monoclonal or polyclonal which retains sufficient specificity to bind specifically to the glycan structure according to Formula (I) which is indicative of stem cells. As used herein, the terms “antibody” or “antibodies” include the entire antibody and antibody fragments containing functional portions thereof. The term “antibody” includes any monospecific or bispecific compound comprised of a sufficient portion of the light chain variable region and/or the heavy chain variable region to effect binding to the epitope to which the whole antibody has binding specificity. The fragments can include the variable region of at least one heavy or light chain immunoglobulin polypeptide, and include, but are not limited to, Fab fragments, F(ab′).sub.2 fragments, and Fv fragments.

The antibodies can be conjugated to other suitable molecules and compounds including, but not limited to, enzymes, magnetic beads, colloidal magnetic beads, haptens, fluorochromes, metal compounds, radioactive compounds, chromatography resins, solid supports or drugs. The enzymes that can be conjugated to the antibodies include, but are not limited to, alkaline phosphatase, peroxidase, urease and .beta.-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P. Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m, .sup.125 I and amino acids comprising any radionuclides, including, but not limited to .sup.14 C, .sup.3 H and .sup.35 S.

Antibodies to glycan structure(s) of Formula (I) may be obtained from any source. They may be commercially available. Effectively, any means which detects the presence of glycan structure(s) on the stem cells is with the scope of the present invention. An example of such an antibody is a H type 1 (clone 17-206; GF 287) antibody from Abcam.

### HSCs

The methods outlined herein are particularly useful for identifying HSCs or progeny thereof from a population of cells. However, additional markers may be used to further distinguish subpopulations within the general HSC, or stem cell, population.

The various sub-populations may be distinguished by levels of binders to glycan structures of Formula (I) on stem cells. This may manifest on the stem cell surface (or on feeder cell if feeder cell specific binder is used) which may be detected by the methods outlined herein. However, the present invention may be used to distinguish between various phenotypes of the stem cell or HSC population including, but not limited to, the CD34.sup.+, CD38.sup.−, CD90.sup.+ (thy1) and Lin.sup.− cells. Preferably the cells identified are selected from the group including, but not limited to, CD34.sup.+, CD38.sup.−, CD90+ (thy 1), or Lin.sup.−.

The present invention thus encompasses methods of enriching a population for stem and/or HSCs or progeny thereof. The methods involve combining a mixture of HSCs or progeny thereof with an antibody or marker or binding protein/agent or binder that recognizes and binds to glycan structure according to Formula (I) on stem cell(s) under conditions which allow the antibody or marker or binder to bind to glycan structure according to Formula (I) on stem cell(s) and separating the cells recognized by the antibody or marker to obtain a population substantially enriched in stem cells or progeny thereof. The methods can be used as a diagnostic assay for the number of HSCs or progeny thereof in a sample. The cells and antibody or marker are combined under conditions sufficient to allow specific binding of the antibody or marker to glycan structure according to Formula (I) on stem cell(s) which are then quantitated. The HSCs or stem cells or progeny thereof can be isolated or further purified.

As discussed above the cell population may be obtained from any source of stem cells or HSCs or progeny thereof including those samples discussed above.

The detection for the presence of glycan structure(s) according to Formula (I) on stem cell(s) may be conducted in any way to identify glycan structure according to Formula (I) on stem cell(s). Preferably the detection is by use of a marker or binding protein for glycan structure according to Formula (I) on stem cell(s). The binder/marker for glycan structure according to Formula (I) on stem cell(s) may be any of the markers discussed above. However, antibodies or binding proteins to glycan structure according to Formula (I) on stem cell(s) are particularly useful as a marker for glycan structure according to Formula (I) on stem cell(s).

Various techniques can be employed to separate or enrich the cells by initially removing cells of dedicated lineage. Monoclonal antibodies, binding proteins and lectins are particularly useful for identifying cell lineages and/or stages of differentiation. The antibodies can be attached to a solid support to allow for crude separation. The separation techniques employed should maximize the retention of viability of the fraction to be collected. Various techniques of different efficacy can be employed to obtain “relatively crude” separations. The particular technique employed will depend upon efficiency of separation, associated cytotoxicity, ease and speed of performance, and necessity for sophisticated equipment and/or technical skill.

Procedures for separation or enrichment can include, but are not limited to, magnetic separation, using antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique.

The use of separation or enrichment techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye, Hoescht 33342).

Techniques providing accurate separation include, but are not limited to, FACS, which can have varying degrees of sophistication, e.g., a plurality of color channels, low angle and obtuse light scattering detecting channels, impedence channels, etc. Any method which can isolate and distinguish these cells according to levels of expression of glycan structure according to Formula (I) on stem cell(s) may be used.

In a first separation, typically starting with about 1.times.10.sup.10, preferably at about 5.times.10.sup.8-9 cells, antibodies or binding proteins or lectins to glycan structure according to Formula (I) on stem cell(s) can be labeled with at least one fluorochrome, while the antibodies or binding proteins for the various dedicated lineages, can be conjugated to at least one different fluorochrome. While each of the lineages can be separated in a separate step, desirably the lineages are separated at the same time as one is positively selecting for glycan structure according to Formula (I) on stem cell markers. The cells can be selected against dead cells, by employing dyes associated with dead cells (including but not limited to, propidium iodide (PI)).

To further enrich for any cell population, specific markers for those cell populations may be used. For instance, specific markers for specific cell lineages such as lymphoid, myeloid or erythroid lineages may be used to enrich for or against these cells. These markers may be used to enrich for HSCs or progeny thereof by removing or selecting out mesenchymal or keratinocyte stem cells.

The methods described above can include further enrichment steps for cells by positive selection for other stem cell specific markers. Suitable positive stem cell markers include, but are not limited to, SSEA-3, SSEA-4, Tra 1-60, CD34.sup.+, Thy-1.sup.+, and c-kit.sup.+. By appropriate selection with particular factors and the development of bioassays which allow for self-regeneration of HSCs or progeny thereof and screening of the HSCs or progeny thereof as to their markers, a composition enriched for viable HSCs or progeny thereof can be produced for a variety of purposes.

Once the stem cells or HSC or progeny thereof population is isolated, further isolation techniques may be employed to isolate sub-populations within the HSCs or progeny thereof. Specific markers including cell selection systems such as FACS for cell lineages may be used to identify and isolate the various cell lineages.

In yet another aspect of the present invention there is provided a method of measuring the content of stem cells or HSC or their progeny said method comprising

obtaining a cell population comprising stem cells or progeny thereof;
combining the cell population with a binding protein or binder for glycan structure according to Formula (I) on stem cell(s) thereof;
selecting for those cells which are identified by the binding protein for glycan structure according to Formula (I) on stem cell(s) thereof; and
quantifying the amount of selected cells relative to the quantity of cells in the cell population prior to selection with the binding protein.

### Binder-Label Conjugates

The present invention is specifically directed to the binding of the structures according to the present invention, when the binder is conjugated with “a label structure”. The label structure means a molecule observable in a assay such as for example a fluorescent molecule, a radioactive molecule, a detectable enzyme such as horse radish peroxidase or biotin/streptavidin/avidin. When the labelled binding molecule is contacted with the cells according to the invention, the cells can be monitored, observed and/or sorted based on the presence of the label on the cell surface. Monitoring and observation may occur by regular methods for observing labels such as fluorescence measuring devices, microscopes, scintillation counters and other devices for measuring radioactivity.

### Use of Binder and Labelled Binder-Conjugates for Cell Sorting

The invention is specifically directed to use of the binders and their labelled conjugates for sorting or selecting human stem cells from biological materials or samples including cell materials comprising other cell types. The preferred cell types includes cord blood, peripheral blood and embryonal stem cells and associated cells. The labels can be used for sorting cell types according to invention from other similar cells. In another embodiment the cells are sorted from different cell types such as blood cells or in context of cultured cells preferably feeder cells, for example in context of embryonal stem cells corresponding feeder cells such as human or mouse feeder cells. A preferred cell sorting method is FACS sorting. Another sorting methods utilized immobilized binder structures and removal of unbound cells for separation of bound and unbound cells.

### Use of Immobilized Binder Structures

In a preferred embodiment the binder structure is conjugated to a solid phase. The cells are contacted with the solid phase, and part of the material is bound to surface. This method may be used to separation of cells and analysis of cell surface structures, or study cell biological changes of cells due to immobilization. In the analytics involving method the cells are preferably tagged with or labelled with a reagent for the detection of the cells bound to the solid phase through a binder structure on the solid phase. The methods preferably further include one or more steps of washing to remove unbound cells.

Preferred solid phases include cell suitable plastic materials used in contacting cells such as cell cultivation bottles, petri dishes and microtiter wells; fermentor surface materials, etc.

### Specific Recognition Between Preferred Stem Cells and Contaminating Cells

The invention is further directed to methods of recognizing stem cells from differentiated cells such as feeder cells, preferably animal feeder cells and more preferably mouse feeder cells. It is further realized, that the present reagents can be used for purification of stem cells by any fractionation method using the specific binding reagents.

Preferred fractionation methods includes fluorecense activated cell sorting (FACS), affinity chromatography methods, and bead methods such as magnetic bead methods.

Preferred reagents for recognition between preferred cells, preferably embryonal type cells, and contaminating cells, such as feeder cells, most preferably mouse feeder cells, include reagents according to the Table 23, more preferably proteins with similar specificity with lectins PSA, MAA, and PNA.

The invention is further directed to positive selection methods including specific binding to the stem cell population but not to contaminating cell population. The invention is further directed to negative selection methods including specific binding to the contaminating cell population but not to the stem cell population. In yet another embodiment of recognition of stem cells the stem cell population is recognized together with a homogenous cell population such as a feeder cell population, preferably when separation of other materials is needed. It is realized that a reagent for positive selection can be selected so that it binds stem cells as in the present invention and not to the contaminating cell population and a reagent for negative selection by selecting opposite specificity. In case of one population of cells according to the invention is to be selected from a novel cell population not studied in the present invention, the binding molecules according to the invention maybe used when verified to have suitable specificity with regard to the novel cell population (binding or not binding). The invention is specifically directed to analysis of such binding specificity for development of a new binding or selection method according to the invention.

The preferred specificities according to the invention include recognition of:
i) mannose type structures, especially alpha-Man structures like lectin PSA, preferably on the surface of contaminating cells
ii) α3-sialylated structures similarily as by MAA-lectin, preferably for recognition of embryonal type stem cells
iii) Gal/GalNAc binding specificity, preferably Galβ1-3/GalNAc1-3 binding specificity, more preferably Galβ1-3/GalNAcβ1-3 binding specificity similar to PNA, preferably for recognition of embryonal type stem cells

### Manipulation of Cells by Binders

The invention is specifically directed to manipulation of cells by the specific binding proteins. It is realized that the glycans described have important roles in the interactions between cells and thus binders or binding molecules can be used for specific biological manipulation of cells. The manipulation may be performed by free or immobilized binders. In a preferred embodiment cells are used for manipulation of cell under cell culture conditions to affect the growth rate of the cells.

### Stem Cell Nomenclature

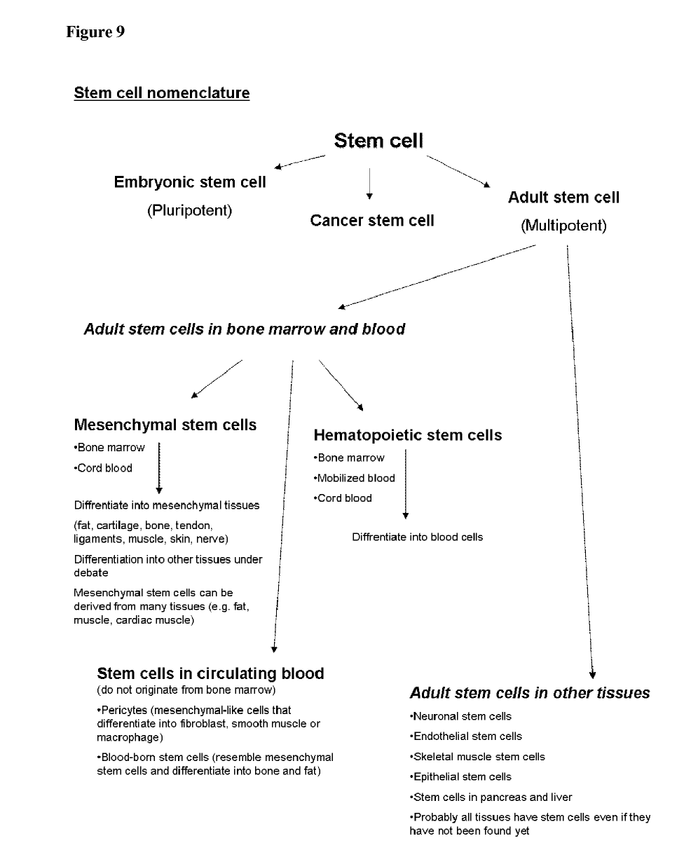

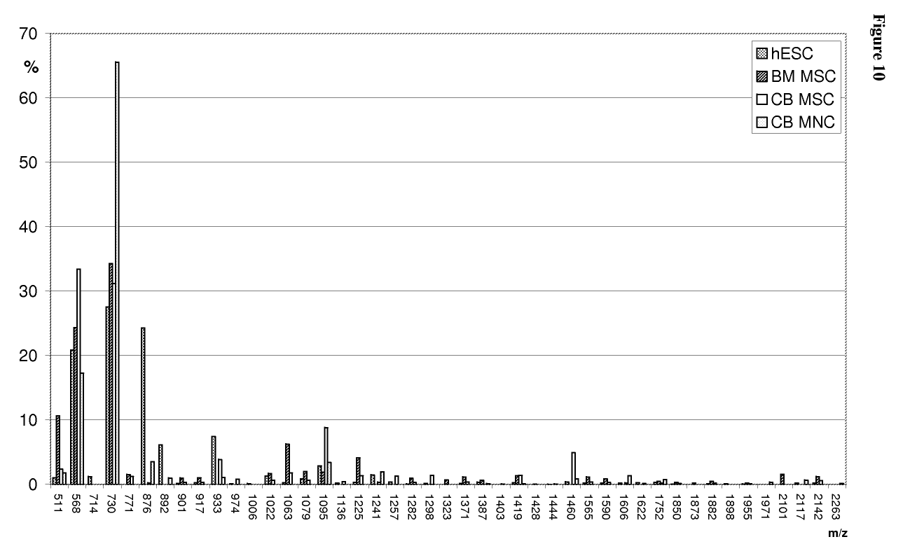

The present invention is directed to analysis of all stem cell types, preferably human stem cells. A general nomenclature of the stem cells is described in FIG. 9. The alternative nomenclature of the present invention describe early human cells which are in a preferred embodiment equivalent of adult stem cells (including cord blood type materials) as shown in FIG. 9. Adult stem cells in bone marrow and blood is equivalent for stem cells from “blood related tissues”.

### Lectins for Manipulation of Stem Cells, Especially Under Cell Culture Conditions

The present invention is especially directed to use of lectins as specific binding proteins for analysis of status of stem cells and/or for the manipulation of stems cells.

The invention is specifically directed to manipulation of stem cells under cell culture conditions growing the stem cells in presence of lectins. The manipulation is preferably performed by immobilized lectins on surface of cell culture vessels. The invention is especially directed to the manipulation of the growth rate of stem cells by growing the cells in the presence of lectins, as show in Table 24.

The invention is in a preferred embodiment directed to manipulation of stem cells by specific lectins recognizing specific glycan marker structures according to invention from the cell surfaces. The invention is in a preferred embodiment directed to use of Gal recognizing lectins such as ECA-lectin or similar human lectins such as galectins for recognition of galectin ligand glycans identified from the cell surfaces. It was further realized that there is specific variations of galectin expression in genomic levels in stem cells, especially for galectins-1, -3, and -8. The present invention is especially directed to methods of testing of these lectins for manipulation of growth rates of embryonal type stem cells and for adult stem cells in bone marrow and blood and differentiating derivatives thereof.

### Sorting of Stem Cells by Specific Lectins

The invention revealed use of specific lectin types recognizing cell surface glycan epitopes according to the invention for sorting of stem cells, especially by FACS methods, most preferred cell types to be sorted includes adult stem cells in blood and bone marrow, especially cord blood cells. Preferred lectins for sorting of cord blood cells include GNA, STA, GS-II, PWA, HHA, PSA, RCA, and others as shown in Example 12. The relevance of the lectins for isolating specific stem cell populations was demonstrated by double labeling with known stem cells markers, as described in Example 12.

### Preferred Structures of O-Glycan Glycomes of Stem Cells

The present invention is especially directed to following O-glycan marker structures of stem cells:

Core 1 type O-glycan structures following the marker composition NeuAc₂Hex₁HexNAc₁, preferably including structures SAα3Galβ3GalNAc and/or SAα3Galβ3(Saα6)GalNAc;
and Core 2 type O-glycan structures following the marker composition NeuAc₀₋₂Hex₂HexNAc₂dHex₀₋₁, more preferentially further including the glycan series NeuAc₀₋₂Hex₂₊ₙHexNAc₂₊ₙdHex₀₋₁, wherein n is either 1, 2, or 3 and more preferentially n is 1 or 2, and even more preferentially n is 1;
more specifically preferably including R₁Galβ4(R₃)GlcNAcβ6(R₂Galβ3)GalNAc,
wherein R₁ and R₂ are independently either nothing or sialic acid residue, preferably α2,3-linked sialic acid residue, or an elongation with HexₙHexNAcₙ, wherein n is independently an integer at least 1, preferably between 1-3, most preferably between 1-2, and most preferably 1, and the elongation may terminate in sialic acid residue, preferably α2,3-linked sialic acid residue; and
R₃ is independently either nothing or fucose residue, preferably α1,3-linked fucose residue.

It is realized that these structures correlate with expression of β6GlcNAc-transferases synthesizing core 2 structures.

### Preferred Branched N-Acetyllactosamine Type Glycosphingolipids

The invention further revealed branched, 1-type, poly-N-acetyllactosamines with two terminal Galβ4-residues from glycolipids of human stem cells. The structures correlate with expression of β6GlcNAc-transferases capable of branching poly-N-acetyllactosamines and further to binding of lectins specific for branched poly-N-acetyllactosamines. It was further noticed that PWA-lectin had an activity in manipulation of stem cells, especially the growth rate thereof.

### Preferred Qualitative and Quantitative Complete N-Glycomes of Stem Cells

### Preferred Binders for Stem Cell Sorting and Isolation

As described in the Examples, the inventors found that especially the mannose-specific and especially α1,3-linked mannose-binding lectin GNA was suitable for negative selection enrichment of CD34+ stem cells from CB MNC. In addition, the poly-LacNAc specific lectin STA and the fucose-specific and especially α1,2-linked fucose-specific lectin UEA were suitable for positive selection enrichment of CD34+ stem cells from CB MNC.

The present invention is specifically directed to stem cell binding reagents, preferentially proteins, preferentially mannose-binding or α1,3-linked mannose-binding, poly-LacNAc binding, LacNAc-binding, and/or fucose- or preferentially α1,2-linked fucose-binding; in a preferred embodiment stem cell binding or nonbinding lectins, more preferentially GNA, STA, and/or UEA; and in a further preferred embodiment combinations thereof; to uses described in the present invention taking advantage of glycan-binding reagents that selectively either bind to or do not bind to stem cells.

Preferred Uses for Stem Cell Type Specific Galectins and/or Galectin Ligands

As described in the Examples, the inventors also found that different stem cells have distinct galectin expression profiles and also distinct galectin (glycan) ligand expression profiles. The present invention is further directed to using galactose-binding reagents, preferentially galactose-binding lectins, more preferentially specific galectins; in a stem cell type specific fashion to modulate or bind to certain stem cells as described in the present invention to the uses described. In a further preferred embodiment, the present invention is directed to using galectin ligand structures, derivatives thereof, or ligand-mimicking reagents to uses described in the present invention in stem cell type specific fashion.

### EXAMPLES

### Example 1

### MALDI-TOF Mass Spectrometric N-Glycan Profiling, Glycosidase and Lectin Profiling of Cord Blood Derived and Bone Marrow Derived Mesenchymal Stem Cell Lines

### Examples of Cell Sample Production

### Cord Blood Derived Mesenchymal Stem Cell Lines

Collection of umbilical cord blood. Human term umbilical cord blood (UCB) units were collected after delivery with informed consent of the mothers and the UCB was processed within 24 hours of the collection. The mononuclear cells (MNCs) were isolated from each UCB unit diluting the UCB 1:1 with phosphate-buffered saline (PBS) followed by Ficoll-Paque Plus (Amersham Biosciences, Uppsala, Sweden) density gradient centrifugation (400 g/40 min) The mononuclear cell fragment was collected from the gradient and washed twice with PBS.

Umbilical cord blood cell isolation and culture. CD45/Glycophorin A (GlyA) negative cell selection was performed using immunolabeled magnetic beads (Miltenyi Biotec). MNCs were incubated simultaneously with both CD45 and GlyA magnetic microbeads for 30 minutes and negatively selected using LD columns following the manufacturer's instructions (Miltenyi Biotec). Both CD45/GlyA negative elution fraction and positive fraction were collected, suspended in culture media and counted. CD45/GlyA positive cells were plated on fibronectin (FN) coated six-well plates at the density of 1×10⁶/cm². CD45/GlyA negative cells were plated on FN coated 96-well plates (Nunc) about 1×10⁴ cells/well. Most of the non-adherent cells were removed as the medium was replaced next day. The rest of the non-adherent cells were removed during subsequent twice weekly medium replacements.

The cells were initially cultured in media consisting of 56% DMEM low glucose (DMEM-LG, Gibco, http://www.invitrogen.com) 40% MCDB-201 (Sigma-Aldrich) 2% fetal calf serum (FCS), 1× penicillin-streptomycin (both form Gibco), 1×ITS liquid media supplement (insulin-transferrin-selenium), 1× linoleic acid-BSA, 5×10⁻⁸ M dexamethasone, 0.1 mM L-ascorbic acid-2-phosphate (all three from Sigma-Aldrich), 10 nM PDGF (R&D systems, http://www.RnDSystems.com) and 10 nM EGF (Sigma-Aldrich). In later passages (after passage 7) the cells were also cultured in the same proliferation medium except the FCS concentration was increased to 10%.

Plates were screened for colonies and when the cells in the colonies were 80-90% confluent the cells were subcultured. At the first passages when the cell number was still low the cells were detached with minimal amount of trypsin/EDTA (0.25%/1 mM, Gibco) at room temperature and trypsin was inhibited with FCS. Cells were flushed with serum free culture medium and suspended in normal culture medium adjusting the serum concentration to 2%. The cells were plated about 2000-3000/cm². In later passages the cells were detached with trypsin/EDTA from defined area at defined time points, counted with hematocytometer and replated at density of 2000-3000 cells/cm².

### Bone Marrow Derived Mesenchymal Stem Cell Lines

Isolation and culture of bone marrow derived stem cells. Bone marrow (BM) derived MSCs were obtained as described by Leskelä et al. (2003). Briefly, bone marrow obtained during orthopedic surgery was cultured in Minimum Essential Alpha-Medium (α-MEM), supplemented with 20 mM HEPES, 10% FCS, 1× penicillin-streptomycin and 2 mM L-glutamine (all from Gibco). After a cell attachment period of 2 days the cells were washed with Ca²⁺ and Mg²⁺ free PBS (Gibco), subcultured further by plating the cells at a density of 2000-3000 cells/cm2 in the same media and removing half of the media and replacing it with fresh media twice a week until near confluence.

### Experimental Procedures

Flow cytometric analysis of mesenchymal stem cell phenotype. Both UBC and BM derived mesenchymal stem cells were phenotyped by flow cytometry (FACSCalibur, Becton Dickinson). Fluorescein isothiocyanate (FITC) or phycoerythrin (PE) conjugated antibodies against CD13, CD14, CD29, CD34, CD44, CD45, CD49e, CD73 and HLA-ABC (all from BD Biosciences, San Jose, Calif., http://www.bdbiosciences.com), CD105 (Abeam Ltd., Cambridge, UK, http://www.abcam.com) and CD133 (Miltenyi Biotec) were used for direct labeling. Appropriate FITC- and PE-conjugated isotypic controls (BD Biosciences) were used. Unconjugated antibodies against CD90 and HLA-DR (both from BD Biosciences) were used for indirect labeling. For indirect labeling FITC-conjugated goat anti-mouse IgG antibody (Sigma-aldrich) was used as a secondary antibody.

The UBC derived cells were negative for the hematopoietic markers CD34, CD45, CD14 and CD133. The cells stained positively for the CD13 (aminopeptidase N), CD29 (β1-integrin), CD44 (hyaluronate receptor), CD73 (SH3), CD90 (Thy1), CD105 (SH2/endoglin) and CD 49e. The cells stained also positively for HLA-ABC but were negative for HLA-DR. BM-derived cells showed to have similar phenotype. They were negative for CD14, CD34, CD45 and HLA-DR and positive for CD13, CD29, CD44, CD90, CD105 and HLA-ABC.

Adipogenic differentiation. To assess the adipogenic potential of the UCB-derived MSCs the cells were seeded at the density of 3×10³/cm² in 24-well plates (Nunc) in three replicate wells. UCB-derived MSCs were cultured for five weeks in adipogenic inducing medium which consisted of DMEM low glucose, 2% FCS (both from Gibco), 10 μg/ml insulin, 0.1 mM indomethacin, 0.1 μM dexamethasone (Sigma-Aldrich) and penicillin-streptomycin (Gibco) before samples were prepared for glycome analysis. The medium was changed twice a week during differentiation culture.

Osteogenic differentiation. To induce the osteogenic differentiation of the BM-derived MSCs the cells were seeded in their normal proliferation medium at a density of 3×10³/cm² on 24-well plates (Nunc). The next day the medium was changed to osteogenic induction medium which consisted of α-MEM (Gibco) supplemented with 10% FBS (Gibco), 0.1 μM dexamethasone, 10 mM β-glycerophosphate, 0.05 mM L-ascorbic acid-2-phosphate (Sigma-Aldrich) and penicillin-streptomycin (Gibco). BM-derived MSCs were cultured for three weeks changing the medium twice a week before preparing samples for glycome analysis.

Cell harvesting for glycome analysis. 1 ml of cell culture medium was saved for glycome analysis and the rest of the medium removed by aspiration. Cell culture plates were washed with PBS buffer pH 7.2. PBS was aspirated and cells scraped and collected with 5 ml of PBS (repeated two times). At this point small cell fraction (10 μl) was taken for cell-counting and the rest of the sample centrifuged for 5 minutes at 400 g. The supernatant was aspirated and the pellet washed in PBS for an additional 2 times.

The cells were collected with 1.5 ml of PBS, transferred from 50 ml tube into 1.5 ml collection tube and centrifuged for 7 minutes at 5400 rpm. The supernatant was aspirated and washing repeated one more time. Cell pellet was stored at −70° C. and used for glycome analysis.

Lectin stainings. FITC-labeled Maackia amurensis agglutinin (MAA) was purchased from EY Laboratories (USA) and FITC-labeled Sambucus nigra agglutinin (SNA) was purchased from Vector Laboratories (UK). Bone marrow derived mesenchymal stem cell lines were cultured as described above. After culturing, cells were rinsed 5 times with PBS (10 mM sodium phosphate, pH 7.2, 140 mM NaCl) and fixed with 4% PBS-buffered paraformaldehyde pH 7.2 at room temperature (RT) for 10 minutes. After fixation, cells were washed 3 times with PBS and non-specific binding sites were blocked with 3% HSA-PBS (FRC Blood Service, Finland) or 3% BSA-PBS (>99% pure BSA, Sigma) for 30 minutes at RT. According to manufacturers' instructions cells were washed twice with PBS, TBS (20 mM Tris-HCl, pH 7.5, 150 mM NaCl, 10 mM CaCl₂) or HEPES-buffer (10 mM HEPES, pH 7.5, 150 mM NaCl) before lectin incubation. FITC-labeled lectins were diluted in 1% HSA or 1% BSA in buffer and incubated with the cells for 60 minutes at RT in the dark. Furthermore, cells were washed 3 times 10 minutes with PBS/TBS/HEPES and mounted in Vectashield mounting medium containing DAPI-stain (Vector Laboratories, UK). Lectin stainings were observed with Zeiss Axioskop 2 plus-fluorescence microscope (Carl Zeiss Vision GmbH, Germany) with FITC and DAPI filters. Images were taken with Zeiss AxioCam MRc-camera and with AxioVision Software 3.1/4.0 (Carl Zeiss) with the 400× magnification.

### Results

Glycan isolation from mesenchymal stem cell populations. The present results are produced from two cord blood derived mesenchymal stem cell lines and cells induced to differentiate into adipogenic direction, and two marrow derived mesenchymal stem cell lines and cells induced to differentiate into osteogenic direction. The characterization of the cell lines and differentiated cells derived from them are described above. N-glycans were isolated from the samples, and glycan profiles were generated from MALDI-TOF mass spectrometry data of isolated neutral and sialylated N-glycan fractions as described in the preceding examples.

### Cord Blood Derived Mesenchymal Stem Cell (CB MSC) Lines

Neutral N-glycan structural features. Neutral N-glycan groupings proposed for the two CB MSC lines resemble each other closely, indicating that there are no major differences in their neutral N-glycan structural features. However, CB MSCs differ from the CB mononuclear cell populations, and they have for example relatively high amounts of neutral complex-type N-glycans, as well as hybrid-type or monoantennary neutral N-glycans, compared to other structural groups in the profiles.

Identification of soluble glycan components. Similarly to CB mononuclear cell populations, in the present analysis neutral glycan components were identified in all the cell types that were assigned as soluble glycans based on their proposed monosaccharide compositions including components from the glycan group Hex₂₋₁₂HexNAc₁ (see Figures). The abundancies of these glycan components in relation to each other and in relation to the other glycan signals vary between individual samples and cell types.

Sialylated N-glycan profiles. Sialylated N-glycan profiles obtained from two CB MSC lines resemble closely each other with respect to their overall sialylated N-glycan profiles. However, minor differences between the profiles are observed, and some glycan signals can only be observed in one cell line, indicating that the two cell lines have glycan structures that differ them from each other. The analysis revealed in each cell type the relative proportions of about 50-70 glycan signals that were assigned as acidic N-glycan components. Typically, significant differences in the glycan profiles between cell populations are consistent throughout multiple experiments.

Differentiation-associated changes in glycan profiles. Neutral N-glycan profiles of CB MSCs change upon differentiation in adipogenic cell culture medium. The present results indicate that relative abundancies of several individual glycan signals as well as glycan signal groups change due to cell culture in differentiation medium. The major change in glycan structural groups associated with differentiation is increase in amounts of neutral complex-type N-glycans, such as signals at m/z 1663 and m/z 1809, corresponding to the Hex₅HexNAc₄ and Hex₅HexNAc₄dHex₁ monosaccharide compositions, respectively. Changes were also observed in sialylated glycan profiles.

Glycosidase analyses of neutral N-glycans. Specific exoglycosidase digestions were performed on isolated neutral N-glycan fractions from CB MSC lines as described in Examples. The results of α-mannosidase analysis show in detail which of the neutral N-glycan signals in the neutral N-glycan profiles of CB MSC lines are susceptible to α-mannosidase digestion, indicating for the presence of non-reducing terminal α-mannose residues in the corresponding glycan structures. As an example, the major neutral N-glycan signals at m/z 1257, 1419, 1581, 1743, and 1905, which were preliminarily assigned as high-mannose type N-glycans according to their proposed monosaccharide compositions Hex₅₋9HexNAc₂, were shown to contain terminal α-mannose residues thus confirming the preliminary assignment. The results indicate for the presence of non-reducing terminal β1,4-galactose residues in the corresponding glycan structures. As an example, the major neutral complex-type N-glycan signals at m/z 1663 and m/z 1809 were shown to contain terminal β1,4-linked galactose residues.

### Bone Marrow Derived Mesenchymal Stem Cell (BM MSC) Lines

Neutral N-glycan profiles and differentiation-associated changes in glycan profiles. Neutral N-glycan profiles obtained from a BM MSC line, grown in proliferation medium and in osteogenic medium resemble CB MSC lines with respect to their overall neutral N-glycan profiles. However, differences between cell lines derived from the two sources are observed, and some glycan signals can only be observed in one cell line, indicating that the cell lines have glycan structures that differ them from each other. The major characteristic structural feature of BM MSCs is even more abundant neutral complex-type N-glycans compared to CB MSC lines. Similarly to CB MSCs, these glycans were also the major increased glycan signal group upon differentiation of BM MSCs. The analysis revealed in each cell type the relative proportions of about 50-70 glycan signals that were assigned as non-sialylated N-glycan components. Typically, significant differences in the glycan profiles between cell populations are consistent throughout multiple experiments.

Sialylated N-glycan profiles. Sialylated N-glycan profiles obtained from a BM MSC line, grown in proliferation medium and in osteogenic medium. The undifferentiated and differentiated cells resemble closely each other with respect to their overall sialylated N-glycan profiles. However, minor differences between the profiles are observed, and some glycan signals can only be observed in one cell line, indicating that the two cell types have glycan structures that differ them from each other. The analysis revealed in each cell type the relative proportions of about 50 glycan signals that were assigned as acidic N-glycan components. Typically, significant differences in the glycan profiles between cell populations are consistent throughout multiple experiments.

Sialidase analysis. The sialylated N-glycan fraction isolated from BM MSCs was digested with broad-range sialidase as described in the preceding Examples. After the reaction, it was observed by MALDI-TOF mass spectrometry that the vast majority of the sialylated N-glycans were desialylated and transformed into corresponding neutral N-glycans, indicating that they had contained sialic acid residues (NeuAc and/or NeuGc) as suggested by the proposed monosaccharide compositions. Glycan profiles of combined neutral and desialylated (originally sialylated) N-glycan fractions of BM MSCs grown in proliferation medium and in osteogenic medium correspond to total N-glycan profiles isolated from the cell samples (in desialylated form). It is calculated that in undifferentiated BM MSCs (grown in osteogenic medium), approximately 53% of the N-glycan signals correspond to high-mannose type N-glycan monosaccharide compositions, 8% to low-mannose type N-glycans, 31% to complex-type N-glycans, and 7% to hybrid-type or monoantennary N-glycan monosaccharide compositions. In differentiated BM MSCs (grown in osteogenic medium), approximately 28% of the N-glycan signals correspond to high-mannose type N-glycan monosaccharide compositions, 9% to low-mannose type N-glycans, 50% to complex-type N-glycans, and 11% to hybrid-type or monoantennary N-glycan monosaccharide compositions.

Lectin binding analysis of mesenchymal stem cells. As described under Experimental procedures, bone marrow derived mesenchymal stem cells were analyzed for the presence of ligands of α2,3-linked sialic acid specific (MAA) and α2,6-linked sialic acid specific (SNA) lectins on their surface. It was revealed that MAA bound strongly to the cells whereas SNA bound weakly, indicating that in the cell culture conditions, the cells had significantly more α2,3-linked than α2,6-linked sialic acids on their surface glycoconjugates. The present results suggest that lectin staining can be used as a further means to distinguish different cell types and complements mass spectrometric profiling results.

Detection of Potential Glycan Contaminations from Cell Culture Reagents

In the sialylated N-glycan profiles of MSC lines, specific N-glycan signals were observed that indicated contamination of mesenchymal stem cell glycoconjugates by abnormal sialic acid residues. First, when the cells were cultured in cell culture media with added animal sera, such as bovine of equine sera, potential contamination by N-glycolylneuraminic acid (Neu5Gc) was detected. The glycan signals at m/z 1946, corresponding to the [M-H]⁻ ion of NeuGc₁Hex₅HexNAc₄, as well as m/z 2237 and m/z 2253, corresponding to the [M-H]⁻ ions of NeuGc₁NeuAc₁Hex₅HexNAc₄ and NeuGc₂Hex₅HexNAc₄, respectively, were indicative of the presence of Neu5Gc, i.e. a sialic acid residue with 16 Da larger mass than N-acetylneuraminic acid (Neu5Ac). Moreover, when the cells were cultured in cell culture media with added horse serum, potential contamination by O-acetylated sialic acids was detected. Diagnostic signals used for detection of O-acetylated sialic acid containing sialylated N-glycans included [M-H]⁻ ions of Ac₁NeuAc₁Hex₅HexNAc₄, Ac₁NeuAc₂Hex₅HexNAc₄, and Ac₂NeuAc₂Hex₅HexNAc₄, at calculated m/z 1972.7, 2263.8, and 2305.8, respectively.

### Conclusions

Uses of the glycan profiling method. The results indicate that the present glycan profiling method can be used to differentiate CB MSC lines and BM MSC lines from each other, as well as from other cell types such as cord blood mononuclear cell populations. Differentiation-induced changes as well as potential glycan contaminations from e.g. cell culture media can also be detected in the glycan profiles, indicating that changes in cell status can be detected by the present method. The method can also be used to detect MSC-specific glycosylation features including those discussed below.

Differences in glycosylation between cultured cells and native human cells. The present results indicate that BM MSC lines have more high-mannose type N-glycans and less low-mannose type N-glycans compared to the other N-glycan structural groups than mononuclear cells isolated from cord blood. Taken together with the results obtained from cultured human embryonal stem cells in the following Examples, it is indicated that this is a general tendency of cultured stem cells compared to native isolated stem cells. However, differentiation of BM MSCs in osteogenic medium results in significantly increased amounts of complex-type N-glycans and reduction in the amounts of high-mannose type N-glycans.

Mesenchymal stem cell line specific glycosylation features. The present results indicate that mesenchymal stem cell lines differ from the other cell types studied in the present study with regard to specific features of their glycosylation, such as:
1) Both CB MSC lines and BM MSC lines have unique neutral and sialylated N-glycan profiles;
2) The major characteristic structural feature of both CB and BM MSC lines is abundant neutral complex-type N-glycans;
3) An additional characteristic feature is low sialylation level of complex-type N-glycans.

### Example 2

### MALDI-TOF Mass Spectrometric N-Glycan Profiling of Human Embryonic Stem Cell Lines

### Examples of Cell Material Production

Human Embryonic Stem Cell Lines (hESC)

Undifferentiated hESC. Processes for generation of hESC lines from blastocyst stage in vitro fertilized excess human embryos have been described previously (e.g. Thomson et al., 1998). Two of the analysed cell lines in the present work were initially derived and cultured on mouse embryonic fibroblasts feeders (MEF; 12-13 pc fetuses of the ICR strain), and two on human foreskin fibroblast feeder cells (HFF; CRL-2429 ATCC, Mananas, USA). For the present studies all the lines were transferred on HFF feeder cells treated with mitomycin-C (1 μg/ml; Sigma-Aldrich) and cultured in serum-free medium (Knockout™ D-MEM; Gibco® Cell culture systems, Invitrogen, Paisley, UK) supplemented with 2 mM L-Glutamin/Penicillin streptomycin (Sigma-Aldrich), 20% Knockout Serum Replacement (Gibco), 1× non-essential amino acids (Gibco), 0.1 mM β-mercaptoethanol (Gibco), 1×ITSF (Sigma-Aldrich) and 4 ng/ml bFGF (Sigma/Invitrogen).

Stage 2 differentiated hESC (embryoid bodies). To induce the formation of embryoid bodies (EB) the hESC colonies were first allowed to grow for 10-14 days whereafter the colonies were cut in small pieces and transferred on non-adherent Petri dishes to form suspension cultures. The formed EBs were cultured in suspension for the next 10 days in standard culture medium (see above) without bFGF.

Stage 3 differentiated hESC. For further differentiation EBs were transferred onto gelatin-coated (Sigma-Aldrich) adherent culture dishes in media consisting of DMEM/F12 mixture (Gibco) supplemented with ITS, Fibronectin (Sigma), L-glutamine and antibiotics. The attached cells were cultured for 10 days whereafter they were harvested.

Sample preparation. The cells were collected mechanically, washed, and stored frozen prior to glycan analysis.

### Results

Neutral N-glycan profiles—effect of differentiation status. Neutral N-glycan profiles obtained from a human embryonal stem cell (hESC) line, its embryoid body (EB) differentiated form, and its stage 3 (st.3) differentiated form. Although the cell types resemble each other with respect to the major neutral N-glycan signals, the neutral N-glycan profiles of the two differentiated cell forms differ significantly from the undifferentiated hESC profile. In fact, the farther differentiated the cell type is, the more its neutral N-glycan profile differs from the undifferentiated hESC profile. Multiple differences between the profiles are observed, and many glycan signals can only be observed in one or two out of three cell types, indicating that differentiation induces the appearance of new glycan types. The analysis revealed in each cell type the relative proportions of about 40-55 glycan signals that were assigned as non-sialylated N-glycan components. Typically, significant differences in the glycan profiles between cell populations are consistent throughout multiple experiments.

Neutral N-glycan profiles—comparison of hESC lines. Neutral N-glycan profiles obtained from four hESC lines closely resemble each other. Individual profile characteristics and cell line specific glycan signals are present in the glycan profiles, but it is concluded that hESC lines resemble more each other with respect to their neutral N-glycan profiles and are different from differentiated EB and st.3 cell types. hESC lines 3 and 4 are derived from sibling embryos, and their neutral N-glycan profiles resemble more each other and are different from the two other cell lines, i.e. they contain common glycan signals. The analysis revealed in each cell type the relative proportions of about 40-55 glycan signals that were assigned as non-sialylated N-glycan components. Typically, significant differences in the glycan profiles between cell populations are consistent throughout multiple experiments.

Neutral N-glycan structural features. Neutral N-glycan groupings proposed for analysed cell types are presented in Table 6. Again, the analysed three major cell types, namely undifferentiated hESCs, differentiated cells, and human fibroblast feeder cells, differ from each other significantly. Within each cell type, however, there are minor differences between individual cell lines. Moreover, differentiation-associated neutral N-glycan structural features are expressed more strongly in st.3 differentiated cells than in EB cells. Cell-type specific glycosylation features are discussed below in Conclusions.

Glycosidase analysis of neutral N-glycan fractions. Specific exoglycosidase digestions were performed on isolated neutral N-glycan fractions from hESC lines as described in the preceding Examples. In α-mannosidase analysis, several neutral glycan signals were shown to be susceptible to α-mannosidase digestion, indicating for potential presence of non-reducing terminal α-mannose residues in the corresponding glycan structures. In hESC and EB cells, these signals included m/z 917, 1079, 1095, 1241, 1257, 1378, 1393, 1403, 1444, 1555, 1540, 1565, 1581, 1606, 1622, 1688, 1743, 1768, 1905, 1996, 2041, 2067, 2158, and 2320. In β1,4-galactosidase analysis, several neutral glycan signals were shown to be susceptible to β1,4-galactosidase digestion, indicating for potential presence of non-reducing terminal β1,4-galactose residues in the corresponding glycan structures. In hESC and EB cells, these signals included m/z 609, 771, 892, 917, 1241, 1378, 1393, 1555, 1565, 1606, 1622, 1647, 1663, 1704, 1809, 1850, 1866, 1955, 1971, 1996, 2012, 2028, 2041, 2142, 2174, and 2320. In α1,3/4-fucosidase analysis, several neutral glycan signals were shown to be susceptible to α1,3/4-fucosidase digestion, indicating for potential presence of non-reducing terminal α1,3- and/or α1,4-fucose residues in the corresponding glycan structures. In hESC and EB cells, these signals included m/z 1120, 1590, 1784, 1793, 1955, 1996, 2101, 2117, 2142, 2158, 2190, 2215, 2247, 2263, 2304, 2320, 2393, and 2466.

Identification of soluble glycan components. Similarly to the cell types described in the preceding examples, in the present analysis neutral glycan components were identified in all the cell types that were assigned as soluble glycans based on their proposed monosaccharide compositions including components from the glycan group Hex₂₋₁₂HexNAc₁ (see Figures). The abundancies of these glycan components in relation to each other and in relation to the other glycan signals vary between individual samples and cell types.

Sialylated N-glycan profiles—effect of differentiation status. Sialylated N-glycan profiles obtained from a human embryonal stem cell (hESC) line, its embryoid body (EB) differentiated form, and its stage 3 (st.3) differentiated form. Although the cell types resemble each other with respect to the major sialylated N-glycan signals, the sialylated N-glycan profiles of the two differentiated cell forms differ significantly from the undifferentiated hESC profile. In fact, the farther differentiated the cell type is, the more its sialylated N-glycan profile differs from the undifferentiated hESC profile. Multiple differences between the profiles are observed, and many glycan signals can only be observed in one or two out of three cell types, indicating that differentiation induces the appearance of new glycan types as well as decrease in amounts of stem cell specific glycan types. For example, there is significant differentiation-associated decrease in relative amounts of glycan signals at m/z 1946 and 2222, corresponding to monosaccharide compositions NeuGc₁Hex₅HexNAc₄ and NeuAc₁Hex₅HexNAc₄dHex₂, respectively. The analysis revealed in each cell type the relative proportions of about 50-70 glycan signals that were assigned as acidic N-glycan components. Typically, significant differences in the glycan profiles between cell populations are consistent throughout multiple experiments.

Sialylated N-glycan profiles—comparison of hESC lines. Sialylated N-glycan profiles obtained from four hESC lines closely resemble each other. Individual profile characteristics and cell line specific glycan signals are present in the glycan profiles, but it is concluded that hESC lines resemble more each other with respect to their sialylated N-glycan profiles and are different from differentiated EB and st.3 cell types. The analysis revealed in each cell type the relative proportions of about 50-70 glycan signals that were assigned as acidic N-glycan components. Typically, significant differences in the glycan profiles between cell populations are consistent throughout multiple experiments.

Human fibroblast feeder cell lines. Sialylated N-glycan profiles obtained from human fibroblast feeder cell lines differ from hESC, EB, and st.3 differentiated cells, and that feeder cells grown separately and with hESC cells differ from each other.

Sialylated N-glycan structural features. Sialylated N-glycan groupings proposed for analysed cell types are presented in Table 7. Again, the analysed three major cell types, namely undifferentiated hESCs, differentiated cells, and human fibroblast feeder cells, differ from each other significantly. Within each cell type, however, there are minor differences between individual cell lines. Moreover, differentiation-associated sialylated N-glycan structural features are expressed more strongly in st.3 differentiated cells than in EB cells. Cell-type specific glycosylation features are discussed below in Conclusions.

### Conclusions

Comparison of glycan profiles. Differences in the glycan profiles between cell types were consistent throughout multiple samples and experiments, indicating that the present method of glycan profiling and the differences in the present glycan profiles can be used to identify hESCs or cells differentiated therefrom, or other cells such as feeder cells, or to determine their purity, or to identify cell types present in a sample. The present method and the present results can also be used to identify cell-type specific glycan structural features or cell-type specific glycan profiles. The method proved especially useful in determination of differentiation stage, as demonstrated by comparing analysis results between hESC, EB, and st.3 differentiated cells. Furthermore, hESCs were shown to have unique glycosylation profiles, which can be differentiated from differentiated cell types as well as from other stem cell types such as MSCs, indicating that stem cells in general and also specific stem cell types can be identified by the present method. The present method could also detect glycan structures common to hESC lines derived from sibling embryos, indicating that related structural features can be identified in different cell lines or their similarity be estimated by the present method.

Comparison of neutral N-glycan structural features. Differences in glycosylation profiles between analyzed cell types were identified based on proposed structural features, which can be used to identify cell-type specific glycan structural features. Identified cell-type specific features of neutral N-glycan profiles are concluded below:

### HESC Lines:

1) Increased amounts of fucosylated neutral N-glycans, especially glycans with two or more deoxyhexose residues per chain, indicating increased expression of neutral N-glycans containing α1,2-, α1,3-, or α1,4-linked fucose residues; and
2) Increased amounts of larger neutral N-glycans.

### EBs and St.3 Differentiated Cells (St.3 Cells Expressing the Features More Strongly):

1) Lower amounts of neutral N-glycans containing two or more deoxyhexose residues per chain, indicating reduced expression of neutral N-glycans containing α1,2-, α1,3-, or α1,4-linked fucose residues;
2) Increased amounts of hybrid-type, monoantennary, and complex-type neutral N-glycans.
3) Increased amounts of terminal HexNAc residues; and
4) Potentially increased amounts of bisecting GlcNAc structures.

### Human Fibroblast Feeder Cells:

1) Increased amounts of larger neutral N-glycans;
2) Lower amounts of neutral N-glycans containing two or more deoxyhexose residues per chain, indicating reduced expression of neutral N-glycans containing α1,2-, α1,3-, or α1,4-linked fucose residues;
3) Increased amounts of terminal HexNAc residues; and
4) Potentially no bisecting GlcNAc structures.

Comparison of sialylated N-glycan structural features. Differences in glycosylation profiles between analyzed cell types were identified based on proposed structural features, which can be used to identify cell-type specific glycan structural features. Identified cell-type specific features of sialylated N-glycan profiles are concluded below:

### HESC Lines:

1) Increased amounts of fucosylated sialylated N-glycans, especially glycans with two or more deoxyhexose residues per chain, indicating increased expression of sialylated N-glycans containing α1,2-, α1,3-, or α1,4-linked fucose residues;
2) Increased amounts of terminal HexNAc residues; and
3) Increased amounts of Neu5Gc containing sialylated N-glycans.

### EBs and St.3 Differentiated Cells (St.3 Cells Expressing the Features More Strongly):

1) Lower amounts of sialylated N-glycans containing two or more deoxyhexose residues per chain, indicating reduced expression of sialylated N-glycans containing α1,2-, α1,3-, or α1,4-linked fucose residues;
2) Increased amounts of hybrid-type or monoantennary sialylated N-glycans; and
3) Potentially increased amounts of bisecting GlcNAc structures.

### Human Fibroblast Feeder Cells:

1) Increased amounts of larger sialylated N-glycans;
2) Lower amounts of terminal HexNAc residues; and
3) Potentially lower amounts of bisecting GlcNAc structures.

### Example 3

### Lectin and Antibody Profiling of Human Embryonic Stem Cells

### Experimental Procedures

Cell samples. Human embryonic stem cell (hESC) lines FES 22 and FES 30 (Family Federation of Finland) were propagated on mouse feeder cell (mEF) layers as described above.

FITC-labeled lectins. Fluorescein isothiocyanate (FITC) labeled lectins were purchased from several manufacturers: FITC-GNA, -HHA, -MAA, -PWA, -STA and -LTA were from EY Laboratories (USA); FITC-PSA and UEA and biotin-labelled WFA were from Sigma (USA); and FITC-RCA, -PNA and -SNA were from Vector Laboratories (UK).

Fluorescence microscopy labeling experiments were conducted essentially as described in the preceding Examples. Biotin label was visualized by fluorescein-conjugated streptavidin.

### Results

Table 19 shows the tested FITC-labelled lectins and antibodies, examples of their target saccharide sequences, and the graded lectin binding intensities as described in the Table legend, in fluorescence microscopy of fixed cells grown on microscopy slides. Multiple binding specificities for the used lectins are described in the art and in general the binding of a lectin in the present experiments means that the cells express specific ligands for the lectin on their surface, but does not exclude the presence of also other ligands that are recognized by the lectin. See Example 14 for specificities for GF antibodies.

α-linked mannose and core Fucα6-eptopes. Abundant labelling of mEF by Pisum sativum (PSA) lectins suggests that they express mannose, more specifically α-linked mannose residues and core Fucα6-eptopes on their surface (or intracellular) glycoconjugates such as N-glycans. The results further suggest that the both hESC lines do not express these ligands at as high concentrations as mEF on their surface.

β-linked galactose. Abundant labelling of hESC by peanut lectin (PNA) and less intense labelling by Ricinus communis lectin I (RCA-I) suggests that hESC express β-linked non-reducing terminal galactose residues on their surface glycoconjugates such as N- and/or O-glycans. More specifically, RCA-I binding suggests that the cells contain high amounts of unsubstituted Galβ epitopes on their surface. PNA binding suggests for the presence of unsubstituted Galβ, and the absence of specific binding of PNA to mEF suggests that the binding epitopes for this lectin are less abundant in mEF.

Sialic acids. Specific labelling of hESC by both Maackia amurensis (MAA) and Sambucus nigra (SNA) lectins suggests that the cells express sialic acid residues on their surface glycoconjugates such as N- and/or O-glycans and/or glycolipids. More specifically, the specific MAA binding of hESC suggests that the cells contain high amounts of α2,3-linked sialic acid residues. In contrast, the results suggest that these epitopes are less abundant in mEF. SNA binding in both cell types suggests for the presence of also α2,6-linkages in the sialic acid residues on the cell surface.

Poly-N-acetyllactosamine sequences. Labelling of the cells by pokeweed (PWA) and less intense labelling by Solanum tuberosum (STA) lectins suggests that the cells express poly-N-acetyllactosamine sequences on their surface glycoconjugates such as N- and/or O-glycans and/or glycolipids. The results further suggest that cell surface poly-N-acetyllactosamine chains contain both linear and branched sequences.

β-linked N-acetylgalactosamine. Abundant labelling of hESC by Wisteria floribunda lectin (WFA) suggests that hESC express β-linked non-reducing terminal N-acetylgalactosamine residues on their surface glycoconjugates such as N- and/or O-glycans. The absence of specific binding of WFA to mEF suggests that the lectin ligand epitopes are less abundant in mEF.

Fucosylation. Labelling of the cells by Ulex europaeus (UEA) and less intense labelling by Lotus tetragonolobus (LTA) lectins suggests that the cells express fucose residues on their surface glycoconjugates such as N- and/or O-glycans and/or glycolipids. More specifically, the UEA binding suggests that the cells contain α-linked fucose residues including α1,2-linked fucose residues. LTA binding suggests for the presence of α-linked fucose residues including α1,3- or α1,4-linked fucose residues on the cell surface.

The specific antibody anti-Lex and anti-sLex antibody binding results indicate that the hESC samples contain Galβ4(Fucα3)GlcNAcβ and SAα3Galβ4(Fucα3)GlcNAcβ carbohydrate epitopes on their surface, respectively.

Taken together, in the present experiments the lectins PNA, MAA, and WFA as well as the antibodies anti-Lex and anti-sLex bound specifically to hESC but not to mEF. In contrast, the lectin PSA bound specifically to mEF but not to hESC. This suggests that the glycan epitopes that these reagents recognize have hESC or mEF specific expression patterns. On the other hand, other reagents in the tested reagent panel bound differentially to the two hESC lines FES 22 and FES 30, indicating cell line specific glycosylation of the hESC cell surfaces (Table 19).

### Discussion

Venable, A., et al. (2005 BMC Dev. Biol.) have previously described lectin binding profiles of SSEA-4 enriched human embryonic stem cells (hESC) grown on mouse feeder cells. The lectins used were Lycopersicon esculentum (LEA, TL), RCA, Concanavalin A (ConA), WFA, PNA, SNA, Hippeastrum hybrid (HHA, HHL), Vicia villosa (VVA), UEA, Phaseolus vulgaris (PHA-L and PHA-E), MAA, LTA (LTL), and Dolichos biflorus (DBA) lectins. In FACS and cytochemistry analysis, four lectins were found to have similar binding percentage as SSEA-4 (LEA, RCA, ConA, and WFA) and in addition two lectins also had high binding percentage (PNA and SNA). Two lectins did not bind to hESCs (DBA and LTA). Six lectins were found to partially bind to hESC (PHA-E, VVA, UEA, PHA-L, MAA, and HHA). The authors suggested that the differential lectin binding specificities can be used to distinguish hESC and differentiated hESC types based on carbohydrate presentation.

Venable et al. (2005) discuss some carbohydrate structures that they claim to have high expression on the surface of pluripotent SSEA-4 hESC (corresponding lectins according to Venable et al. in parenthesis): α-Man (ConA, HHA), Glc (ConA), Galβ3GalNAcβ (PNA), non-reducing terminal Gal (RCA), non-reducing terminal β-GalNAc (RCA), GalNAcβ4Gal (WFA), GlcNAc (LEA), and SAα6GalNAc (SNA). In addition, Venable et al. discuss some carbohydrate structures that they claim to have expression on surface of a proportion of pluripotent SSEA-4 hESC (corresponding lectins according to Venable et al. in parenthesis): Gal (PHA-L, PHA-E, MAA), GalNAc (VVA) and Fuc (UEA). However, based on the monosaccharide specificities oligosaccharide specificities on the target cannot be known e.g. ConA is not easily assigned to any specific to Glc or Man-structure and our MAA has no specificity to Gal residues, but SAα3-structures; it is realized that large differences exist between often numerous isolectins of a plant species and Venable did not disclose the exact lectins used. Technical problems avoiding exact interpretation is Background section.

In the present experiments, RCA binding was observed on both hESC line FES 22 and mEF, but not on FES 30. This suggests that RCA binding specificity in hESC varies from cell line to another. The present experiments also show other lectins to be expressed on only one out of the two hESC lines (Table 19), suggesting that there is individual variation in binding of some lectins.

Based on LTA not binding to hESC in their experiments, Venable et al. (2005) suggest that on hESC surface there are no non-modified fucose residues that are α-linked to GlcNAc. However, in the present experiments LTA as well as anti-Lex and anti-sLex monoclonal antibodies were found to bind to the hESC line FES 22. The present antibody binding results indicate that FucαGlcNAc epitopes, specifically Galβ4(Fucα3)GlcNAc sequences, are present on hESC surface.

Venable et al. (2005) describe that PNA recognizes in their hESC samples specifically Galβ3GalNAc structures, wherein the GalNAcresidue is β-linked. In the present experiments, PNA was used to recognize carbohydrate structures generally including β-linked galactose residues and without β-linkage requirement for the GalNAc residue.

Venable et al. (2005) describe that SNA recognizes in their hESC samples specifically SAα6GalNAc structures. In the present experiments, SNA was used to recognize α2,6-linked sialic acids in general and its ligands were also found on mEF.

Inhibition of MAA binding by 200 mM lactose in the experiments described by Venable et al. (2005) suggests non-specific binding of their MAA with respect to sialic acids. According to the present experiments, our MAA can recognize α2,3-linked sialic acid residues on hESC surface and differentiate between hESC and mEF.

### Example 4

### Lectin and Antibody Profiling of Human Mesenchymal Stem Cells

### Experimental Procedures

Cell samples. Bone marrow derived human mesenchymal stem cell lines (MSC) were generated and cultured in proliferation medium as described above.

FITC-labeled lectins. Fluorescein isothiocyanate (FITC) labelled lectins were purchased from several manufacturers: FITC-GNA, -HHA, -MAA, -PWA, -STA and -LTA were from EY Laboratories (USA); FITC-PSA and -UEA were from Sigma (USA); and FITC-RCA, -PNA and -SNA were from Vector Laboratories (UK). Lectins were used in dilution of 5 μg/10⁵ cells in 1% human serum albumin (HSA; FRC Blood Service, Finland) in phosphate buffered saline (PBS).

Flow cytometry. Flow cytometric analysis of lectin binding was used to study the cell surface carbohydrate expression of MSC. 90% confluent MSC layers on passages 9-11 were washed with PBS and harvested into single cell suspensions by 0.25% trypsin-1 mM EDTA solution (Gibco). The trypsin treatment was aimed to gentle, but it is realized that part of the structures recognized when compared to experiments by antibodies may be partially lost or reduced. Detached cells were centrifuged at 600 g for five minutes at room temperature. Cell pellet was washed twice with 1% HSA-PBS, centrifuged at 600 g and resuspended in 1% HSA-PBS. Cells were placed in conical tubes in aliquots of 70000-83000 cells each. Cell aliquots were incubated with one of the FITC labelled lectin for 20 minutes at room temperature. After incubation cells were washed with 1% HSA-PBS, centrifuged and resuspended in 1% HSA-PBS. Untreated cells were used as controls. Lectin binding was detected by flow cytometry (FACSCalibur, Becton Dickinson). Data analysis was made with Windows Multi Document Interface for Flow Cytometry (WinMDI 2.8). Two independent experiments were carried out.

Fluorescence Microscopy Labeling Experiments were Conducted as Described in the Preceding Examples.

### Results and Discussion

Table 20 shows the tested FITC-labelled lectins, examples of their target saccharide sequences, and the amount of cells showing positive lectin binding (%) in FACS analysis after mild trypsin treatment. Table 21 shows the tested FITC-labelled lectins, examples of their target saccharide sequences, and the graded lectin binding intensities as described in the Table legend, in fluorescence microscopy of fixed cells grown on microscopy slides. Binding specificities of the used lectins are described in the art and in general the binding of a lectin in the present experiments means that the cells express specific ligands for the lectin on their surface. The examples of some of the specificities discussed below and those marked in the Tables are therefore non-exclusive in nature.

α-linked mannose. Abundant labelling of the cells by both Hippeastrum hybrid (HHA) and Pisum sativum (PSA) lectins suggests that they express mannose, more specifically α-linked mannose residues on their surface glycoconjugates such as N-glycans. Possible α-mannose linkages include α1→2, α1→3, and α1→6. The lower binding of Galanthus nivalis (GNA) lectin suggests that some α-mannose linkages on the cell surface are more prevalent than others.

β-linked galactose. Abundant labelling of the cells by Ricinus communis lectin I (RCA-I) and less intense labelling by peanut lectin (PNA) suggests that the cells express β-linked non-reducing terminal galactose residues on their surface glycoconjugates such as N- and/or O-glycans. More specifically, the intense RCA-I binding suggests that the cells contain high amounts of unsubstituted Galβ epitopes on their surface. The binding of RCA-I was increased by sialidase treatment of the cells before lectin binding, indicating that the ligands of RCA-I on MSC were originally partly covered by sialic acid residues. PNA binding suggests for the presence of another type of unsubstituted Galβ3 epitopes such as Core 1 O-glycan epitopes on the cell surface. The binding of PNA was also increased by sialidase treatment of the cells before lectin binding, indicating that the ligands of PNA on MSC were originally mostly covered by sialic acid residues. These results suggest that both RCA-I and PNA can be used to assess the amount of their specific ligands on the cell surface of BM MSC, and with or without conjunction with sialidase treatment to assess the sialylation level of their specific epitopes.

Sialic acids. Abundant labelling of the cells by Maackia amurensis (MAA) and less intense labelling by Sambucus nigra (SNA) lectins suggests that the cells express sialic acid residues on their surface glycoconjugates such as N- and/or O-glycans and/or glycolipids. More specifically, the intense MAA binding suggests that the cells contain high amounts of α2,3-linked sialic acid residues on their surface. SNA binding suggests for the presence of also α2,6-linked sialic acid residues on the cell surface, however in lower amounts than α2,3-linked sialic acids. Both of these lectin binding activities could be reduced by sialidase treatment, indicating that the specificities of the lectins in BM MSC are mostly targeted to sialic acids.

Poly-N-acetyllactosamine sequences. Labelling of the cells by Solanum tuberosum (STA) and less intense labelling by pokeweed (PWA) lectins suggests that the cells express poly-N-acetyllactosamine sequences on their surface glycoconjugates such as N- and/or O-glycans and/or glycolipids. Higher intensity labelling with STA than with PWA suggests that most of the cell surface poly-N-acetyllactosamine sequences are linear and not branched or substituted chains.

Fucosylation. Labelling of the cells by Ulex europaeus (UEA) and less intense labelling by Lotus tetragonolobus (LTA) lectins suggests that the cells express fucose residues on their surface glycoconjugates such as N- and/or O-glycans and/or glycolipids. More specifically, the UEA binding suggests that the cells contain α-linked fucose residues, including α1,2-linked fucose residues, on their surface. LTA binding suggests for the presence of also α-linked fucose residues, including α1,3-linked fucose residues on the cell surface, however in lower amounts than UEA ligand fucose residues.

Mannose-binding lectin labelling. Low labelling intensity was also detected with human serum mannose-binding lectin (MBL) coupled to fluorescein label, suggesting that ligands for this innate immunity system component may be expressed on in vitro cultured BM MSC cell surface.

Binding of a NeuGc polymeric probe (Lectinity Ltd., Russia) to non-fixed hESC indicates the presence of NeuGc-specific lectin on the cell surfaces. In contrast, polymeric NeuAc probe did not bind to the cells with same intensity in the present experiments.

The binding of the specific antibodies to hESC indicates the presence of Lex and sialyl-Lewis x epitopes on their surfaces, and binding of NeuGc-specific antibody to hESC indicates the presence of NeuGc epitopes on their surfaces.

### Example 5

### Lectin and Antibody Profiling of Human Cord Blood Cell Populations

### Results and Discussion

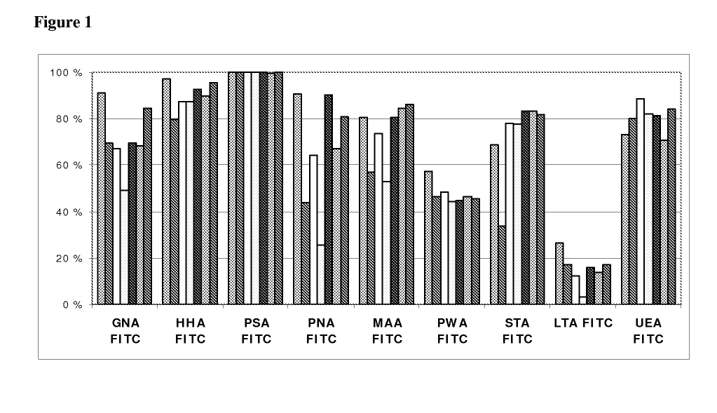

FIG. 1 shows the results of FACS analysis of FITC-labelled lectin binding to seven individual cord blood mononuclear cell (CB MNC) preparations (experiments performed as described above). Strong binding was observed in all samples by GNA, HHA, PSA, MAA, STA, and UEA FITC-labelled lectins, indicating the presence of their specific ligand structures on the CB MNC cell surfaces. Also mediocre binding (PWA), variable binding between CB samples (PNA), and low binding (LTA) was observed, indicating that the ligands for these lectins are either variable or more rare on the CB MNC cell surfaces as the lectins above.

### Example 6

### Analysis of Total N-Glycomes of Human Stem Cells and Cell Populations

### Experimental Procedures

Cell and glycan samples were prepared as described in the preceding Examples.

Relative proportions of neutral and acidic N-glycan fractions were studied by desialylating isolated acidic glycan fraction with A. ureafaciens sialidase as described in the preceding Examples and then combining the desialylated glycans with neutral glycans isolated from the same sample. Then the combined glycan fractions were analyzed by positive ion mode MALDI-TOF mass spectrometry as described in the preceding Examples. The proportion of sialylated N-glycans of the combined N-glycans was calculated by calculating the percentual decrease in the relative intensity of neutral N-glycans in the combined N-glycan fraction compared to the original neutral N-glycan fraction, according to the equation:

$$proportion = \frac{{I}^{neutral} - {I}^{combined}}{{I}^{neutral}} \times 100 \% ,$$

wherein I^{neutral} and I^{combined} correspond to the sum of relative intensities of the five high-mannose type N-glycan [M+Na]⁺ ion signals at m/z 1257, 1419, 1581, 1743, and 1905 in the neutral and combined N-glycan fractions, respectively.

### Results and Discussion

The relative proportions of acidic N-glycan fractions in studied stem cell types were as follows: in human embryonic stem cells (hESC) approximately 35% (proportion of sialylated and neutral N-glycans is approximately 1:2), in human bone marrow derived mesenchymal stem cells (BM MSC) approximately 19% (proportion of sialylated and neutral N-glycans is approximately 1:4), in osteoblast-differentiated BM MSC approximately 28% (proportion of sialylated and neutral N-glycans is approximately 1:3), and in human cord blood (CB) CD133+ cells approximately 38%

(proportion of sialylated and neutral N-glycans is approximately 2:3).

In conclusion, BM MSC differ from hESC and CB CD133+ cells in that they contain significantly lower amounts of sialylated N-glycans compared to neutral N-glycans. However, after osteoblast differentiation of the BM MSC the proportion of sialylated N-glycans increases.

### Example 7

### Analysis of the Human Embryonic Stem Cell N-Glycome

### Experimental Procedures

Human embryonic stem cell lines (hESC). Four Finnish hESC lines, FES 21, FES 22, FES 29, and FES 30, were used in the present study. Generation of the lines has been described (Skottman et al., 2005, and M.M., C.O., T.T., and T.O., manuscript submitted for publication). Two of the analysed cell lines in the present work were initially derived and cultured on mouse embryonic fibroblast feeders, and two on human foreskin fibroblast feeder cells. For the mass spectrometry studies all of the lines were transferred on HFF feeder cells treated with mitomycin-C (1 μg/ml, Sigma-Aldrich, USA) and cultured in serum-free medium (Knockout™ D-MEM; Gibco® Cell culture systems, Invitrogen, UK) supplemented with 2 mM L-Glutamin/Penicillin streptomycin (Sigma-Aldrich), 20% Knockout Serum Replacement (Gibco), 1× non-essential amino acids (Gibco), 0.1 mM β-mercaptoethanol (Gibco), 1×ITS (Sigma-Aldrich) and 4 ng/ml bFGF (Sigma/Invitrogen). To induce the formation of embryoid bodies (EB) the hESC colonies were first allowed to grow for 10-14 days whereafter the colonies were cut in small pieces and transferred on non-adherent Petri dishes to form suspension cultures. The formed EBs were cultured in suspension for the next 10 days in standard culture medium (see above) without bFGF. For further differentiation (into stage 3 differentiated cells) EBs were transferred onto gelatin-coated (Sigma-Aldrich) adherent culture dishes in media consisting of DMEM/F12 mixture (Gibco) supplemented with ITS, Fibronectin (Sigma), L-glutamine and antibiotics. The attached cells were cultured for 10 days whereafter they were harvested. For glycan analysis, the cells were collected mechanically, washed, and stored frozen until the analysis. In FACS analyses 70-90% of cells from mechanically isolated hESC colonies were typically Tra 1-60 and Tra 1-81 positive (not shown). Cells differentiated into embryoid bodies (EB) and further differentiated cells grown out of the EB as monolayers (stage 3 differentiated) were used for comparison against hESC. The differentiation protocol favors the development of neuroepithelial cells while not directing the differentiation into distinct terminally differentiated cell types (Okabe et al., 1996). Stage 3 cultures consisted of a heterogenous population of cells dominated by fibroblastoid and neuronal morphologies.

Glycan isolation. Asparagine-linked glycans were detached from cellular glycoproteins by F. meningosepticum N-glycosidase F digestion (Calbiochem, USA) essentially as described (Nyman et al., 1998). The detached glycans were divided into sialylated and non-sialylated fractions based on the negative charge of sialic acid residues. Cellular contaminations were removed by precipitating the glycans with 80-90% (v/v) aqueous acetone at −20° C. and extracting them with 60% (v/v) ice-cold methanol essentially as described previously (Verostek et al., 2000). The glycans were then passed in water through C₁₈ silica resin (BondElut, Varian, USA) and adsorbed to porous graphitized carbon (Carbograph, Alltech, USA) based on previous method (Davies et al., 1993). The carbon column was washed with water, then the neutral glycans were eluted with 25% acetonitrile in water (v/v) and the sialylated glycans with 0.05% (v/v) trifluoroacetic acid in 25% acetonitrile in water (v/v). Both glycan fractions were additionally passed in water through strong cation-exchange resin (Bio-Rad, USA) and C₁₈ silica resin (ZipTip, Millipore, USA). The sialylated glycans were further purified by adsorbing them to microcrystalline cellulose in n-butanol:ethanol:water (10:1:2, v/v), washing with the same solvent, and eluting by 50% ethanol:water (v/v). All the above steps were performed on miniaturized chromatography columns and small elution and handling volumes were used. The glycan analysis method was validated by subjecting human cell samples to analysis by five different persons. The results were highly comparable, especially by the terms of detection of individual glycan signals and their relative signal intensities, showing that the reliability of the present methods is suitable for comparing analysis results from different cell types.

Mass spectrometry and data analysis. MALDI-TOF mass spectrometry was performed with a Bruker Ultraflex TOF/TOF instrument (Bruker, Germany) essentially as described (Saarinen et al., 1999). Relative molar abundancies of both neutral and sialylated glycan components can be accurately assigned based on their relative signal intensities in the mass spectra (Naven and Harvey, 1996; Papac et al., 1996; Saarinen et al., 1999; Harvey, 1993). Each step of the mass spectrometric analysis methods were controlled for their reproducibility by mixtures of synthetic glycans or glycan mixtures extracted from human cells. The mass spectrometric raw data was transformed into the present glycan profiles by carefully removing the effect of isotopic pattern overlapping, multiple alkali metal adduct signals, products of elimination of water from the reducing oligosaccharides, and other interfering mass spectrometric signals not arising from the original glycans in the sample. The resulting glycan signals in the presented glycan profiles were normalized to 100% to allow comparison between samples. Quantitative difference between two glycan profiles (%) was calculated according to the equation:

$$difference = \frac{1}{2} {\sum }_{i = 1}^{n} \vert {p}_{i , a} - {p}_{i , b} \vert , \qquad ( 2 )$$

wherein p is the relative abundance (%) of glycan signal i in profile a or b, and n is the total number of glycan signals.

Glycosidase analysis. The neutral N-glycan fraction was subjected to digestion with Jack bean α-mannosidase (Canavalia ensiformis; Sigma, USA) essentially as described (Saarinen et al., 1999). The specificity of the enzyme was controlled with glycans isolated from human tissues as well as purified oligosaccharides.

NMR methods. For NMR analysis, larger amounts of hESC were grown on mouse feeder cell (MEF) layers. The purity of the collected hESC sample (about 70%), was lower than in the mass spectrometry samples grown on HFF. However, the same H₅₋₉N₂ glycans were the major neutral N-glycan signals in both MEF and hESC. The isolated glycans were further purified for the analysis by gel filtration high-pressure liquid chromatography in a column of Superdex peptide HR 10/30 (Amersham), with water (neutral glycans) or 50 mM NH₄HCO₃ (sialylated glycans) as the eluant at a flow rate of 1 ml/min. The eluant was monitored at 214 nm, and oligosaccharides were quantified against external standards. The amount of N-glycans in NMR analysis was below five nanomoles.

Statistical procedures. Glycan score distributions of all three differentiation stages (hESC, EB, and st.3) were analyzed by the Kruskal-Wallis test. Pairwise comparisons were performed by the 2-tailed Student's t-test with Welch's approximation and 2-tailed Mann-Whitney U test. A p value less than 0.05 was considered significant.

Lectin staining. Fluorescein-labeled lectins were from EY Laboratories (USA) and the stainings were performed essentially after manufacturer's instructions. The specificity of the staining was controlled in parallel experiments by inhibiting lectin binding with specific oligo- and monosaccharides.

### Results

Mass Spectrometric Profiling of the hESC N-Glycome

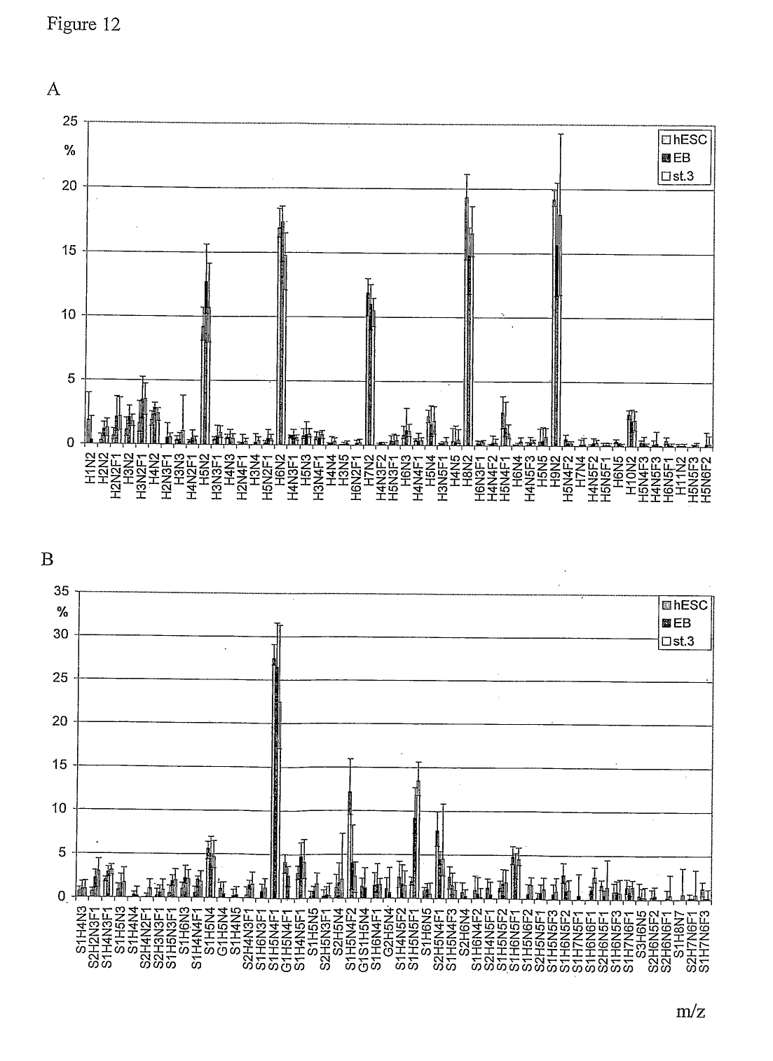

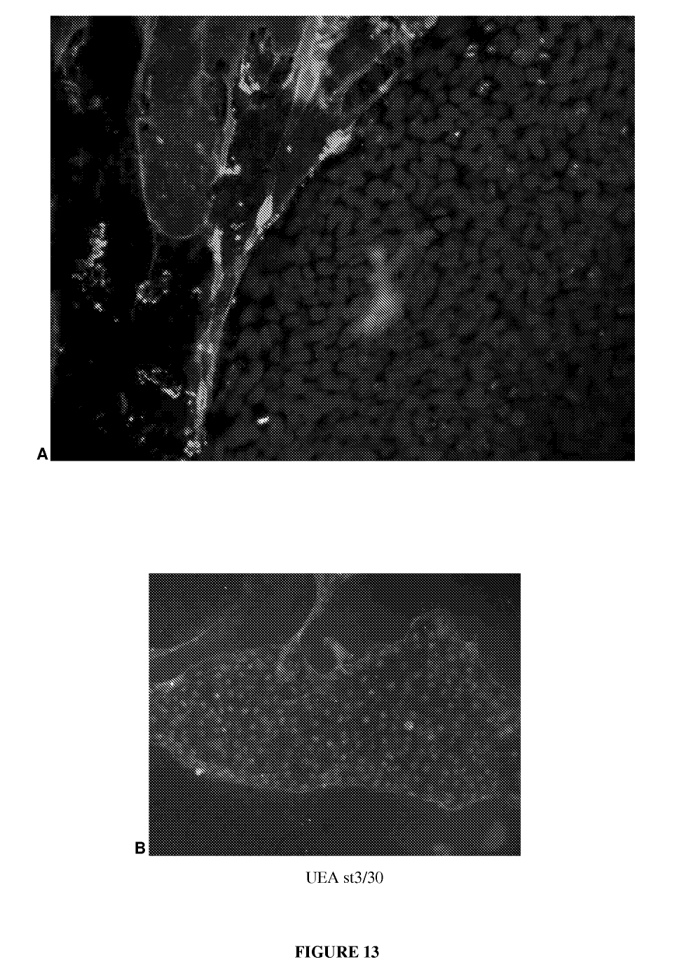

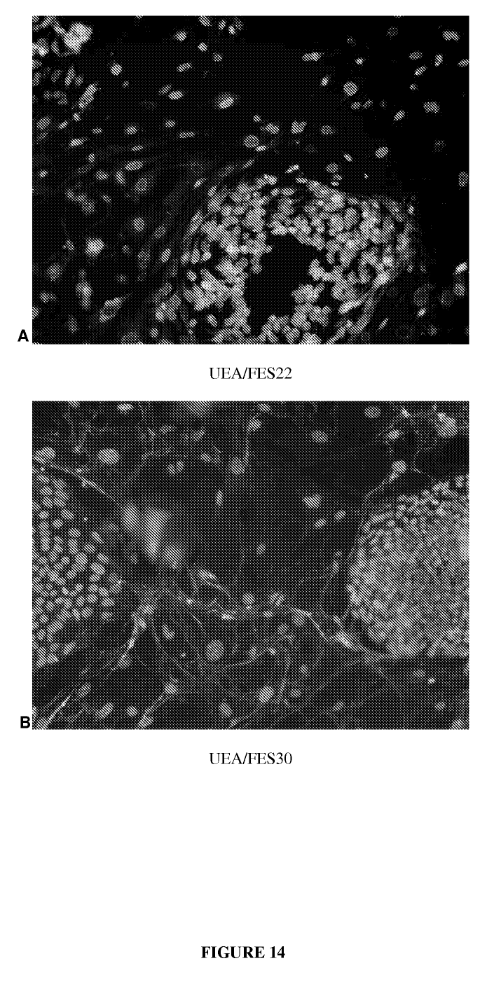

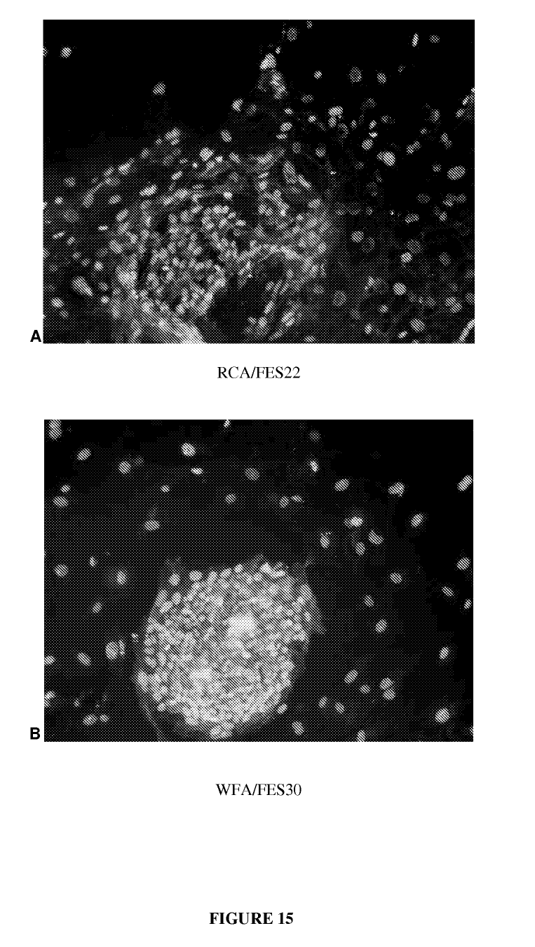

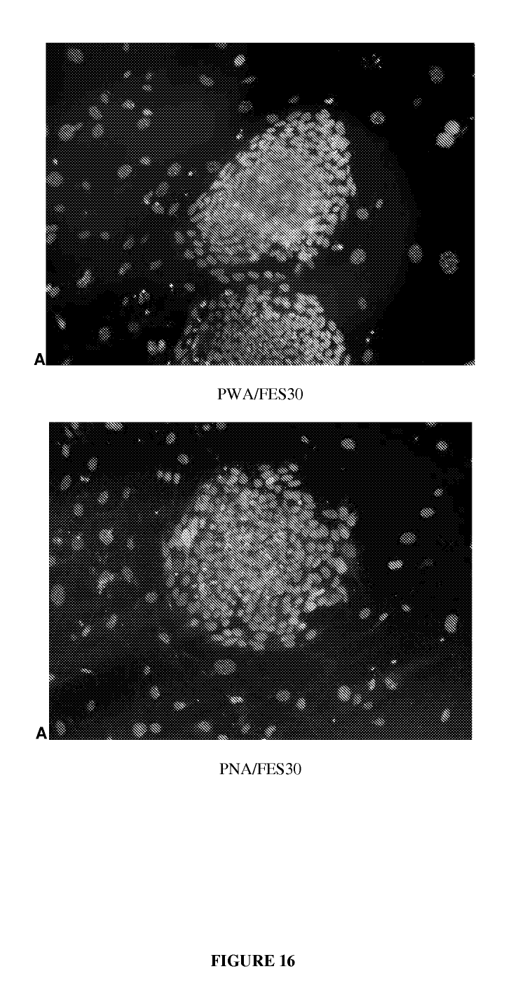

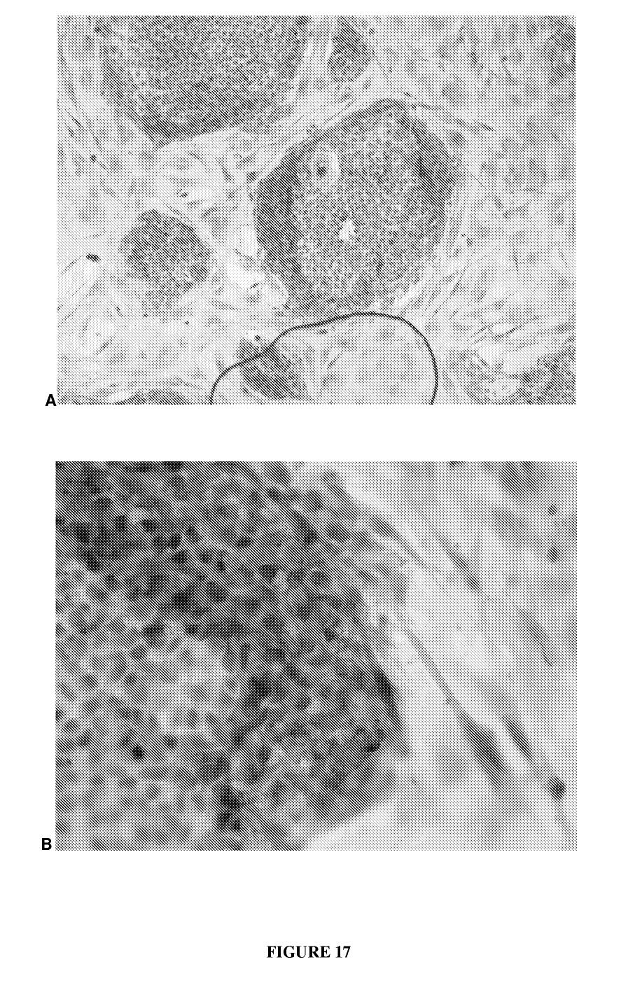

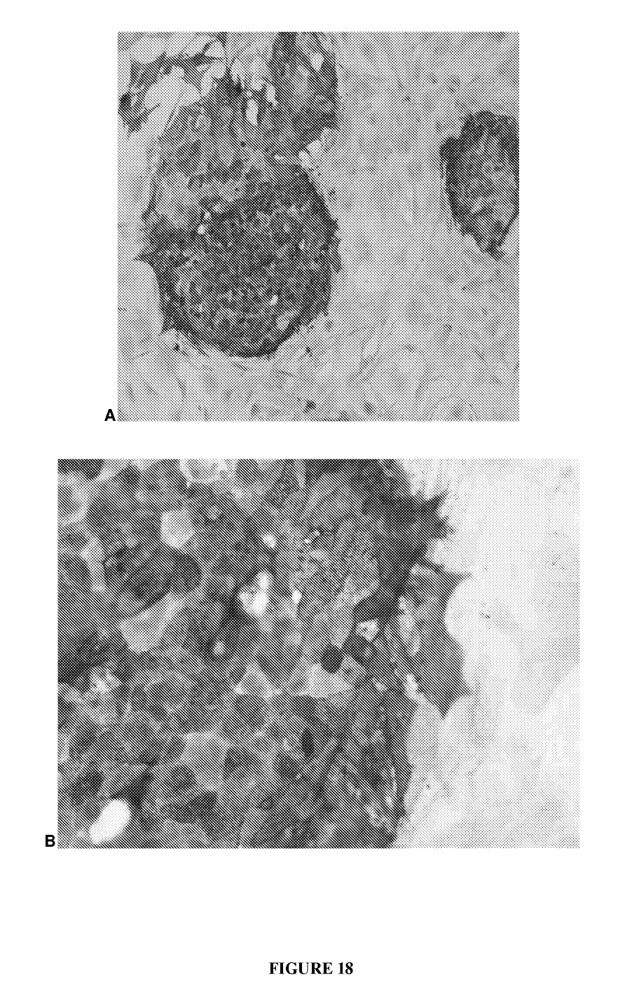

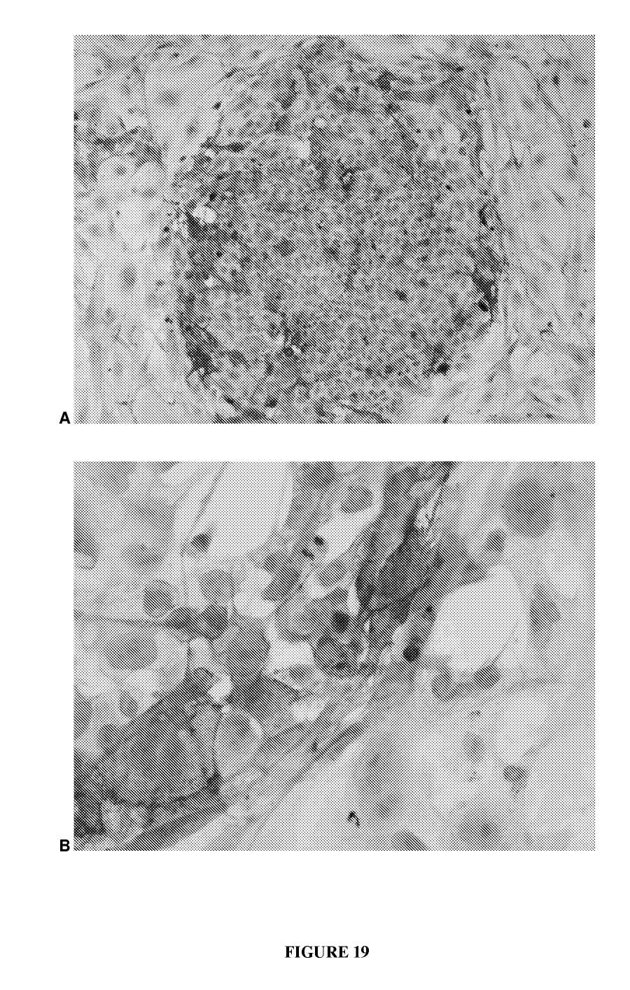

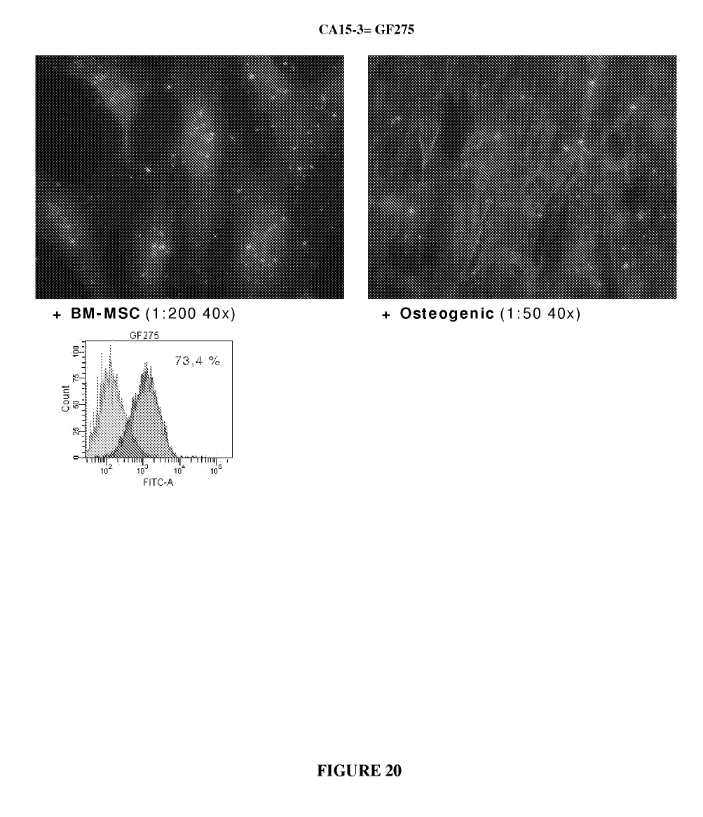

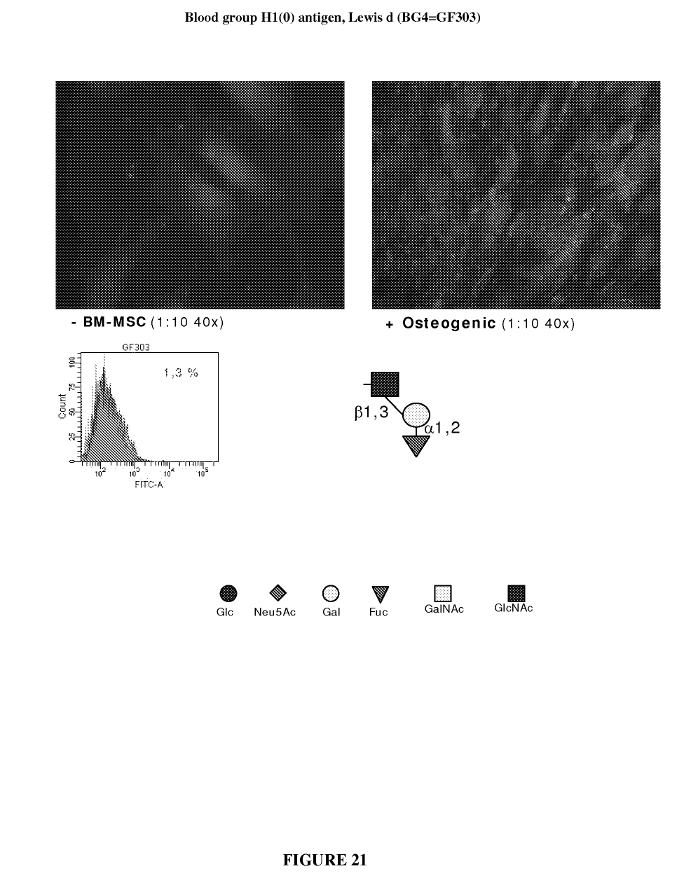

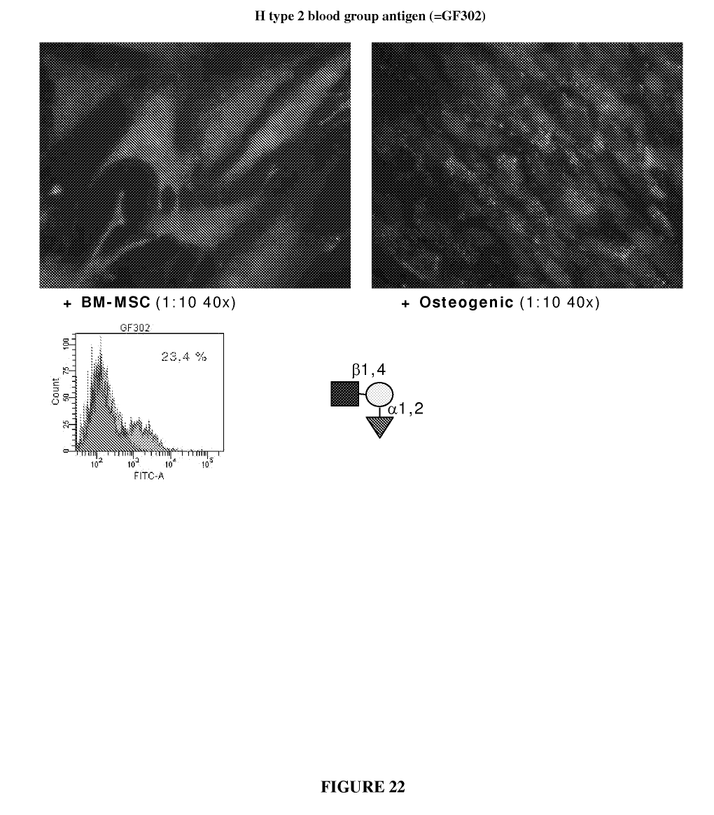

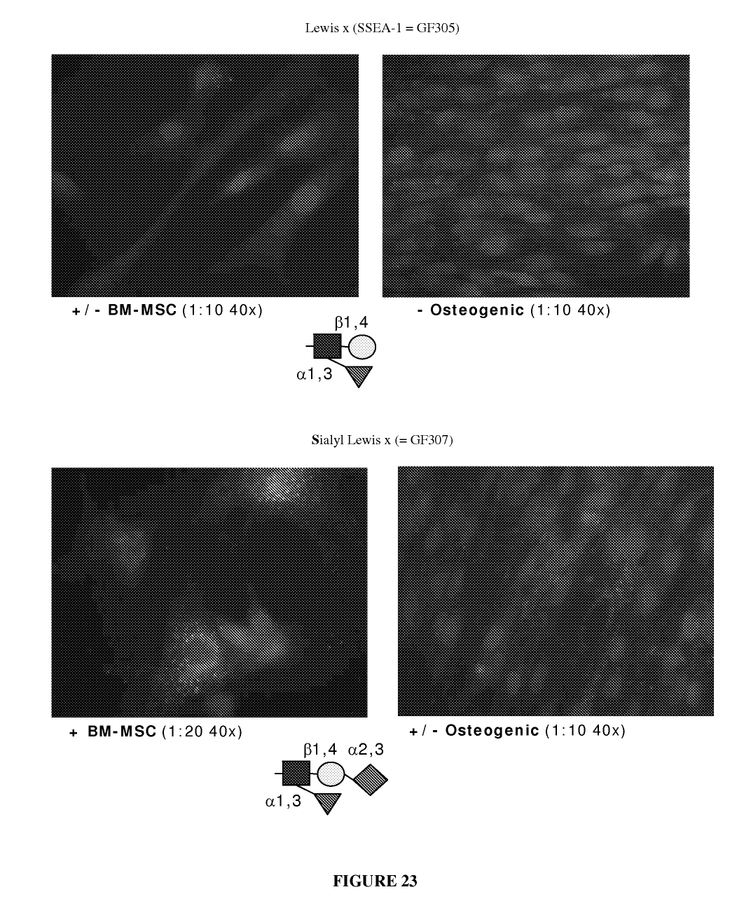

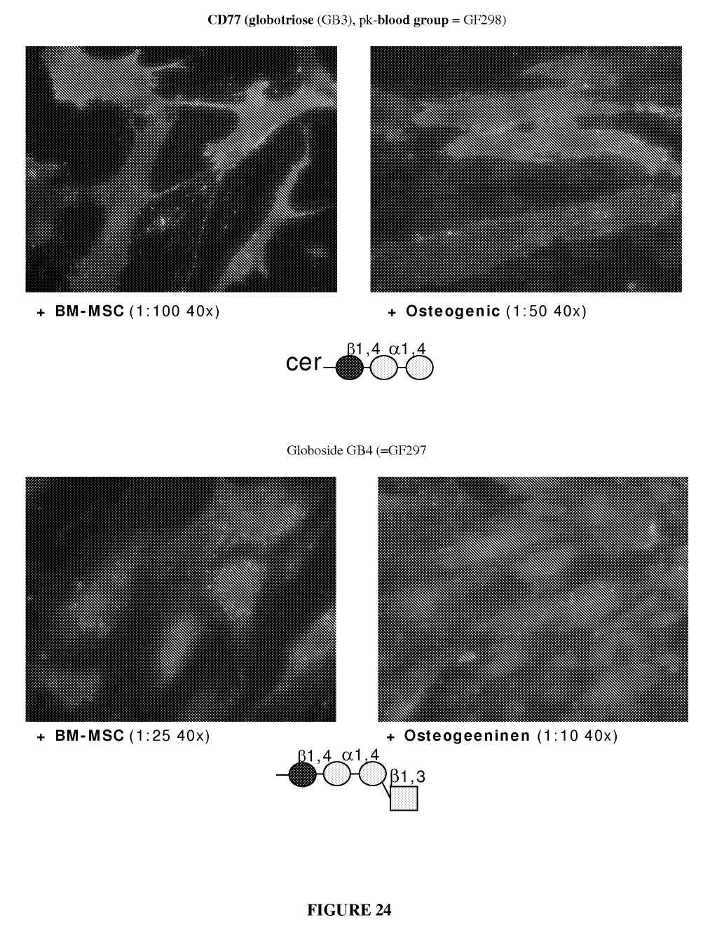

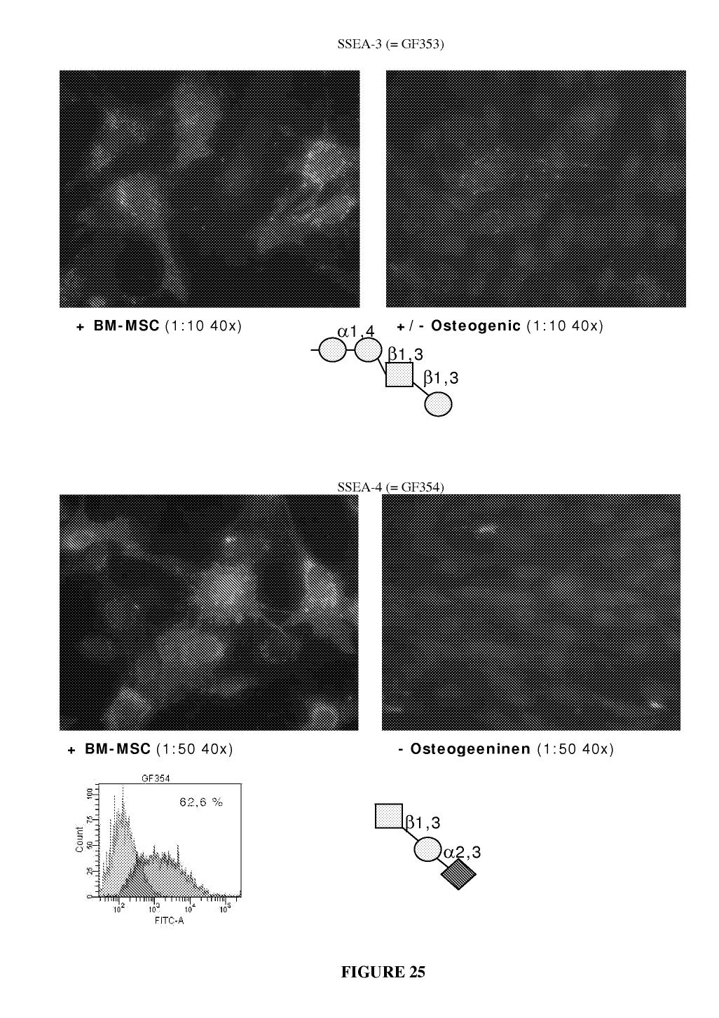

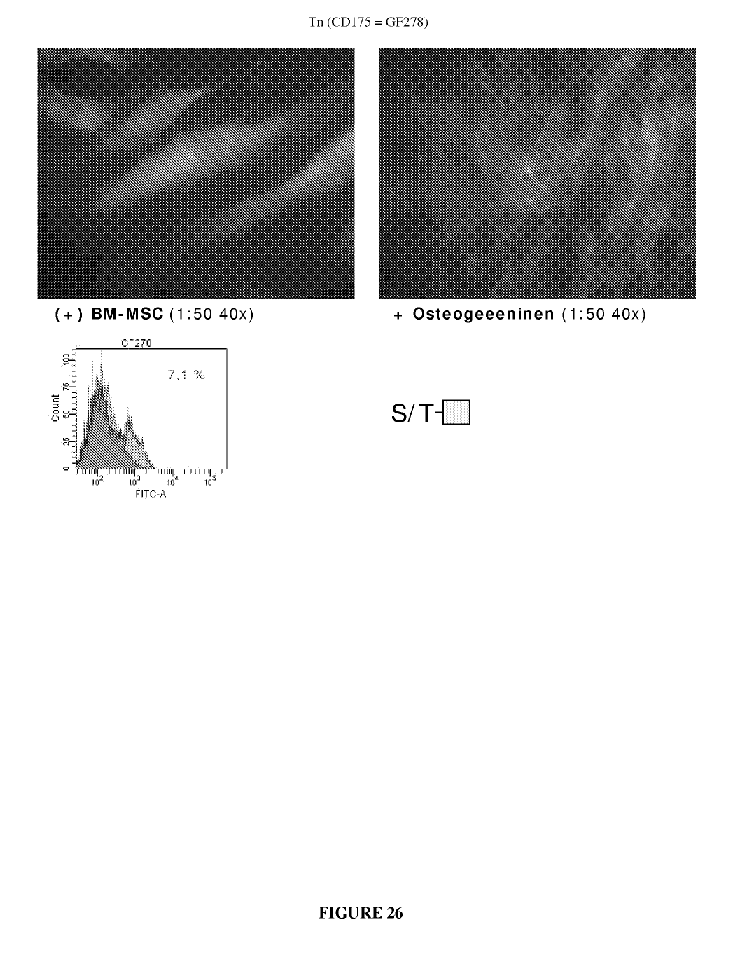

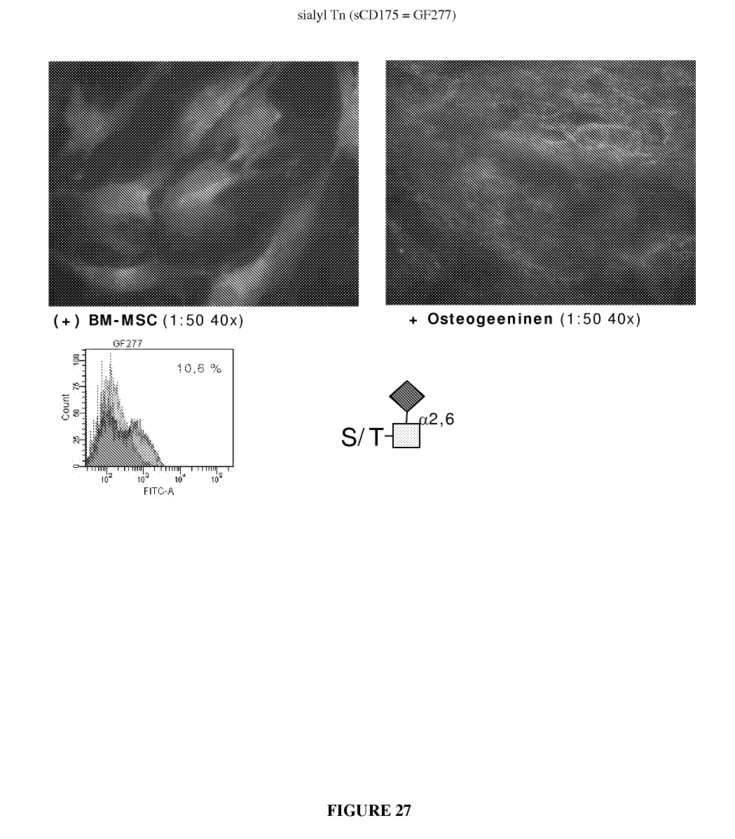

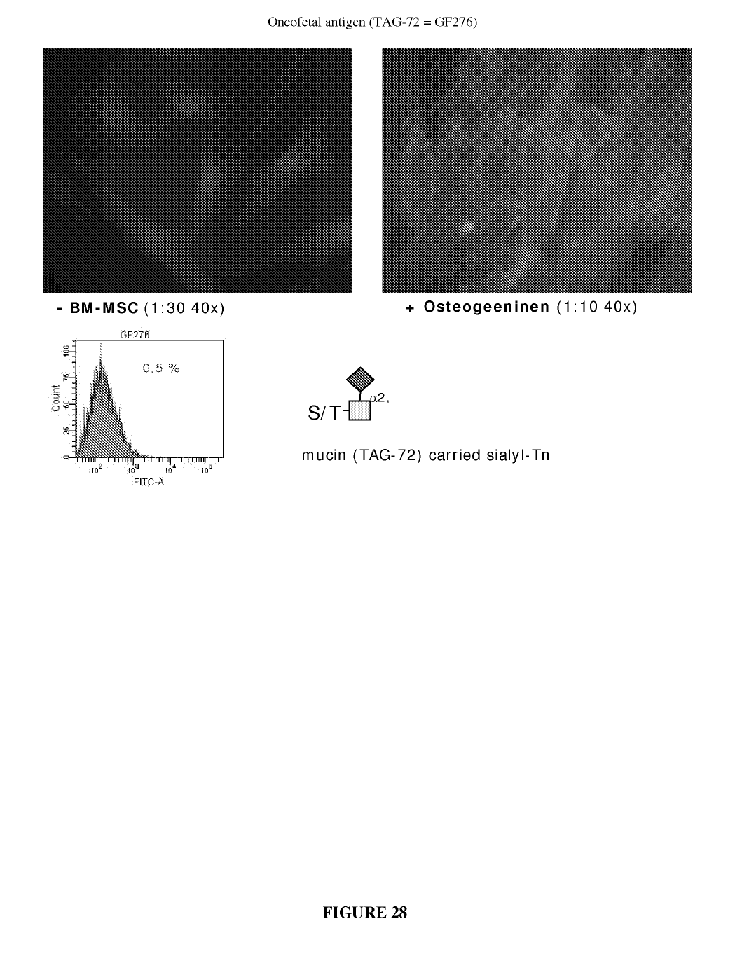

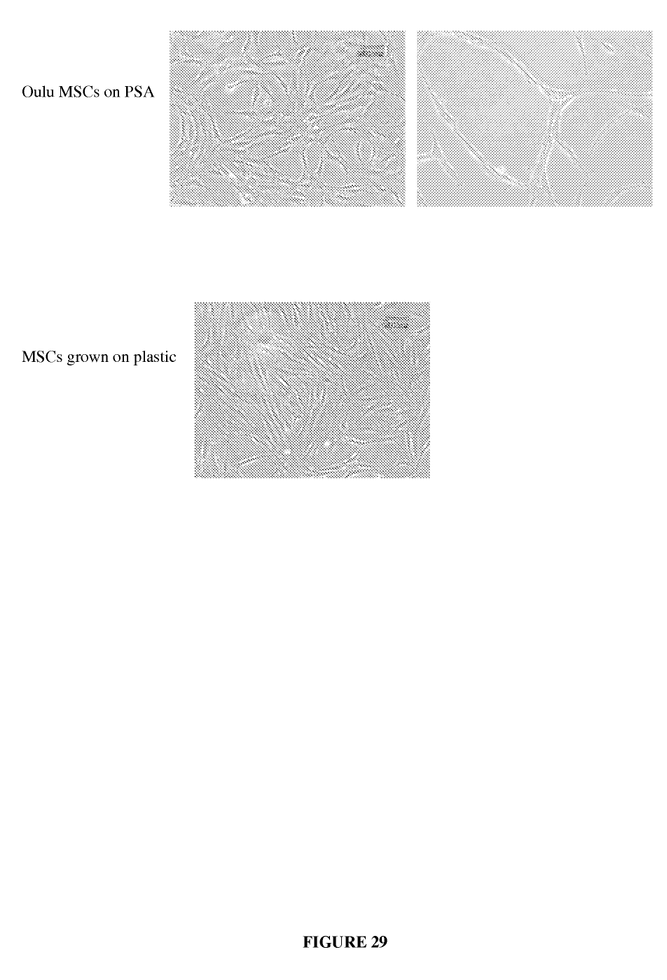

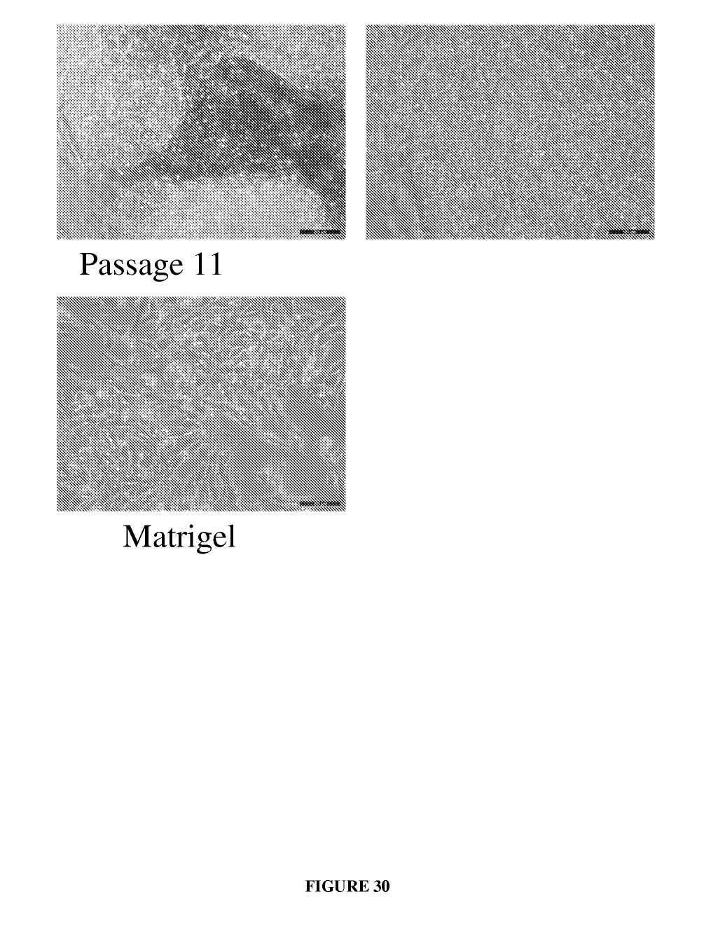

In order to generate glycan profiles of hESC, embryonic bodies, and further differentiated cells, a MALDI-TOF mass spectrometry based analysis was performed. We focused on the most common type of protein post-translational modifications, the asparagine-linked glycans (N-glycans), which were enzymatically released from cellular glycoproteins. During glycan isolation and purification, the total N-glycan pool was separated by an ion-exchange step into neutral N-glycans and sialylated N-glycans. These two glycan fractions were then analyzed separately by mass spectrometric profiling (FIG. 12), which yielded a global view of the N-glycan repertoire of the samples. The relative abundances of the observed glycan signals were determined based on their relative signal intensities (Naven and Harvey, 1996; Papac et al., 1996; Saarinen et al., 1999), which allowed quantitative comparison of glycome differences between samples. Over one hundred N-glycan signals were detected from each cell type.

The proposed monosaccharide compositions corresponding to the detected masses of each individual signal in FIG. 12 is indicated by letter code. However, it is important to realize that many of the mass spectrometric signals in the present analyses include multiple isomeric structures and the 100 most abundant signals very likely represent hundreds of different molecules. For example, the common hexoses (H) occurring in human N-glycans include D-mannose, D-galactose, and D-glucose (which all have a residue mass of 162.05 Da), and common N-acetylhexosamines (N) include both N-acetyl-D-glucosamine and N-acetyl-D-galactosamine (203.08 Da); deoxyhexoses (F) are typically L-fucose residues (146.06 Da).

In most of the previous glycomic studies of other mammalian tissues the isolated glycans have been derivatized (permethylated) prior to mass spectrometric profiling (Sutton-Smith et al., 2002; Dell and Morris, 2001; Consortium for Functional Glycomics, http://www.functionalglycomics.org) or chromatographic separation (Callewaert et al., 2004). However, in the present study we chose to directly analyze picomolar quantities of unmodified glycans and increased sensitivity was attained by omitting the derivatization and the subsequent additional purification steps. Further, instead of studying the glycan signals one at a time, we were able to simultaneously study all the glycans present in the unmodified glycomes by nuclear magnetic resonance spectroscopy (NMR) and specific glycosidase enzymes. The present data demonstrate that mass spectrometric profiling can be used in the quantitative analysis of total glycomes, especially to pin-point the major glycosylation differences between related samples.

Overview of the hESC N-Glycome: Neutral N-Glycans

Neutral N-glycans comprised approximately two thirds of the combined neutral and sialylated N-glycan pools. The 50 most abundant neutral N-glycan signals of the hESC lines are presented in FIG. 12a (grey columns). The similarity of the profiles, which is indicated by the minor variation in the glycan signals, suggest that the four cell lines closely resemble each other. For example, 15 of the 20 most abundant glycan signals were the same in every hESC line. The five most abundant signals comprised 76% of the neutral N-glycans of hESC and dominated the profile.

### Sialylated N-Glycans

All N-glycan signals in the sialylated N-glycan fraction (FIG. 12b, grey columns) contain sialic acid residues (S: N-acetyl-D-neuraminic acid, or G: N-glycolyl-D-neuraminic acid). The 50 most abundant sialylated N-glycans in the four hESC lines showed more variation between individual cell lines than the neutral N-glycans. However, the four cell lines again resembled each other. The group of five most abundant sialylated N-glycan signals was the same in every cell line: S₁H₅N₄F₁, S₁H₅N₄F₂, S₂H₅N₄F₁, S₁H₅N₄, and S₁H₆N₅F₁ (for abbreviations see FIG. 12). The majority (61%, in eight signals) of the sialylated glycan signals contained the H₅N₄ core composition and differed only by variable amounts of sialic acid (S or G) and deoxyhexose (F) residues. Similarly, another common core structure was H₆N₅ (12%, in seven signals). This highlights the biosynthetic mechanisms leading to the total spectrum of N-glycan structures in cells: N-glycans typically consist of common core structures that are modified by the addition of variable epitopes.

Importantly, we were able to detect N-glycans containing N-glycolylneuraminic acid (G), for example glycans G₁H₅N₄, G₁S₁H₅N₄, and G₂H₅N₄, in the hESC samples. N-glycolylneuraminic acid has previously been reported in hESC as an antigen transferred from culture media containing animal-derived materials (Martin et al., 2005). Accordingly, the serum replacement medium used in the present experiments contained bovine serum proteins.

### Variation Between Individual Cell Lines

Although the four hESC lines shared the same overall N-glycan profile, there was cell line specific variation within the profiles. Individual glycan signals unique to each cell line were detected, indicating that every cell line was slightly different from each other with respect to the approximately one hundred most abundant N-glycan structures they synthesized.

In general, the 30 most common N-glycan signals in each hESC line accounted for circa 85% of the total detected N-glycans, and represent a useful approximation of the hESC N-glycome. In other words, more than five out of six glycoprotein molecules isolated from any of the present hESC lines would carry such N-glycan structures.

Transformation of the N-Glycome During hESC Differentiation

A major goal of the present study was to identify glycan structures that would be specific to either stem cells or differentiated cells, and could therefore serve as differentiation stage markers. In order to determine whether the hESC N-glycome undergoes changes during differentiation, the N-glycan profiles obtained from hESC, EB, and stage 3 differentiated cells were compared (FIG. 12). The profiles of the differentiated cell types (EB and st.3) were significantly different from the profiles of undifferentiated hESC, indicated by non-overlapping distribution bars in many glycan signals. Further, there were many signals present in both hESC and EB that were not detected in stage 3 differentiated cells. Overall, 10% of the glycan signals present in hESC had disappeared in stage 3 differentiated cells. Simultaneously numerous new signals appeared in EB and stage 3 differentiated cells. Their proportion in EB and stage 3 differentiated cells was 14% and 16%, respectively. The glycan signals that were characteristic for hESC were typically decreased in the EB and had further decreased or totally disappeared in stage 3 differentiated cells. However, among the most common one hundred glycan signals there were no hESC signals that would not have been expressed in EB, suggesting that the EB N-glycome is an intermediate between hESC and stage 3 differentiated cells.

Taken together, differentiation induced the appearance of new N-glycan types while earlier glycan types disappeared. Further, we found that the major hESC-specific N-glycosylation features were not expressed as discrete glycan signals, but instead as glycan signal groups that were characterized by a specific monosaccharide composition feature (see below). In other words, differentiation of hESC into EB induced the disappearance of not only one but multiple glycan signals with hESC-associated features, and simultaneously also the appearance of glycan signal groups with other features associated with the differentiated cell types.

The N-glycan profiles of the differentiated cells were also quantitatively different from the undifferentiated hESC profiles. A practical way of quantifying the differences between individual glycan profiles is to calculate the sum of the signal intensity differences between two cell profiles (see Methods). According to this method, the EB neutral and sialylated N-glycan profiles had undergone a quantitative change of 14% and 29% from the hESC profiles, respectively. Similarly, the stage 3 differentiated cell neutral and sialylated N-glycan profiles had changed by 15% and 43% from the hESC profiles, respectively. This indicates that upon differentiation of hESC into stage 3 differentiated cells, nearly half of the total sialylated N-glycans present in the cells were transformed into different molecular structures, while significantly smaller proportion of the neutral N-glycan molecules were changed during the differentiation process. Taking into account that the proportion of sialylated to neutral N-glycans in hESC was approximately 1:2, the total N-glycome change was approximately 25% during the transition from hESC to stage 3 differentiated cells. Again, the N-glycan profile of EB appeared to lie between hESC and stage 3 differentiated cells.

The data indicated that the hESC N-glycome consisted of two discrete parts regarding propensity to change during hESC differentiation—a constant part of circa 75% and a changing part of circa 25%. In order to characterize the associated N-glycan structures, and to identify the potential biological roles of the constant and changing parts of the N-glycome, we performed structural analyses of the isolated hESC N-glycan samples.

Structural Analyses of the Major hESC N-Glycans: Preliminary Structure Assignment Based on Monosaccharide Compositions

Human N-glycans can be divided into the major biosynthetic groups of high-mannose type, hybrid-type, and complex-type N-glycans. To determine the presence of these N-glycan groups in hESC and their progeny, assignment of probable structures matching the monosaccharide compositions of each individual signal was performed utilizing the established pathways of human N-glycan biosynthesis (Kornfeld and Kornfeld, 1985; Schachter, 1991). Here, the detected N-glycan signals were classified into four N-glycan groups according to the number of N and H residues: 1) high-mannose type and 2) low-mannose type N-glycans, which are both characterized by two N residues (N=2), 3) hybrid-type or monoantennary N-glycans, which are classified by three N residues (N=3), and 4) complex-type N-glycans, which are characterized by four or more N residues (N≧4) in their proposed monosaccharide compositions. This is an approximation: for example, in addition to complex-type N-glycans also hybrid-type and monoantennary N-glycans may contain more than three N residues.

The data was analyzed quantitatively by calculating the percentage of glycan signals in the total N-glycome belonging to each structure group (Table 22, rows A-E and J-L). The quantitative changes in the structural groups reflect the relative activities of different biosynthetic pathways in each cell type. For example, the proportion of hybrid-type or monoantennary N-glycans was increased when hESC differentiated into EB. In general, the relative proportions of most glycan structure classes remained approximately constant through the hESC differentiation process, which indicated that both hESC and the differentiated cell types were capable of equally sophisticated N-glycosylation. The high proportion of N-glycans classified as low-mannose N-glycans in all the studied cell types was somewhat surprising in the light of earlier published studies of human N-glycosylation. However, previous studies had not explored the total N-glycan profiles of living cells. We have detected significant amounts of low-mannose N-glycans also in other human cells and tissues, and they are not specific to hESC (T.S., A.H., M.B., A.O., J.H., J.N, J. S. et al., unpublished results).

### Verification of Structure Assignments by Enzymatic Degradation and Nuclear Magnetic Resonance Spectroscopy

In order to verify the validity of the glycan structure assignments made based on the detected mass and the probable monosaccharide compositions we performed enzymatic degradation and proton nuclear magnetic resonance spectroscopic analyses (¹H-NMR) of selected neutral and sialylated N-glycans.

For the validation of neutral N-glycans we chose glycans with 5-9 hexose (H) and two N-acetylhexosamine (N) residues in their monosaccharide compositions (H₅N₂, H₆N₂, H₇N₂, H₈N₂, and H₉N₂) which were the most abundant N-glycans in all studied cell types (FIG. 12a). The monosaccharide compositions suggested that these glycans were high-mannose type N-glycans (Kornfeld and Kornfeld, 1985). To test this hypothesis, neutral N-glycans from stem cell and differentiated cell samples were treated with α-mannosidase, and analyzed both before and after the enzymatic treatment (data not shown). The glycans in question were degraded and the corresponding signals disappeared from the mass spectra, indicating that they contained α-linked mannose residues.

The neutral N-glycan fraction was further analyzed by nanoscale proton nuclear magnetic resonance spectroscopic analysis (¹H-NMR). In the obtained ¹H-NMR spectrum of the hESC neutral N-glycans signals consistent with high-mannose type N-glycans were detected, supporting the conclusion that they were the major glycan components in the sample.

Both α-mannosidase and NMR experiments indicated that the H₅₋₉N₂ glycan signals corresponded to high-mannose type N-glycans. From the data in FIG. 12a it could be estimated that they constituted half of all the detected glycoprotein N-glycans in hESC. This is in accordance with the established role of high-mannose type N-glycans in human cells (Helenius and Aebi, 2001, 2004). The presence of such constitutively expressed N-glycans also explained why the neutral N-glycan profiles did not change to the same extent as the sialylated N-glycan profiles during differentiation.

For the validation of structure assignments among the sialylated N-glycans we noted that the majority of the sialylated N-glycan signals isolated from hESC were characterized by the N≧4 monosaccharide composition (FIG. 12a), which suggested that they were complex-type N-glycans. In the ¹H-NMR analysis N-glycan backbone signals consistent with biantennary complex-type N-glycans were the major detected signals, in line with the assignment made based on the experimental monosaccharide compositions. The present results indicated that the classification of the glycan signals within the total N-glycome data could be used to construct an approximation of the whole N-glycome. However, such classification should not be applied to the analysis of single N-glycan signals.

### Differentiation Stage Associated Structural Glycosylation Features

The glycan signal classification described above indicated changes in the core sequences of N-glycans. The present data also suggested that there were differences in variable epitopes added to the N-glycan core structures i.e. glycan features present in many individual glycan signals. In order to quantify such glycan structural features, the N-glycome data were further classified into glycan signal groups that share similar features in their proposed monosaccharide compositions (Table 22, rows F-I and M-P). As a result, the majority of the differentiation-associated glycan signals in the EB and stage 3 differentiated cell samples fell into different groups than the hESC specific glycans. Glycan signals with complex fucosylation (Table 22, row N) were associated with undifferentiated hESC, whereas glycan signals with potential terminal N-acetylhexosamine (Table 22, rows H and P) were associated with the differentiated cells.

Complex Fucosylation of N-Glycans is Characteristic of hESC

Differentiation stage associated changes in the sialylated N-glycan profile were more drastic than in the neutral N-glycan fraction and the group of five most abundant sialylated N-glycan signals was different at every differentiation stage (FIG. 12b). In particular, there was a significant differentiation-associated decrease in the relative amounts of glycans S₁H₅N₄F₂ and S₁H₅N₄F₃ as well as other glycan signals that contained at least two deoxyhexose residues (F≧2) in their proposed monosaccharide compositions. In contrast, glycan signals such as S₂H₅N₄ that contained no F were increased in the differentiated cell types. The results suggested that sialylated N-glycans in undifferentiated hESC were subject to more complex fucosylation than in the differentiated cell types (Table 22, row N).

The most common fucosylation type in human N-glycans is α1,6-fucosylation of the N-glycan core structure. The NMR analysis of the sialylated N-glycan fraction of hESC also revealed α1,6-fucosylation of the N-glycan core as the most abundant type of fucosylation. In the N-glycans containing more than one fucose residue, there must have been other fucose linkages in addition to the α1,6-linkage (Staudacher et al., 1999). The F≧2 structural feature decreased as the cells differentiated, indicating that complex fucosylation was characteristic of undifferentiated hESC.

N-Glycans with Terminal N-Acetylhexosamine Residues Become More Common with Differentiation

A group of N-glycan signals which increased during differentiation contained equal amounts of N-acetylhexosamine and hexose residues (N=H) in their monosaccharide composition, e.g. S₁H₅N₅F₁. This was consistent with structures containing non-reducing terminal N-acetylhexosamine residues. Usually N-glycan core structures contain more hexose than N-acetylhexosamine residues. However, if complex-type N-glycans contain terminal N-acetylhexosamine residues that are not capped by hexoses, their monosaccharide compositions change to either the N=H or the N>H. EB and stage 3 differentiated cells showed increased amounts of potential terminal N-acetylhexosamine structures, of which the N=H structural feature was increased in both neutral and sialylated N-glycan pools (Table 22, rows I and P), whereas the N>H structural feature was elevated in the neutral N-glycan pool, but decreased in the sialylated N-glycan pool during differentiation (Table 22, rows H and O).

Glycome Profiling can Identify the Differentiation Stage of hESC

The analysis of glycome profiles indicated that the studied hESC lines and differentiated cells had differentiation stage specific N-glycan features. However, the data also demonstrated that N-glycan profiles of the individual hESC lines were different from each other and in particular the hESC line FES 22 was different from the other three stem cell lines (Table 22, rows C and I). To test whether the obtained N-glycan profiles could be used to generate an algorithm that would discriminate between hESC and differentiated cells even taking into account cell line specific variation, an analysis was performed using the data of Table 22. The hESC line FES 29 and embryoid bodies derived from it (EB 29) were selected as the training group for the calculation. The algorithm glycan score (Equation 1) was defined as the sum of those structural features that were at least two times greater in FES 29 than in EB 29 (row N in Table 22), from which the sum of the structural feature percentages that were at least two times greater in EB 29 than in FES 29 was subtracted (rows C, I, J, and P in Table 22):

glycan score=N−(C+I+J+P), (1)

wherein the letters refer to the row numbering of Table 22.
The Identified hESC Glycans can be Targeted at the Cell Surface

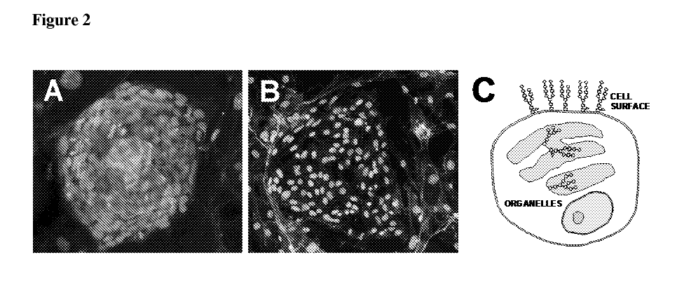

From a practical perspective stem cell research would be best served by the identification of target structures on cell surface. To investigate whether individual glycan structures we had identified would be accessible to reagents targeting them at the cell surface we performed lectin labelling of two candidate structure types. Lectins are proteins that recognize glycans with specificity to certain glycan structures also in hESC (Venable et al., 2005). To study the localization of glycan components in hESC, stem cell colonies grown on mouse feeder cell layers were labeled in vitro by fluorescein-labelled lectins (FIG. 2). The hESC cell surfaces were clearly labeled by Maackia amurensis agglutinin (MAA) that recognizes structures containing α2,3-linked sialylation, indicating that sialylated glycans are abundant on the hESC cell surface (FIG. 2a). Such glycans would thus be available for recognition by more specific glycan-recognizing reagents such as antibodies. In contrast, the cell surfaces were not labelled by Pisum sativum agglutinin (PSA) that recognizes α-mannosylated glycans (FIG. 2b). However, PSA labelled the cells after permeabilization (data not shown), suggesting that the mannosylated N-glycans in hESC were localized in intracellular cell compartments such as the endoplasmic reticulum (ER) or the Golgi complex (FIG. 2c). Interestingly, the mouse fibroblast cells showed complementary staining patterns, suggesting that these lectin reagents efficiently discriminated between hESC and feeder cells. Together the results suggested that the glycan structures we identified could be utilized to design specific reagents targeting hESC.

### Comparative Analysis of the N-Glycome

Although the N-glycan profiles of the four hESC lines share a similar overall profile shape, there was cell line specific variation in the N-glycan profiles. Individual glycan signals unique to each cell line were found, indicating that every cell line was slightly different from each other with respect to the approximately one hundred most abundant glycan structures they synthesize. This is represented in .34a as Venn diagrams combining all the detected glycan signals from both the neutral and the acidic N-glycan fractions. FES 29 and FES 30 were derived from sibling embryos, but their N-glycan profiles did not resemble each other more than they resembled FES 21 in the Venn diagram. Furthermore, FES 30 that has the karyotype XX did not differ significantly from the three XY hESC lines.

In order to determine whether the hESC N-glycome undergoes changes during differentiation, N-glycan profiles obtained from hESC, EB, and stage 3 differentiated cells were compared (FIG. 12). The N-glycan profiles of the differentiated cell types (EB and st.3) differed significantly from the profiles of undifferentiated hESC, which is indicated by non-overlapping distribution bars in many glycan signals. There were many signals in common between hESC and EB that disappeared in stage 3 differentiated cells. Overall, 17% of the glycan signals present in hESC disappeared in EB, and in stage 3 differentiated cells 58% of the original N-glycan signals disappeared. Simultaneously numerous new signals appeared in EB and stage 3 differentiated cells. Their proportion in EB and stage 3 differentiated cells was 24% and 10%, respectively. This indicates that differentiation induced the appearance of new N-glycan types while earlier glycan types disappeared.

The major hESC specific glycosylation feature we identified was the presence of more than one deoxyhexose residue in N-glycans, indicating complex fucosylation. Fucosylation is known to be important in cell adhesion and signalling events (Becker and Lowe, 2003) as well as essential for embryonic development. Knock-out of the N-glycan core α1,6-fucosyltransferase gene FUT8 leads to postnatal lethality in mice (Wang et al., 2005), and mice completely deficient in fucosylated glycan biosynthesis do not survive past early embryonic development (Smith et al., 2002). Fucosylation defects in humans cause a disease known as leukocyte adhesion deficiency (LAD; Luhn et al., 2001).

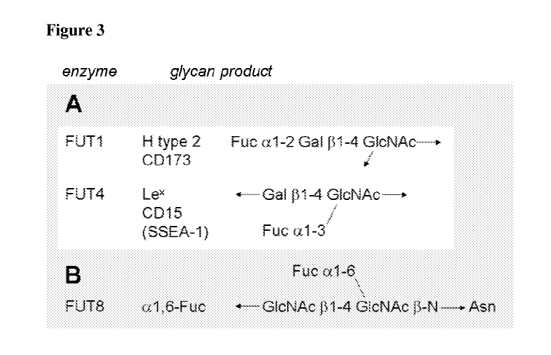

Fucosylated glycans such as the SSEA-1 antigen have previously been associated with both mouse embryonic stem cells (mESC) and human embryonic carcinoma cells (EC; Muramatsu and Muramatsu, 2004), but not with hESC. In addition, structurally related Lex oligosaccharides are able to inhibit embryonic compaction (Fenderson et al., 1984), suggesting that fucosylated glycans are directly involved in cell-to-cell contacts during embryonic development. The α1,3-fucosyltransferase genes indicated in the synthesis of the embryonic Lex and SSEA-1 antigens are FUT4 and FUT9 (Nakayama et al., 2001; Kudo et al., 2004). Interestingly, the published gene expression profiles for the same hESC lines as studied here (Skottman et al., 2005) have demonstrated that three human fucosyltransferase genes, FUT1, FUT4, and FUT8 are expressed in hESC, and that FUT1 and FUT4 are overexpressed in hESC when compared to EB. The known specificities of these fucosyltransferases (Mollicone et al., 1995) correlate with our findings of simple fucosylation in EB and complex fucosylation in hESC (FIG. 3). Taken together, although hESC do not express the specific glycolipid antigen recognized by the SSEA-1 antibody, they share with mESC the characteristic feature of complex fucosylation and may have conserved the biological functions of fucosylated glycan epitopes.

New N-glycan forms emerged in EB and stage 3 differentiated cells. These structural features included additional N-acetylhexosamine residues, potentially leading to new N-glycan terminal epitopes. Another differentiation-associated feature was an increase in the molar proportions of hybrid-type or monoantennary N-glycans. Biosynthesis of hybrid-type and complex-type N-glycans has been demonstrated to be biologically significant for embryonic and postnatal development in the mouse (Ioffe and Stanley, 1994 PNAS; Metzler et al., 1994 EMBO J; Wang et al., 2001 Glycobiology; Akama et al., 2006 PNAS). The preferential expression of complex-type N-glycans in hESC and then the change in the differentiating EB to express more hybrid-type or monoantennary N-glycans may thus be significant for the process of stem cell differentiation.

In conclusion, hESC have a unique glycome which undergoes major changes when the cells differentiate. Information regarding the specific glycome may be utilized in developing reagents for the targeting of these cells and their progeny. Future studies investigating the developmental and molecular regulatory processes resulting in the observed glycan profiles may provide significant insight into mechanisms of human development and regulation of glycosylation.

### REFERENCES

Abeyta, M. J. et al (2004). Hum. Mol. Genet. 13, 601-608.
Apweiler, R. et al (1999). Biochim. Biophys. Acta 1473, 4-8.
Badcock, G. et al (1999). Cancer Res. 59, 4715-4719.
Becker, D. J., and Lowe, J. B. (2003). Glycobiology. 13:41R-5R.
Bhattacharya, B. et al (2004). Blood 103, 2956-2964.
Callewaert, N. et al (2004). Nat Med. 10:429-34.
Carson, D. D., and Lennarz, W. J. (1979). Proc. Natl. Acad. Sci. U.S.A. 76, 5709-5713.
Cooper, D. K. (1998). Xenotransplantation 5, 6-17.
Davies, M. J. et al (1993). J. Chromatogr. 646, 317-326.
Dell, A., and Morris, H. R. (2001). Science 291, 2351-2356.
Fenderson, B. A. et al (1984). J. Exp. Med. 160, 1591-1596.
Goldberg, D. et al (2005). Proteomics 5, 865-875.
Gooi, H. C. et al (1981). Nature 292, 156-158.
Haltiwanger, R. S., and Lowe, J. B. (2004). Annu. Rev. Biochem. 73, 491-537.
Handel, T. M. et al (2005). Annu. Rev. Biochem. 74:385-410.
Harvey, D. J. (1993). Mass Spectrom. 7, 614-619.
Helenius, A., and Aebi, M. (2001). Science 291, 2364-2369.
Helenius, A., and Aebi, M. (2004). Annu. Rev. Biochem. 73, 1019-1049.
Homeister, J. W. et al. (2001). Immunity. 15:115-26.
Imperiali, B., and O′Connor, S. E. (1999). Curr. Opin. Chem. Biol. 3, 643-649.
Kannagi, R. et al. (1983). EMBO J. 2, 2355-2361.
Kilpatrick, D. C. (2002). Biochim. Biophys. Acta 1572, 187-197.
Kornfeld, R., and Kornfeld, S. (1985). Annu. Rev. Biochem. 54, 631-664.
Kornfeld, S. (1986). J. Clin. Invest. 77:1-6.
Kudo, T. et al. (2004). Mol. Cell. Biol. 24:4221-4228.
Lowe, J. B. (2002). Immunol. Rev. 186:19-36.
Luhn, K. et al. (2001). Nat. Genet. 28:69-72.
Martin, M. J. et al. (2005). Nat. Med. 11, 228-232.
Mollicone, R. et al. (1995). Transfusion Clin. Biol. 4:235-242.
Muramatsu, T., and Muramatsu, H. (2004). Glycoconj. J. 21, 41-45.
Nakayama, F. et al. (2001). J. Biol. Chem. 276:16100-16106.
Naven, T. J., and Harvey, D. J. (1996). Rapid Commun. Mass Spectrom. 10, 1361-1366.
Nguyen, D. H. et al. (2005) J. Immunol. 175, 228-236.
Nyman, T. A. et al. (1998). Eur. J. Biochem. 253, 485-493.
Okabe, S. et al. (1996). Mech. Dev. 59:89-102.
Papac, D. I et al. (1996). Anal. Chem. 68, 3215-3223.
Saarinen, J. et al. (1999). Eur. J. Biochem. 259, 829-840.
Sato, N. et al. (2003). Dev. Biol. 260, 404-413.
Schachter, H. (1991). Glycobiology 1, 453-461.
Schneider, E. G. et al. (1978). J. Biol. Chem. 253, 2348-2355.
Shriver, Z. et al. (2004). Nat. Rev. Drug Disc. 3, 863-873.
Skottman, H. et al. (2005). Stem cells 23, 1343-1356.
Smith, P. L. et al. (2002). J. Cell Biol. 158:801-815.
Solter, D., and Knowles, B. B. (1978). Proc. Natl. Acad. Sci. U.S.A. 75, 5565-5569.
Staudacher, E. et al. (1999). Biochim. Biophys. Acta 1473, 216-346.
Sutton-Smith, M. et al. (2002). Biochem. Soc. Symp. 69, 105-115.
Thomson, J. A. et al. (1998). Science 282, 1145-1147.
Varki, A. (1993). Glycobiology 3, 97-130.
Venable, A. et al. (2005). BMC Dev. Biol. 2005 5, 15.
Verostek, M. F. et al. (2000). Anal. Biochem. 278, 111-122.
Wang, X. et al. (2005). Proc. Natl. Acad. Sci. U.S.A. 102:15791-15796.
Wobus, A. M., and Boheler, K. R. (2005). Physiol. Rev. 85, 635-678.
Zanetta, J. P., and Vergoten, G. (2003). Adv. Exp. Med. Biol. 535, 107-124.

### Example 8

### Analysis of Human and Murine Fibroblast Feeder Cells

Murine (mEF) and human (hEF) fibroblast feeder cells were prepared and their N-glycan fractions analyzed as described in the preceding Examples.

### Results and Discussion

The results showed that mEF and hEF cellular N-glycan fractions differ significantly from each other. The differences include differential proportions of glycan groups, major glycan signals, and the glycan profiles obtained from the cell samples. In addition, the major difference is the presence of Galα3Gal epitopes in the mEF cells, as discussed in the preceding Examples of the present invention.

### Example 9

### The Glycome of Human Embryonic Stem Cells Reflects their Differentiation Stage

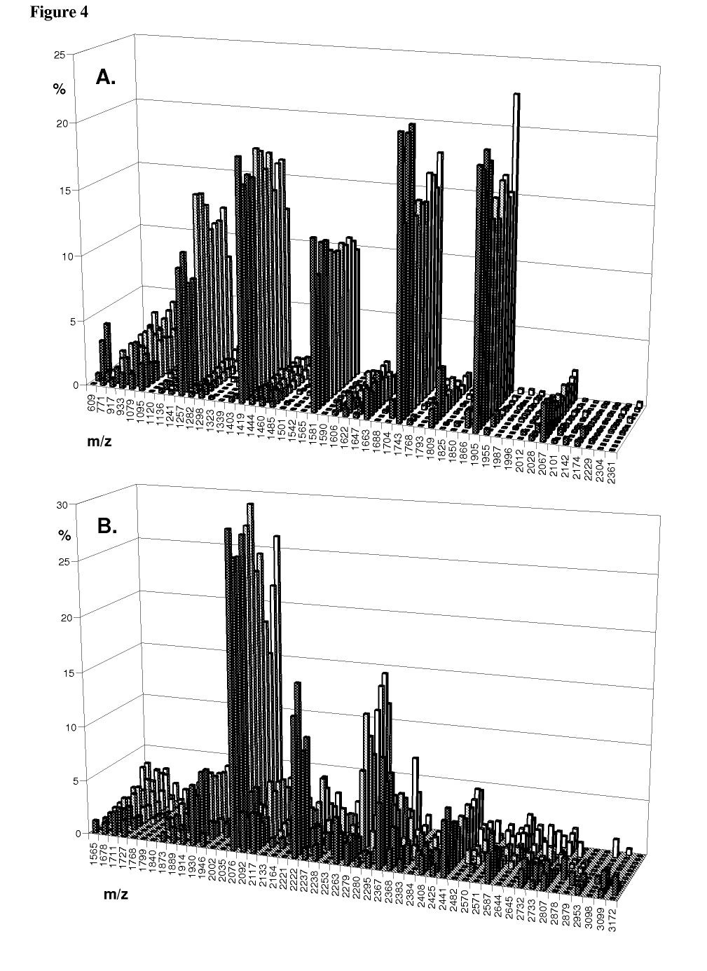

In the present study, we analyzed the N-glycome profiles of hESC, EB, and st.3 differentiated cells (FIG. 4).

The similarity of the N-glycan profiles within the group of four hESC lines suggested that the obtained N-glycan profiles are a description of the characteristic N-glycome of hESC. Overall, 10% of the 100 most abundant N-glycan signals present in hESC disappeared in st.3 differentiated cells, and 16% of the most abundant signals in st.3 differentiated cells were not present in hESC. This indicates that differentiation induced the appearance of new N-glycan types while earlier glycan types disappeared. In quantitative terms, the differences between the glycan profiles of hESC, EB, and st.3 differentiated cells were: hESC vs. EB 19%, hESC vs. st.3 24%, and EB vs. st.3 12%.

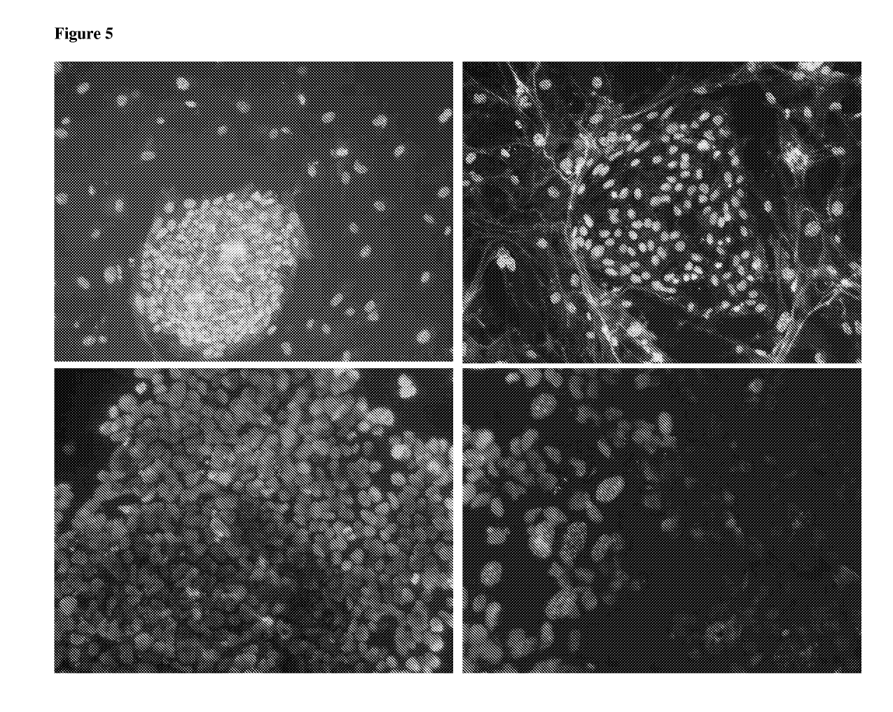

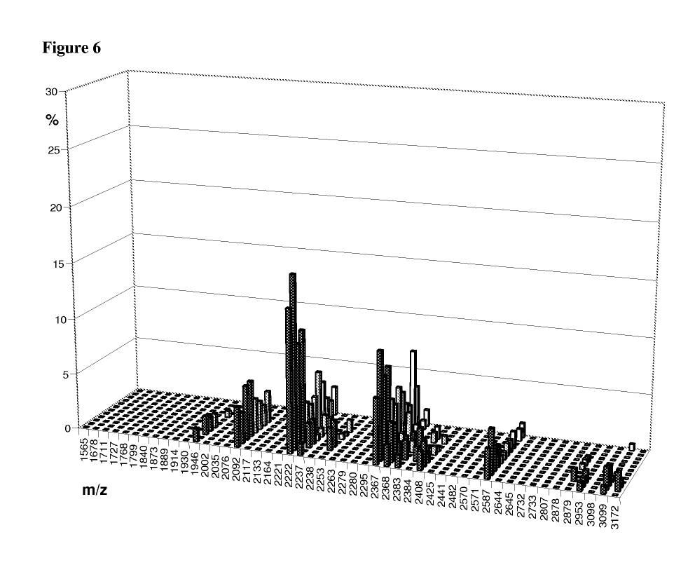

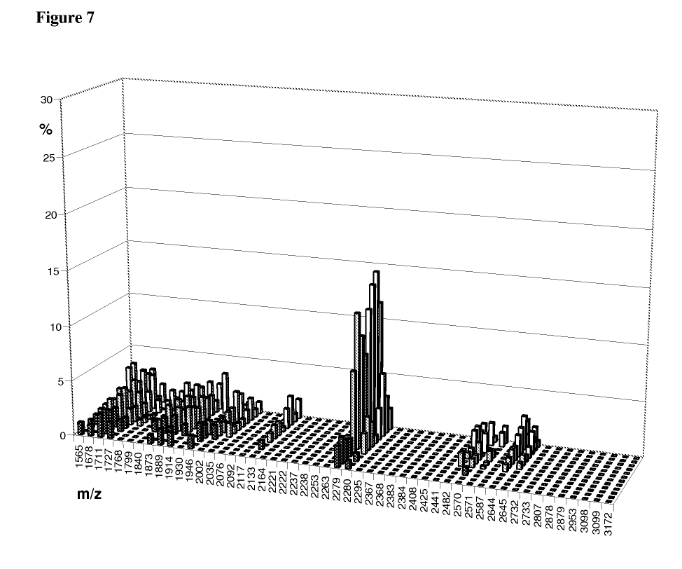

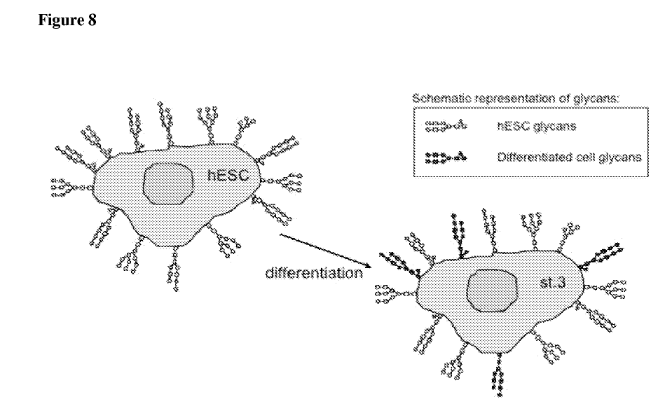

The glycome profile data was used to design glycan-specific labeling reagents for hESC. The most interesting glycan types were chosen to study their expression profiles by lectin histochemistry as exemplified in FIG. 5 for the lectins that recognize either α2,3-sialylated (MAA-lectin, FIG. 5A.) binding to the hESC cells or α-mannosylated glycans (PSA-lectin, FIG. 5B.) binding to the surfaces of feeder cells (MEF). The binding of the lectin reagents was inhibited by specific carbohydrate inhibitors, sialylα2-lactose and mannose, respectively (FIG. 5C. and 5D.). The results are summarized in Table 23.

Table 23 further represent differential recognition feeder and stem cells by two other lectins, Ricinus communis agglutinin (RCA, ricin lectin), known to recognize especially terminal Galβ-structures, especially Galβ4Glc(NAc)-type structures and peanut agglutinin (PNA) recognizing Gal/GalNAc structures. The cell surface expression of ligand for two other lectin RCA and PNA on hESC cells, but only RCA ligands of feeder cells.

The present results indicate and the invention is directed to the hESC glycans are potential targets for recognition by stem cell specific reagents. The invention is further directed to methods of specific recognition and/or separation of hESC and differentiated cells such as feeder cells by glycan structure specific reagents such as lectins. Human embryonic stem cells have a unique glycome that reflects their differentiation stage. The invention is specifically directed to analysis of cells according to the invention with regard to differentiation stage.

### Conclusions

The Present Data Represent the Glycome Profiling of hESC:
hESC have a unique N-glycome comprising of over 100 glycan components
Differentiation induces a major change in the N-glycome and the cell surface molecular landscape of hESC
   Utility of hESC Glycome Data:
Identification of new stem cell markers for e.g. antibody development
Quality control of stem cell products
Identification of hESC differentiation stage
Control of variation between hESC lines
Effect of external factors and culture conditions on hESC status

Use of the hESC glycome for identification of specific cell surface markers characteristic for the pluripotent hESCs. The invention is directed to further analysis and production of present and analogous glycome data and use of the methods for further identification of novel stem cell specific glycosylation features and form the basis for studies of hESC glycobiology and its eventual applications according to the invention

### Example 10

### Influence of Lectins on Stem Cell Proliferation Rate

### Experimental Procedures

Lectins (EY laboratories, USA) were passively adsorbed on 48-well plates (Nunclon surface, catalog No 150687, Nunc, Denmark) by overnight incubation in phosphate buffered saline.

### 12-Well Plates:

Lectins (EY laboratories, USA) were dissolved in phosphate buffered saline (140 μg/1 ml). Lectin dilutions were sterile filtrated using Millex-GV syringe driven filter units (0.22 μm, SLGV 013 SL, Millipore, Ireland) and lectins were allowed to passively adsorb on 12-well plates (Costar 3513, Corning Inc., USA) by overnight incubation in phosphate buffered saline at +4° C. After incubation the wells were washed three times with phosphate buffered saline and stem cell were plated on them.

### 48-Well Plates:

Lectins (EY laboratories, USA) were dissolved in phosphate buffered saline (100 μg/1 ml). Lectin dilutions were sterile filtrated using Millex-GV syringe driven filter units (0.22 μm, SLGV 004 SL, Millipore, Ireland) and lectins were allowed to passively adsorb on 48-well plates (Nunclon surface, catalog No 150687 Nunc, Denmark) by overnight incubation in phosphate buffered saline at +4° C. After incubation the wells were washed three times with phosphate buffered saline and stem cell were plated on them.

Human bone marrow derived mesenchymal stem cells (BM MSC) were cultured in minimum essential α-medium (α-MEM) supplemented with 20 mM HEPES, 10% FCS, penicillin-streptomycin, and 2 mM L-glutamine (all from Gibco) on 48-well plates coated with different lectins. Cells were cultivated in Cell IQ (ChipMan Technologies, Tampere, Finland) at +37° C. with 5% CO₂. Images were taken every 15 minutes. Data were analyzed with Cell IQ Analyzer software by analyzer protocol built by Dr. Ulla Impola (Finnish Red Cross Blood Service, Helsinki, Finland).

### Results and Discussion

The growth rates of BM MSC varied on different lectin-coated surfaces compared to each other and uncoated plastic surface (Table 24), indicating that proteins with different glycan binding specificities binding to stem cell surface glycans specifically influence their proliferation rate.

Lectins that had an enhancing effect on BM MSC growth rate included in order of relative efficacy:

GS II(β-GlcNAc)>ECA(LacNAc/(β-Gal)>PWA(I-branched poly-LacNAc)>LTA(α1,3-Fuc)>PSA(α-Man),

wherein the preferred oligosaccharide specificities of the lectins are indicated in parenthesis.

However, PSA was nearly equal to plastic in the present experiments.

Lectins that had an inhibitory effect on BM MSC growth rate included in order of relative efficacy:

RCA(β-Gal/LacNAc)>>UEA(α1,2-Fuc)>WFA(β-GalNAc)>STA(linear poly-LacNAc)>

NPA(α-Man)>SNA(α2,6-linked sialic acids)=MAA(α2,3-linked sialic acids/α3′-sialyl LacNAc),

wherein the preferred oligosaccharide specificities of the lectins are indicated in parenthesis. However, NPA, SNA, and MAA were nearly equal to plastic in the present experiments.

### Results

### Cell Proliferation

Cells proliferated perhaps most efficiently on MAA and ECA when compared to plastic or other types of surfaces. All wells reached confluency within a week. Cells cultivated on WFA and PWA seemed to loose their proliferation capacity during 5 weeks period and on WFA coating there were some morphologically different cells.

### Cell Morphology and Attachment

Morphologically cells growing on PSA coating differed from the others by their way of forming a netlike monolayer. Cells on MAA and PSA were also more tightly attached to the surface and their detachment with trypsin was not possible, those cells needed to be scratched off mechanically.

### Example 11

### Glycosphingolipid Glycans of Human Stem Cells

### Experimental Procedures

Samples from MSC, CB MNC, and hESC grown on mouse fibroblast feeder cells were produced as described in the preceding Examples. Neutral and acidic glycosphingolipid fractions were isolated from cells essentially as described (Miller-Podraza et al., 2000). Glycans were detached by Macrobdella decora endoglycoceramidase digestion (Calbiochem, USA) essentially according to manuacturer's instructions, yielding the total glycan oligosaccharide fractions from the samples. The oligosaccharides were purified and analyzed by MALDI-TOF mass spectrometry as described in the preceding Examples for the protein-linked oligosaccharide fractions.

### Results and Discussion

Human Embryonic Stem Cells (hESC)

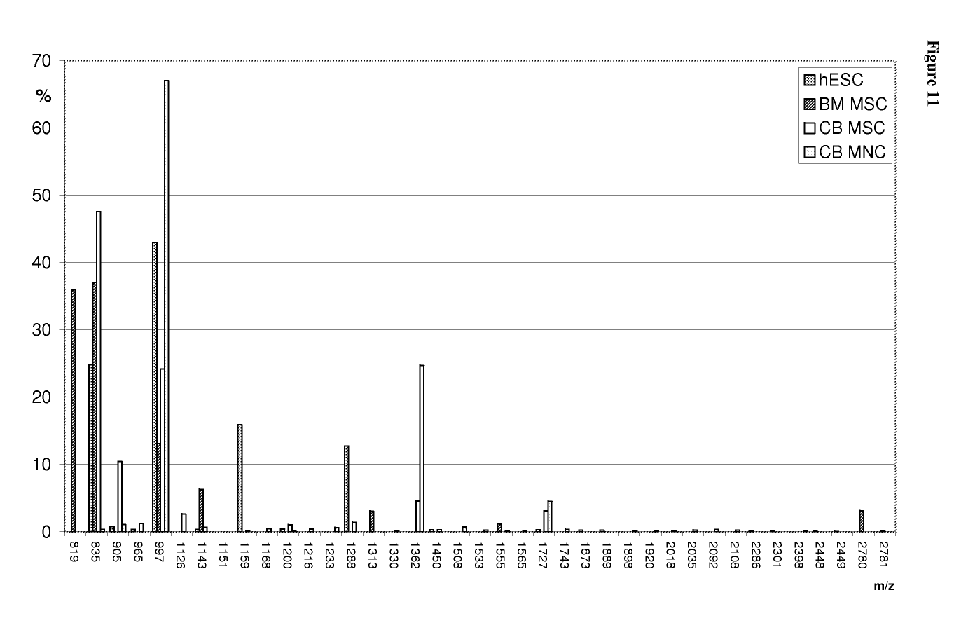

hESC neutral lipid glycans. The analyzed mass spectrometric profile of the hESC glycosphingolipid neutral glycan fraction is shown in FIG. 10.

Structural analysis of the major neutral lipid glycans. The six major glycan signals, together comprising more than 90% of the total glycan signal intensity, corresponded to monosaccharide compositions Hex₃HexNAc₁ (730), Hex₃HexNAc₁dHex₁ (876), Hex₂HexNAc₁ (568), Hex₃HexNAc₂ (933), Hex₄HexNAc₁ (892), and Hex₄HexNAc₂ (1095).

In β1,4-galactosidase digestion, the relative signal intensities of 1095 and 730 were reduced by about 30% and 10%, respectively. This suggests that 730 and 1095 contain minor components with non-reducing terminal β1,4-Gal epitopes, preferably including the structures Galβ4GlcNAcLac and Galβ4GlcNAc[Hex₁HexNAc₁]Lac. The other major components were thus shown to contain other terminal epitopes. Further, the glycan signal Hex₅HexNAc₃ (1460) was digested to Hex₃HexNAc₃ (1136), indicating that the original signal contained glycan structures containing two β1,4-Gal.

The major glycan signals were not sensitive to α-galactosidase digestion.

In α1,3/4-fucosidase digestion, the signal intensity of 876 was reduced by about 10%, indicating that only a minor proportion of the glycan signal corresponded to glycans with α1,3- or α1,4-linked fucose residue. The major affected signal in the total profile was Hex₃HexNAc₁dHex₂ (1022), indicating that it included glycans with either α1,3-Fuc or α1,4-Fuc. 511 was reduced by about 30%, indicating that the signal contained a minor component with α1,2-Fuc, preferentially including Fucα2Galβ4Glc (Fucα2′Lac, 2′-fucosyllactose).

When the α1,3/4 fucosidase reaction product was further digested with α1,2-fucosidase, 876 was completely digested into 730, indicating that the structure of the majority of the signal intensity contained non-reducing terminal α1,2-Fuc, preferably including the structure Fucα2[Hex₁HexNAc₁]Lac, more preferably including Fucα2GalHexNAcLac. Another partly digested glycan signal was Hex₄HexNAc₂dHex₁ (1241) that was thus indicated to contain α1,2-Fuc, preferably including the structure Fucα2[Hex₂HexNAc₂]Lac, more preferably including Fucα2Gal[Hex₁HexNAc₂]Lac. 511 was completely digested, indicating that the original signal contained a major component with α1,3/4-Fuc, preferentially including Galβ4(Fucα3)Glc (3-fucosyllactose).

When the α1,3/4 fucosidase and α1,2-fucosidase reaction product was further digested with β1,4-galactosidase, the majority of the newly formed 730 was not digested, i.e. the relative proportion of 568 was not increased compared to β1,4-galactosidase digestion without preceding fucosidase treatments. This indicated that the majority of 876 did not contain β1,4-Gal subterminal to Fuc. Further, 892 was not digested, indicating that it did not contain non-reducing terminal β1,4-Gal.

When the α1,3/4-fucosidase, α1,2-fucosidase, and β1,4-galactosidase reaction product was further digested with β1,3-galactosidase, the signal intensity of 892 was reduced, indicating that it included glycans with terminal β1,3-Gal. The signal intensity of 568 was increased relative to 730, indicating that also 730 included glycans with terminal β1,3-Gal.

The experimental structures of the major hESC glycosphingolipid neutral glycan signals were thus determined (‘>’ indicates the order of preference among the lipid glycan structures of hESC; ‘[ ]’ indicates that the oligosaccharide sequence in brackets may be either branched or unbranched; ‘( )’ indicates a branch in the structure):

| | |
|---|---|
| 730 | Hex₃HexNAc₁ > Hex₁HexNAc₁Lac > Galβ4GlcNAcLac |
| 876 | Hex₃HexNAc₁dHex₁ > Fucα2[Hex₁HecNAc₁]Lac > |
| | Fucα2Galβ4GlcNAcLac > Fucα3/4[Hex₁HecNAc₁]Lac |
| 568 | Hex₂HexNAc₁ > HecNAcLac |
| 933 | Hex₃HexNAc₂ > [Hex₁HecNAc₂]Lac |
| 892 | Hex₄HexNAc₁ > [Hex₂HecNAc₁]Lac > Galβ3[Hex₁HecNAc₁]Lac |
| 1095 | Hex₄HexNAc₂ > [Hex₂HecNAc₂]Lac > |
| | Galβ3HexNAc[Hex₁HecNAc₁]Lac > |
| | Galβ4GlcNAc[Hex₁HecNAc₁]Lac |
| 1460 | Hex₅HexNAc₃ > [Hex₃HecNAc₃]Lac > |
| | Galβ4GlcNAc(Galβ4GlcNAc)[Hex₁HecNAc₁]Lac |

Acidic lipid glycans. The analyzed mass spectrometric profile of the hESC glycosphingolipid sialylated glycan fraction is shown in FIG. 11. The four major glycan signals, together comprising more than 96% of the total glycan signal intensity, corresponded to monosaccharide compositions NeuAc₁Hex₃HexNAc₁ (997), NeuAc₁Hex₂HexNAc₁ (835), NeuAc₁Hex₄HexNAc₁ (1159), and NeuAc₂Hex₃HexNAc₁ (1288).

The acidic glycan fraction was subjected to α2,3-sialidase digestion and the resulting neutral and acidic glycan fractions were purified and analyzed separately. In the acidic fraction, signals 1159 and 1288 were digested and 835 was partly digested. In the neutral fraction, signals 730 and 892 were the major appeared signals. These results indicated that: 1159 consisted mainly of glycans with α2,3-NeuAc, 1288 contained at least one α2,3-NeuAc, a major proportion of glycans in 835 contained α2,3-NeuAc, and in the original sample a major proportion of NeuAc₁₋₂Hex₃HexNAc₁ contained solely α2,3-linked NeuAc.

### Human Mesenchymal Stem Cells (MSC)

Bone marrow derived (BM) MSC neutral lipid glycans. The analyzed mass spectrometric profile of the BM MSC glycosphingolipid neutral glycan fraction is shown in FIG. 10. The six major glycan signals, together comprising more than 94% of the total glycan signal intensity, corresponded to monosaccharide compositions Hex₃HexNAc₁ (730), Hex₂HexNAc₁ (568), Hex₂dHex₁ (511), Hex₂HexNAc₂dHex₂ (1063), Hex₃HexNAc₂dHex₂ (1225), and Hex₃HexNAc₂dHex₁ (1079). The four most abundant signals (730, 568, 511, and 1063) together comprised more than 75% of the total intensity.

Cord blood derived (CB) MSC neutral lipid glycans. The analyzed mass spectrometric profile of the CB MSC glycosphingolipid neutral glycan fraction is shown in FIG. 10. The ten major glycan signals, together comprising more than 92% of the total glycan signal intensity, corresponded to monosaccharide compositions Hex₂HexNAc₁ (568), Hex₃HexNAc₁ (730), Hex₄HexNAc₂ (1095), Hex₅HexNAc₃ (1460), Hex₃HexNAc₂ (933), Hex₂dHex₁ (511), Hex₂HexNAc₂dHex₂ (1063), Hex₄HexNAc₃ (1298), Hex₃HexNAc₂dHex₂ (1225), and Hex₂HexNAc₂ (771). The five most abundant signals (568, 730, 1095, 1460, and 933) together comprised more than 82% of the total intensity.

In β1,4-galactosidase digestion, the relative signal intensities of 1095, 1460, and 730 were reduced by about 90%, 95%, and 20%, respectively. This suggests that CB MSC contained major glycan components with non-reducing terminal β1,4-Gal epitopes, preferably including the structures Galβ4GlcNAcβ[Hex₁HexNAc₁]Lac, Galβ4GlcNAc[Hex₂HexNAc₂]Lac, and Galβ4GlcNAcLac. Further, the glycan signal Hex₅HexNAc₃ (1460) was digested into Hex₄HexNAc₃ (1298) and mostly into Hex₃HexNAc₃ (1136), indicating that the original signal contained glycan structures containing either one or two β1,4-Gal, and that the majority of the original glycans contained two β1,4-Gal, preferentially including the structure Galβ4GlcNAc(Galβ4GlcNAc)[Hex₁HexNAc₁]Lac. Similarly, 1095 was digested into Hex₂HexNAc₂ (771) in addition to 933, indicating that the original signal contained glycan structures containing either one or two β1,4-Gal, and that the minority of the original glycans contained two β1,4-Gal, preferentially including the structure Galβ4GlcNAc(Galβ4GlcNAc)Lac.

The experimental structures of the major CB MSC glycosphingolipid neutral glycan signals were thus determined (‘>’ indicates the order of preference among the lipid glycan structures of hESC; ‘[ ]’ indicates that the oligosaccharide sequence in brackets may be either branched or unbranched; ‘( )’ indicates a branch in the structure):

| | |
|---|---|
| 568 | Hex₂HexNAc₁ > HecNAcLac |
| 730 | Hex₃HexNAc₁ > Hex₁HexNAc₁Lac > Galβ4GlcNAcLac |
| 1095 | Hex₄HexNAc₂ > [Hex₂HecNAc₂]Lac > |
| | Galβ4GlcNAc[Hex₁HecNAc₁]Lac > |
| | Galβ4GlcNAc(Galβ4GlcNAc)Lac |
| 1460 | Hex₅HexNAc₃ > [Hex₃HecNAc₃]Lac > |
| | Galβ4GlcNAc[Hex₂HecNAc₂]Lac > |
| | Galβ4GlcNAc(Galβ4GlcNAc)[Hex₁HecNAc₁]Lac |
| 933 | Hex₃HexNAc₂ > Hex₁HexNAc₂Lac |

Sialylated lipid glycans. The analyzed mass spectrometric profile of the hESC glycosphingolipid sialylated glycan fraction is shown in FIG. 11. The five major glycan signals of BM MSC, together comprising more than 96% of the total glycan signal intensity, corresponded to monosaccharide compositions NeuAc₁Hex₂HexNAc₁ (835), NeuAc₁Hex₁ HexNAc₁dHex₁ (819), NeuAc₁Hex₃HexNAc₁ (997), NeuAc₁Hex₃HexNAc₁dHex₁ (1143), and NeuAc₂Hex₁HexNAc₂dHex₁ (1313). The six major glycan signals of CB MSC, together comprising more than 92% of the total glycan signal intensity, corresponded to monosaccharide compositions NeuAc₁Hex₂HexNAc₁ (835), NeuAc₁Hex₃HexNAc₁ (997), NeuAc₂Hex₂ (905), NeuAc₁Hex₄HexNAc₂ (1362), NeuAc₁Hex₅HexNAc₃ (1727), and NeuAc₂Hex₂HexNAc₁ (1126).

### Human Cord Blood Mononuclear Cells (CB MNC)

CB MNC neutral lipid glycans. The analyzed mass spectrometric profile of the CB MNC glycosphingolipid neutral glycan fraction is shown in FIG. 10. The five major glycan signals, together comprising more than 91% of the total glycan signal intensity, corresponded to monosaccharide compositions Hex₃HexNAc₁ (730), Hex₂HexNAc₁ (568), Hex₃HexNAc₁dHex₁ (876), Hex₄HexNAc₂ (1095), and Hex₄HexNAc₂dHex₁ (1241).

In β1,4-galactosidase digestion, the relative signal intensities of 730 and 1095 were reduced by about 50% and 90%, respectively. This suggests that the signals contained major components with non-reducing terminal β1,4-Gal epitopes, preferably including the structures Galβ4GlcNAcβLac and Galβ4GlcNAcβ[Hex₁HexNAc₁]Lac. Further, the glycan signal Hex₅HexNAc₃ (1460) was digested to Hex₄HexNAc₃ (1298) and Hex₃HexNAc₃ (1136), indicating that the original signal contained glycan structures containing either one or two β1,4-Gal.

The experimental structures of the major CB MNC glycosphingolipid neutral glycan signals were thus determined (‘>’ indicates the order of preference among the lipid glycan structures of hESC; ‘[ ]’ indicates that the oligosaccharide sequence in brackets may be either branched or unbranched; ‘( )’ indicates a branch in the structure):

| | |
|---|---|
| 730 | Hex₃HexNAc₁ > Hex₁HexNAc₁Lac > Galβ4GlcNAcLac |
| 568 | Hex₂HexNAc₁ > HecNAcLac |
| 876 | Hex₃HexNAc₁dHex₁ > [Hex₁HecNAc₁dHex₁]Lac > |
| | Fuc[Hex₁HecNAc₁]Lac |
| 1095 | Hex₄HexNAc₂ > [Hex₂HecNAc₂]Lac > |
| | Galβ4GlcNAc[Hex₁HecNAc₁]Lac |
| 1241 | Hex₄HexNAc₂dHex₁ > [Hex₂HecNAc₂dHex₁]Lac > |
| | Fuc[Hex₂HecNAc₂]Lac |
| 1460 | Hex₅HexNAc₃ > [Hex₃HecNAc₃]Lac > |
| | Galβ4GlcNAc[Hex₂HecNAc₂]Lac > |
| | Galβ4GlcNAc(Galβ4GlcNAc)[Hex₁HecNAc₁]Lac |

Sialylated lipid glycans. The analyzed mass spectrometric profile of the CB MNC glycosphingolipid sialylated glycan fraction is shown in FIG. 11. The three major glycan signals of CB MNC, together comprising more than 96% of the total glycan signal intensity, corresponded to monosaccharide compositions NeuAc₁Hex₃HexNAc₁ (997), NeuAc₁Hex₄HexNAc₂ (1362), and NeuAc₁Hex₅HexNAc₃ (1727).

### Overview of Human Stem Cell Glycosphingolipid Glycan Profiles

The neutral glycan fractions of all the present sample types altogether comprised 45 glycan signals. The proposed monosaccharide compositions of the signals were composed of 2-7 Hex, 0-5 HexNAc, and 0-4 dHex. Glycan signals were detected at monoisotopic m/z values between 511 and 2263 (for [M+Na]⁺ ion).

Major neutral glycan signals common to all the sample types were 730, 568, 1095, and 933, corresponding to the glycan structure groups Hex₀₋₁HexNAc₁Lac (568 or 730) and Hex₁₋₂HexNAc₂Lac (933 or 1095), of which the former glycans were more abundant and the latter less abundant. A general formula of these common glycans is HexₘHexNAcₙLac, wherein m is either n or n−1, and n is either 1 or 2.

### Neutral Glycolipid Profiles of Human Stem Cell Types:

Glycan signals typical to hESC preferentially include 876 and 892 (especially compared to MSC); the former preferentially corresponds to FucHexHexNAcLac, wherein α1,2-Fuc is preferential to α1,3/4-Fuc, and the latter preferentially corresponds to Hex₂HexNAc₁Lac, and more preferentially to Galβ3[Hex₁HexNAc₁]Lac; the glycan core composition Hex₄HexNAc₁ was especially characteristic of hESC compared to other human stem cell types, in addition to fucosylation and more preferentially α1,2-linked fucosylation.

Glycan signals typical to both CB and BM MSC preferentially include 771, 1063, 1225; more preferentially including compositions dHex_{0/2}Hex₀₋₁HexNAc₂Lac.

Glycan signals typical to especially BM MSC preferentially include 511 and fucosylated structures, preferentially multifucosylated structures.

Glycan signals typical to especially CB MSC preferentially include 1460 and 1298, as well as large neutral glycolipids, especially Hex₂₋₃HexNAc₃Lac. In addition, low fucosylation and/or high expression of terminal β1,4-Gal was typical to especially CB MSC.

Glycan signals typical to CB MNC preferentially include compositions dHex₀₋₁[HexHexNAc]₁₋₂Lac, more preferentially high relative amounts of 730 compared to other signals; and fucosylated structures; and glycan profiles with less variability and/or complexity than other stem cell types.

The acidic glycan fractions of all the present sample types altogether comprised 38 glycan signals. The proposed monosaccharide compositions of the signals were composed of 0-2 NeuAc, 2-9 Hex, 0-6 HexNAc, 0-3 dHex, and/or 0-1 sulphate or phosphate esters. Glycan signals were detected at monoisotopic m/z values between 786 and 2781 (for [M-H]⁻ ion).

The acidic glycosphingolipid glycans of CB MNC were mainly composed of NeuAc₁Hexₙ₊₂HexNAcₙ, wherein 1≦n≦3, indicating that their structures were NeuAc₁[HexHexNAc]₁₋₃Lac.

Terminal glycan epitopes that were demonstrated in the present experiments in stem cell glycosphingolipid glycans include:

### Gal

### Galβ4Glc (Lac)

Galβ4GlcNAc (LacNAc type 2)

### Galβ3

Non-reducing terminal HexNAc

### Fuc

### α1,2-Fuc

### α1,3-Fuc

### Fucα2Gal

Fucα2Galβ4GlcNAc (H type 2)
Fucα2Galβ4Glc (2′-fucosyllactose)

### Fucα3 GlcNAc

### Galβ4(Fucα3)GlcNAc (Lex)

### Fucα3Glc

Galβ4(Fucα3)Glc (3-fucosyllactose)

### Neu5Ac

### Neu5Acα2,3

### Neu5Acα2,6

Development-related glycan epitope expression. According to the present invention, the glycosphingolipid glycan composition Hex₄HexNAc₁ preferentially corresponds to (iso)globo structures. The glycan sequence of the SSEA-3 glycolipid antigen has been determined to be Galβ3GalNAcβ3Galα4Galβ4Glc, which corresponds to the glycan signal Hex₄HexNAc₁ (892) detected in the present experiments in hESC. Similarly, the glycan sequence of the SSEA-4 glycolipid antigen has been determined to be NeuAcα3Galβ3GalNAcβ3Galα4Galβ4Glc, which corresponds to the glycan signal NeuAc₁Hex₄HexNAc₁ (1159) detected in the present experiments in hESC. Consistent with the present glycan structure analyses, the hESC samples were determined to be SSEA-3 and SSEA-4 positive by monoclonal antibody staining as described in the preceding Examples. In higher-resolution analysis the glycan signals Hex₄HexNAc₁ and NeuAc₁Hex₄HexNAc₁ were detected in small amounts also in MSC, indicating that globoside-type glycosphingolipids were relatively minor but yet significant structures in MSC (Table 29). In contrast to mouse ES cells, hESC do not express the SSEA-1 antigen; consistent with this we found only low expression levels of α1,3/4-fucosylated neutral glycolipid glycans. In contrast, we were able to show that the major fucosylated structures of hESC glycosphingolipid glycans contain α1,2-Fuc, which is a molecular level explanation to the mouse-human difference in SSEA-1 reactivity.

### Example 12

### Lectin Based Selection of CB MNC Cell Populations

The FACS experiments with fluorescein-labeled lectins and CB MNC were performed essentially similarly to Example 4. Double stainings were performed with CD34 specific monoclonal antibody (Jaatinen et al., 2006) with complementary fluorescent dye. Erythroblast depletion from CD MNC fraction was performed by anti-glycophorin A (GlyA) monoclonal antibody negative selection.

### Results and Discussion

Compared to the CB MNC fraction, GlyA depleted CB MNC showed decreased staining in FACS with the following lectins (the decrease in % in parenthesis): PWA (48%), LTA (59%), UEA (34%), STA, MAA, and PNA (all latter three less than 23%); indicating that GlyA depletion increased the resolving power of the lectins in cell sorting.

In FACS double staining with both fluorescein-labeled lectins and anti-CD34 antibody, the following lectins colocalized with CD34+ cells: STA (3/3 samples), HHA(3/3 samples), PSA (3/3 samples), RCA (3/3 samples), and partly also NPA (2/3 samples). In contrast, the following lectins did not colocalize with CD34+ cells: GNA (3/3 samples) and PWA (3/3 samples), and partly also LTA (2/3 samples), WFA (2/3 samples), and GS-II (2/3 samples).

Taken together with the results of Example 5, the present results indicate that lectins can enrich CD34+ cells from CB MNC by both negative and positive selection, for example:
1) GNA binds to about 70% of CB MNC but not to CD34+ cells, leading to about 3× enrichment in negative selection of CB MNC in CD34+ cell isolation.
2) STA binds to about 50% of CB MNC and also to CD34+ cells, leading to about 2× enrichment in positive selection of CB MNC in CD34+ cell isolation.
3) UEA binds to about 50% of CB MNC and also to CD34+ cells, leading to about 2× enrichment in positive selection of CB MNC in CD34+ cell isolation.

### Example 13

### Galectin Gene Expression Profiles of Stem Cells

### Experimental Procedures

Gene expression analysis of CB CD133+ cells has been described (Jaatinen et al., 2006) and the present analysis was performed essentially similarly. The galectins whose gene expression profile was analyzed included (corresponding Affymetrix codes in parenthesis): Galectin-1 (201105_at), galectin-2 (208450_at), galectin-3 (208949_s_at), galectin-4 (204272_at), galectin-6 (200923_at), galectin-7 (206400_at), galectin-8 (208933_s_at), galectin-9 (203236_s_at), galectin-10 (206207_at), galectin-13 (220158_at).

### Results and Discussion

In CB CD133+ versus CD133−, as well as CD34+ versus CD34− CB MNC cells, the galectin gene expression profile was as follows: Overall, galectins 1, 2, 3, 6, 8, 9, and 10 showed gene expression in both CD34+/CD133+ cells. Galectins 1, 2, and 3 were downregulated in both CD34+/CD133+ cells with respect to CD34−/CD133− cells, and in addition galectin 10 was downregulated in CD133+ cells with respect to CD133− cells. In contrast, in both CD34+/CD133+ cells galectin 8 was upregulated with respect to CD34−/CD133− cells.

In hESC versus EB samples, the galectin gene expression profile was as follows: Overall, galectins 1, 3, 6, 8, and 13 showed gene expression in hESC. Galectin 3 was clearly downregulated with respect to EB, and in addition galectin 13 was downregulated in 2 out of 4 hESC lines. In contrast, galectin 1 was clearly upregulated in all hESC lines.

The results indicate that both CB CD34+/CD133+ stem cell populations and hESC have an interesting and distinct galectin expression profiles, leading to different galectin ligand affinity profiles (Hirabayashi et al., 2002). The results further correlate with the glycan analysis results showing abundant galectin ligand expression in these stem cells, especially non-reducing terminal β-Gal and type II LacNAc, poly-LacNAc, β1,6-branched poly-LacNAc, and complex-type N-glycan expression.

### Example 14

### Immunohistochemical Staining of Stem Cells

### Immunohistochemical Studies of Embryonic Stem Cells (in Culture) (GF Series of Stainings)

hESC cells were cultured as described In Examples. The cells were fixed and after rinsing with PBS the stem cell cultures/sections were incubated in 3% highly purified BSA in PBS for 30 minutes at RT to block nonspecific binding sites. Primary antibodies (GF279, 288, 287, 284, 285, 283, 286, 290 and 289) were diluted (1:10) in PBS containing 1% BSA-PBS and incubated 1 hour at RT. After rinsing three times with PBS, the sections were incubated with biotinylated rabbit anti-mouse, secondary antibody (Zymed Laboratories, San Francisco, Calif., USA) in PBS for 30 minutes at RT, rinsed in PBS and incubated with peroxidase conjugated streptavidin (Zymed Laboratories) diluted in PBS. The sections were finally developed with AEC substrate (3-amino-9-ethyl carbazole; Lab Vision Corporation, Fremont, Calif., USA). After rinsing with water counterstaining was performed with Mayer's hemalum solution.

Antibodies, their antigens/epitopes and codes used in the immunostainings. See also Table 19 for results.

| Producer code | Manufact | Clone | Specificity | Code | Target stucture(s) | Host/isotype |
|---|---|---|---|---|---|---|
| MAB-S206 (Globo-H) | Glycotope | A69-A/E8 | Globo-H | GF288 | Fucα2Galβ3GalNAcβ3GalαLacCer | mouse/IgM |
| MAB-S201 CD174 | Glycotope | A70-C/C8 | CD174 | GF289 | Fucα2Galβ4(Fucα3)GlcNAc | mouse/IgM |
| (Lewis y) | | | (Lewis y) | | | |
| MAB-S204 H type 2 | Glycotope | A51-B/A6 | H type 2 | GF290 | Fucα2Galβ4GlcNAc | mouse/IgA |
| DM3122: 0.1 mg | Acris | 2-25LE | Lewis b | GF283 | Fucα2Galβ3(Fucα4)GlcNAc | mouse/IgG |
| (Lewis b) | | | | | | |
| DM3015: 0.15 mg | Acris | B393 | H Type 2 | GF284 | Fucα2Galβ4GlcNAc | mouse/IgM |
| DM3014: 0.15 mg | Acris | B389 | H Type 2, | GF285 | Fucα2Galβ4GlcNAc, | mouse/IgG1 |
| | | | Le b, Ley | | Fucα2Galβ3(Fucα4)GlcNAc, | |
| | | | | | Fucα2Galβ4(Fucα3)GLcNAc | |
| BM258P: 0.2 mg | Acris | BRIC 231 | H Type 2 | GF286 | Fucα2Galβ4GlcNAc | mouse/IgG1 |
| ab3355 (blood group | Abcam | 17-206 | H type 1 | GF287 | Fucα2Galβ3GlcNAc | mouse/IgG3 |
| antigen H1) | | | | | | |
| ab3352 (pLN) | Abcam | K21 | Lewis c | GF279 | Galβ3GlcNAcβ(3Lac) | mouse/IgM |
| | | | Gb3GN | | | |

### Detection of Carbohydrate Structures on Cell Surface in Stem Cell Samples by Specific Antibodies

### Materials and Methods

Cell samples. Mesenchymal stem cells (MSCs) from bone marrow were generated and cultured in proliferation medium as described above. MSCs were cultured in differentiation medium (proliferation medium including 4 ng/ml dexamethasone, 10 mmol/L β-glycerophosphate, and 50 μmol/L ascorbic acid) for 6 weeks to induce osteogenic differentiation. Differentiation medium was refreshed twice a week throughout the differentiation period.

### Antibodies.

Immunostainings. Bone-marrow derived mesenchymal stem cells on passages 9-12 were grown on 0.01% poly-L-lysine (Sigma, USA) coated glass 8-chamber slides (Lab-TekII, Nalge Nunc, Denmark) at 37° C. with 5% CO₂ for 2-4 days. Osteogenic cells were cultured with same 8-chamber slides in differentiation medium for 6 weeks. After culturing, cells were rinsed 5 times with PBS (10 mM sodium phosphate, pH 7.2, 140 mM NaCl) and fixed with 4% PBS-buffered paraformaldehyde pH 7.2 at room temperature (RT) for 10-15 minutes, followed by washings 3 times 5 minutes with PBS. Non-specific binding sites were blocked with 3% HSA-PBS (FRC Blood Service, Finland) for 30 minutes at RT. Primary antibodies were diluted in 1% HSA-PBS (1:10-1:200) and incubated for 60 minutes at RT, followed by washings 3 times 10 minutes with PBS. Secondary antibodies, Alexa Fluor 488 goat anti-mouse IgG (H+L; 1:1000) (Invitrogen), Alexa Fluor 488 goat anti-rabbit IgG (H+L; 1:1000) (Invitrogen) or FITC-conjugated rabbit anti-rat IgG (1:320) (Sigma) in 1% HSA-PBS and incubated for 60 minutes at RT in the dark. Furthermore, cells were washed 3 times 10 minutes with PBS and mounted in Vectashield mounting medium containing DAPI-stain (Vector Laboratories, UK). Immunostainings were observed with Zeiss Axioskop 2 plus-fluorescence microscope (Carl Zeiss Vision GmbH, Germany) with FITC and DAPI filters. Images were taken with Zeiss AxioCam MRc-camera and with AxioVision Software 3.1/4.0 (Carl Zeiss) with the 400× magnification.

Fluorescence activated cell sorting (FACS) analysis. Proliferating MSCs on passage 12 were detached from culture plates by 0.02% Versene solution (pH 7.4) for 45 minutes at 37° C. Cells were washed twice with 0.3% HSA-PBS solution before antibody labelling. Primary antibodies were incubated (4 μl/100 μl cell suspension/50 000 cells) for 30 minutes at RT and washed once with 0.3% HSA-PBS before secondary antibody detection with Alexa Fluor 488 goat anti-mouse (1:500) for 30 minutes at RT in the dark. As a negative control cells were incubated without primary antibody and otherwise treated similar to labelled cells. Cells were analysed with BD FACSAria (Becton Dickinson) using FITC detector at wavelength 488. Results were analysed with BD FACSDiva software version 5.0.1 (Becton Dickinson).

Antibodies, their antigens/epitopes and codes used in the immunostainings. See also Table 19 for results.

| Code | Antigen | Host | Dilution IHC | Class | Manufact | Cat No |
|---|---|---|---|---|---|---|
| GF274 | PNAd (peripheral lymph node addressin; | Rat anti- | 5-20 μg/ml | IgM, κ | BD | 553863 |
| | CD62L ligand) closely associated with L- | mouse | | | Pharmingen | |
| | selectin (CD34, GlyCAM-1, MAdCAM-1), | | | | | |
| | sulfo-mucin | | | | | |
| GF275 | CA15-3 (Cancer antigen 15-3; sialylated | Mouse anti- | | IgG1 | Acris | BM3359 |
| | carbohydrate epitope of the MUC-1 | human | | | Antibodies | |
| | glycoprotein) | | | | | |
| GF276 | oncofetal antigen, tumor associated | Mouse anti- | 1:20-1:50 | IgG1 | Acris | DM288 |
| | glycoprotein (TAG-72) or CA 72-4 | human | | | Antibodies | |
| GF277 | human sialosyl-Tn antigen (STn, | Mouse anti- | 1:50-1:100 | IgG1 | Acris | DM3197 |
| | sCD175) | human | (4-8 μg/ml) | | Antibodies | |
| GF278 | human Tn antigen (Tn, CD175 B1.1) | Mouse anti- | 1:50 | IgM | Acris | DM3218 |
| | | human | (4 μg/ml) | | Antibodies | |

| | | | Dilution | | | |
|---|---|---|---|---|---|---|
| Koodi | Antigen | Host | IHC | Class | Manufact | Cat No |
| GF295 | Blood group antigen precursor (BG1), | Mouse anti- | 01:40 | IgM | Abcam | ab3352 |
| | Lewis c Gb3GN (pLN) | human | | | | |
| GF280 | TF-antigen isoform (Nemod TF2) | Mouse anti-? | | IgM | | MAB- |
| | | | | | | S301 |
| GF281 | TF-antigen isoform (A68-E/E3) | Mouse anti-? | | IgG1 | | MAB- |
| | | | | | | S305 |
| GF296 | asialoganglioside GM1 | Rabbit anti- | 1:100-1:400 | polycl. | Acris | BP282 |
| | | bovine | ELISA | | Antibodies | |
| GF297 | Globoside GL4 | Rabbit anti- | 1:50-1:100 | polycl. | Abcam | ab23949 |
| | | several | ELISA | IgG | | |
| | | species | | | | |
| GF298 | Human CD77 (=blood group substance | Rat anti- | | IgM | Acris | SM1160P |
| | pk), GB3 | human | | | Antibodies | |
| GF299 | Forssman antigen, glycosphingolipid (FOGSL) | Rat anti- | 1:100-1:1000 | IgG | Acris | BM4091 |
| | differentiation ag | mouse | | | Antibodies | |
| | | (human ??) | | | | |
| GF300 | Asialo GM2 | Rabbit anti- | 1:100-1:400 | polycl. | Acris | BP283 |
| | | bovine | ELISA | | Antibodies | |

| | | | Dilution | | | |
|---|---|---|---|---|---|---|
| Code | Antigen | Host | IHC | Class | Producer | Cat no |
| GF301 | Lewis b blood | Mouse anti- | | IgG1 | Acris | SM3092P |
| | group antigen | human | | | Antibodies | |
| GF302 | H type 2 blood | Mouse anti- | | IgM | Acris | DM3015 |
| | group antigen | human | | | Antibodies | |
| GF303 | Blood group | Mouse anti- | | IgG3 | Abcam | ab3355 |
| | H1(O) antigen (BG4) | human | | | | |
| GF288 | Globo-H | Mouse anti-? | | IgM | | MAB- |
| | | | | | | S206 |

| | | | Dilution | | | |
|---|---|---|---|---|---|---|
| Code | Antigen | Host | IHC | Class | Producer | Cat no |
| GF304 | Lewis a | Mouse anti- | | IgG1 | Chemicon int. | CBL205 |
| | | human | | | | |
| GF305 | Lewis x, CD15, 3-FAL, SSEA-1,3- | Mouse anti- | | IgM | Chemicon int. | CBL144 |
| | fucosyl-N-acetyllactosamine | human | | | | |
| GF306 | Sialyl Lewis a | Mouse anti- | 01:40 | IgG1 | Chemicon int. | MAB2095 |
| | | human | | | | |
| GF307 | Sialyl Lewis x | Mouse anti- | 01:40 | IgM | Chemicon int. | MAB2096 |
| | | human | | | | |

| | | | Dilution | | | |
|---|---|---|---|---|---|---|
| Code | Antigen | Host | IHC | Class | Producer | Cat no |
| GF353 | SSEA-3 (stage-specific embryonic | Rat anti- | 10-20 μg/ml | IgM | Chemicon int. | MAB4303 |
| | antigen-3) | mouse/human | | | | |
| GF354 | SSEA-4 (stage-specific embryonic | Mouse anti- | 10-20 μg/ml | IgG3 | Chemicon int. | MAB4304 |
| | antigen-4) | human | | | | |
| GF355 | Galactose-a(1,3)galactose | Baboon | 1:500 | serum | Chemicon int. | AB2052 |
| | | anti- | | | | |
| | | porcine/rat | | | | |
| GF365 | Nemod TF1, DC176, GalB1-3GalNAc | Mouse anti- | | IgM, k | Glycotope | Lot 31-2006 |
| | | human | | | | |

### Example 15

### Glycosidase Profiling of Cord Blood Mononuclear Cell N-Glycans

### Experimental Procedures

Exoglycosidase digestions. Neutral N-glycan fractions were isolated from cord blood mononuclear cell populations as described above. Exoglycosidase reactions were performed essentially after manufacturers' instructions and as described in (Saarinen et al., 1999). The different reactions were; α-Man: α-mannosidase from Jack beans (C. ensiformis; Sigma, USA); β1,4-Gal: β1,4-galactosidase from S. pneumoniae (recombinant in E. coli; Calbiochem, USA); β1,3-Gal: recombinant β1,3-galactosidase (Calbiochem, USA); β-GlcNAc: β-glucosaminidase from S. pneumoniae (Calbiochem, USA); α2,3-SA: α2,3-sialidase from S. pneumoniae (Calbiochem, USA). The analytical reactions were carefully controlled for specificity with synthetic oligosaccharides in parallel control reactions that were analyzed by MALDI-TOF mass spectrometry. The sialic acid linkage specificity of α2,3-SA was controlled with synthetic oligosaccharides in parallel control reactions, and it was confirmed that in the reaction conditions the enzyme hydrolyzed α2,3-linked but not α2,6-linked sialic acids. The analysis was performed by MALDI-TOF mass spectrometry as described in the preceding examples. Digestion results were analyzed by comparing glycan profiles before and after the reaction.

RESULTS Glycosidase profiling of neutral N-glycans. Neutral N-glycan fractions from affinity-purified CD34+, CD34−, CD133+, CD133−, Lin+, and Lin− cell samples from cord blood mononuclear cells were isolated as described above. The glycan samples were subjected to parallel glycosidase digestions as described under Experimental procedures. Profiling results are summarized in Table 2 (CD34+ and CD34− cells), Table 3 (CD133+ and CD133− cells), and Table 4 (Lin− and Lin+ cells). The present results show that several neutral N-glycan signals are individually sensitive towards all the exoglycosidases, indicating that in all the cell types several neutral N-glycans contain specific substrate glycan structures in their non-reducing termini. The results also show clear differences between the cell types in both the sensitivity of individual glycan signals towards each enzyme and also profile-wide differences between cell types, as detailed in the Tables cited above.

Glycosidase profiling of sialylated N-glycans. Sialylated N-glycan fractions from affinity-purified CD133+ and CD133− cell samples from cord blood mononuclear cells were isolated as described above. The glycan samples were subjected to parallel glycosidase digestions as described under Experimental procedures. Profiling results by α2,3-sialidase are shown in Table 5. The results show significant differences between the glycan profiles of the analyzed cell types in the sialylated and neutral glycan fractions resulting in the reaction. The present results show that differences are seen in multiple signals in a profile-wide fashion. Also individual signals differ between cell types, as discussed below.

Cord blood CD133⁺ and CD133⁻ cell N-glycans are differentially α2,3-sialylated. Sialylated N-glycans from cord blood CD133⁺ and CD133⁻ cells were treated with α2,3-sialidase, after which the resulting glycans were divided into sialylated and non-sialylated fractions, as described under Experimental procedures. Both α2,3-sialidase resistant and sensitive sialylated N-glycans were observed, i.e. after the sialidase treatment sialylated glycans were observed in the sialylated N-glycan fraction and desialylated glycans were observed in the neutral N-glycan fraction. The results indicate that cord blood CD133⁺ and CD133⁻ cells are differentially α2,3-sialylated. For example, after α2,3-sialidase treatment the relative proportions of monosialylated (SA₁) glycan signal at m/z 2076, corresponding to the [M-H]⁻ ion of NeuAc₁Hex₅HexNAc₄dHex₁, and the disialylated (SA₂) glycan signal at m/z 2367, corresponding to the [M-H]⁻ ion of NeuAc₂Hex₅HexNAc₄dHex₁, indicate that α2,3-sialidase resistant disialylated N-glycans are relatively more abundant in CD133⁻ than in CD133⁺ cells, when compared to α2,3-sialidase resistant monosialylated N-glycans. It is concluded that N-glycan α2,3-sialylation in relation to other sialic acid linkages including especially α2,6-sialylation, is more abundant in cord blood CD133⁺ cells than in CD133⁻ cells.

In cord blood CD133⁻ cells, several sialylated N-glycans were observed that were resistant to α2,3-sialidase treatment, i.e. neutral glycans were not observed that would correspond to the desialylated forms of the original sialylated glycans. The results revealing differential α2,3-sialylation of individual N-glycan structures between cord blood CD133⁺ and CD133⁻ cells are presented in Table 5. The present results indicate that N-glycan α2,3-sialylation in relation to other sialic acid linkages is more abundant in cord blood CD133⁺ cells than in CD133⁻ cells.

Sialidase analysis. The sialylated N-glycan fraction isolated from a cord blood mononuclear cell population (CB MNC) was digested with broad-range sialidase as described in the preceding Examples. After the reaction, it was observed by MALDI-TOF mass spectrometry that the vast majority of the sialylated N-glycans were desialylated and transformed into corresponding neutral N-glycans, indicating that they had contained sialic acid residues (NeuAc and/or NeuGc) as suggested by the proposed monosaccharide compositions. Combined glycan profiles of neutral and desialylated (originally sialylated) N-glycan fractions of a CB MNC population was produced. The profiles correspond to total N-glycan profiles isolated from the cell samples (in desialylated form). It is calculated that approximately 25% of the N-glycan signals correspond to high-mannose type N-glycan monosaccharide compositions, and 28% to low-mannose type N-glycans, 34% to complex-type N-glycans, and 13% to hybrid-type or monoantennary N-glycans monosaccharide compositions.

CONCLUSIONS The present results suggest that 1) the glycosidase profiling method can be used to analyze structural features of individual glycan signals, as well as differences in individual glycans between cell types, 2) different cell types differ from each other with respect to both individual glycan signals' and glycan profiles' susceptibility to glycosidases, and 3) glycosidase profiling can be used as a further means to distinguish different cell types, and in such case the parameters for comparison are both individual signals and profile-wide differences.

### Example 16

### Enzymatic Modification of Cell Surface Glycan Structures

### Experimental Procedures

Enzymatic modifications. Sialyltransferase reaction: Human cord blood mononuclear cells (3×10⁶ cells) were modified with 60 mU α2,3-(N)-sialyltransferase (rat, recombinant in S. frugiperda, Calbiochem), 1.6 μmol CMP-Neu5Ac in 50 mM sodium 3-morpholinopropanesulfonic acid (MOPS) buffer pH 7.4, 150 mM NaCl at total volume of 100 μl for up to 12 hours. Fucosyltransferase reaction: Human cord blood mononuclear cells (3×10⁶ cells) were modified with 4 mU α1,3-fucosyltransferase VI (human, recombinant in S. frugiperda, Calbiochem), 1 mol GDP-Fuc in 50 mM MOPS buffer pH 7.2, 150 mM NaCl at total volume of 100 μl for up to 3 hours. Broad-range sialidase reaction: Human cord blood mononuclear cells (3×10⁶ cells) were modified with 5 mU sialidase (A. ureafaciens, Glyko, UK) in 50 mM sodium acetate buffer pH 5.5, 150 mM NaCl at total volume of 100 μl for up to 12 hours. α2,3-specific sialidase reaction: Cells were modified with α2,3-sialidase (S. pneumoniae, recombinant in E. coli) in 50 mM sodium acetate buffer pH 5.5, 150 mM NaCl at total volume of 100 μl. α-mannosidase reaction: α-mannosidase was from Jack beans and reaction was performed essentially similarly as with other enzymes described above. Sequential enzymatic modifications: Between sequential reactions cells were pelleted with centrifugation and supernatant was discarded, after which the next modification enzyme in appropriate buffer and substrate solution was applied to the cells as described above. Washing procedure: After modification, cells were washed with phosphate buffered saline. Glycan analysis. After washing the cells, total cellular glycoproteins were subjected to N-glycosidase digestion, and sialylated and neutral N-glycans isolated and analyzed with mass spectrometry as described above. For O-glycan analysis, the glycoproteins were subjected to reducing alkaline β-elimination essentially as described previously (Nyman et al., 1998), after which sialylated and neutral glycan alditol fractions were isolated and analyzed with mass spectrometry as described above.

### Results

Sialidase digestion. Upon broad-range sialidase catalyzed desialylation of living cord blood mononuclear cells, sialylated N-glycan structures as well as O-glycan structures (data not shown) were desialylated, as indicated by increase in relative amounts of corresponding neutral N-glycan structures, for example Hex₆HexNAc₃, Hex₅HexNAc₄dHex₀₋₂, and Hex₆HexNAc₅dHex₀₋₁ monosaccharide compositions (Table 9). In general, a shift in glycosylation profiles towards glycan structures with less sialic acid residues was observed in sialylated N-glycan analyses upon broad-range sialidase treatment. The shift in glycan profiles of the cells upon the reaction served as an effective means to characterize the reaction results. It is concluded that the resulting modified cells contained less sialic acid residues and more terminal galactose residues at their surface after the reaction.

α2,3-specific sialidase digestion. Similarly, upon α2,3-specific sialidase catalyzed desialylation of living mononuclear cells, sialylated N-glycan structures were desialylated, as indicated by increase in relative amounts of corresponding neutral N-glycan structures (data not shown). In general, a shift in glycosylation profiles towards glycan structures with less sialic acid residues was observed in sialylated N-glycan analyses upon α2,3-specific sialidase treatment. The shift in glycan profiles of the cells upon the reaction served as an effective means to characterize the reaction results. It is concluded that the resulting modified cells contained less α2,3-linked sialic acid residues and more terminal galactose residues at their surface after the reaction.

Sialyltransferase reaction. Upon α2,3-sialyltransferase catalyzed sialylation of living cord blood mononuclear cells, numerous neutral (Table 9) and sialylated N-glycan (Table 8) structures as well as O-glycan structures (data not shown) were sialylated, as indicated by decrease in relative amounts of neutral N-glycan structures (Hex₅HexNAc₄dHex₀₋₃ and Hex₆HexNAc₅dHex₀₋₂ monosaccharide compositions in Table 9) and increase in the corresponding sialylated structures (for example the NeuAc₂Hex₅HexNAc₄dHex₁ glycan in Table 8). In general, a shift in glycosylation profiles towards glycan structures with more sialic acid residues was observed both in N-glycan and O-glycan analyses. It is concluded that the resulting modified cells contained more α2,3-linked sialic acid residues and less terminal galactose residues at their surface after the reaction.

Fucosyltransferase reaction. Upon α1,3-fucosyltransferase catalyzed fucosylation of living cord blood mononuclear cells, numerous neutral (Table 9) and sialylated N-glycan structures as well as O-glycan structures (see below) were fucosylated, as indicated by decrease in relative amounts of nonfucosylated glycan structures (without dHex in the proposed monosaccharide compositions) and increase in the corresponding fucosylated structures (with n_{dHex}>0 in the proposed monosaccharide compositions). For example, before fucosylation O-glycan alditol signals at m/z 773, corresponding to the [M+Na]⁺ ion of Hex₂HexNAc₂ alditol, and at m/z 919, corresponding to the [M+Na]⁺ ion of Hex₂HexNAc₂dHex₁ alditol, were observed in approximate relative proportions 9:1, respectively (data not shown). After fucosylation, the approximate relative proportions of the signals were 3:1, indicating that significant fucosylation of neutral O-glycans had occurred. Some fucosylated N-glycan structures were even observed after the reaction that had not been observed in the original cells, for example neutral N-glycans with proposed structures Hex₆HexNAc₅dHex₁ and Hex₆HexNAc₅dHex₂ (Table 9), indicating that in α1,3-fucosyltransferase reaction the cell surface of living cells can be modified with increased amounts or extraordinary structure types of fucosylated glycans, especially terminal Lewis x epitopes in protein-linked N-glycans as well as in O-glycans.

Sialidase digestion followed by sialyltransferase reaction. Cord blood mononuclear cells were subjected to broad-range sialidase reaction, after which α2,3-sialyltransferase and CMP-Neu5Ac were added to the same reaction, as described under Experimental procedures. The effects of this reaction sequence were observable on the N-glycan profiles. The sialylated N-glycan profile was also analyzed between the reaction steps, and the result clearly indicated that sialic acids were first removed from the sialylated N-glycans (indicated for example by appearance of increased amounts of neutral N-glycans), and then replaced by α2,3-linked sialic acid residues (indicated for example by disappearance of the newly formed neutral N-glycans; data not shown). It is concluded that the resulting modified cells contained more α2,3-linked sialic acid residues after the reaction.

Sialyltransferase reaction followed by fucosyltransferase reaction. Cord blood mononuclear cells were subjected to α2,3-sialyltransferase reaction, after which α1,3-fucosyltransferase and GDP-fucose were added to the same reaction, as described under Experimental procedures. The effects of this reaction sequence were observable on the sialylated N-glycan profiles of the cells. The results show that a major part of the glycan signals (examples in Tables 8 and 9) have undergone changes in their relative intensities, indicating that a major part of the sialylated N-glycans present in the cells were substrates of the enzymes. It was also clear that the combination of the enzymatic reaction steps resulted in different result than either one of the reaction steps alone.

Different from the α1,3-fucosyltransferase reaction described above, sialylation before fucosylation apparently sialylated the neutral fucosyltransferase acceptor glycan structures present on cord blood mononuclear cell surfaces, resulting in no detectable formation of the neutral fucosylated N-glycan structures that had emerged after α1,3-fucosyltransferase reaction alone (discussed above; Table 9).

α-mannosidase reaction. α-mannosidase reaction of whole cells showed a minor reduction of glycan signals including those indicated to contain α-mannose residues in the preceding examples.

Glycosyltransferase-derived glycan structures. We detected that glycosylated glycosyltransferase enzymes can contaminate cells in modification reactions. For example, when cells were incubated with recombinant fucosyltransferase or sialyltransferase enzymes produced in S. frugiperda cells, N-glycosidase and mass spectrometric analysis of cellular and/or cell-associated glycoproteins resulted in detection of an abundant neutral N-glycan signal at m/z 1079, corresponding to [M+Na]⁺ ion of Hex₃HexNAc₂dHex₁ glycan component (calc. m/z 1079.38). Typically, in recombinant glycosyltransferase treated cells, this glycan signal was more abundant than or at least comparable to the cells' own glycan signals, indicating that insect-derived glycoconjugates are a very potent contaminant associated with recombinant glycan-modified enzymes produced in insect cells. Moreover, this glycan contamination persisted even after washing of the cells, indicating that the insect-type glycoconjugate corresponding to or associated with the glycosyltransferase enzymes has affinity towards cells or has tendency to resist washing from cells. To confirm the origin of the glycan signal, we analyzed glycan contents of commercial recombinant fucosyltransferase and sialyltransferase enzyme preparations and found that the m/z 1079 glycan signal was a major N-glycan signal associated with these enzymes. Corresponding N-glycan structures, e.g. Manα3(Manα6)Manβ4GlcNAc(Fucα3/6)GlcNAc(β-N-Asn), have been described previously from glycoproteins produced in S. frugiperda cells (Staudacher et al., 1992; Kretzchmar et al., 1994; Kubelka et al., 1994; Altmann et al., 1999). As described in the literature, these glycan structures, as well as other glycan structures potentially contaminating cells treated with recombinant or purified enzymes, especially insect-derived products, are potentially immunogenic in humans and/or otherwise harmful to the use of the modified cells. It is concluded that glycan-modifying enzymes must be carefully selected for modification of human cells, especially for clinical use, not to contain immunogenic glycan epitopes, non-human glycan structures, and/or other glycan structures potentially having unwanted biological effects.

### Example 17

### Exoglycosidase Analysis of Human Embryonic Stem Cells

### Experimental Procedures

hESC and differentiated cell samples. The human embryonic stem cell (hESC) and embryoid body (EB) samples were prepared from hESC line FES 29 (Skottman et al., 2005) essentially as described in the preceding Examples, however in the present Example the hESCs were propagated on murine fibroblast feeder cells (mEF) and the hESC samples contained some mEF cells.

Exoglycosidase digestions were performed essentially as described (Saarinen et al., 1999) and as described in the preceding Examples. The enzymes used were α-mannosidase and β-hexosaminidase from Jack beans (C. ensiformis, Sigma, USA), β-glucosaminidase and β1,4-galactosidase from S. pneumoniae (rec. in E. coli, Calbiochem, USA), α2,3-sialidase from S. pneumoniae (Glyko, UK), α1,3/4-fucosidase from Xanthomonas sp. (Calbiochem, USA), α1,2-fucosidase from X. manihotis (Glyko), β1,3-galactosidase (rec. in E. coli, Calbiochem), and α2,3/6/8/9-sialidase from A. ureafaciens (Glyko). The specific activities of the enzymes were controlled in parallel reactions with purified oligosaccharides or oligosaccharide mixtures, and analyzed similarly as the analytic reactions. The changes in the exoglycosidase digestion result Tables are relative changes in the recorded mass spectra and they do not reflect absolute changes in the glycan profiles resulting from glycosidase treatments.

### Results and Discussion

hESC. Neutral and acidic N-glycan fractions were isolated from hESC grown on both murine and human fibroblast feeder cells as described in the preceding Examples. The results of parallel exoglycosidase digestions of the neutral (Tables 10 and 11) and acidic (Table 12) glycan fractions are discussed below. In the following chapters, the glycan signals are referred to by their proposed monosaccharide compositions according to the Tables of the present invention and the corresponding m/z values can be read from the Tables.

α-mannosidase sensitive structures. All the glycan signals that showed decrease upon α-mannosidase digestion of the neutral N-glycan fraction (Tables 10 and 11) are indicated to correspond to glycans that contain terminal α-mannose residues. The present results indicate that the majority of the neutral N-glycans of hESC contain terminal α-mannose residues. On the other hand, increased signals correspond to their reaction products. Structure groups that form series of α-mannosylated glycans in the neutral N-glycan fraction as well as individual α-mannosylated glycans are discussed below in detail.

The Hex₁₋₉HexNAc₁ glycan series was digested so that Hex₃₋₉HexNAc₁ were digested and transformed into Hex₁HexNAc₁ (data not shown), indicating that they had contained terminal α-mannose residues. Because they were transformed into Hex₁HexNAc₁, their experimental structures were (Manα)₁₋₈Hex₁HexNAc₁.

The Hex₁₋₁₂HexNAc₂ glycan series was digested so that Hex₃₋₁₂HexNAc₂ were digested and transformed into Hex₁₋₇HexNAc₂ and especially into Hex₁HexNAc₂ that had not existed before the reaction and was the major reaction product. This indicates that 1) glycans Hex₃₋₁₂HexNAc₂ include glycans containing terminal α-mannose residues, 2) glycans Hex₁₋₇HexNAc₂ could be formed from larger α-mannosylated glycans, and 3) majority of the glycans Hex₃₋₁₂HexNAc₂ were transformed into newly formed Hex₁HexNAc₂ and therefore had the experimental structures (Manα)ₙHex₁HexNAc₂, wherein n≦1. The fact that the α-mannosidase reaction was only partially completed for many of the signals suggests that also other glycan components are included in the Hex₁₋₁₂HexNAc₂ glycan series. In particular, the Hex₁₀₋₁₂HexNAc₂ components contain 1-3 hexose residues more than the largest typical mammalian high-mannose type N-glycan, suggesting that they contains glucosylated structures including (Glcα)₁₋₃Hex₈HexNAc₂, preferentially α2- and/or α3-linked Glc and even more preferentially present in the glucosylated N-glycans Glcα3→Man₉GlcNAc₂, Glcα2Glcα3→Man₉GlcNAc₂, and/or Glcα2Glcα2Glcα3→Man₉GlcNAc₂. The corresponding glucosylated fragments were observed after the α-mannosidase digestion, preferentially corresponding to Glc₁₋₃Man₄GlcNAc₂ (Hex₅₋₇HexNAc₂).

The Hex₁₋₆HexNAc₁dHex₁ glycan series was digested so that Hex₃₋₉HexNAc₁dHex₁ were digested and transformed into Hex₁HexNAc₁dHex₁, indicating that they had contained terminal α-mannose residues and their experimental structures were (Manα)₂₋₅Hex₁HexNAc₁dHex₁. Hex₁HexNAc₁dHex₁ appeared as a new signal indicating that glycans with structures (Manα)ₙHex₁HexNAc₁dHex₁, wherein n≧1, had existed in the sample.

The Hex₂₋₇HexNAc₃ glycan series was digested so that Hex₅₋₇HexNAc₃ were digested and transformed into other glycans in the series, indicating that they had contained terminal α-mannose residues. Hex₂HexNAc₃ appeared as a new signal indicating that glycans with structures (Manα)ₙHex₂HexNAc₃, wherein n≧1, had existed in the sample.

The Hex₂₋₇HexNAc₃dHex₁ glycan series was digested so that Hex₅₋₇HexNAc₃dHex₁ were digested and transformed into other glycans in the series, indicating that they had contained terminal α-mannose residues. Hex₂HexNAc₃dHex₁ was increased significantly indicating that glycans with structures (Manα)ₙHex₂HexNAc₃dHex₁, wherein n≧1, had existed in the sample.

Hex₃HexNAc₃dHex₂ appeared as a new signal indicating that glycans with structures (Manα)ₙHex₃HexNAc₃dHex₂, wherein n≧1, had existed in the sample. β-glucosaminidase sensitive structures. The Hex₃HexNAc₂₋₅ and Hex₃HexNAc₂₋₅dHex₁ glycan series were digested so that Hex₃₋₅HexNAc₁dHex₀₋₁ were digested and transformed into Hex₃HexNAc₂dHex₀₋₁, indicating that they had contained terminal β-GlcNAc residues and their experimental structures were (GlcNAcβ→)₁₋₃Hex₃HexNAc₂ and (GlcNAcβ)₁₋₃Hex₃HexNAc₂dHex₁, respectively.

Hex₄HexNAc₄, Hex₄HexNAc₄dHex₁, Hex₄HexNAc₄dHex₂, and Hex₅HexNAc₅dHex₁ were also digested indicating they contained structures including (GlcNAcβ→)Hex₄HexNAc₃, (GlcNAcβ→)Hex₄HexNAc₃dHex₁, (GlcNAcβ→)Hex₄HexNAc₃dHex₂, and (GlcNAcβ→)Hex₅HexNAc₄dHex₁, respectively.

Hex₄HexNAc₅dHex₁ and Hex₄HexNAc₅dHex₂ were digested by β-glucosaminidase and indicated to contain two β-GlcNAc residues each. In contrast, Hex₄HexNAc₅ was not digested with β-glucosaminidase.

β-hexosaminidase sensitive structures. The Hex₄HexNAc₅ glycan signal was sensitive to β-hexosaminidase but not to β-glucosaminidase indicating that it corresponded to glycan structures containing terminal β-N-acetylhexosamine residues other than β-GlcNAc, preferentially β-GalNAc. Upon β-hexosaminidase digestion, the signal was transformed into Hex₄HexNAc₃ indicating that the enzyme liberated two HexNAc residues from the corresponding glycan structures.

β1,4-galactosidase sensitive structures. Glycan signals that were sensitive to β1,4-galactosidase comprised a major proportion of hESC glycans, indicating that β1,4-linked galactose is a common terminal epitope in hESC neutral N-glycans. Hex₅HexNAc₄ and Hex₅HexNAc₄dHex₁ were digested into Hex₃HexNAc₄ and Hex₃HexNAc₄dHex₁ indicating they had the structures (Galβ4GlcNAcβ→)₂Hex₃HexNAc₂ and (Galβ4GlcNAcβ→)₂Hex₃HexNAc₂dHex₁, respectively. In contrast, Hex₅HexNAc₄dHex₂ was digested into Hex₄HexNAc₄dHex₂ indicating that it had the structure (Galβ4GlcNAcβ→)Hex₄HexNAc₃dHex₂, and Hex₅HexNAc₄dHex₃ was not digested at all. Taken together, in hESC, hexose residues are protected by deoxyhexose residues from the action of β1,4-galactosidase in the N-glycan structures. Such dHex-protected structures containing β1,4-linked galactose include Galβ4(Fucα3)GlcNAc and Fucα2Galβ4GlcNAc.

Hex₄HexNAc₅ that also included a β-hexosaminidase sensitive component was digested by β1,4-galactosidase. Taken together, the results suggest that the Hex₄HexNAc₅ glycan signal includes glycan structures including Galβ4GlcNAc(GalNAcβHexNAcβ)Hex₃HexNAc₂. β1,3-galactosidase sensitive structures. Because only few structures in hESC neutral N-glycan fraction were sensitive to the action of β1,3-galactosidase, the majority of terminal galactose residues appear to be β1,4-linked.

Glycosidase resistant structures. In the present experiments, Hex₄HexNAc₃, Hex₄HexNAc₃dHex₂, and Hex₅HexNAc₅ were resistant to the tested exoglycosidases. The second monosaccharide composition contains more than one deoxyhexose residues suggesting that it is protected from glycosidase digestions by dHex residues such as α2-, α3-, or α4-linked fucose residues, preferentially present in Fucα2Gal, Fucα3GlcNAc, and/or Fucα4GlcNAc epitopes.

The compiled neutral N-glycan fraction glycan structures based on the exoglycosidase digestions of hESC are presented in Table 13.

Acidic N-glycan fraction. The acidic N-glycan fraction of hESC grown on mEF cell layers were characterized by parallel α2,3-sialidase and A. ureafaciens sialidase treatments as well as sequential digestions with α1,3/4-fucosidase and α1,2-fucosidase. The results from these reactions as analyzed by MALDI-TOF mass spectrometry are described in Table 12. The results suggest that multiple N-glycan components in the hESC sample contain the specific glycan substrates for these enzymes, namely α2,3-linked and other sialic acid residues, and both α1,2- and α1,3/4-linked fucose residues. Some glycan signals showed the presence of many of these epitopes, such as the glycan signal at m/z 2222 (corresponding to NeuAc₁Hex₅HexNAc₄dHex₂) that was suggested to contain all these epitopes, preferentially in multiple glycan structures. The compiled acidic N-glycan fraction glycan structures based on the exoglycosidase digestions of hESC are presented in Table 25.

EB. Differentiation specific changes between embryoid bodies (EB; FES 29 st 2 in Table 10) and hESC (FES 29 st 1 in Table 10) were reflected in their neutral N-glycan fraction exoglycosidase digestion profiles, as described in Table 10. Differential exoglycosidase digestion results were observed in glycan signals including m/z 1688, 1704, 1793, 1866, 1955, 1971, 2012, 2028, 2142, 2158, and 2320, corresponding to different neutral N-glycan fraction glycan profiles.

mEF. By comparison of Table 26 and Table 10, murine feeder cell (mEF) specific neutral N-glycan fraction glycan components were identified and they are listed in Table 27. These glycan components are characterized by additional hexose residues compared to hESC or hEF specific structures according to the present invention. The exoglycosidase experiments also suggest that β1,4-linked galactose epitopes are protected from β1,4-galactosidase digestion by any additional hexose residues in the monosaccharide compositions. Taken together with the NMR analysis results of the present invention, the additional hexose residues are suggested to be α-linked galactose residues, more specifically including Galα3Gal epitopes in the N-glycan antennae, as described in Table 27.

### Example 18

### Exoglycosidase Analysis of Human Mesenchymal Stem Cells

The changes in the exoglycosidase digestion result Tables are relative changes in the recorded mass spectra and they do not reflect absolute changes in the glycan profiles resulting from glycosidase treatments. The experimental procedures are described in the preceding Example.

### Results

### Undifferentiated BM MSC

Neutral and acidic N-glycan fractions were isolated from BM MSC as described. The results of parallel exoglycosidase digestions of the neutral (Table 14) and acidic (data not shown) glycan fractions are discussed below. In the following chapters, the glycan signals are referred to by their proposed monosaccharide compositions according to the Tables of the present invention and the corresponding m/z values can be read from the Tables.

α-mannosidase sensitive structures. All the glycan signals that showed decrease upon α-mannosidase digestion of the neutral N-glycan fraction (Table 14) are indicated to correspond to glycans that contain terminal α-mannose residues. The present results indicate that the majority of the neutral N-glycans of BM MSC contain terminal α-mannose residues. On the other hand, increased signals correspond to their reaction products. Structure groups that form series of α-mannosylated glycans in the neutral N-glycan fraction as well as individual α-mannosylated glycans are discussed below in detail.

The Hex₁₋₉HexNAc₁ glycan series was digested so that Hex₃₋₉HexNAc₁ were digested and transformed into Hex₁HexNAc₁ (data not shown), indicating that they had contained terminal α-mannose residues. Because they were transformed into Hex₁HexNAc₁, their experimental structures were (Manα)₁₋₈Hex₁HexNAc₁.

The Hex₁₋₁₀HexNAc₂ glycan series was digested so that Hex₄₋₁₀HexNAc₂ were digested and transformed into Hex₁₋₄HexNAc₂ and especially into Hex₁HexNAc₂ that had not existed before the reaction and was the major reaction product. This indicates that 1) glycans Hex₄₋₁₀HexNAc₂ include glycans containing terminal α-mannose residues, 2) glycans Hex₁₋₄HexNAc₂ could be formed from larger α-mannosylated glycans, and 3) majority of the glycans Hex₄₋₁₀HexNAc₂ were transformed into newly formed Hex₁HexNAc₂ and therefore had the experimental structures (Manα)ₙHex₁HexNAc₂, wherein n≧1. The fact that the α-mannosidase reaction was only partially completed for many of the signals suggests that also other glycan components are included in the Hex₁₋₁₀HexNAc₂ glycan series. In particular, the Hex₁₀HexNAc₂ component contains one hexose residue more than the largest typical mammalian high-mannose type N-glycan, suggesting that it contains glucosylated structures including (Glcα→)Hex₈HexNAc₂, preferentially α3-linked Glc and even more preferentially present in the glucosylated N-glycan (Glcα3→)Man₉GlcNAc₂.

The Hex₁₋₆HexNAc₁dHex₁ glycan series was digested so that Hex₃₋₉HexNAc₁dHex₁ were digested and transformed into Hex₁HexNAc₁dHex₁, indicating that they had contained terminal α-mannose residues and their experimental structures were (Manα)₂₋₅Hex₁HexNAc₁dHex₁. Hex₁HexNAc₁dHex₁ appeared as a new signal indicating that glycans with structures (Manα)ₙHex₁HexNAc₁dHex₁, wherein n≧1, had existed in the sample. The Hex₂₋₇HexNAc₃ glycan series was digested so that Hex₆₋₇HexNAc₃ were digested and transformed into other glycans in the series, indicating that they had contained terminal α-mannose residues. Hex₂HexNAc₃ appeared as a new signal indicating that glycans with structures (Manα)ₙHex₂HexNAc₃, wherein n≧1, had existed in the sample.

The Hex₂₋₇HexNAc₃dHex₁ glycan series was digested so that Hex₆₋₇HexNAc₃dHex₁ were digested and transformed into other glycans in the series, indicating that they had contained terminal α-mannose residues. Hex₂HexNAc₃dHex₁ appeared as a new signal indicating that glycans with structures (Manα)ₙHex₂HexNAc₃dHex₁, wherein n≧1, had existed in the sample. Hex₃HexNAc₃dHex₂ and Hex₃HexNAc₄ appeared as new signals indicating that glycans with structures (Manα)ₙHex₃HexNAc₃dHex₂ and (Manα)ₙHex₃HexNAc₄, respectively, wherein n≧1, had existed in the sample.

β-glucosaminidase sensitive structures. The Hex₃HexNAc₂₋₅dHex₁ glycan series was digested so that Hex₃₋₉HexNAc₁dHex₁ were digested and transformed into Hex₁HexNAc₁dHex₁, indicating that they had contained terminal α-mannose residues and their experimental structures were (Manα)₂₋₅Hex₁HexNAc₁dHex₁. Hex₁HexNAc₁dHex₁ appeared as a new signal indicating that glycans with structures (Manα)ₙHex₁HexNAc₁dHex₁, wherein n≧1, had existed in the sample. However, Hex₃HexNAc₆dHex₁ was not digested indicating that it contained other terminal HexNAc residues than β-linked GlcNAc residues.

Hex₂HexNAc₃ and Hex₂HexNAc₃dHex₁ were digested into Hex₂HexNAc₂ and Hex₂HexNAc₂dHex₁ indicating they had the structures (GlcNAcβ→)Hex₂HexNAc₂ and (GlcNAcβ→)Hex₂HexNAc₂dHex₁, respectively.

Hex₄HexNAc₄dHex₁, Hex₄HexNAc₄dHex₂, Hex₄HexNAc₅dHex₂, and Hex₅HexNAc₅dHex₁ were also digested indicating they contained structures including (GlcNAcβ→)Hex₄HexNAc₃dHex₁, (GlcNAcβ→)Hex₄HexNAc₃dHex₂, (GlcNAcβ→)Hex₄HexNAc₄dHex₂, and (GlcNAcβ→)Hex₅HexNAc₄dHex₁, respectively.

β1,4-galactosidase sensitive structures. Glycan signals that were sensitive to β1,4-galactosidase comprised a major proportion of BM MSC glycans, indicating that β1,4-linked galactose is a common terminal epitope in BM MSC neutral N-glycans.

Hex₅HexNAc₄ and Hex₅HexNAc₄dHex₁ were digested into Hex₃HexNAc₄ and Hex₃HexNAc₄dHex₁ indicating they had the structures (Galβ4GlcNAcβ→)₂Hex₃HexNAc₂ and (Galβ4GlcNAcβ→)₂Hex₃HexNAc₂dHex₁, respectively. In contrast, Hex₅HexNAc₄dHex₂ was digested into Hex₄HexNAc₄dHex₂ indicating that it had the structure (Galβ4GlcNAcβ→)Hex₄HexNAc₃dHex₂, respectively, and Hex₅HexNAc₄dHex₃ was not digested at all. Taken together, in BM MSC, n−1 hexose residues are protected by deoxyhexose residues from the action of β1,4-galactosidase in the N-glycan structures Hex₅HexNAc₄dHexₙ, wherein 0≦n≦3. Such dHex-protected structures containing β1,4-linked galactose include Galβ4(Fucα3)GlcNAc and Fucα2Galβ4GlcNAc.

Similarly, Hex₆HexNAc₅, Hex₅HexNAc₅dHex₁, Hex₆HexNAc₅, and Hex₅HexNAc₅dHex₁ were digested into Hex₃HexNAc₅, Hex₃HexNAc₅dHex₁, and Hex₃HexNAc₆dHex₁ indicating they had the structures (Galβ4GlcNAcβ→)₃Hex₃HexNAc₂, (Galβ4GlcNAcβ→)₂Hex₃HexNAc₃dHex₁, and (Galβ4GlcNAcβ→)₃Hex₃HexNAc₃dHex₁, respectively. In contrast, Hex₄HexNAc₅dHex₂, Hex₅HexNAc₅dHex₃, Hex₆HexNAc₅dHex₂, and Hex₆HexNAc₅dHex₃ were not digested, indicating that hexose residues in these structures were protected by deoxyhexose residues. Such dHex-protected structures containing β1,4-linked galactose include Galβ4(Fucα3)GlcNAc and Fucα2Galβ4GlcNAc. However, Hex₄HexNAc₅dHex₃ was digested indicating that it contained one or more terminal β1,4-linked galactose residues.

Hex₇HexNAc₃, Hex₆HexNAc₃dHex₁, Hex₆HexNAc₃, and Hex₅HexNAc₃dHex₁ were digested into products including Hex₅HexNAc₃ and Hex₄HexNAc₃dHex₁, indicating they had the structures (Galβ4GlcNAcβ→)Hex₅₋₆HexNAc₂ and (Galβ4GlcNAcβ→)Hex₄₋₅HexNAc₃dHex₁, respectively. The relative amounts of Hex₃HexNAc₃, and Hex₃HexNAc₃dHex₁ were increased indicating that they were products of (Galβ4GlcNAcβ→)Hex₃HexNAc₂ and (Galβ4GlcNAcβ→)Hex₃HexNAc₂dHex₁, respectively. β1,3-galactosidase sensitive structures. Because only few structures in BM MSC neutral N-glycan fraction are sensitive to the action of β1,3-galactosidase, the majority of terminal galactose residues appear to be β1,4-linked. The glycan signals corresponding to β1,3-galactosidase sensitive glycans include Hex₅HexNAc₅dHex₁ and Hex₄HexNAc₅dHex₃. Glycosidase resistant structures. In the present experiments, Hex₂HexNAc₃dHex₂, Hex₄HexNAc₃dHex₂, and Hex₁₁HexNAc₂ were resistant to the tested exoglycosidases. The first two proposed monosaccharide compositions contain more than one deoxyhexose residues suggesting that they are protected from glycosidase digestions by the second dHex residues such as α2-, α3-, or α4-linked fucose residues, preferentially present in Fucα2Gal, Fucα3GlcNAc, and/or Fucα4GlcNAc epitopes. The last proposed monosaccharide composition contains two hexose residues more than the largest typical mammalian high-mannose type N-glycan, suggesting that it contains glucosylated structures including (Glcα→)₂Hex₉HexNAc₂, preferentially α2- and/or α3-linked Glc and even more preferentially present in the diglucosylated N-glycan (GlcαGlcα→)Man₉GlcNAc₂. The compiled neutral N-glycan fraction glycan structures based on the exoglycosidase digestions of BM MSC are presented in Table 15.

### Osteoblast-Differentiated BM MSC

The analysis of osteoblast differentiated BM MSC are presented in Table 16, allowing comparison of differentiation specific changes in CB MSC. The exoglycosidase profiles produced for BM MSC and osteoblast differentiated BM MSC are characteristic for the two cell types. For example, signals at m/z 1339, 1784, and 2466 are digested differentially in the two experiments. Specifically, the presence of β1,3-galactosidase sensitive neutral N-glycan signals in osteoblast differentiated BM MSC indicate that the differentiated cells contain more β1,3-linked galactose residues than the undifferentiated cells.

The sialidase analysis performed for the acidic N-glycan fraction of BM MSC supported the proposed monosaccharide compositions based on sialylated (NeuAc or NeuGc containing) N-glycans in the acidic N-glycan fraction.

### Analysis of CB MSC Neutral Glycan Fraction by Exoglycosidases

The results of the analysis by β1,4-galactosidase and β-glucosaminidase are presented in Table 17. The results suggest that also in CB MSC neutral N-glycans containing non-reducing terminal β1,4-linked galactose residues are abundant, and they suggest the presence of characteristic non-reducing terminal epitopes for most of the observed glycan signals. The analysis of adipocyte differentiated CB MSC are presented in Table 18, allowing comparison of differentiation specific changes in CB MSC, similarly as described above for BM MSC. The sialidase analysis performed for the acidic N-glycan fraction of CB MSC supported the proposed monosaccharide compositions based on sialylated (NeuAc or NeuGc containing) N-glycans in the acidic N-glycan fraction.

### Example 19

### Isolation of Subset Expressing Glycan Structures of Formula (I) on Human Embryonic Stem Cells

### Cell Culture and Passaging

FES hESC lines with normal karyotypes are obtained and grown as described in Mikkola et al. (2006; Distinct differentiation characteristics of individual human embryonic stem cell lines, BMC Dev Biol. 2006; 6: 40).

Human ESCs are maintained on mitotically inactivated primary mouse embryonic fibroblasts (MEF) feeder layers for routine maintenance. Cells are grown in tissue culture treated dishes (Corning Incorporated). Cells are passaged every 6 days using either a pretreatment with 10 mg/ml collagenase 5 minutes or manual dissection with a fire pulled Pasteur pipette.

Immunocytochemistry is performed on routinely maintained adherent hESC colonies, and flow cytometry is performed using routinely maintained hESC colonies that are stained for antibodies, lectins or glycosidases of the present invention.

### Enrichment of Glycan Structure of Formula (I) Expressing Stem Cells

The FACS analysis is performed essentially as described in Venable et al. (2005) but living cells are used instead and FACSAria™ cell sorter (BD).

Human ESCs are harvested into single cell suspensions using collagenase and cell dissociation solution (Sigma). Then, cells are placed in sterile tube in aliquots 10⁶ cells each and stained with one of the GF antibody in 1:100 solution. Cells are washed 3 times with PBS and then stained with secondary antibodies (antigoat mouse IgG or IgM FITC conjugated). Unstained FES used as control. The FITC positive cells are collected into cell culture media (in +4° C.) (according to BD instructions).

Then, cells are placed on MEF or HHF feeder layers and monitored for clonal or cell lineage. To check the undifferentiation stage, the gene expression of sorted cells are analyzed with real-time PCR.

Alternatively, FACS enriched cells are let to spontaneously differentiate on gelatin. Immunohistochemistry is performed with various tissue specific antibodies as described in Mikkola et al. (2006) or analysed with PCR.

### Example 20

### Revealing Protease Sensitive and Insensitive Antibody Target Structures

Bone marrow mesenchymal stem cells as described in examples above were analyzed by FACS analysis. Several antigen structures are essentially not observed or these are observed in reduced amount in FACS analysis of cell surface antigens when cells are treated (released from cultivation) by trypsin but observable after Versene treatment (0.02% EDTA in PBS). This was observed for example by labelling of the mesenchymal stem cells by the antibody GF354, and GF275, with major part trypsin sensitive target structures and by the antibody GF302, which target structure is practically totally trypsin sensitive.

### Example 21

### Isolation and Characterization of Protease Released Glycopeptides Comprising Specific Binder Target Structures

Glycopeptides are released by treatment of stem cells by protease such as trypsin. The glycopeptides are isolated chromatographically, a preferred method uses gel filtration chromatography in Superdex (Amersham Pharmacia(GE)) column (Superdex peptide or superdex 75), the peptides can be observed in chromatogram by tagging the peptides with specific labels or by UV absorbance of the peptide (or glycans). Preferred samples for the method includes mesenchymal stem cells in relatively large amounts (millions of cells) and preferred antibodies, which are used in this example includes antibodies GF354, GF275 or GF 302 or antibodies or other binders such as lectins with similar specificity.

The isolated glycopeptides are then run through a column of immobilized antibody (e.g. antibody immobilized to cyanogens provide activated column of Amersham Pharmacia(GE healthcare division or antibody immobilized as described by Pierce catalog)). The bound and/or weakly bound and chromatographically retarded fraction(s) is(are) collected as target peptide fraction. In case of high affinity binding the glycan is eluted with 100-1000 mM monosaccharide or monosaccharides corresponding to the target epitope of the antibody or by mixture of monosaccharides or oligosaccharides and/or with high salt concentration such as 500-1000 mM NaCl. The glycopeptides are analysed by glycoproteomic methods using mass spectrometry to obtain molecular mass and preferably also fragmentation mass spectrometry in order to sequence the peptide and/or the glycan of the glycopeptide.

In alternative method the glycopeptides are isolated by single affinity chromatography step by the binder affinity chromatography and analysed by mass spectrometry essentially similarily as described e.g. in Wang Y et al (2006) Glycobiology 16 (6) 514-23, but lectin affinity chromatography is replaced by affinity chromatography by immobilized antibodies, such as preferred antibodies or binder described above in this example.

### Example 22

### Growth of Human Embryonic Stem Cells on Lectin Coated Culture Plate

FES 30 hESC line was used. hESCs were transferred from mEF to Matrigel™ and cultured according to the protocol found on the Geron Corporation website at: http://www.geron.com/showpage.asp?code=prodstprot

All passages were done using collagenase. The passages were also done using PBS without collagenase treatment.

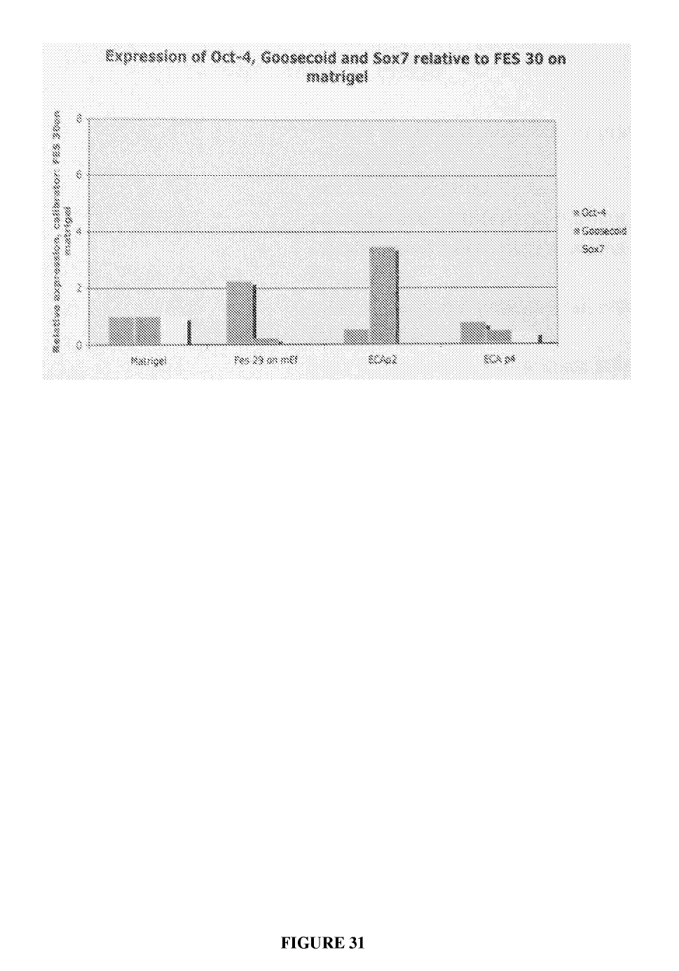

The cells were transferred to ECA-coated 12-well plates (Corning) and cultured in mEF-conditioned media for 5-11 days, whereafter they were divided 1:2 or 3:4. RNA samples were extracted from a cell sample every second or third passage and analyzed for expression of stem cell and differentiation markers (FIG. 31).

### Results

The cells grew on ECA-coated and Matrigel™-coated plates with similar efficiency and with similar morphology when observed by microscopy. The expression profiles of studied stem cell and differentiation markers were similar (see FIG. 31).

The cells grew more evenly on ECA-coated than on Matrigel™-coated plates (Figure) with no apparent batch-to-batch variation in growing density. They formed small colonies, which was different from Matrigel. The colonies were smaller than those formed by hESC grown on feeder cells.

### Example 23

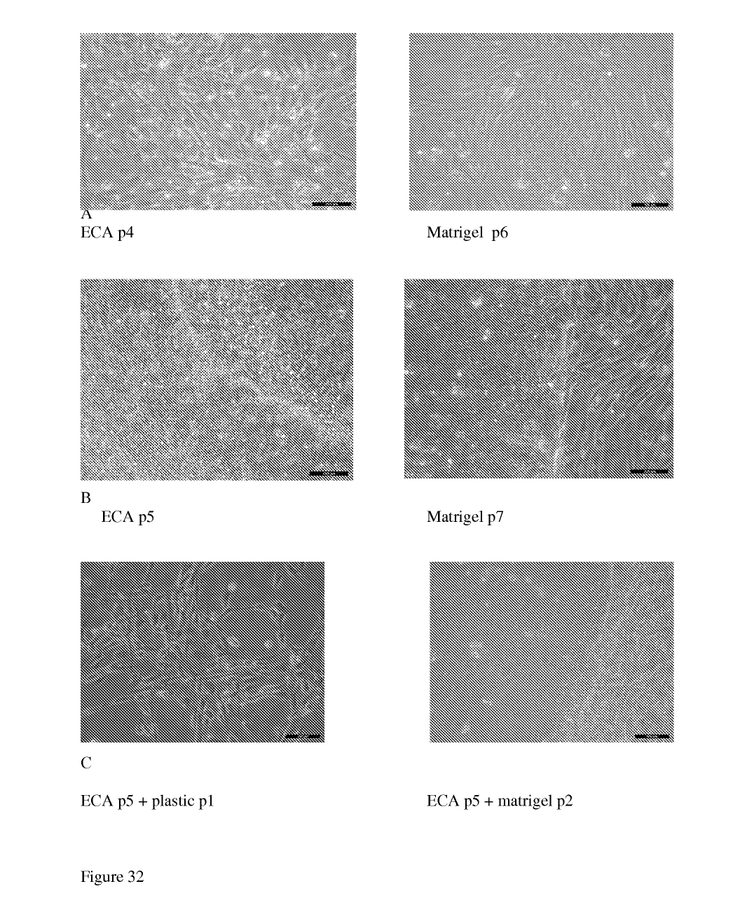

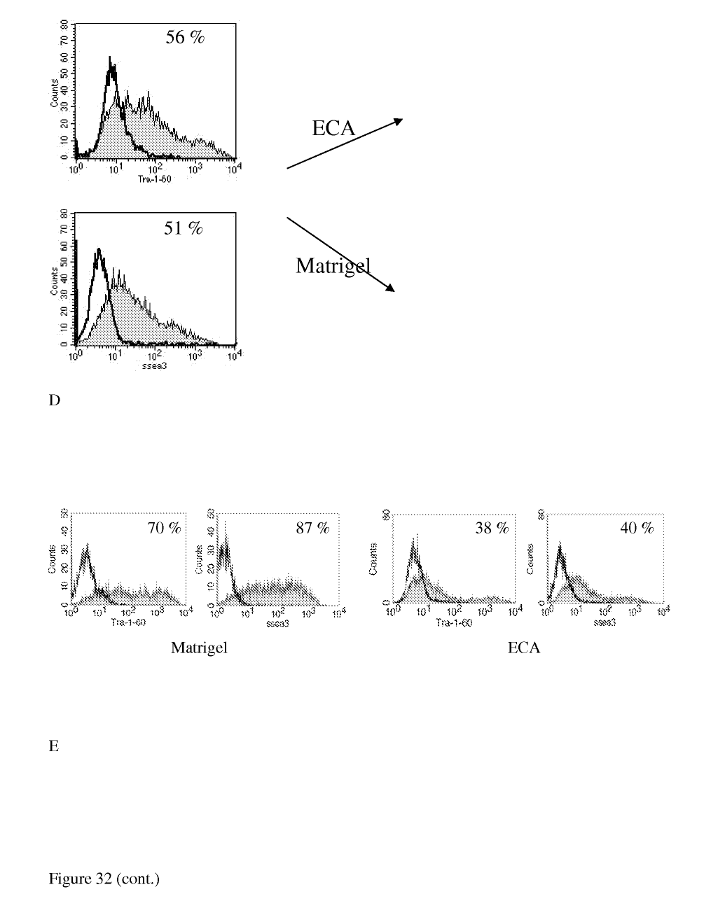

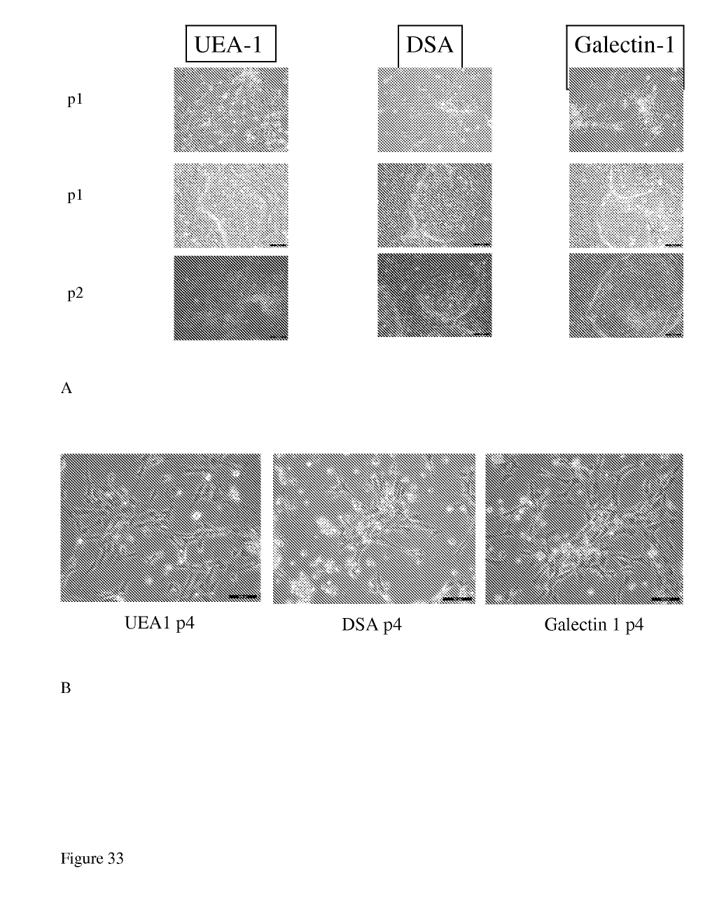

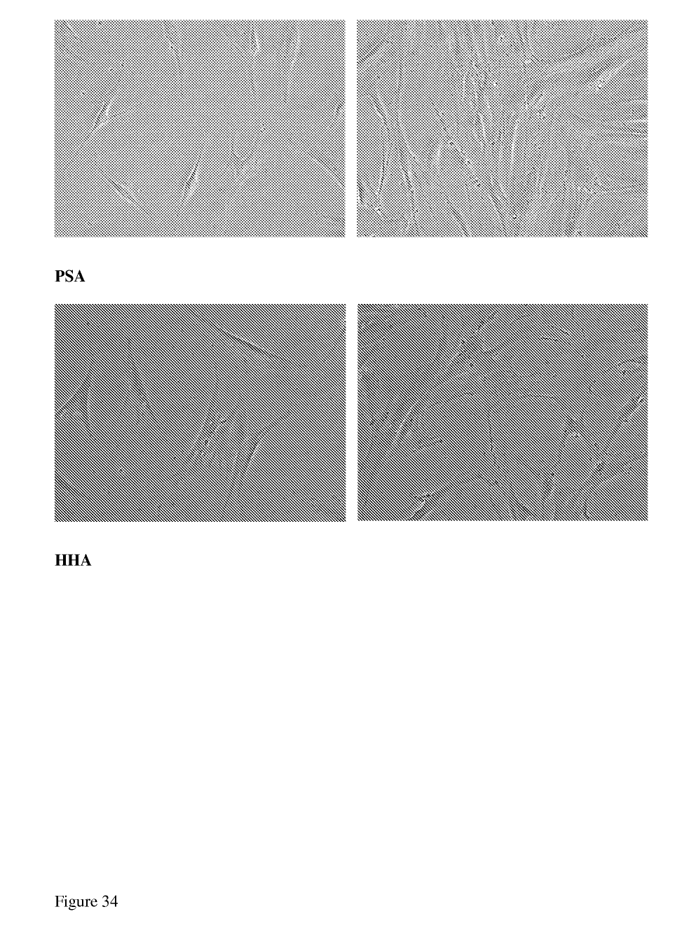

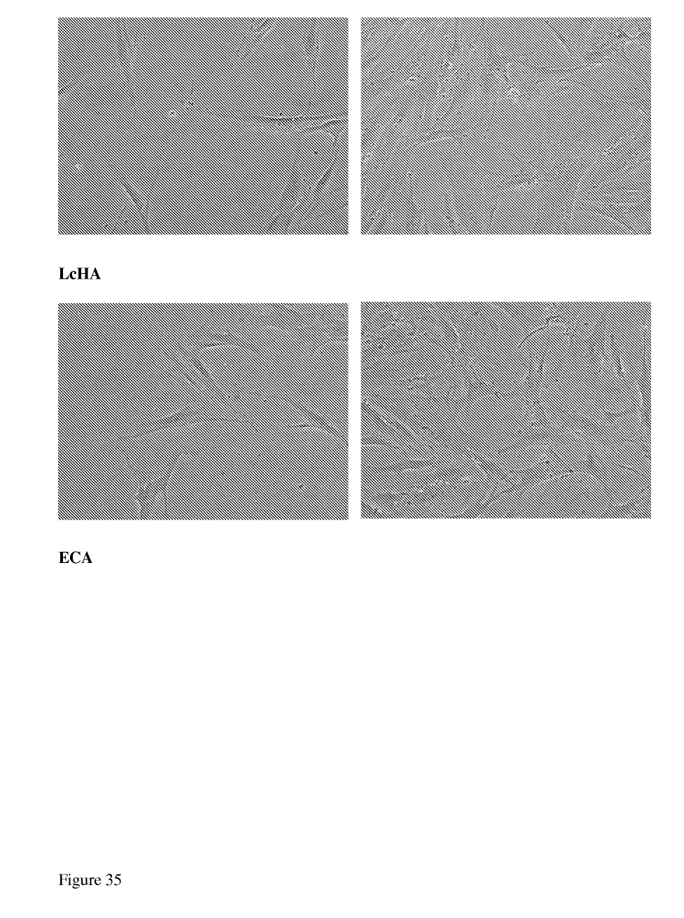

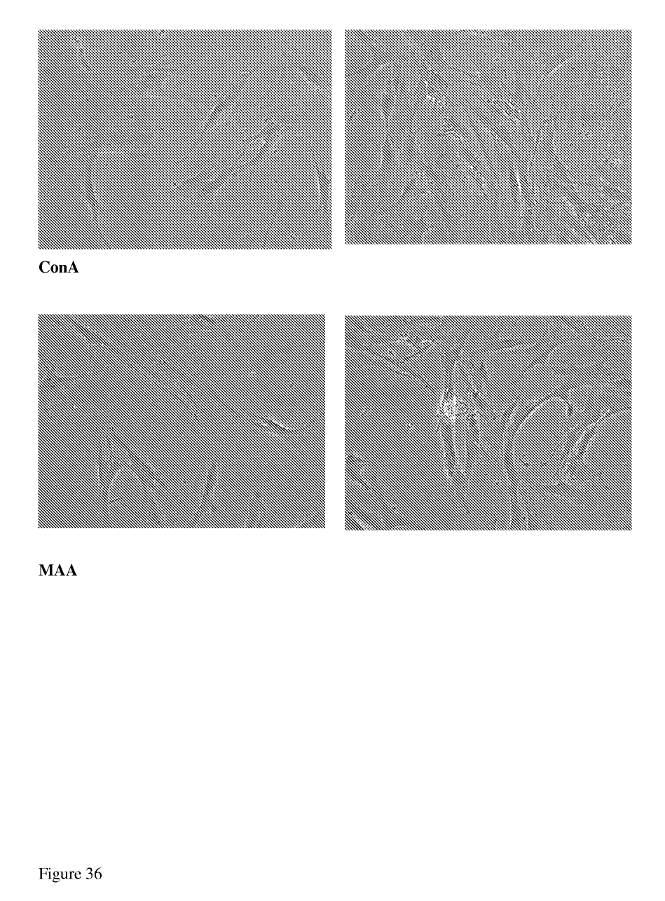

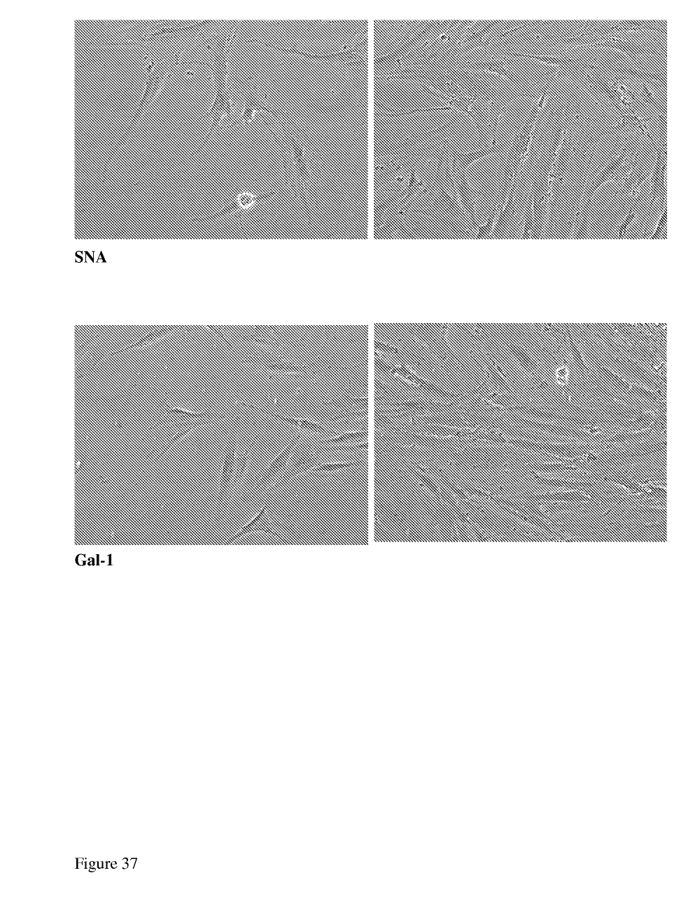

### hESC (FES 29 Cells, Passage (p)36) were Grown on Plastic or in the Presence of ECA, UEA1, DSA, or Galectin-1 for Time Period Indicated in FIGS. 32 and 33 and as Described in Example 22

The ECA (EY Laboratories, USA; L-5901-5) coating was performed as follows for 12 well plates. ECA lectin was stored frozen as stock solution of 1 mg/ml. It was thawed on ice and diluted as 100 μl lectin stock+600 μl PBS, sterile filtered (Millex-GV, SLGV 013 SL, 0.22 μm) in laminar hood. This solution, i.e. 100 μg/700 μl PBS solution was applied to each well and incubated overnight at +4 C. On the following day lectin solution was removed and wells were washed with 3×1 ml of sterile PBS.

### Materials and Methods

Human Embryonic Stem Cells (hESC)

Two furnish human embryonic stem cell (hESC) lines, FES29 and FES30, were used to study hESC culturing on lectin-coated wells (described by Mikkola M. et al. BMC Dev. Biol. 6:40, 2006).

hESC were cultured at least two passages feeder-free on Matrigel (BD Biosciences, Bedford, Mass., USA) before plating on lectin. Matrigel-culturing was continued side by side lectin-culturing as comparison. Cells were cultured during the whole experiment in standard Knockout™ DMEM media with 20% Knockout™ Serum Replacement and 8 ng/ml of recombinant basicFGF (all from Gibco/Invitrogen, Paisley, UK; Mikkola M. et al. BMC Dev. Biol. 6:40, 2006) conditioned on mouse feeder cells for 24 h. Cells were detached with collagenase IV while splitting.

### Lectin Coating

Lectins were diluted in PBS and sterile filtered before applying on Nunclon cell culture plates (Nunc, Roskilde, Denmark). The amount of lectin was 27 μg/cm² and plates were incubated over night at +4° C. Before splitting cells on lectin wells were washed three times with PBS.

### Results and Discussion

### Culturing on ECA Lectin

FES30 hESC were splitted from Matrigel on ECA, MAA, WFA and PWA lectins and only on ECA cells grew and could be splitted further. FES30 cells were cultured totally for 23 passages on ECA. ECA culturing was confirmed with another hESC-line FES29 for six passages.

hESC cultured on ECA were morphologically changed, looked differentiated and did not form typical hESC colonies. ECA culturing seemed to favour “feeder-like” cells and the expression of pluripotency markers, Tra-1-60 and SSEA-3, also decreased. FES29 cells were splitted back to Matrigel after 5 passages on ECA and after 5-6 passages on Matrigel the cells started to make typical hESC colonies again. Thus, hESC can be maintained on ECA over 20 passages and even they look different from typical hESC-culture they do not loose their ability to grow as typical undifferentiated hESC.

### Culturing on Other Lectins

FES29 hESC were also maintained for 7 passages on UEA-1, DSA and bovine Galectin-1 in mouse feeder cell conditioned media. hESC looked morphologically similar on these lectins as on ECA. After 7 passages Galectin-1 cultured cells had highest expression of Tra-1-60 and SSEA-3 among these three lectins, although the expressions were low (21.6% and 32.3%, respectively).

### Example 24

### Expression and Purification of Recombinant Erythrina cristagalli Agglutinin (ECA) and its Non-Glycosylated Form in Yeast

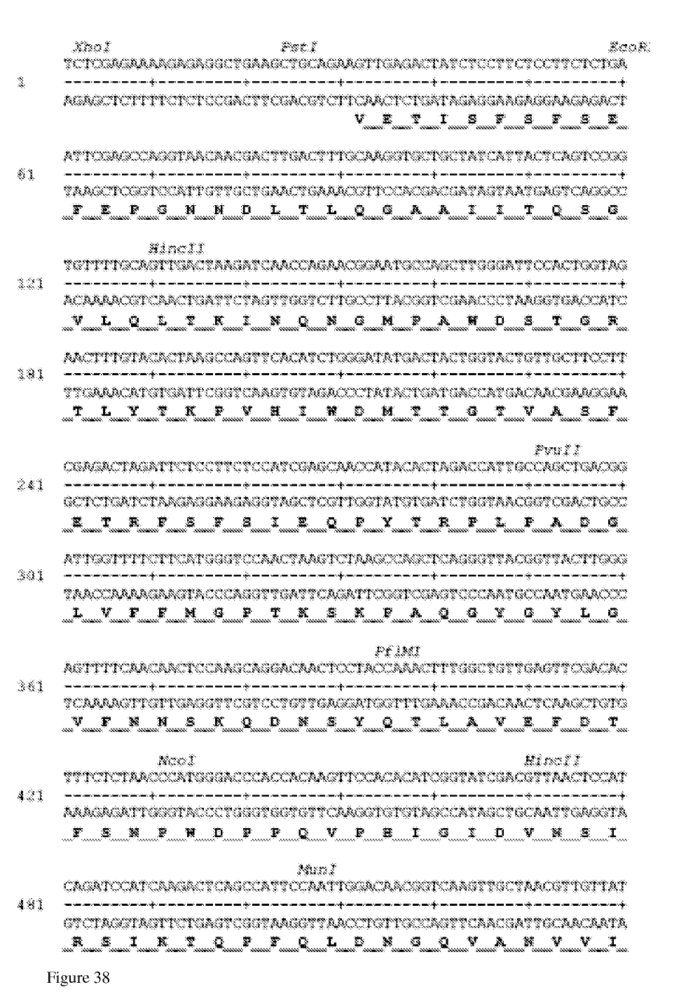

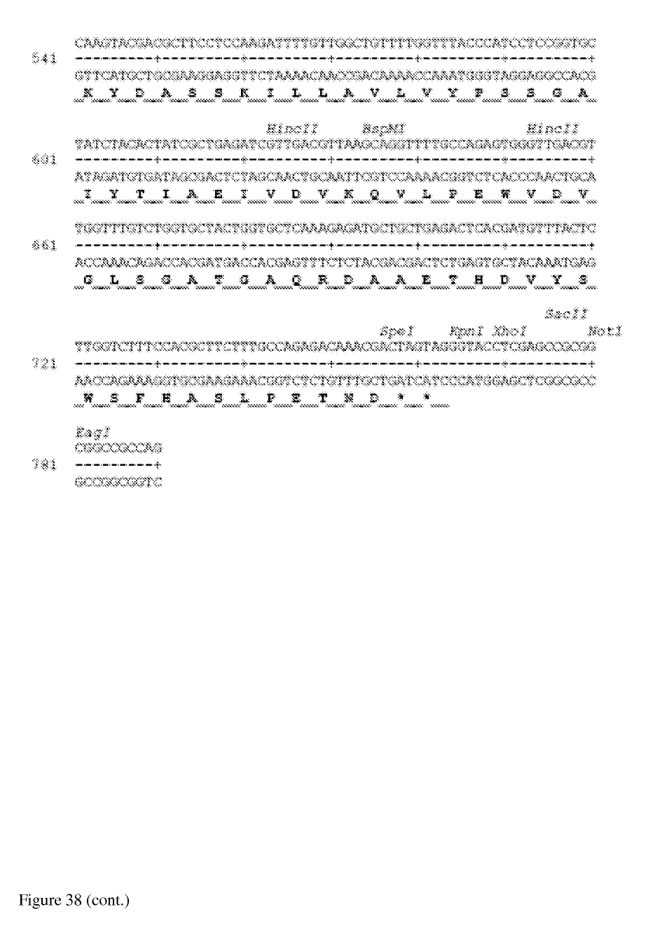

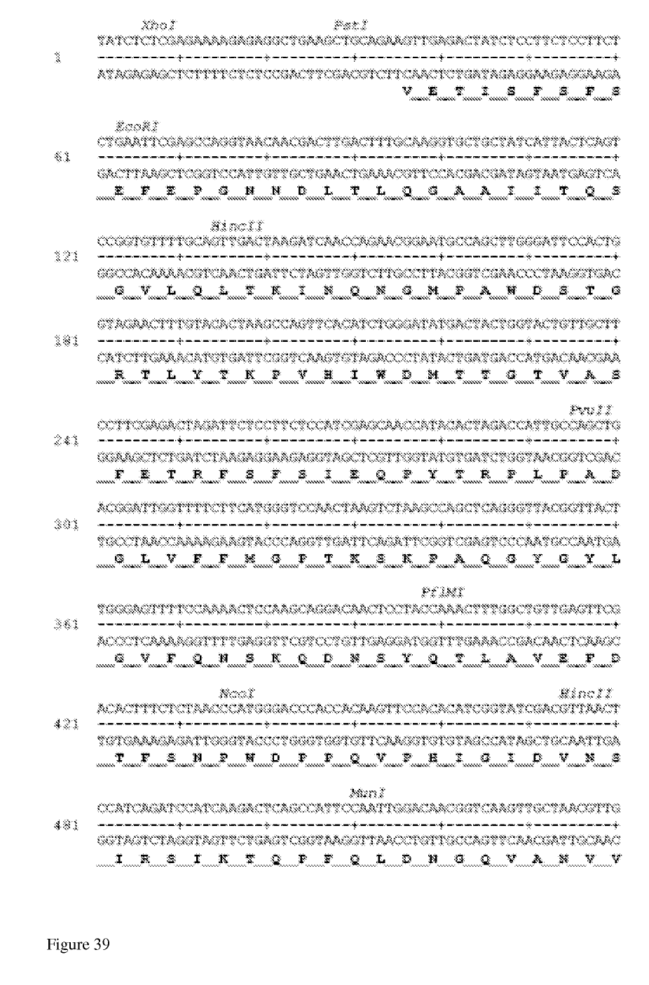

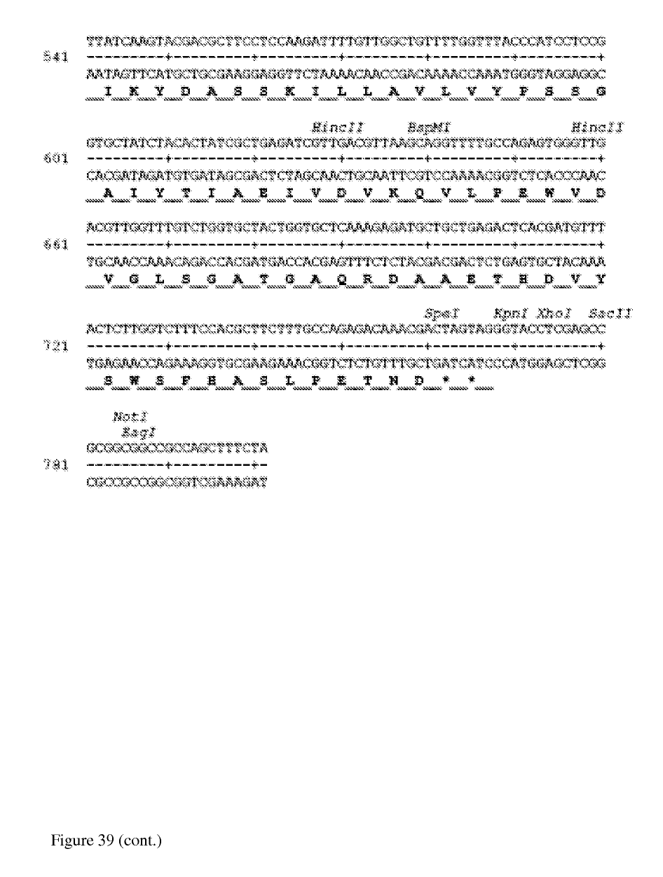

Synthetic nucleotide sequences optimized with Pichia pastoris codon preference were constructed according to gene bank accession number AY158072 (partial coding sequence for Erythrina cristacalli agglutinin gene). Genes coding for both natural amino acid sequence (FIG. 38; Gene seq No 899) and non-glycosylated form (FIG. 39; Gene seq No 900) were constructed. DNA synthesis and gene construction were acquired as commercial service from GeneArt AG.

Synthetic sequences for recombinant ECA (rECA) and non-glycosylated recombinant ECA (ngECA) were cloned into Pichia pastoris expression vector pBLURA-SX (Lin Cereghino et al. 2001, Gene 263:159-169) by single cloning step as a fragment cleaved with restriction endonucleases PstI/KpnI according to standard cloning procedures. Sequences were placed under the control of AOX1 promoter and AOX1 3′UTR regions, adjusting the sequence in the correct reading frame with MATα secretion signal which targets the synthesized protein to the growth media. Expression vectors were transferred to Pichia pastoris by homologous recombination according to standard procedures. Expression of recombinant protein was likewise performed according to commonly known standard procedure. Yeast cells were cultured on glycerol-containing media to the log-phase in the appropriate temperature not exceeding +30° C., harvested and placed to induction media at the optical density A600=1. Induction was achieved with methanol addition.

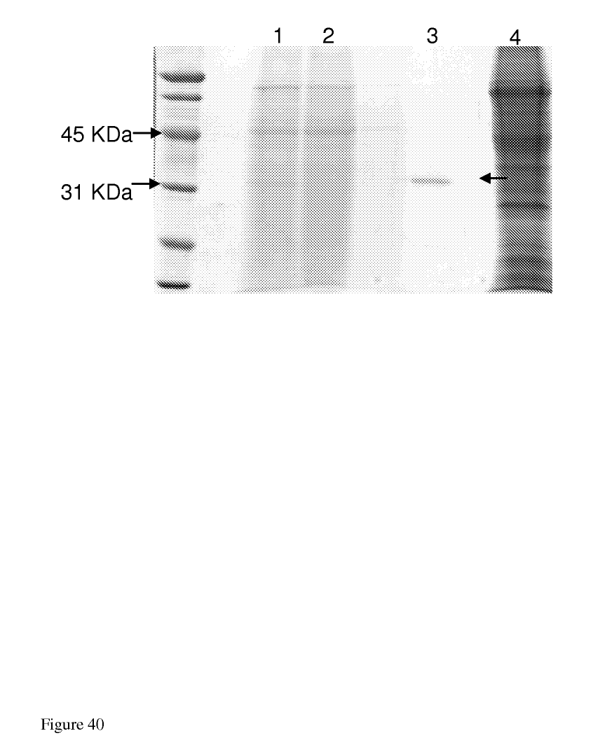

Both rECA and ngECA were purified from concentrated protein expression culture supernatant by the following steps: 1. Ammonium sulfate precipitation (30-60% precipitate, adopted from Iglesias et al. 1982, Eur. J. Biochem. 123, 247-252), 2. Dialysis into Binding buffer (150 mM NaCl, 20 mM Tris-HCl pH 8, 1 mM MnCl₂, 1 mM CaCl₂; adopted from Stancombe et al. 2003, Protein Expr Purif. 30, 283-292), 3. Lactose-affinity chromatography (see below), 4. Dialysis into water, and 5. Lyophilization. Lactose-affinity chromatography was adopted from Stancombe et al. (2003) with modifications: Lac-agarose was used as affinity matrix (Sigma-Aldrich), washing was done with Binding buffer, and bound ECA was eluted with 0.3 M lactose in Binding buffer. The fractions containing ECA as detected by SDS-PAGE (FIG. 40) were pooled, dialysed and lyophilized. The purified protein was determined active by affinity to lactose-agarose, and essentially pure by SDS-PAGE (FIG. 40, Lane 3).

### Example 25

### Oxidation and Biotinylation of the Glycans of ECA

ECA, Erythrina cristagalli lectin was dissolved in PBS. The concentration of the ECA sample was determined by subjecting 0.7% of the sample to size-exclusion chromatography on a Superdex 200 10/300 GL column. The concentration of ECA sample was defined as 0.31 μg/μl by comparing the UV absorbance of the 0.7% ECA sample to the BSA standard.

The glycans of the ECA sample were oxidized by adding sodium metaperiodate at the final reaction concentration of 8 mM. The reaction mixture was incubated at +4° C. in dark over night. The reaction was stopped by destroying the unreacted periodate with ethyleneglycol at the final reaction concentration of 8 mM for 2 h. The reaction mixture was purified on PD-10 desalting column. The modified ECA was eluted with 3.5 ml of PBS.

The oxidatized glycans of the ECA were biotinylated by adding biotin-amidohexanoic acid hydrazide (Sigma, Mw=371.5 g/mol) at the final reaction concentration of 0.28 mM. The reaction mixture was incubated at room temperature over night. The sample solution was subjected to PD-10 desalting column. The modified ECA was eluted with 3.5 ml of PBS. 2.5% of the sample was subjected to size-exclusion chromatography on a Superdex 200 10/300 GL column to define the existence and the amount of the modified ECA. The modified ECA eluted in a same fraction as the native ECA dimer. The yield of the oxidation-biotinylation reactions was over 70%. MALDI TOF mass spectrum of the modified ECA showed molecular ions [M+Na]⁺ centered at m/z 29236 while spectrum of the native ECA showed molecular ions [M+Na]⁺ centered at m/z 27545, indicating addition of 4-5 biotin/ECA molecule. No degradation products were detected in the analyses.

Cell Culture with Different ECA Forms

Human embryonic stem cells (hESC) were propagated and transferred and conditioned to Matrigel (BD Biosciences) and Knockout serum replacement cell culture medium (Invitrogen) as described in the preceding Examples, whereafter they were transferred to cell culture plates adsorption-coated with different forms of ECA (replacing Matrigel surface): native ECA (EY Laboratories; Sigma-Aldrich), protein-biotinylated ECA (EY Laboratories), or glycan-biotinylated ECA (see above) in parallel experiments. By following cell proliferation level, stem cell marker expression and stem cell specific morphology features for several passages, it was concluded that glycan-biotinylated ECA was better (+++) at supporting hESC culture than either native ECA (++) or protein-biotinylated ECA (++); with growth supporting capacity evaluated by −, +, ++, or +++(from no growth =−, to excellent growth=+++) in parenthesis.

### Example 26

### MSC

Cell samples

### Mesenchymal Stem Cell Samples

Human Bone marrow—derived mesenchymal stem cells (MSC) were generated as described by Leskelä et al. (Leskelä H, Risteli J, Niskanen S, et al. Osteoblast recruitment from stem cells does not decrease at late adulthood; Biochemical and Biophysical Research Communications 311:1008-1013, 2003). Briefly, bone marrow obtained during orthopedic surgery was cultured in Minimum Essential Alpha-Medium (α-MEM), supplemented with 20 mM HEPES, 10% FCS, 1× penicillin-streptomycin and 2 mM L-glutamine (all from Gibco). After a cell attachment period of 2 days the cells were washed with Ca₂+ and Mg₂+ free PBS (Gibco), and subcultured at a density of 2000-3000 cells/cm2 in the same media on 24-well chamber slides coated with lectin molecules. Cells were crown at 37° C. with 5% CO₂, fresh media was changed twice a week until near confluence. MSCs were cultured on lectin coated well plates for five passages. MSC passages 5-10 were used in the experiment.

### Molecules Used in MSC Culture Assay

GF 606 Lectin Pisum sativum PSA
GF 607 HHA, Hippocastrum hybrid
GF608 LcHA, Lens culinaris aggllutinin
GF 609 ECA, erythrina cristagalli
GF 610 ConA
GF 611 MAA, Maackia amuriensis
GF 612 SNA, Sambucus nigra
GF 613, Galectin-1
streptavidin
stretavidin + biotin

### Flow Cytometry

Proliferating MSCs in passage 5 were grown on different lectins for 5 days. Cells were washed with PBS and harvested into single cell suspensions by Versene solution. Detached cells were centrifuged at 600×g for five minutes at room temperature. Cell pellet was washed twice with 0.3% BSA-PBS, centrifuged at 600×g and resuspended in 0.3% BSA-PBS. Cells were placed in conical tubes in aliquots of 50 000 cells each. Cell aliquots were incubated with antibodies in dilution of 2 μl/10⁵ cells for 30 minutes at +4° C. in the dark. After incubation cells were washed with 0.3% BSA-PBS, centrifuged and resuspended in 0.3% BSA-PBS.

Unlabeled cells and cells grown on plastic were used as controls. Antibody binding was detected by flow cytometry (FACSAria, Becton Dickinson). Data analysis was made with FACSDiva™ Flow Cytometry Software Version 5.02.

TABLE
Antibodies and their fluorogenic labels used to characterize MSC

| | |
|---|---|
| CD 105 | FITC |
| CD 45 | APC |
| CD 73 | PE |
| HLA-DR | FITC |

### RNA Purification and Quantitative Reverse Transcription (RT)-Polymerase Chain Reaction (PCR)

Total cellular RNA from the BM derived mesenchymal stem cells grown for five passages on selected lectins was extracted by using RNeasy miniprep-kit (Qiagen, Chatsworth, Calif.) according to the manufacturers's instructions. RNA was then reverse transcribed to cDNA with High Capacity cDNA Reverse Transcription reagents according to manufacturers instructions (Applied Biosystems) and used as a template in TaqMan® PCR reaction. TaqMan® PCR reaction was used to estimate the quantitative levels of stem cell differentiation markers. TaqMan® PCR reaction performed in standard conditions using TaqMan® Universal Gene Expression Master Mix (Applied Biosystems) and Pre-developed Inventored Gene Expression Assays for FABP4 (Hs00609791_m1) and RUNX2 (Hs00231692_m1) (Applied Biosystems).

Real time quantitative PCR reactions were performed with the ABI PRISM 7000 Sequence Detector System (Applied Biosystem) in standard conditions. PCR amplifications were performed in a total volume of 50 μl, containing 1 μl cDNA sample. TaqMan® Universal Gene Expression Master Mix (Applied Biosystems) was used in all experiments. Pre-developed Inventored Gene Expression Assays for FABP4 (Hs00609791_m1) and RUNX2 (Hs00231692_m1) (Applied Biosystems) were used to estimate the quantitative levels of stem cell differentiation markers. Pre-developed TaqMan® assay reagents for endogenous control human TATA-box binding gene labeled with VIC reporter dye (Hs 999999_m19) was used for amplification of control gene.

PCR was started with 2 min at 50° C. and the initial 10 min denaturing temperature was 94° C., followed by a total of 40 cycles of 15 s of denaturing and 1 min of annealing and elongation at 60° C.

### Results

### Relative Gene Expression of MSC Cells Grown on Different Lectins

Our results show that MSCs grown on lectins express osteogenic differentiation marker RUNX2 less than same cells grown on plastic. However, these cells express a slightly more adipogenetic marker fattyacid binding protein4 (FABP4) compared to cells grown on plastic, as shown in below.

Detection of expression

| | | | ΔCt − | |
|---|---|---|---|---|
| RUNX- Endogenous control gene | | | Δctplastic | |
| | avarage | StdDev | relative | Expression relative |
| Culture | Δct | Ct | ΔΔCt | to plastic 2e − ΔΔCt |
| GF 611 and RUNX | 3.02 | 0.12 | 2.13 | 0.24 |
| GF 609 and RUNX | 4.79 | 0.115 | 3.9 | 0.04 |
| GF 607 and RUNX | 2.77 | 0.062 | 1.88 | 0.31 |
| GF 613 and RUNX | 2.76 | 0.141 | 1.87 | 0.31 |
| GF 610 and RUNX | 2.77 | 0.217 | 1.88 | 0.31 |
| plastic RUNX | 0.89 | 0.158 | 0 | 2.00 |
| GF 611 and FABP | 4.79 | 0.33 | −2.31 | 20.15 |
| GF 609 and FABP | 5.6 | 0.031 | −1.5 | 8.96 |
| GF 607 and FABP | 5.89 | 0.062 | −1.21 | 6.71 |
| GF 613 and FABP | 5.24 | 0.069 | −1.86 | 12.85 |
| GF 610 and FABP | 6.22 | 0.119 | −0.88 | 4.82 |
| plasticFABP | 7.1 | | 0 | 2.00 |

The data indicates that the lectins mostly preserve the non-differentiated status of the cells. Lectins with specificity of MAA for sialylated structures, especially NeuNAcα3Galβ4GlcNAc, and galectin-1/ECA with N-acetyllactosamine Galβ4GlcNAc binding are espe are preferred for induction of some differentiation to adipocytic direction, while the N-glycan core specific lectin Con A is most preferred for maintaining the non-differentiated status. The invention reveals that terminal mannose specific HHA lectin has also potency to support the non differentiated status of the cells. It is realized that the results are in contrast to results of other indicating that Con A would be especially useful for cultivating animal mesenchymal stem cells when the cells would need to be differentiated.

In another experiment HLA-DR marker for differentiation of mesenchymal stem cells is determined, there Con A also showed lowest values together with MAA. The study includes two reducing end terminal fucose epitope recognizing lectins PSA and LcHA/LCA, which also show clear difference to ConA, or somewhat increased HLA-DR values. The data indicates that the midglycan recognizing conA is different in activation of human mesenchymal stem cells. In a preferred embodiment the invention is directed to cultivation of human mesenchymal stem cells, with con A N-glycan recognising type lectins immobilized on surface for maintenance of non-differentiated status of cells, and terminal epitope recognizing lectins in condition or for conditions inducing differentiation.

### Example 27

### Cultivation of hESC Cells of Lectins

FIG. 32 shows A passages p4 and p6 of hESC cells grown either on ECA lectin or matrigel, respectively. After 4 passages FACS analysis revealed embryonic stem cell markers for ECA cultivated cells Tra-1-60 32% and SSEA3 83%, while on Matrigel values were 49% and 79%. C, passages p5. D, FACS analysis of markers and hESC (FES29 p36) for culturing on ECA. E, FACS analysis of Matrigel p4 vs. Matrigel p2+ECA.

FIG. 33 shows FES29 p38 cells grown on Matrigel p3, and lectin p1. FACS: Tra-1-60 70% and SSEA3 89%. B, passage 4 images of cells grown on lectins. UEA, DSA and galectin.

Culture of hESC Cells on Various Lectins and their Derivatives

The example reveals that the N-glycosylation site mutated recombinant ECA function effectively under the cell culture conditions. Other N-acetyllactosamine recognizing lectins DSA ja galectin-1 were also effective, similarily as Fucα2Galb4GlcNAc recognizing UEA lectin, UEA-1 was initially not so effective as the LacNAc specific lectins. The initial cell attachment and growth was weak for lectin PHA-E not recognizing terminal, but N-glycan core epitope. The immobilization of the lectins is essential for the effects as soluble galectin could not support the cell growth, and soluble ECA was also worse than plastic control. Lectins with other specificities (MAA, WFA and PWA) were not effective.

culture experiment in mEF-conditioned media

| | Growth | Culture | Factor |
|---|---|---|---|
| ECA | +++ | p23, p6, p2 | MM + MT |
| rec. ECA | +++ | p2 | MT |
| UEA-1 | +++ | p7 | MT |
| DSA | +++ | p7 | MT |
| Galectin-1 | +++ | p7 | MT |
| MAA | − | — | MM |
| WFA | − | — | MM |
| PWA | − | — | MM |
| PHA-E | +++# | p2 | MT |
| ECA in solution | + | p2 | MT |
| Galectin-1 in solution | − | — | MT |
| no coating (plastic) | ++ | p2 | MT |

+++ = cells attach and can grow
++ = cells attach and can grow but less cells/slower growth than +++
+ = only few cells attach, one split
− = no cells after p1, cannot be splitted
# less cells attach and slower early growth

### Comparision of ECA Types and Conjugates

The data reveals that the glycan biotinylated ECA is more effective than randomly protein biotinylated lectin. The assay of initial adhesion reveals that the adherence as such is not sufficient for effective cell culture.

| short (5 days) culting | MM 9-14.3.07 |
|---|---|
| ECA (EY Labs) | ++ |
| ECA (Sigma) | +++ |
| ECA-biotin | + |
| streptav + ECA-biotin | + |
| streptav + ECA glycan | +++ |
| biotinylated | |
| RCA | − |
| rGal-1 | − |

| adhesion assay | MM 6.3.07 |
|---|---|
| matrigel | 21% |
| ECA | 16% |
| MAA | 53% |
| WFA | 8% |
| PWA | 6% |
| no coating (plastic) | 0% |

+++ = many cells attached on day 5
++ = scattered cells on day 5
+ = few cells left on day 5
− = no attached cells after day 5

Stem Cell Marker Levels of UEA-1, DSA and Galectin-1 Cultivated Cells

The data indicated that on these lectins the markers are reduced in comparison to the ECA lectin, which after an initial drop would give values comparable to Matgel culture.

| | UEA-1 | | DSA | | Galectin 1 | |
|---|---|---|---|---|---|---|
| | Tra-1-60 | SSEA-3 | Tra-1-60 | SSEA-3 | Tra-1-60 | SSEA-3 |
| start | 69.6 | 89.2 | 69.6 | 89.2 | 69.6 | 89.2 |
| point | | | | | | |
| p7 | 3.3 | 7.2 | 9.9 | 17.9 | 21.6 | 32.3 |

| | |
|---|---|
| ECA | Matrigel |

| | Tra-1- | | Tra-1- | |
|---|---|---|---|---|
| | 60 | SSEA-3 | 60 | SSEA-3 |
| start | 56.1 | 51.0 | 56.1 | 51.0 |
| point | | | | |
| p2 | 38.0 | 39.7 | 70.3 | 87.0 |
| p4 | 32.2 | 83.9 | 49.3 | 78.8 |

TABLE 1
Neutral N-glycan grouping of cord blood cell populations, cord blood mononuclear
cells (CB MNC), and peripheral blood mononuclear cells (PB MNC).

| Neutral N-glycan Grouping: | | | | | | | | | |
|---|---|---|---|---|---|---|---|---|---|
| Composition | Glycan Grouping | CD 34+ | CD 34− | CD 133+ | CD 133− | LIN− | LIN+ | CB MNC | PB MNC |
| General N-glycan grouping: | | | | | | | | | |
| Hex₅₋₁₂HexNAc₂ | high-mannose | 56.3 | 52.9 | 67.0 | 55.1 | 58.9 | 61.2 | 65.4 | 62.7 |
| Hex₁₋₄HexNAc₂dHex₀₋₁ | low-mannose | 33.1 | 35.5 | 25.6 | 32.8 | 21.1 | 24.5 | 26.5 | 29.6 |
| n_{HexNAc} = 3 and n_{Hex} ≧ 2 | hybrid/monoant. | 5.5 | 6.4 | 2.4 | 5.6 | 8.6 | 5.5 | 4.3 | 3.7 |
| n_{HexNAc} ≧ 4 and n_{Hex} ≧ 2 | complex | 4.3 | 4.8 | 4.5 | 5.9 | 11.0 | 8.0 | 3.1 | 3.3 |
| Other types | — | 0.8 | 0.4 | 0.6 | 0.7 | 0.5 | 0.7 | 0.7 | 0.7 |
| Complex/hybrid/monoantennary N-glycan grouping: | | | | | | | | | |
| n_{dHex} ≧ 1 | fucosylated | 67.8 | 70.6 | 81.2 | 66.4 | 49.0 | 66.8 | 58.8 | 56.4 |
| n_{dHex} ≧ 2 | α2/3/4-linked Fuc | 18.8 | 21.3 | 0.5 | 11.5 | 0 | 5.4 | 12.2 | 4.9 |
| n_{HexNAc} > n_{Hex} ≧ 2 | terminal HexNAc | 21.3 | 18.3 | 50.8 | 32.1 | 38.7 | 34.2 | 22.7 | 26.9 |
| ⁿHexNAc = n_{Hex} ≧ 5 | bisecting GlcNAc | 0 | 0 | 0.8 | 0.8 | 0.4 | 2.0 | 0.4 | 0 |
| Complex N-glycan grouping: | | | | | | | | | |
| n_{HexNAc} ≧ 5 and n_{Hex} ≧ 6 | large N-glycans | 1.8 | 6.0 | 0 | 2.5 | 0 | 4.0 | 3.8 | 2.4 |

TABLE 2
Exoglycosidase profiling of cord blood CD34+ and CD34− cell neutral N-glycan fraction.

| | | α-Man | | β1,4-Gal | | β1,3-Gal | | β-GlcNAc | |
|---|---|---|---|---|---|---|---|---|---|
| Proposed composition | m/z | CD 34+ | CD 34− | CD 34+ | CD 34− | CD 34+ | CD 34− | CD 34+ | CD 34− |
| Hex2HexNAc | 568 | | −− | +++ | +++ | +++ | +++ | | |
| HexHexNAc2 | 609 | +++ | +++ | | +++ | | +++ | | |
| Hex3HexNAc | 730 | | −−− | −− | | − | | | |
| HexHexNAc2dHex | 755 | +++ | ++ | − | − | − | −− | | |
| Hex2HexNAc2 | 771 | ++ | −− | −− | −− | −− | −− | −− | |
| Hex4HexNAc | 892 | −−− | −−− | − | | − | | | |
| Hex2HexNAc2dHex | 917 | −− | −− | −− | | −− | −− | −− | |
| Hex3HexNAc2 | 933 | −−− | −− | − | −− | −− | −− | | |
| HexHexNAc3dHex | 958 | | +++ | | | | | | |
| Hex2HexNAc3 | 974 | | | | +++ | | +++ | | |
| Hex5HexNAc | 1054 | −−− | −− | + | | | + | − | |
| Hex3HexNAc2dHex | 1079 | −− | −− | | −− | − | −− | + | |
| Hex4HexNAc2 | 1095 | −−− | −−− | | | | | | |
| Hex2HexNAc3dHex | 1120 | | + | | + | | | | |
| Hex3HexNAc3 | 1136 | −−− | | | | | − | −−− | |
| Hex6HexNAc | 1216 | −−− | −− | | − | | − | − | |
| Hex4HexNAc2dHex | 1241 | | −−− | | − | − | − | − | |
| Hex5HexNAc2 | 1257 | −−− | −− | + | + | + | + | | |
| Hex3HexNAc3dHex | 1282 | −−− | + | | | − | − | −− | |
| Hex4HexNAc3 | 1298 | −−− | | −−− | | | − | | |
| Hex2HexNAc4dHex | 1323 | | | +++ | | | | | |
| Hex3HexNAc4 | 1339 | | +++ | | +++ | | | | |
| Hex7HexNAc | 1378 | | −−− | + | | + | | | |
| Hex5HexNAc2dHex | 1403 | | −−− | | | | | +++ | |
| Hex6HexNAc2 | 1419 | −−− | −− | ++ | ++ | ++ | ++ | ++ | |
| Hex3HexNAc3dHex2 | 1428 | −−− | ++ | | +++ | | +++ | | |
| Hex4HexNAc3dHex | 1444 | −−− | | − | −− | | −− | + | |
| Hex5HexNAc3 | 1460 | −−− | − | +++ | | +++ | | −−− | |
| Hex3HexNAc4dHex | 1485 | − | | | + | | | −−− | |
| Hex4HexNAc4 | 1501 | −−− | | | −−− | | −−− | −−− | |
| Hex8HexNAc | 1540 | −−− | −−− | −−− | +++ | −−− | +++ | −−− | |
| Hex3HexNAc5 | 1542 | | +++ | | +++ | | +++ | | |
| Hex6HexNAc2dHex | 1565 | | | | | | +++ | | |
| Hex7HexNAc2 | 1581 | −−− | −− | | ++ | ++ | ++ | ++ | |
| Hex4HexNAc3dHex2 | 1590 | −−− | | −−− | − | | − | + | |
| Hex5HexNAc3dHex | 1606 | | −−− | −−− | +++ | | +++ | +++ | |
| Hex6HexNAc3 | 1622 | −−− | −−− | −−− | −−− | | | −−− | |
| Hex4HexNAc4dHex | 1647 | −−− | | | | | − | −−− | |
| Hex5HexNAc4 | 1663 | −−− | | −−− | −−− | −−− | −− | −−− | |
| Hex3HexNAc5dHex | 1688 | | | | +++ | | +++ | | |
| Hex9HexNAc | 1702 | −−− | −−− | | +++ | +++ | +++ | | |
| Hex4HexNAc5 | 1704 | | +++ | | | | | | |
| Hex8HexNAc2 | 1743 | −−− | −−− | +++ | + | +++ | ++ | ++ | |
| Hex5HexNAc3dHex2 | 1752 | | −−− | | +++ | | +++ | +++ | |
| Hex6HexNAc3dHex | 1768 | | | | | +++ | | +++ | |
| Hex7HexNAc3 | 1784 | −−− | | | | | | −−− | |
| Hex4HexNAc4dHex2 | 1793 | | | −− | +++ | −− | +++ | | |
| Hex5HexNAc4dHex | 1809 | −−− | | | −−− | +++ | − | | |
| Hex6HexNAc4 | 1825 | | +++ | | | | | | |
| Hex3HexNAc6dHex | 1891 | | +++ | | | | | | |
| Hex9HexNAc2 | 1905 | −−− | −−− | − | | + | ++ | ++ | |
| Hex5HexNAc4dHex2 | 1955 | −−− | | −−− | −− | | −− | | |
| Hex10HexNAc2 | 2067 | | −−− | − | | | | +++ | |
| Hex5HexNAc4dHex3 | 2101 | | | − | − | | − | +++ | |
| Hex5HexNAc5dHex2 | 2158 | +++ | | | +++ | | | | |
| Hex6HexNAc5dHex | 2174 | | | | | | +++ | | |
| Hex6HexNAc5dHex3 | 2466 | | | | +++ | | | | |

α-Man, β1,4-Gal, β1,3-Gal, and β-GlcNAc refer to specific exoglycosidase enzymes as described in the text.
Code for profiling results, when compared to the profile before the reaction;
+++: new signal appears;
++: signal is significantly increased;
+: signal is increased;
−: signal is decreased;
−−: signal is significantly decreased;
−−−: signal disappears;
blank: no change.

TABLE 3
Exoglycosidase profiling of cord blood CD133+ and CD133− cell neutral N-glycan fraction.

| | | α-Man | | β1,4-Gal | | β1,3-Gal | | β-GlcNAc | |
|---|---|---|---|---|---|---|---|---|---|
| Proposed composition | m/z | CD 133+ | CD 133− | CD 133+ | CD 133− | CD 133+ | CD 133− | CD 133+ | CD 133− |
| Hex2HexNAc | 568 | | | | + | | + | | +++ |
| HexHexNAc2 | 609 | +++ | ++ | | | | | | −−− |
| Hex3HexNAc | 730 | −−− | −−− | +++ | ++ | +++ | ++ | | ++ |
| HexHexNAc2dHex | 755 | +++ | ++ | −−− | | −−− | | | |
| Hex2HexNAc2 | 771 | + | −− | ++ | ++ | + | + | | + |
| Hex4HexNAc | 892 | −−− | −−− | + | ++ | | ++ | | + |
| Hex2HexNAc2dHex | 917 | −−− | −− | ++ | ++ | ++ | + | | |
| Hex3HexNAc2 | 933 | | −− | + | + | − | | | + |
| Hex2HexNAc3 | 974 | | | | +++ | | | | |
| Hex5HexNAc | 1054 | −−− | −− | + | ++ | + | ++ | | + |
| Hex3HexNAc2dHex | 1079 | −−− | −− | ++ | + | + | | | ++ |
| Hex2HexNAc3dHex | 1120 | +++ | ++ | ++ | + | ++ | + | | −−− |
| Hex3HexNAc3 | 1136 | +++ | + | | + | | | | −−− |
| Hex6HexNAc | 1216 | −−− | − | + | | + | + | | |
| Hex4HexNAc2dHex | 1241 | −−− | −−− | + | | | | | |
| Hex5HexNAc2 | 1257 | −− | −− | − | | | | | |
| Hex3HexNAc3dHex | 1282 | | | | | | | | −− |
| Hex4HexNAc3 | 1298 | ++ | + | + | | + | | | |
| Hex3HexNAc4 | 1339 | | | | +++ | | | | −−− |
| Hex7HexNAc | 1378 | −−− | −−− | | − | +++ | | | + |
| Hex5HexNAc2dHex | 1403 | −−− | −−− | −−− | | − | | | |
| Hex6HexNAc2 | 1419 | −− | −− | −− | − | − | −− | | |
| Hex3HexNAc3dHex2 | 1428 | | +++ | | − | | − | | |
| Hex4HexNAc3dHex | 1444 | | − | − | − | | | | |
| Hex5HexNAc3 | 1460 | −−− | − | + | + | | | | |
| Hex3HexNAc4dHex | 1485 | −− | | + | + | | − | | −−− |
| Hex4HexNAc4 | 1501 | | −−− | | | | +++ | | −−− |
| Hex8HexNAc | 1540 | −−− | −−− | −−− | | | | | ++ |
| Hex3HexNAc5 | 1542 | | −−− | | + | | − | | −−− |
| Hex6HexNAc2dHex | 1565 | | | −−− | | −−− | +++ | | |
| Hex7HexNAc2 | 1581 | −−− | −− | −− | −− | − | −− | | |
| Hex4HexNAc3dHex2 | 1590 | | −−− | − | − | − | − | | + |
| Hex5HexNAc3dHex | 1606 | −−− | −−− | + | | | | | −−− |
| Hex6HexNAc3 | 1622 | −−− | −−− | −−− | −− | | | | − |
| Hex4HexNAc4dHex | 1647 | −−− | | −−− | − | | | | −−− |
| Hex5HexNAc4 | 1663 | | | −−− | − | −− | − | | − |
| Hex3HexNAc5dHex | 1688 | −−− | + | | | | −−− | | −−− |
| Hex9HexNAc | 1702 | | | | | | | | + |
| Hex4HexNAc5 | 1704 | −−− | | | −−− | | | | |
| Hex8HexNAc2 | 1743 | −−− | −−− | −− | −− | − | −− | | |
| Hex5HexNAc3dHex2 | 1752 | | | | − | | | | +++ |
| Hex6HexNAc3dHex | 1768 | | | | | | | | |
| Hex4HexNAc4dHex2 | 1793 | | | | | | | | |
| Hex5HexNAc4dHex | 1809 | −−− | | −−− | −−− | | − | | − |
| Hex6HexNAc4 | 1825 | | | | − | | | | −−− |
| Hex5HexNAc5 | 1866 | −−− | −−− | | −−− | | | | −−− |
| Hex3HexNAc6dHex | 1891 | | | | | | | | −−− |
| Hex9HexNAc2 | 1905 | −−− | −−− | −− | −− | − | −− | | |
| Hex6HexNAc3dHex2 | 1914 | | | | −−− | | −−− | | |
| Hex5HexNAc4dHex2 | 1955 | | | | −− | | − | | −−− |
| Hex6HexNAc4dHex | 1971 | | −−− | | −−− | | | | −−− |
| Hex7HexNAc4 | 1987 | | | | −−− | | | | −−− |
| Hex5HexNAc5dHex | 2012 | | | | | | +++ | | |
| Hex6HexNAc5 | 2028 | | −−− | | −−− | | | | −−− |
| Hex10HexNAc2 | 2067 | −−− | −−− | − | − | | | | |
| Hex5HexNAc4dHex3 | 2101 | | | − | − | | − | | |
| Hex6HexNAc4dHex2 | 2117 | | −−− | | −−− | −−− | | | −−− |
| Hex7HexNAc4dHex | 2133 | | | | −−− | | | | |
| Hex6HexNAc5dHex | 2174 | | −−− | | −−− | | | | −−− |
| Hex5HexNAc6dHex | 2215 | | | | −−− | | | | |
| Hex6HexNAc4dHex3 | 2263 | | | | −−− | | −−− | | |
| Hex6HexNAc5dHex2 | 2320 | | | | −−− | | | | |
| Hex6HexNAc5dHex3 | 2466 | | | | −−− | | | | |

α-Man, β1,4-Gal, β1,3-Gal, and β-GlcNAc refer to specific exoglycosidase enzymes as described in the text.
Code for profiling results, when compared to the profile before the reaction;
+++: new signal appears;
++: signal is significantly increased;
+: signal is increased;
−: signal is decreased;
−−: signal is significantly decreased;
−−−: signal disappears;
blank: no change.

TABLE 4
Exoglycosidase profiling of cord blood Lin+ and
Lin− cell neutral N-glycan fraction.

| | | α-Man | | β1,4-Gal | | β1,3-Gal | | β-GlcNAc | |
|---|---|---|---|---|---|---|---|---|---|
| Proposed composition | m/z | LIN+ | LIN− | LIN+ | LIN− | LIN+ | LIN− | LIN+ | LIN− |
| Hex2HexNAc | 568 | −−− | +++ | + | | + | | − | |
| HexHexNAc2 | 609 | +++ | +++ | | | | +++ | | |
| Hex2HexNAcdHex | 714 | | | +++ | | | | | |
| Hex3HexNAc | 730 | −−− | +++ | ++ | +++ | + | +++ | + | |
| HexHexNAc2dHex | 755 | +++ | +++ | + | | + | | +++ | |
| Hex2HexNAc2 | 771 | + | + | + | + | + | | + | |
| Hex4HexNAc | 892 | −−− | −−− | ++ | + | ++ | | + | + |
| Hex2HexNAc2dHex | 917 | −− | −−− | + | ++ | | − | | − |
| Hex3HexNAc2 | 933 | − | | + | + | + | − | | + |
| Hex2HexNAc3 | 974 | +++ | | | | | | | |
| Hex5HexNAc | 1054 | −− | −−− | ++ | | − | | − | |
| Hex3HexNAc2dHex | 1079 | −− | −−− | | ++ | | − | ++ | ++ |
| Hex4HexNAc2 | 1095 | −− | −−− | | | | − | | |
| Hex2HexNAc3dHex | 1120 | +++ | | | | | | | |
| Hex3HexNAc3 | 1136 | +++ | + | + | + | | − | +++ | −−− |
| Hex6HexNAc | 1216 | − | −−− | + | | + | + | | + |
| Hex4HexNAc2dHex | 1241 | −−− | −−− | | + | | + | | −−− |
| Hex5HexNAc2 | 1257 | −− | −−− | | ++ | − | − | − | + |
| Hex3HexNAc3dHex | 1282 | + | | | | | | −− | −−− |
| Hex4HexNAc3 | 1298 | + | | | | | | | |
| Hex2HexNAc4dHex | 1323 | | | | +++ | | +++ | | |
| Hex3HexNAc4 | 1339 | | −−− | ++ | + | | −− | | −−− |
| Hex7HexNAc | 1378 | −−− | −−− | | | + | ++ | | |
| Hex5HexNAc2dHex | 1403 | −−− | −−− | | | | + | | |
| Hex6HexNAc2 | 1419 | −− | −− | | −− | − | − | − | |
| Hex3HexNAc3dHex2 | 1428 | +++ | | −−− | | −−− | | +++ | |
| Hex4HexNAc3dHex | 1444 | | −−− | − | + | | + | | |
| Hex5HexNAc3 | 1460 | | −−− | | | | | | |
| Hex3HexNAc4dHex | 1485 | | | | | | −− | −−− | −−− |
| Hex4HexNAc4 | 1501 | + | −−− | + | − | −−− | −− | −−− | −−− |
| Hex8HexNAc | 1540 | −−− | −−− | | −−− | + | ++ | | |
| Hex3HexNAc5 | 1542 | +++ | | ++ | + | ++ | − | | |
| Hex6HexNAc2dHex | 1565 | | −−− | −−− | | | | | −−− |
| Hex7HexNAc2 | 1581 | −− | −−− | −− | −− | | − | | |
| Hex4HexNAc3dHex2 | 1590 | | | − | | | | +++ | |
| Hex5HexNAc3dHex | 1606 | −−− | −−− | − | −−− | | −−− | | −−− |
| Hex2HexNAc4dHex3 | 1615 | | | | | | +++ | | |
| Hex6HexNAc3 | 1622 | −−− | −−− | −−− | −−− | | | | |
| Hex4HexNAc4dHex | 1647 | | −−− | −− | −−− | | | −−− | −−− |
| Hex5HexNAc4 | 1663 | | −−− | −− | −− | | − | − | −− |
| Hex3HexNAc5dHex | 1688 | | | − | | | | −−− | −−− |
| Hex9HexNAc | 1702 | −−− | −−− | | | | | | |
| Hex4HexNAc5 | 1704 | +++ | | | −−− | | | | |
| Hex8HexNAc2 | 1743 | −− | −−− | −− | −− | | − | | |
| Hex5HexNAc3dHex2 | 1752 | | | −−− | | | | +++ | |
| Hex6HexNAc3dHex | 1768 | | | −−− | | | | | |
| Hex3HexNAc4dHex3 | 1777 | | | | | | +++ | | |
| Hex7HexNAc3 | 1784 | | | −−− | | | | | |
| Hex4HexNAc4dHex2 | 1793 | +++ | | | | | | | |
| Hex5HexNAc4dHex | 1809 | + | −−− | −− | −−− | | | | −− |
| Hex6HexNAc4 | 1825 | +++ | | − | −−− | | −− | +++ | |
| Hex4HexNAc5dHex | 1850 | | | +++ | | +++ | | | |
| Hex5HexNAc5 | 1866 | +++ | | −−− | | | | | |
| Hex3HexNAc6dHex | 1891 | | −−− | − | | | | | |
| Hex9HexNAc2 | 1905 | −−− | −−− | −− | −− | | − | | |
| Hex4HexNAc4dHex3 | 1939 | | | | | | +++ | | |
| Hex5HexNAc4dHex2 | 1955 | | | −−− | | | | +++ | |
| Hex6HexNAc4dHex | 1971 | | | −−− | | | | | |
| Hex7HexNAc4 | 1987 | | | −−− | | | | +++ | |
| Hex5HexNAc5dHex | 2012 | +++ | | −−− | | | | | |
| Hex6HexNAc5 | 2028 | | | −−− | | | | | |
| Hex10HexNAc2 | 2067 | −−− | −−− | − | | | ++ | + | |
| Hex5HexNAc4dHex3 | 2101 | | | | | | | +++ | |
| Hex8HexNAc4 | 2149 | | −−− | | | | | | |
| Hex6HexNAc5dHex | 2174 | | | −−− | | | | − | |
| Hex5HexNAc6dHex | 2215 | | | −−− | | −−− | | | |
| Hex11HexNAc2 | 2229 | | | | | | +++ | | |
| Hex6HexNAc6 | 2231 | | | −−− | | −−− | | | |
| Hex6HexNAc5dHex2 | 2320 | | | −−− | | −−− | | | |
| Hex12HexNAc2 | 2391 | | | +++ | | +++ | +++ | | |
| Hex7HexNAc6 | 2393 | | | −−− | | −−− | | | |
| Hex6HexNAc5dHex3 | 2466 | | | −−− | | −−− | | | |
| Hex7HexNAc6dHex | 2539 | +++ | | | | | | | |

TABLE 5
Differential effect of α2,3-sialidase treatment on isolated sialylated
N-glycans from cord blood CD133⁺ and CD133⁻ cells.

| | Proposed monosaccharide | Sialylated N-glycan | | Neutral N-glycan | |
|---|---|---|---|---|---|
| m/z | composition | CD133⁺ | CD133⁻ | CD133⁺ | CD133⁻ |
| 1768 | (NeuAc₁)Hex₄HexNAc₄ | + | + | + | − |
| 2156 | (NeuAc₁)Hex₈HexNAc₂dHex₁/ | + | + | + | − |
| | (NeuAc₁Hex₅HexNAc₄dHex₁SO₃) | | | | |
| 2222 | (NeuAc₁)Hex₅HexNAc₄dHex₂ | + | + | + | − |
| 2238 | (NeuAc₁Hex₆HexNAc₄dHex₁/ | + | + | + | − |
| | (NeuGc₁)Hex₅HexNAc₄dHex₂ | | | | |
| 2254 | (NeuAc₁)Hex₇HexNAc₄/ | + | + | + | − |
| | (NeuGc₁)Hex₆HexNAc₄dHex₁ | | | | |
| 2368 | (NeuAc₁)Hex₅HexNAc₄dHex₃ | + | + | + | − |
| 2447 | (NeuAc₂)Hex₈HexNAc₂dHex₁/ | + | + | + | − |
| | (NeuAc₂Hex₅HexNAc₄dHex₁SO₃) | | | | |
| 2448 | (NeuAc₁)Hex₈HexNAc₂dHex₃/ | + | + | + | − |
| | (NeuAc₁Hex₅HexNAc₄dHex₃SO₃) | | | | |
| 2513 | (NeuAc₂)Hex₅HexNAc₄dHex₂ | + | + | + | − |
| 2733 | (NeuAc₁)Hex₆HexNAc₅dHex₃ | + | + | + | − |
| 2953 | (NeuAc₁)Hex7HexNAc₆dHex₂ | + | + | + | − |

The neutral N-glycan columns show that neutral N-glycans corresponding to the listed sialylated N-glycans appear in analysis of CD133⁺ cell N-glycans but not CD133⁻ cell N-glycans. Proposed glycan compositions outside parenthesis are visible in the neutral N-glycan fraction after α2,3-sialidase digestion of CD133⁺ cell sialylated N-glycans.

TABLE 6
Proposed neutral N-glycan grouping of the samples; hESC, human embryonal stem cell
line, lines 1-4, EB, embryoid bodies derived from hESC lines 3 and 4, st.3 3, stage
3 differentiated cells from hESC line 3, HEF human fibroblasts used as feeder cells.

| Neutral N-glycan Grouping: | | | | | | | | | | |
|---|---|---|---|---|---|---|---|---|---|---|
| Composition | Glycan Grouping | hESC 1 | hESC 2 | hESC 3 | hESC 4 | EB 3 | EB 4 | st. 3 3 | HEF1 | HEF2 |
| General N-glycan grouping: | | | | | | | | | | |
| Hex₅₋₁₂HexNAc₂ | high-mannose | 84.4 | 73.2 | 80.0 | 79.0 | 64.4 | 79.1 | 73.6 | 82.6 | 77.5 |
| Hex₁₋₄HexNAc₂dHex₀₋₁ | low-mannose | 5.6 | 10.9 | 6.8 | 7.8 | 11.5 | 9.2 | 9.4 | 7.1 | 8.0 |
| n_{HexNAc} = 3 and n_{Hex} ≧ 2 | hybrid/monoantennary | 3.4 | 6.7 | 3.2 | 3.2 | 9.0 | 6.7 | 6.5 | 5.4 | 5.1 |
| n_{HexNAc} ≧ 4 and n_{Hex} ≧ 2 | complex | 6.2 | 8.9 | 10.1 | 10.0 | 14.5 | 5.0 | 10.3 | 4.9 | 9.1 |
| Other types | | 0.3 | 0.3 | 0.0 | 0.0 | 0.7 | 0.0 | 0.3 | 0.0 | 0.2 |
| Complex/hybrid/monoantennary N-glycan grouping: | | | | | | | | | | |
| n_{dHex} ≧ 1 | fucosylated | 52.3 | 40.4 | 65.3 | 62.4 | 46.1 | 27.9 | 36.9 | 51.6 | 56.6 |
| n_{dHex} ≧ 2 | α2/3/4-linked Fuc | 11.7 | 1.8 | 11.7 | 13.9 | 6.9 | 9.9 | 2.2 | 0.0 | 3.4 |
| n_{HexNAc} > n_{Hex} ≧ 2 | terminal HexNAc | 9.4 | 17.4 | 6.8 | 6.0 | 17.7 | 15.5 | 18.4 | 27.2 | 16.2 |
| n_{HexNAc} = n_{Hex} ≧ 5 | bisecting GlcNAc | 0.0 | 10.2 | 0.0 | 0.0 | 7.8 | 4.2 | 9.7 | 0.0 | 0.0 |
| Complex N-glycan grouping: | | | | | | | | | | |
| n_{HexNAc} ≧ 5 and n_{Hex} ≧ 6 | large N-glycans | 11.3 | 5.4 | 13.7 | 8.7 | 3.3 | 0.0 | 4.6 | 14.1 | 20.5 |

TABLE 7
Proposed sialylated N-glycan grouping of the samples; hESC, human embryonal stem
cell line, lines 2-4, EB, embryoid bodies derived from hESC line 3, st.3 3, stage
3 differentiated cells from hESC line 3, HEF human fibroblasts used as feeder cells.

| Sialylated N-glycan Grouping: | | | | | | | |
|---|---|---|---|---|---|---|---|
| Composition | Glycan Grouping | hESC 2 | hESC 3 | hESC 4 | EB 3 | st3 3 | hEF |
| General N-glycan grouping: | | | | | | | |
| n_{HexNAc} = 3 and n_{Hex} ≧ 5 | hybrid | 0.0 | 3.8 | 4.5 | 9.6 | 3.6 | 3.4 |
| n_{HexNAc} = 3 and n_{Hex} = 3 or 4 | monoantennary | 2.2 | 2.3 | 5.5 | 6.4 | 2.5 | 3.6 |
| n_{HexNAc} ≧ 4 and n_{Hex} ≧ 3 | complex | 97.8 | 92.6 | 89.1 | 79.1 | 93.9 | 92.2 |
| Other types | — | 0.0 | 1.3 | 0.9 | 4.8 | 0.0 | 0.8 |
| Complex/hybrid/monoantennary N-glycan grouping: | | | | | | | |
| n_{dHex} ≧ 1 | fucosylated | 93.0 | 72.6 | 74.6 | 79.3 | 85.3 | 76.2 |
| n_{dHex} ≧ 2 | α2/3/4-linked Fuc | 33.5 | 23.0 | 18.5 | 10.8 | 5.2 | 20.4 |
| n_{HexNAc} > n_{Hex} ≧ 3 | terminal HexNAc | 7.8 | 6.4 | 5.2 | 7.7 | 3.0 | 0.8 |
| n_{HexNAc} = n_{Hex} ≧ 5 | bisecting GlcNAc | 4.3 | 3.9 | 2.2 | 12.5 | 25.8 | 1.4 |
| n_{NeuGc} ≧ 1 | NeuGc-containing | 0.0 | 6.8 | 5.6 | 1.5 | 0.0 | 0.0 |
| Complex N-glycan grouping: | | | | | | | |
| n_{HexNAc} ≧ 5 and n_{Hex} ≧ 6 | large N-glycans | 22.7 | 18.7 | 14.9 | 12.4 | 26.6 | 44.5 |

| | | | | | | |
|---|---|---|---|---|---|---|
| sialylation degree SD_{HexNAc} = n_{NeuAc/Gc}: (n_{HexNAc} − 2) | 51.6 | 60.4 | 63.0 | 60.7 | 56.6 | 60.3 |

TABLE 8
Mass spectrometric analysis results of sialylated N-glycans with
monosaccharide compositions NeuAc₁₋₂Hex₅HexNAc₄dHex₀₋₃
in sequential enzymatic modification steps of human cord blood
mononuclear cells. The columns show relative glycan signal
intensities (% of the tabled signals) before the modification
reactions (MNC), after α2,3-sialyltransferase reaction (α2,3SAT),
and after sequential α2,3-sialyltransferase and α1,3-fucosyltransferase
reactions (α2,3SAT + α1,3FucT). The sum of the glycan signal
intensities in each column has been normalized to 100% for clarity.

| | calc m/z | | | |
|---|---|---|---|---|
| Proposed monosaccharide | [M − | | | α2,3SAT + |
| composition | H]⁻ | MNC | α2,3SAT | α1,3FucT |
| NeuAcHex5HexNAc4 | 1930.68 | 24.64 | 12.80 | 13.04 |
| NeuAcHex5HexNAc4dHex | 2076.74 | 39.37 | 30.11 | 29.40 |
| NeuAcHex5HexNAc4dHex2 | 2222.8 | 4.51 | 8.60 | 6.83 |
| NeuAcHex5HexNAc4dHex3 | 2368.85 | 3.77 | 6.34 | 6.45 |
| NeuAc2Hex5HexNAc4 | 2221.78 | 13.20 | 12.86 | 17.63 |
| NeuAc2Hex5HexNAc4dHex | 2367.83 | 14.04 | 29.28 | 20.71 |
| NeuAc2Hex5HexNAc4dHex2 | 2513.89 | 0.47 | n.d. | 5.94 |

TABLE 9
Mass spectrometric analysis results of selected neutral N-glycans in
enzymatic modification steps of human cord blood mononuclear cells.

| Proposed monosaccharide | calc m/z | | | | | α2,3SAT + |
|---|---|---|---|---|---|---|
| composition | [M + H]⁺ | MNC | SA'ase | α2,3SAT | α1,3FucT | α1,3FucT |
| Hex5HexNAc2 | 1257.42 | 11.94 | 14.11 | 14.16 | 13.54 | 9.75 |
| Hex3HexNAc4dHex | 1485.53 | 0.76 | 0.63 | 0.78 | 0.90 | 0.78 |
| Hex6HexNAc3 | 1622.56 | 0.61 | 1.99 | 0.62 | 0.51 | 0.40 |
| Hex5HexNAc4 | 1663.58 | 0.44 | 4.81 | 0.00 | 0.06 | 0.03 |
| Hex5HexNac4dHex | 1809.64 | 0.19 | 1.43 | 0.00 | 0.25 | 0.00 |
| Hex5HexNac4dHex2 | 1955.7 | 0.13 | 0.22 | 0.00 | 0.22 | 0.00 |
| Hex6HexNAc5 | 2028.71 | 0.07 | 1.14 | 0.00 | 0.00 | 0.00 |
| Hex5HexNAc4dHex3 | 2101.76 | 0.12 | 0.09 | 0.00 | 0.22 | 0.00 |
| Hex6HexNAc5dHex | 2174.77 | 0.00 | 0.51 | 0.00 | 0.14 | 0.00 |
| Hex6HexNAc5dHex2 | 2320.83 | 0.00 | 0.00 | 0.00 | 0.08 | 0.00 |

The columns show relative glycan signal intensities (% of the total glycan signals) before the modification reactions (MNC), after broad-range sialidase reaction (SA'se), after α2,3-sialyltransferase reaction (α2,3SAT), after α1,3-fucosyltransferase reaction (α1,3FucT), and after sequential α2,3-sialyltransferase and α1,3-fucosyltransferase reactions (α2,3SAT + α1,3FucT).

TABLE 10
Exoglycosidase analysis results of hESC line FES 29 grown on mEF.

| | | FES 29 | | | | | | |
|---|---|---|---|---|---|---|---|---|
| Proposed composition | m/z | α-Man | β-GlcNAc | β-HexNAc | β1,4Gal | β1,3-Gal | α1,3/4-Fuc | α1,2-Fuc |
| Hex2HexNAc | 568 | | +++ | +++ | | +++ | +++ | +++ |
| HexHexNAc2 | 609 | +++ | | | | +++ | +++ | |
| Hex3HexNAc | 730 | −− | + | ++ | ++ | + | | + |
| HexHexNAc2dHex | 755 | +++ | | | | | | |
| Hex2HexNAc2 | 771 | + | + | −−− | | + | + | + |
| Hex4HexNAc | 892 | −−− | + | + | −−− | + | + | + |
| Hex2HexNAc2dHex | 917 | −− | + | | | + | + | + |
| Hex3HexNAc2 | 933 | −− | ++ | + | | + | + | + |
| Hex2HexNAc3 | 974 | +++ | | | | +++ | +++ | +++ |
| Hex5HexNAc | 1054 | −− | + | + | + | + | + | + |
| Hex3HexNAc2dHex | 1079 | −− | ++ | | + | + | + | + |
| Hex4HexNAc2 | 1095 | −− | | + | | + | + | + |
| Hex2HexNAc3dHex | 1120 | ++ | | − | | + | −− | |
| Hex3HexNAc3 | 1136 | + | −− | −− | | + | ++ | + |
| Hex6HexNAc | 1216 | −− | + | ++ | + | + | + | + |
| Hex4HexNAc2dHex | 1241 | −− | + | | | + | | |
| Hex5HexNAc2 | 1257 | −− | | | | | | |
| Hex3HexNAc3dHex | 1282 | | − | −− | | + | + | + |
| Hex4HexNAc3 | 1298 | + | ++ | ++ | ++ | + | ++ | ++ |
| Hex3HexNAc4 | 1339 | | −−− | −−− | ++ | ++ | −−− | −−− |
| Hex7HexNAc | 1378 | −− | + | + | + | + | + | + |
| Hex5HexNAc2dHex | 1403 | −−− | + | | | | | + |
| Hex6HexNAc2 | 1419 | −− | − | | | − | − | − |
| Hex3HexNAc3dHex2 | 1428 | +++ | | | | +++ | | |
| Hex4HexNAc3dHex | 1444 | | ++ | + | | + | + | |
| Hex5HexNAc3 | 1460 | − | | | | + | + | + |
| Hex3HexNAc4dHex | 1485 | | −− | −−− | + | + | | + |
| Hex4HexNAc4 | 1501 | | −−− | −−− | −−− | + | −−− | −−− |
| Hex8HexNAc | 1540 | −−− | + | ++ | + | | + | |
| Hex3HexNAc5 | 1542 | ++ | −−− | −−− | ++ | ++ | | + |
| Hex6HexNAc2dHex | 1565 | −−− | | −−− | | | | |
| Hex7HexNAc2 | 1581 | −− | | | | − | | |
| Hex4HexNAc3dHex2 | 1590 | | ++ | + | | | | |
| Hex5HexNAc3dHex | 1606 | − | + | | | + | | |
| Hex6HexNAc3 | 1622 | −− | | | −− | + | + | + |
| Hex4HexNAc4dHex | 1647 | | −− | −−− | −− | + | + | |
| Hex5HexNAc4 | 1663 | | + | | − | + | | + |
| Hex3HexNAc5dHex | 1688 | | −−− | −−− | | + | | + |
| Hex9HexNAc | 1702 | −−− | + | ++ | + | + | | |
| Hex4HexNAc5 | 1704 | + | − | −−− | −−− | | | |
| Hex8HexNAc2 | 1743 | −−− | − | | | − | − | − |
| Hex5HexNAc3dHex2 | 1752 | +++ | | | | | | |
| Hex6HexNAc3dHex | 1768 | −− | | −− | | | | |
| Hex7HexNAc3 | 1784 | −− | | | −− | + | | |
| Hex4HexNAc4dHex2 | 1793 | | −−− | −−− | −−− | ++ | −−− | |
| Hex5HexNAc4dHex | 1809 | | | | − | + | + | + |
| Hex6HexNAc4 | 1825 | | | | −− | | | |
| Hex4HexNAc5dHex | 1850 | | −−− | −−− | −−− | | ++ | |
| Hex5HexNAc5 | 1866 | + | + | | | ++ | ++ | ++ |
| Hex3HexNAc6dHex | 1891 | +++ | | | | | +++ | +++ |
| Hex9HexNAc2 | 1905 | −−− | − | − | | − | − | − |
| Hex7HexNAc3dHex | 1930 | | | | | | | +++ |
| Hex5HexNAc4dHex2 | 1955 | | | | | | − | |
| Hex6HexNAc4dHex | 1971 | | | | −− | | | |
| Hex7HexNAc4 | 1987 | | | | | + | | + |
| Hex4HexNAc5dHex2 | 1996 | | −−− | −−− | | | −−− | |
| Hex5HexNAc5dHex | 2012 | | −−− | −−− | | | + | |
| Hex6HexNAc5 | 2028 | | | | − | | | |
| Hex10HexNAc2 | 2067 | −−− | + | | | + | + | + |
| Hex5HexNAc6 | 2069 | +++ | | | | | | |
| Hex5HexNAc4dHex3 | 2101 | | | | | | −− | |
| Hex6HexNAc4dHex2 | 2117 | +++ | | | | +++ | | |
| Hex7HexNAc4dHex | 2311 | | | | | | | |
| Hex4HexNAc5dHex3 | 2142 | +++ | | | | +++ | | +++ |
| Hex8HexNAc4 | 2149 | | | | | +++ | | |
| Hex5HexNAc5dHex2 | 2158 | +++ | | | | +++ | | |
| Hex6HexNAc5dHex | 2174 | | | | −− | | | |
| Hex3HexNAc6dHex3 | 2183 | +++ | | | | +++ | | +++ |
| Hex7HexNAc5 | 2190 | | | | | | | |
| Hex11HexNAc2 | 2229 | −−− | | | | | | |
| Hex6HexNAc6 | 2231 | +++ | | | | | | |
| Hex5HexNAc4dHex4 | 2247 | +++ | | | | | | |
| Hex7HexNAc4dHex2 | 2279 | +++ | | | | +++ | | +++ |
| Hex5HexNAc5dHex3 | 2304 | +++ | | | | +++ | | |
| Hex6HexNAc5dHex2 | 2320 | +++ | +++ | | | +++ | +++ | +++ |
| Hex7HexNAc5dHex | 2336 | | | | − | | | |
| Hex8HexNAc5 | 2352 | | | | −−− | | | |
| Hex12HexNAc2 | 2391 | −−− | | | | | | |
| Hex7HexNAc6 | 2393 | +++ | | | | | +++ | |
| Hex7HexNAc4dHex3 | 2425 | +++ | | | | +++ | | |
| Hex6HexNAc5dHex3 | 2466 | +++ | | | | +++ | | |
| Hex8HexNAc5dHex | 2498 | | | | −−− | | | |
| Hex9HexNAc5 | 2514 | | | | | | | |
| Hex7HexNAc6dHex | 2539 | +++ | | | | | +++ | +++ |
| Hex13HexNAc2 | 2553 | | | | | | | +++ |
| Hex8HexNAc6 | 2555 | | | | | +++ | +++ | |
| Hex9HexNAc5dHex | 2660 | | | | | | | |
| Hex7HexNAc6dHex4 | 2978 | | | | | +++ | | |
| Hex8HexNAc6dHex4 | 3140 | | | | | +++ | | +++ |
| Hex9HexNAc6dHex4 | 3302 | | | | | +++ | +++ | +++ |
| Hex10HexNAc6dHex4 | 3464 | | | | | +++ | +++ | +++ |
| Hex11HexNAc6dHex4 | 3626 | | | | | +++ | +++ | +++ |
| Hex12HexNAc6dHex4 | 3788 | | | | | +++ | | +++ |

TABLE 11
Exoglycosidase analysis results of hESC line FES 29 (st 1) grown on
hEF and embryoid bodies (EB, st 2).

| | | FES | FES | FES | FES |
|---|---|---|---|---|---|
| | | 29 st 1 | 29 st 2 | 29 st 1 | 29 st 2 |
| Proposed composition | m/z | α-Man | α-Man | β1,4-Gal | β1,4-Gal |
| HexHexNAc2 | 609 | ++ | ++ | −−− | −− |
| HexHexNAc2dHex | 755 | +++ | +++ | | |
| Hex2HexNAc2 | 771 | +++ | ++ | | |
| Hex4HexNAc | 892 | | | | −−− |
| Hex2HexNAc2dHex | 917 | −−− | | −−− | |
| Hex3HexNAc2 | 933 | ++ | ++ | + | + |
| Hex5HexNAc | 1054 | | | | |
| Hex3HexNAc2dHex | 1079 | −−− | −− | | − |
| Hex4HexNAc2 | 1095 | −−− | −− | + | + |
| Hex2HexNAc3dHex | 1120 | | + | | |
| Hex3HexNAc3 | 1136 | + | ++ | ++ | ++ |
| Hex6HexNAc | 1216 | | | | |
| Hex4HexNAc2dHex | 1241 | −−− | −−− | −−− | |
| Hex5HexNAc2 | 1257 | −− | −− | | |
| Hex3HexNAc3dHex | 1282 | | | ++ | ++ |
| Hex4HexNAc3 | 1298 | + | ++ | + | + |
| Hex3HexNAc4 | 1339 | | | +++ | +++ |
| Hex7HexNAc | 1378 | −−− | −−− | −−− | |
| Hex5HexNAc2dHex | 1403 | | −−− | | |
| Hex6HexNAc2 | 1419 | −− | −− | | |
| Hex3HexNAc3dHex2 | 1428 | +++ | +++ | | |
| Hex4HexNAc3dHex | 1444 | − | + | | + |
| Hex5HexNAc3 | 1460 | | | + | + |
| Hex3HexNAc4dHex | 1485 | | | ++ | ++ |
| Hex8HexNAc | 1540 | | −−− | | |
| Hex3HexNAc5 | 1542 | | + | +++ | ++ |
| Hex6HexNAc2dHex | 1565 | | −−− | | −−− |
| Hex7HexNAc2 | 1581 | −− | −− | | |
| Hex5HexNAc3dHex | 1606 | −−− | | −−− | − |
| Hex6HexNAc3 | 1622 | −−− | −− | −−− | −−− |
| Hex4HexNAc4dHex | 1647 | | | | − |
| Hex5HexNAc4 | 1663 | | | −−− | −−− |
| Hex3HexNAc5dHex | 1688 | −−− | | ++ | ++ |
| Hex9HexNAc | 1702 | | | | |
| Hex4HexNAc5 | 1704 | | | +++ | −− |
| Hex8HexNAc2 | 1743 | −− | −− | | |
| Hex6HexNAc3dHex | 1768 | | | | |
| Hex4HexNAc4dHex2 | 1793 | | | | +++ |
| Hex5HexNAc4dHex | 1809 | − | | −− | −− |
| Hex4HexNAc5dHex | 1850 | | | −−− | −− |
| Hex5HexNAc5 | 1866 | | | | −−− |
| Hex3HexNAc6dHex | 1891 | | | | +++ |
| Hex9HexNAc2 | 1905 | −−− | −−− | | |
| Hex5HexNAc4dHex2 | 1955 | − | | − | −−− |
| Hex6HexNAc4dHex | 1971 | | | | −−− |
| Hex4HexNAc5dHex2 | 1996 | −−− | | −−− | −−− |
| Hex5HexNAc5dHex | 2012 | | | | −−− |
| Hex6HexNAc5 | 2028 | | | −−− | |
| Hex10HexNAc2 | 2067 | −−− | −−− | | |
| Hex5HexNAc4dHex3 | 2101 | | | | − |
| Hex4HexNAc5dHex3 | 2142 | | −−− | | −−− |
| Hex5HexNAc5dHex2 | 2158 | | −−− | | −−− |
| Hex6HexNAc5dHex | 2174 | | | −−− | −−− |
| Hex11HexNAc2 | 2229 | | | ++ | ++ |
| Hex6HexNAc5dHex2 | 2320 | | | −−− | |
| Hex12HexNAc2 | 2391 | | | +++ | ++ |
| Hex13HexNAc2 | 2553 | | | +++ | +++ |
| Hex14HexNAc2 | 2715 | | | | +++ |

TABLE 12
Exoglycosidase digestion analyses of hESC acidic N-glycans (cell line FES 29, grown on mEF).

| | | | | α3/4Fuc | |
|---|---|---|---|---|---|
| Proposed composition | m/z | α3SA | α3/4Fuc | →α2Fuc | SA |
| Hex3HexNAc2SP | 989 | + | −−− | −−− | −−− |
| NeuAcHex3HexNAc | 997 | +++ | | | |
| Hex2HexNAc3SP | 1030 | + | −−− | −−− | + |
| Hex4HexNac2SP | 1151 | + | | −−− | + |
| Hex3HexNAc3SP | 1192 | ++ | | ++ | ++ |
| NeuAc2Hex2HexNAcdHex | 1272 | | −−− | −−− | −−− |
| Hex4HexNAc2dHexSP | 1297 | −−− | −−− | −−− | + |
| NeuAc2HexHexNAc2dHex | 1313 | + | | −−− | ++ |
| Hex3HexNAc3dHexSP | 1338 | + | −−− | −−− | ++ |
| Hex4HexNAc3SP | 1354 | ++ | + | ++ | ++ |
| Hex3HexNac4SP | 1395 | + | | + | ++ |
| NeuAcHex3HexNAc3 | 1403 | | | + | −−− |
| NeuGcHex3HexNAc3 | 1419 | | | −−− | |
| NeuAc2Hex2HexNAcdHex | 1475 | + | | + | ++ |
| Hex4HexNAc3dHexSP | 1500 | + | | | + |
| Hex5HexNAc3dHexSP/NeuAc2HexHexNAc3dHex | 1516 | + | | | + |
| Hex3HexNAc4dHexSP | 1541 | + | | ++ | ++ |
| NeuAcHex3HexNAc3dHex | 1549 | + | + | + | −−− |
| Hex4HexNAc4SP | 1557 | ++ | | + | ++ |
| NeuAcHex4HexNAc3 | 1565 | − | | + | −− |
| NeuGcHex4HexNAc3 | 1581 | + | | | |
| NeuAcHex3HexNAc4 | 1606 | +++ | | | |
| NeuAc2Hex3HexNAc2dHex | 1637 | + | | | + |
| Hex4HexNAc3dHex2SP | 1646 | +++ | | | |
| Hex5HexNAc3dHexSP | 1662 | + | −−− | −−− | + |
| NeuAc2Hex2HexNAc3dHex | 1678 | + | − | | + |
| NeuAcHex2HexNAc3dHex3 | 1679 | | +++ | | +++ |
| Hex4HexNAc4dHexSP | 1703 | ++ | | ++ | ++ |
| NeuAcHex4HexNAc3dHex | 1711 | | | + | −− |
| Hex5HexNAc4SP | 1719 | ++ | | + | ++ |
| NeuAcHex5HexNAc3 | 1727 | − | − | | −− |
| NeuGcHex5HexNAc3 | 1743 | −−− | | + | + |
| NeuAcHex3HexNAc4dHex | 1752 | | | −−− | −−− |
| Hex4HexNAc5SP | 1760 | + | + | | ++ |
| NeuAcHex4HexNAc4 | 1768 | | + | + | −− |
| Hex7HexNAc2dHexSP | 1783 | | | | |
| NeuGcHex4HexNAc4 | 1784 | +++ | +++ | +++ | +++ |
| Hex5HexNAc4SP2/NeuAc2Hex4HexNAc2dHex | 1799 | | | ++ | ++ |
| Hex6HexNAc3dHexSP | 1824 | +++ | | | +++ |
| NeuAc2Hex3HexNAc3dHex | 1840 | + | | | + |
| NeuAcHex3HexNAc3dHex3 | 1841 | | | | +++ |
| Hex5HexNAc4dHexSP | 1865 | ++ | | + | ++ |
| NeuAcHex5HexNAc3dHex | 1873 | − | | − | −−− |
| Hex6HexNAc4SP | 1881 | ++ | + | −−− | ++ |
| NeuAcHex6HexNAc3 | 1889 | − | | | −− |
| Hex4HexNAc5dHexSP | 1906 | + | | + | ++ |
| NeuAcHex4HexNAc4dHex | 1914 | − | + | + | −− |
| Hex5HexNAc5SP | 1922 | +++ | | | +++ |
| NeuAcHex5HexNAc4 | 1930 | + | + | + | −− |
| NeuGcHex5HexNAc4 | 1946 | ++ | | + | ++ |
| NeuAcHex3HexNAc5dHex | 1955 | | + | −−− | −−− |
| NeuAc2Hex5HexNAc2dHex/Hex6HexNAc4(SP)2 | 1961 | | | | +++ |
| NeuAcHex4HexNAc5 | 1971 | | + | + | |
| NeuAc2Hex4HexNAc3dHex/Hex8HexNAc3SP | 2002 | + | | − | |
| NeuAcHex4HexNAc3dHex3 | 2003 | −−− | −−− | −−− | −−− |
| NeuAcHex5HexNAc4SP | 2010 | | −−− | −−− | −−− |
| Hex5HexNAc4dHex2SP | 2011 | −−− | | −−− | ++ |
| NeuAc2Hex5HexNAc3 | 2018 | | | | +++ |
| NeuAcHex5HexNAc3dHex2 | 2019 | | | | +++ |
| Hex6HexNAc4dHexSP | 2027 | ++ | | + | ++ |
| NeuAcHex6HexNAc3dHex | 2035 | −−− | + | −−− | −−− |
| NeuAc2Hex3HexNAc4dHex/Hex7HexNAc4SP | 2043 | +++ | | | +++ |
| NeuAcHex7HexNAc3 | 2051 | | − | | −−− |
| Hex4HexNAc5dHex2SP | 2052 | −−− | | −−− | ++ |
| Hex5HexNAc5dHexSP | 2068 | +++ | +++ | | +++ |
| NeuAcHex5HexNAc4dHex | 2076 | | | + | −− |
| NeuGcHex5HexNAc4dHex/NeuAcHex6HexNAc4 | 2092 | − | − | − | |
| NeuGcHex6HexNAc4 | 2108 | − | | | + |
| NeuAcHex4HexNAc5dHex | 2117 | | + | + | − |
| NeuAcHex5HexNAc5 | 2133 | | + | ++ | |
| NeuAcHex5HexNAc4dHexSP/ | 2156 | | + | | −−− |
| NeuAcHex8HexNAc2dHex | | | | | |
| Hex5HexNAc4dHex3SP | 2157 | | +++ | | +++ |
| NeuAc2Hex5HexNAc3dHex | 2164 | | | −−− | |
| NeuAcHex5HexNAc3dHex3 | 2165 | | +++ | | |
| NeuAcHex9HexNAc2/NeuAcHex6HexNAc4SP/ | 2172 | +++ | | | |
| NeuGcHex5HexNAc4dHexSP | | | | | |
| NeuAcHex4HexNAc6 | 2174 | −−− | −−− | −−− | −−− |
| NeuAc2Hex3HexNAc4dHex2/Hex7HexNAc4dHexSP | 2189 | | | −−− | |
| NeuAcHex3HexNAc4dHex4 | 2190 | −−− | −−− | −−− | ++ |
| NeuGcNeuAcHex6HexNAc3/ | 2196 | | +++ | +++ | |
| NeuGc2Hex5HexNAc3dHex | | | | | |
| Hex4HexNAc5dHexSP | 2198 | −−− | −−− | −−− | |
| NeuAc2Hex4HexNAc4(SP)2 | 2219 | | | | +++ |
| NeuAc2Hex5HexNAc4 | 2221 | −− | | | −− |
| NeuAcHex5HexNAc4dHex2 | 2222 | − | −− | −−−?? | −− |
| Hex6HexNAc5dHexSP | 2230 | ++ | −−− | −−− | ++ |
| NeuGcNeuAcHex5HexNAc4 | 2237 | +++ | +++ | | |
| NeuGcHex5HexNAc4dHex2/NeuAcHex6HexNAc4dHex | 2238 | −− | − | − | −− |
| NeuGc2Hex5HexNAc4 | 2253 | + | ++ | −−− | −−− |
| NeuAcHex7HexNAc4/NeuGcHex6HexNAc4dHex | 2254 | ++ | − | ++ | ++ |
| NeuAcHex4HexNAc5dHex2 | 2263 | | −−− | −−− | −−− |
| NeuAcHex5HexNAc5dHex | 2279 | | + | + | − |
| NeuAcHex6HexNAc5 | 2295 | | | + | |
| NeuAcHex5HexNAc3dHex4/NeuGcHex6HexNAc5 | 2311 | +++ | | | +++ |
| Hex6HexNAc4dHex3SP | 2319 | −−− | −−− | ++ | −−− |
| NeuAc2Hex5HexNAc4dHex | 2367 | −− | | − | −−− |
| NeuAcHex5HexNAc4dHex3 | 2368 | −−− | − | −−− | −−− |
| NeuGcNeuAcHex5HexNAc4dHex/ | 2383 | −− | | − | −−− |
| NeuAc2Hex6HexNAc4 | | | | | |
| NeuGcHex5HexNAc4dHex3/NeuAcHex6HexNAc4dHex2 | 2384 | | +++ | | |
| NeuAc3Hex5HexNAx3SP/NeuAc2Hex5HexNAc4Ac4 | 2389 | −−− | + | + | −−− |
| NeuAc2Hex5HexNAc3dHexSP | 2390 | | +++ | | |
| NeuAc2Hex3HexNAc5dHex2 | 2392 | | | | +++ |
| NeuAcHex3HexNAc5dHex4 | 2393 | | | | +++ |
| NeuGc2Hex5HexNAc4dHex | 2399 | −−− | −−− | −−− | −−− |
| NeuAc2Hex6HexNAc3dHexSP | 2406 | −−− | ++ | −−− | −−− |
| NeuAc2Hex4HexNAc5dHex | 2408 | −−− | −−− | −−− | −−− |
| NeuAcHex5HexNAc5dHex2 | 2425 | | +++ | | |
| NeuAcHex6HexNAc5dHex | 2441 | + | + | + | |
| NeuAc2Hex5HexNAc4dHexSP/ | 2447 | −−− | −−− | −−− | −−− |
| NeuAc2Hex8HexNAc2dHex | | | | | |
| NeuAcHex5HexNAc4dHex3SP/ | 2448 | −−− | −−− | −−− | −−− |
| NeuAcHex8HexNAc2dHex3 | | | | | |
| NeuAcHex3HexNAc6dHex3 | 2450 | | | | +++ |
| NeuAcHex7HexNAc5 | 2457 | | | | ++ |
| NeuAc3Hex5HexNAc4 | 2512 | −−− | | −−− | −−− |
| NeuAc2Hex5HexNAc4dHex2 | 2513 | −−− | −−− | −−− | −−− |
| NeuAcHex6HexNAc5dHexSP | 2521 | +++ | | | |
| NeuGcNeuAc2Hex5HexNAc4 | 2528 | −−− | | −−− | −−− |
| NeuGcNeuAcHex5HexNAc4dHex2/ | 2529 | −−− | −−− | −−− | −−− |
| NeuAc2Hex6HexNAc4dHex | | | | | |
| NeuGc2NeuAcHex5HexNAc4 | 2544 | −−− | −−− | −−− | −−− |
| NeuAc2Hex6HexNAc5 | 2586 | −−− | + | −−− | −−− |
| NeuAcHex6HexNAc5dHex2 | 2587 | −−− | | | −−− |
| Hex7HexNAc6dHexSP | 2595 | +++ | | | +++ |
| NeuAcHex7HexNAc5dHex/NeuGcHex6HexNAc5dHex2 | 2603 | | + | | |
| NeuAcHex8HexNAc5/NeuGcHex7HexNAc5dHex | 2619 | | | −−− | |
| NeuAcHex6HexNAc6dHex | 2644 | +++ | | | |
| NeuAcHex7HexNAc6 | 2660 | | −−− | −−− | + |
| NeuAc2Hex6HexNAc5dHex | 2732 | − | | | −−− |
| NeuAcHex6HexNAc5dHex3 | 2733 | −−− | | −−− | −−− |
| NeuAc2Hex4HexNAc6dHex2 | 2758 | +++ | | | +++ |
| NeuAcHex8HexNAc5dHex | 2765 | − | | | −− |
| NeuGcHex8HexNAc5dHex/NeuAcHex9HexNAc5 | 2781 | | −−− | −−− | |
| NeuAc2Hex5HexNAc4dHex4 | 2806 | ++ | | | +++ |
| NeuAcHex7HexNAc6dHex | 2807 | | +++ | +++ | −−− |
| NeuAcHex8HexNAc6 | 2822 | +++ | | | +++ |
| NeuAc3Hex6HexNAc5 | 2878 | −−− | −−− | −−− | −−− |
| NeuGcNeuAc2Hex6HexNAc5 | 2894 | −−− | −−− | −−− | −−− |
| NeuGcNeuAcHex6HexNAc5dHex2/ | 2895 | | +++ | | |
| NeuAc2Hex7HexNAc5dHex | | | | | |
| NeuAc2Hex7HexNAc6 | 2952 | | −−− | −−− | −−− |
| NeuAcHex7HexNAc6dHex2 | 2953 | +++ | | | |
| NeuAc3Hex6HexNAc5dHex | 3024 | −−− | + | −−− | −−− |
| NeuAc2Hex7HexNAc6dHex | 3098 | −−− | −−− | −−− | −−− |
| NeuAcHex8HexNAc7dHex | 3172 | +++ | | | |

¹⁾Code: +++ new signal appeared, ++ highly increased relative signal intensity, ++ increased relative signal intensity, − decreased relative signal intensity, −− greatly decreased relative signal intensity, −−− signal disappeared, blank: no change.

TABLE 13

| | Preferred | | | |
|---|---|---|---|---|
| | monosaccharide | Terminal | Experimental structures included in the glycan | |
| m/z* | compositions | epitopes | signal according to the invention^{§} | Group^{#} |
| 730 | Hex3HexNAc | Manα | (Manα→)₂Hex₁HexNAc₁ | S |
| 771 | Hex2HexNAc2 | Manα | Manα→Hex₁HexNAc₂ | LO |
| 892 | Hex4HexNAc | Manα | (Manα→)₃Hex₁HexNAc₁ | S |
| | | Galβ4 | Galβ4GlcNAc→Hex₃ | |
| 917 | Hex2HexNAc2dHex | Manα | Manα→Hex₁HexNAc₂dHex₁ | LO, F |
| 933 | Hex3HexNAc2 | Manα | (Manα→)₂Hex₁HexNAc₂ | LO |
| 1054 | Hex5HexNAc | Manα | (Manα→)₄Hex₁HexNAc₁ | S |
| 1079 | Hex3HexNAc2dHex | Manα | (Manα→)₂Hex₁HexNAc₂dHex₁ | LO, F |
| 1095 | Hex4HexNAc2 | Manα | (Manα→)₃Hex₁HexNAc₂ | LO |
| 1120 | Hex2HexNAc3dHex | Fucα3/4 | Fucα3/4→Hex₂HexNAc₃ | HY, F, |
| | | | | N > H |
| 1136 | Hex3HexNAc3 | GlcNAcβ | GlcNAcβ→Hex₃HexNAc₂ | HY, |
| | | | | N = H |
| 1216 | Hex6HexNAc | Manα | (Manα→)₅Hex₁HexNAc₁ | S |
| 1241 | Hex4HexNAc2dHex | Manα | (Manα)₃Hex₁HexNAc₂dHex₁ | LO, F |
| 1257 | Hex5HexNAc2 | Manα | (Manα→)₄Hex₁HexNAc₂ | HI |
| 1282 | Hex3HexNAc3dHex | GlcNAcβ | GlcNAcβ→Hex₃HexNAc₂dHex₁ | HY, F, |
| | | | | N = H |
| 1298 | Hex4HexNAc3 | | | HY |
| 1339 | Hex3HexNAc4 | 2 × GlcNAcβ | (GlcNAcβ→)₂Hex₃HexNAc₂ | CO, |
| | | | | N > H |
| 1378 | Hex7HexNAc | Manα | (Manα→)₆Hex₁HexNAc₁ | S |
| 1403 | Hex5HexNAc2dHex | Manα | (Manα→)₄Hex₁HexNAc₂dHex₁ | HF |
| 1419 | Hex6HexNAc2 | Manα | (Manα→)₅Hex₁HexNAc₂ | HI |
| 1444 | Hex4HexNAc3dHex | Manα | Manα→Hex₃HexNAc₃dHex₁ | HY, F |
| 1460 | Hex5HexNAc3 | Manα | Manα→Hex₄HexNAc₃ | HY |
| 1485 | Hex3HexNAc4dHex | 2 × GlcNAcβ | (GlcNAcβ→)₂Hex₃HexNAc₂dHex₁ | CO, F, |
| | | | | N > H |
| 1501 | Hex4HexNAc4 | GlcNAcβ | GlcNAcβ→Hex₄HexNAc₃ | CO, |
| | | Galβ4 | Galβ4GlcNAc→Hex₃HexNAc₃ | N = H |
| 1540 | Hex8HexNAc | Manα | (Manα→)₇Hex₁HexNAc₁ | S |
| 1542 | Hex3HexNAc5 | 3 × GlcNAcβ | (GlcNAcβ→)₃Hex₃HexNAc₂ | CO, |
| | | | | N > H |
| 1565 | Hex6HexNAc2dHex | Manα | (Manα→)₅Hex₁HexNAc₂dHex₁ | HF |
| 1581 | Hex7HexNAc2 | Manα | (Manα→)₆Hex₁HexNAc₂ | HI |
| 1590 | Hex4HexNAc3dHex2 | Fucα | Fucα→Hex₄HexNAc₃dHex₁ | HY, FC |
| 1606 | Hex5HexNAc3dHex | Manα | Manα→Hex₄HexNAc₃dHex₁ | HY, F |
| | | Galβ4 | Galβ4GlcNAc→Hex₄HexNAc₂dHex₁ | |
| | | | Manα→[Galβ4GlcNAc→]Hex₃HexNAc₂dHex₁ | |
| 1622 | Hex6HexNAc3 | Manα | Manα→Hex₅HexNAc₃ | HY |
| | | Galβ4 | Galβ4GlcNAc→Hex₅HexNAc₂ | |
| | | | Manα→[Galβ4GlcNAc→]Hex₄HexNAc₂ | |
| 1647 | Hex4HexNAc4dHex | GlcNAcβ | GlcNAcβ→Hex₄HexNAc₃dHex₁ | CO, F, |
| | | Galβ4 | Galβ4GlcNAc→Hex₃HexNAc₃dHex₁ | N = H |
| | | | GlcNAcβ→[Galβ4GlcNAc→]Hex₃HexNAc₂dHex₁ | |
| 1663 | Hex5HexNAc4 | 2 × Galβ4 | (Galβ4GlcNAc→)₂Hex₃HexNAc₂ | CO |
| 1688 | Hex3HexNAc5dHex | 3 × GlcNAcβ | (GlcNAcβ→)₃Hex₃HexNAc₂dHex₁ | CO, F, |
| | | Manα | Manα→Hex₂HexNAc₅dHex₁ | N > H |
| 1702 | Hex9HexNAc | Manα | (Manα→)₈Hex₁HexNAc₁ | S |
| 1704 | Hex4HexNAc5 | 2 × HexNAcβ | HexNAcβHexNAcβ→Hex₄HexNAc₃dHex₁ | CO, |
| | | (not | Galβ4GlcNAc→Hex₃HexNAc₄dHex₁ | N > H |
| | | GlcNAc) | HexNAcβHexNAcβ→[Galβ4GlcNAc→] | |
| | | Galβ4 | Hex₃HexNAc₂dHex₁ | |
| 1743 | Hex8HexNAc2 | Manα | (Manα→)₇Hex₁HexNAc₂ | HI |
| 1768 | Hex6HexNAc3dHex | Manα | Manα→Hex₅HexNAc₃dHex₁ | HY, F |
| 1784 | Hex7HexNAc3 | Manα | Manα→Hex₆HexNAc₃ | HY |
| | | Galβ4 | Galβ4GlcNAc→Hex₆HexNAc₂ | |
| | | | Manα→[Galβ4GlcNAc→]Hex₅HexNAc₂ | |
| 1793 | Hex4HexNAc4dHex2 | GlcNAcβ | GlcNAcβ→Hex₄HexNAc₃dHex₂ | CO, FC, |
| | | Galβ4 | Galβ4GlcNAc→Hex₃HexNAc₃dHex₂ | N = H |
| | | Fucα3/4 | Fucα3/4→Hex₄HexNAc₄dHex₁ | |
| | | | GlcNAcβ→[Galβ4GlcNAc→]Hex₃HexNAc₂dHex₂ | |
| | | | GlcNAcβ→[Fucα3/4→]Hex₄HexNAc₃dHex₁ | |
| | | | Fucα3/4→[Galβ4GlcNAc→]Hex₃HexNAc₃dHex₁ | |
| | | | GlcNAcβ→[Fucα3/4→][Galβ4GlcNAc→] | |
| | | | Hex₄HexNAc₃dHex₁ | |
| 1809 | Hex5HexNAc4dHex | 2 × Galβ4 | (Galβ4GlcNAc→)₂Hex₃HexNAc₂dHex₁ | CO, F |
| 1850 | Hex4HexNAc5dHex | 2 × GlcNAcβ | (GlcNAcβ→)₂Hex₄HexNAc₃dHex₁ | CO, F, |
| | | Galβ4 | Galβ4GlcNAc→Hex₃HexNAc₄dHex₁ | N > H |
| | | | Galβ4GlcNAc→[GlcNAcβ→]₂Hex₃HexNAc₂dHex₁ | |
| 1866 | Hex5HexNAc5 | | | CO, |
| | | | | N = H |
| 1905 | Hex9HexNAc2 | Manα | (Manα→)₈Hex₁HexNAc₂ | HI |
| 1955 | Hex5HexNAc4dHex2 | Fucα3/4 | Fucα3/4→Hex₅HexNAc₄dHex₁ | CO, FC |
| | | Galβ4 | Galβ4GlcNAc→Hex₄HexNAc₃dHex₂ | |
| | | | Galβ4GlcNAc→[Fucα3/4→]Hex₄HexNAc₃dHex₁ | |
| 1971 | Hex6HexNAc4dHex | Galβ4 | Galβ4GlcNAc→Hex₅HexNAc₃dHex₁ | CO, F |
| 1996 | Hex4HexNAc5dHex2 | 2 × GlcNAcβ | (GlcNAcβ→)₂Hex₄HexNAc₃dHex₂ | CO, FC, |
| | | Fucα3/4 | Fucα3/4→Hex₄HexNAc₅dHex₁ | N > H |
| | | Galβ4 | Galβ4GlcNAc→Hex₃HexNAc₄dHex₂ | |
| | | | (GlcNAcβ→)₂[Fucα3/4→]Hex₄HexNAc₃dHex₁ | |
| | | | Galβ4GlcNAc→[Fucα3/4→]Hex₃HexNAc₄dHex₁ | |
| 2012 | Hex5HexNAc5dHex | GlcNAcβ | GlcNAcβ→Hex₅HexNAc₄dHex₁ | CO, F, |
| | | | | N = H |
| 2028 | Hex6HexNAc5 | Galβ4 | Galβ4GlcNAc→Hex₅HexNAc₄ | CO |
| | | 3 × Galβ4 | (Galβ4GlcNAc→)₃Hex₃HexNAc₂ | |
| 2067 | Hex10HexNAc2 | Manα | Glc→(Manα→)₈Hex₁HexNAc₂ | G |
| | | Glc | | |
| 2101 | Hex5HexNAc4dHex3 | GlcNAcβ | GlcNAcβ→Hex₅HexNAc₃dHex₃ | CO, FC |
| 2174 | Hex6HexNAc5dHex | 3 × Galβ4 | (Galβ4GlcNAc→)₃Hex₃HexNAc₂dHex₁ | CO, F |
| 2229 | Hex11HexNAc2 | Manα | Glc₂→(Manα→)₈Hex₁HexNAc₂ | G |
| | | Glc | | |
| 2320 | Hex6HexNAc5dHex2 | Galβ4 | Galβ4GlcNAc→Hex₅HexNAc₄dHex₂ | CO, FC |
| 2391 | Hex12HexNAc2 | Manα | Glc₃→(Manα→)₈Hex₁HexNAc₂ | G |
| | | Glc | | |

*[M + Na]⁺ ion, first isotope.
^{§}“→” indicates linkage to a monosaccharide in the rest of the structure; “[ ]” indicates branch in the structure.
^{#}Preferred structure group based on monosaccharide compositions according to the present invention. HI, high-mannose; LO, low-mannose; S, soluble mannosylated; HF, fucosylated high-mannose; G, glucosylated high-mannose; HY, hybrid-type or monoantennary; CO, complex-type; F, fucosylation; FC, complex fucosylation; N = H, terminal HexNAc (HexNAc = Hex); N > H, terminal HexNAc (HexNAc > Hex).

TABLE 14

| Proposed composition | m/z | α-Man | β-GlcNAc | β4-Gal | β3-Gal |
|---|---|---|---|---|---|
| Hex2HexNAc | 568 | | −−− | | |
| HexHexNAc2 | 609 | +++ | | | |
| Hex2HexNAcdHex | 714 | +++ | | | |
| Hex3HexNAc | 730 | −− | −−− | | |
| HexHexNAc2dHex | 755 | +++ | | | |
| Hex2HexNAc2 | 771 | ++ | ++ | | |
| Hex4HexNAc | 892 | −−− | + | | |
| Hex2HexNAc2dHex | 917 | | + | | |
| Hex3HexNAc2 | 933 | ++ | ++ | | |
| Hex2HexNAc3 | 974 | +++ | | | |
| Hex5HexNAc | 1054 | −− | | | |
| Hex3HexNAc2dHex | 1079 | −− | + | | |
| Hex4HexNAc2 | 1095 | − | + | | |
| Hex2HexNAc3dHex | 1120 | +++ | −−− | | |
| Hex3HexNAc3 | 1136 | ++ | −− | + | |
| Hex2HexNAc2dHex3 | 1209 | −−− | −−− | | |
| Hex6HexNAc | 1216 | −− | | | |
| Hex4HexNAc2dHex | 1241 | −−− | | | |
| Hex5HexNAc2 | 1257 | −− | | | |
| Hex2HexNAc3dHex2 | 1266 | | | | |
| Hex3HexNAc3dHex | 1282 | ++ | −− | + | |
| Hex4HexNAc3 | 1298 | ++ | − | | |
| Hex3HexNAc4 | 1339 | +++ | | +++ | |
| Hex7HexNAc | 1378 | −− | | | |
| Hex5HexNAc2dHex | 1403 | −−− | | | |
| Hex6HexNAc2 | 1419 | −− | + | | |
| Hex3HexNAc3dHex2 | 1428 | +++ | | | |
| Hex4HexNAc3dHex | 1444 | + | − | + | |
| Hex5HexNAc3 | 1460 | + | − | ++ | |
| Hex3HexNAc4dHex | 1485 | | −− | ++ | |
| Hex4HexNAc4 | 1501 | ++ | | | |
| Hex8HexNAc | 1540 | − | | | |
| Hex3HexNAc5 | 1542 | | | +++ | |
| Hex6HexNAc2dHex | 1565 | −−− | | −−− | −−− |
| Hex7HexNAc2 | 1581 | −− | | | |
| Hex4HexNAc3dHex2 | 1590 | | | | |
| Hex5HexNAc3dHex | 1606 | | −− | −− | |
| Hex6HexNAc3 | 1622 | −− | − | −− | |
| Hex4HexNAc4dHex | 1647 | | −−− | | |
| Hex5HexNAc4 | 1663 | | −− | −−− | |
| Hex3HexNAc5dHex | 1688 | | −−− | ++ | |
| Hex9HexNAc | 1702 | −−− | −−− | | |
| Hex8HexNAc2 | 1743 | −− | + | | |
| Hex6HexNAc3dHex | 1768 | | | −−− | |
| Hex7HexNAc3 | 1784 | −−− | −−− | −−− | |
| Hex4HexNAc4dHex2 | 1793 | | −−− | ++ | |
| Hex5HexNAc4dHex | 1809 | | −− | −−− | |
| Hex3HexNAc6dHex | 1891 | | | +++ | |
| Hex9HexNAc2 | 1905 | −−− | − | | |
| Hex5HexNAc4dHex2 | 1955 | | − | −−− | |
| Hex6HexNAc4dHex | 1971 | | −−− | −−− | |
| Hex4HexNAc5dHex2 | 1996 | | −−− | | |
| Hex5HexNAc5dHex | 2012 | | −−− | −−− | −−− |
| Hex6HexNAc5 | 2028 | | − | −−− | |
| Hex10HexNAc2 | 2067 | −−− | − | | |
| Hex5HexNAc4dHex3 | 2101 | | −−− | | |
| Hex4HexNAc5dHex3 | 2142 | | | −− | −−− |
| Hex6HexNAc5dHex | 2174 | | −− | −−− | |
| Hex11HexNAc2 | 2229 | | | | |
| Hex5HexNAc5dHex3 | 2304 | | −−− | | |
| Hex6HexNAc5dHex2 | 2320 | | −−− | | |
| Hex7HexNAc6 | 2393 | | | −−− | |
| Hex6HexNAc5dHex3 | 2466 | | −−− | | |
| Hex7HexNAc6dHex | 2539 | | −−− | −−− | |

TABLE 15

| | Preferred | | | |
|---|---|---|---|---|
| | monosaccharide | Terminal | Experimental structures included in the glycan | |
| m/z* | compositions | epitopes | signal according to the invention^{§} | Group^{#} |
| 568 | Hex2HexNAc | Manα | Manα→Hex₁HexNAc₁ | S |
| 730 | Hex3HexNAc | Manα | (Manα→)₂Hex₁HexNAc₁ | S |
| | | GlcNAc | GlcNAc→Hex₃ | |
| 771 | Hex2HexNAc2 | Manα | Manα→Hex₁HexNAc₂ | LO |
| 892 | Hex4HexNAc | Manα | (Manα→)₃Hex₁HexNAc₁ | S |
| 917 | Hex2HexNAc2dHex | Manα | Manα→Hex₁HexNAc₂dHex₁ | LO, F |
| 933 | Hex3HexNAc2 | Manα | (Manα→)₂Hex₁HexNAc₂ | LO |
| 1054 | Hex5HexNAc | Manα | (Manα→)₄Hex₁HexNAc₁ | S |
| 1079 | Hex3HexNAc2dHex | Manα | (Manα→)₂Hex₁HexNAc₂dHex₁ | LO, F |
| 1095 | Hex4HexNAc2 | Manα | (Manα→)₃Hex₁HexNAc₂ | LO |
| 1120 | Hex2HexNAc3dHex | GlcNAcβ | GlcNAcβ→Hex₂HexNAc₂dHex₁ | HY, F, |
| | | | | N > H |
| 1136 | Hex3HexNAc3 | GlcNAcβ | GlcNAcβ→Hex₃HexNAc₂ | HY, |
| | | | | N = H |
| 1209 | Hex2HexNAc2dHex3 | Manα | Manα→Hex₁HexNAc₂dHex₃ | FC, |
| | | GlcNAc | GlcNAc→Hex₂HexNAc₁dHex₃ | N = H |
| 1216 | Hex6HexNAc | Manα | (Manα→)₅Hex₁HexNAc₁ | S |
| 1241 | Hex4HexNAc2dHex | Manα | (Manα)₃Hex₁HexNAc₂dHex₁ | LO, F |
| 1257 | Hex5HexNAc2 | Manα | (Manα→)₄Hex₁HexNAc₂ | HI |
| 1266 | Hex2HexNAc3dHex2 | Fuc | Fuc→Hex₂HexNAc₃dHex₁ | HY, FC |
| 1282 | Hex3HexNAc3dHex | GlcNAcβ | GlcNAcβ→Hex₃HexNAc₂dHex₁ | HY, F, |
| | | | | N = H |
| 1298 | Hex4HexNAc3 | | | HY |
| 1378 | Hex7HexNAc | Manα | (Manα→)₆Hex₁HexNAc₁ | S |
| 1403 | Hex5HexNAc2dHex | Manα | (Manα)₄Hex₁HexNAc₂dHex₁ | HF |
| 1419 | Hex6HexNAc2 | Manα | (Manα→)₅Hex₁HexNAc₂ | HI |
| 1444 | Hex4HexNAc3dHex | GlcNAcβ | GlcNAcβ→Hex₄HexNAc₂dHex₁ | HY, F |
| 1460 | Hex5HexNAc3 | GlcNAcβ | GlcNAcβ→Hex₅HexNAc₂ | HY |
| 1485 | Hex3HexNAc4dHex | 2 × GlcNAcβ | (GlcNAcβ→)₂Hex₃HexNAc₂dHex₁ | CO, F, |
| | | | | N > H |
| 1501 | Hex4HexNAc4 | | | CO, |
| | | | | N = H |
| 1540 | Hex8HexNAc | Manα | (Manα→)₇Hex₁HexNAc₁ | S |
| 1565 | Hex6HexNAc2dHex | Manα | (Manα)₅Hex₁HexNAc₂dHex₁ | HF |
| 1581 | Hex7HexNAc2 | Manα | (Manα→)₆Hex₁HexNAc₂ | HI |
| 1590 | Hex4HexNAc3dHex2 | Fucα | Fucα→Hex₄HexNAc₃dHex₁ | HY, FC |
| 1606 | Hex5HexNAc3dHex | GlcNAcβ | GlcNAcβ→Hex₅HexNAc₂dHex₁ | HY, F |
| | | Galβ4 | Galβ4GlcNAc→Hex₄HexNAc₂dHex₁ | |
| 1622 | Hex6HexNAc3 | Manα | Manα→Hex₅HexNAc₃ | HY |
| | | GlcNAcβ | GlcNAcβ→Hex₆HexNAc₂ | |
| | | Galβ4 | Galβ4GlcNAc→Hex₅HexNAc₂ | |
| | | | Manα→[GlcNAcβ→]Hex₅HexNAc₂ | |
| | | | Manα→[Galβ4GlcNAc→]Hex₄HexNAc₂ | |
| 1647 | Hex4HexNAc4dHex | GlcNAcβ | GlcNAcβ→Hex₄HexNAc₃dHex₁ | CO, F, |
| | | | | N = H |
| 1663 | Hex5HexNAc4 | 2 × Galβ4 | (Galβ4GlcNAc→)₂Hex₃HexNAc₂ | CO |
| | | GlcNAcβ | GlcNAcβ→Hex₅HexNAc₃ | |
| 1688 | Hex3HexNAc5dHex | 3 × GlcNAcβ | (GlcNAcβ→)₃Hex₃HexNAc₂dHex₁ | CO, F, |
| | | | | N > H |
| 1702 | Hex9HexNAc | Manα | (Manα→)₈Hex₁HexNAc₁ | S |
| 1743 | Hex8HexNAc2 | Manα | (Manα→)₇Hex₁HexNAc₂ | HI |
| 1768 | Hex6HexNAc3dHex | Galβ4 | Galβ4GlcNAc→Hex₅HexNAc₂dHex₁ | HY, F |
| 1784 | Hex7HexNAc3 | Manα | Manα→Hex₆HexNAc₃ | HY |
| | | GlcNAcβ | GlcNAcβ→Hex₇HexNAc₂ | |
| | | Galβ4 | Galβ4GlcNAc→Hex₆HexNAc₂ | |
| | | | Manα→[GlcNAcβ→]Hex₆HexNAc₂ | |
| | | | Manα→[Galβ4GlcNAc→]Hex₅HexNAc₂ | |
| 1793 | Hex4HexNAc4dHex2 | GlcNAcβ | GlcNAcβ→Hex₄HexNAc₃dHex₂ | CO, FC, |
| | | Fuc | Fuc→Hex₄HexNAc₄dHex₁ | N = H |
| | | | GlcNAcβ→[Fuc→]Hex₄HexNAc₃dHex₁ | |
| 1809 | Hex5HexNAc4dHex | 2 × Galβ4 | (Galβ4GlcNAc→)₂Hex₃HexNAc₂dHex₁ | CO, F |
| | | GlcNAcβ | GlcNAcβ→Hex₅HexNAc₃dHex₁ | |
| 1891 | Hex3HexNAc6dHex | | | CO, F, |
| | | | | N > H |
| 1905 | Hex9HexNAc2 | Manα | (Manα→)₈Hex₁HexNAc₂ | HI |
| 1955 | Hex5HexNAc4dHex2 | Galβ4 | Galβ4GlcNAc→Hex₄HexNAc₃dHex₂ | CO, FC |
| | | Fuc | Fuc→Hex₅HexNAc₄dHex₁ | |
| | | | Galβ4GlcNAc→[Fuc→]Hex₄HexNAc₃dHex₁ | |
| 1971 | Hex6HexNAc4dHex | GlcNAcβ | GlcNAcβ→Hex₆HexNAc₃dHex₁ | CO, F |
| | | Galβ4 | Galβ4GlcNAc→Hex₅HexNAc₃dHex₁ | |
| 1996 | Hex4HexNAc5dHex2 | 2 × GlcNAcβ | (GlcNAcβ→)₂Hex₄HexNAc₃dHex₂ | CO, FC, |
| | | | | N > H |
| 2012 | Hex5HexNAc5dHex | GlcNAcβ | GlcNAcβ→Hex₅HexNAc₄dHex₁ | CO, F, |
| | | 2 × Galβ4 | (Galβ4GlcNAc→)₂Hex₃HexNAc₃dHex₁ | N = H |
| | | Galβ3 | Galβ3GlcNAc→Hex₄HexNAc₄dHex₁ | |
| | | | (Galβ4GlcNAc→)₂[GlcNAcβ→]Hex₃HexNAc₂dHex₁ | |
| 2028 | Hex6HexNAc5 | 3 × Galβ4 | (Galβ4GlcNAc→)₃Hex₃HexNAc₂ | CO |
| 2067 | Hex10HexNAc2 | Manα | Glc→(Manα→)₈Hex₁HexNAc₂ | G |
| | | Glc | | |
| 2101 | Hex5HexNAc4dHex3 | GlcNAcβ | GlcNAcβ→Hex₅HexNAc₃dHex₃ | CO, FC |
| 2142 | Hex4HexNAc5dHex3 | Galβ4 | Galβ4GlcNAc→Hex₃HexNAc₄dHex₃ | CO, FC, |
| | | | | N > H |
| 2174 | Hex6HexNAc5dHex | GlcNAcβ | GlcNAcβ→Hex₆HexNAc₄dHex₁ | CO, F |
| | | 3 × Galβ4 | (Galβ4GlcNAc→)₃Hex₃HexNAc₂dHex₁ | |
| 2229 | Hex11HexNAc2 | Glc | Glc₂→(Manα→)₈Hex₁HexNAc₂ | G |
| | | Manα | | |
| 2304 | Hex5HexNAc5dHex3 | GlcNAcβ | GlcNAcβ→Hex₅HexNAc₄dHex₃ | CO, FC, |
| | | | | N = H |
| 2320 | Hex6HexNAc5dHex2 | GlcNAcβ | GlcNAcβ→Hex₆HexNAc₄dHex₂ | CO, FC |
| 2393 | Hex7HexNAc6 | Galβ4 | Galβ4GlcNAc→Hex₆HexNAc₅ | CO |
| 2466 | Hex6HexNAc5dHex3 | GlcNAcβ | GlcNAcβ→Hex₆HexNAc₄dHex₃ | CO, FC |
| 2539 | Hex7HexNAc6dHex | GlcNAcβ | GlcNAcβ→Hex₇HexNAc₅dHex₁ | CO, F |
| | | 4 × Galβ4 | (Galβ4GlcNAc→)₄Hex₃HexNAc₂dHex₁ | |

*[M + Na]⁺ ion, first isotope.
^{§}“→” indicates linkage to a monosaccharide in the rest of the structure; “[ ]” indicates branch in the structure.
^{#}Preferred structure group based on monosaccharide compositions according to the present invention. HI, high-mannose; LO, low-mannose; S, soluble mannosylated; HF, fucosylated high-mannose; G, glucosylated high-mannose; HY, hybrid-type or monoantennary; CO, complex-type; F, fucosylation; FC, complex fucosylation; N = H, terminal HexNAc (HexNAc = Hex); N > H, terminal HexNAc (HexNAc > Hex).

TABLE 16

| Proposed composition | m/z | α-Man | β-GlcNAc | β4-Gal | β3-Gal |
|---|---|---|---|---|---|
| Hex2HexNAc | 568 | −−− | −−− | | |
| HexHexNAc2 | 609 | +++ | | | −−− |
| Hex2HexNAcdHex | 714 | +++ | | | |
| Hex3HexNAc | 730 | − | | | |
| HexHexNAc2dHex | 755 | +++ | | | |
| Hex2HexNAc2 | 771 | ++ | ++ | − | − |
| Hex4HexNAc | 892 | −−− | −−− | | |
| Hex2HexNAc2dHex | 917 | − | ++ | − | − |
| Hex3HexNAc2 | 933 | ++ | ++ | − | − |
| HexHexNAc3dHex | 958 | | | | |
| Hex2HexNAc3 | 974 | +++ | | ++ | −−− |
| Hex5HexNAc | 1054 | −−− | | | |
| Hex3HexNAc2dHex | 1079 | −− | ++ | − | − |
| Hex4HexNAc2 | 1095 | −− | + | − | − |
| Hex2HexNAc3dHex | 1120 | +++ | | + | −−− |
| Hex3HexNAc3 | 1136 | ++ | −−− | ++ | −− |
| Hex2HexNAc2dHex3 | 1209 | −−− | −−− | | |
| Hex6HexNAc | 1216 | −−− | | +++ | +++ |
| Hex4HexNAc2dHex | 1241 | −−− | | | − |
| Hex5HexNAc2 | 1257 | −− | | | |
| Hex3HexNAc3dHex | 1282 | ++ | −−− | + | − |
| Hex4HexNAc3 | 1298 | +++ | + | − | − |
| Hex3HexNAc4 | 1339 | | | +++ | −−− |
| Hex7HexNAc | 1378 | | +++ | +++ | |
| Hex5HexNAc2dHex | 1403 | −−− | | | − |
| Hex6HexNAc2 | 1419 | −− | + | | |
| Hex3HexNAc3dHex2 | 1428 | +++ | | | |
| Hex4HexNAc3dHex | 1444 | ++ | | − | − |
| Hex5HexNAc3 | 1460 | + | −− | + | − |
| Hex3HexNAc4dHex | 1485 | | −−− | ++ | − |
| Hex4HexNAc4 | 1501 | + | −−− | −− | − |
| Hex8HexNAc | 1540 | | | −−− | − |
| Hex3HexNAc5 | 1542 | | | +++ | |
| Hex6HexNAc2dHex | 1565 | −−− | | | −−− |
| Hex7HexNAc2 | 1581 | −− | | | |
| Hex4HexNAc3dHex2 | 1590 | | | | |
| Hex5HexNAc3dHex | 1606 | | −− | −− | − |
| Hex6HexNAc3 | 1622 | −− | −− | −− | − |
| Hex4HexNAc4dHex | 1647 | | −−− | | − |
| Hex5HexNAc4 | 1663 | | − | −− | |
| Hex3HexNAc5dHex | 1688 | | −−− | ++ | −−− |
| Hex4HexNAc5 | 1704 | | | +++ | |
| Hex8HexNAc2 | 1743 | −− | | | |
| Hex5HexNAc3dHex2 | 1752 | | −−− | −−− | |
| Hex6HexNAc3dHex | 1768 | −− | −− | −− | |
| Hex7HexNAc3 | 1784 | − | | −−− | |
| Hex4HexNAc4dHex2 | 1793 | | −−− | ++ | −−− |
| Hex5HexNAc4dHex | 1809 | | −− | −−− | |
| Hex6HexNAc4 | 1825 | +++ | +++ | −− | |
| Hex4HexNAc5dHex | 1850 | | | +++ | |
| Hex5HexNAc5 | 1866 | | | −−− | −−− |
| Hex3HexNAc6dHex | 1891 | | | ++ | −−− |
| Hex9HexNAc2 | 1905 | −−− | | | |
| Hex5HexNAc4dHex2 | 1955 | | −−− | −− | − |
| Hex6HexNAc4dHex | 1971 | | −−− | −−− | |
| Hex7HexNAc4 | 1987 | | −−− | | −−− |
| Hex4HexNAc5dHex2 | 1996 | | −−− | +++ | |
| Hex5HexNAc5dHex | 2012 | | −−− | −− | |
| Hex6HexNAc5 | 2028 | | − | −−− | − |
| Hex10HexNAc2 | 2067 | −−− | | | − |
| Hex5HexNAc4dHex3 | 2101 | | − | | |
| Hex6HexNAc4dHex2 | 2117 | | | −−− | −−− |
| Hex7HexNAc4dHex | 2133 | | −−− | | −−− |
| Hex4HexNAc5dHex3 | 2142 | | | −−− | −−− |
| Hex6HexNAc5dHex | 2174 | | −− | −−− | − |
| Hex5HexNAc7 | 2272 | | | +++ | |
| Hex5HexNAc5dHex3 | 2304 | | −−− | +++ | |
| Hex6HexNAc5dHex2 | 2320 | | −−− | −−− | |
| Hex7HexNAc6 | 2393 | | −− | −−− | |
| Hex6HexNAc5dHex3 | 2466 | | | −−− | −−− |
| Hex7HexNAc6dHex | 2539 | | −−− | −−− | |
| Hex8HexNAc7 | 2758 | | | −−− | −−− |

TABLE 17

| Proposed composition | m/z | β4-Gal | β-GlcNAc |
|---|---|---|---|
| Hex2HexNAc | 568 | − | −−− |
| HexHexNAc2 | 609 | +++ | |
| Hex3HexNAc | 730 | | |
| Hex2HexNAc2 | 771 | | −− |
| Hex4HexNAc | 892 | | −−− |
| Hex2HexNAc2dHex | 917 | | − |
| Hex3HexNAc2 | 933 | | − |
| Hex2HexNAc3 | 974 | +++ | |
| Hex5HexNAc | 1054 | | |
| Hex3HexNAc2dHex | 1079 | | |
| Hex4HexNAc2 | 1095 | | |
| Hex2HexNAc3dHex | 1120 | +++ | |
| Hex3HexNAc3 | 1136 | ++ | −−− |
| Hex2HexNAc2dHex3 | 1209 | −−− | −−− |
| Hex6HexNAc | 1216 | | |
| Hex4HexNAc2dHex | 1241 | | |
| Hex5HexNAc2 | 1257 | | |
| Hex3HexNAc3dHex | 1282 | + | −− |
| Hex4HexNAc3 | 1298 | | |
| Hex3HexNAc4 | 1339 | +++ | |
| Hex2HexNac2dHex4 | 1355 | +++ | |
| Hex7HexNAc | 1378 | | |
| Hex5HexNAc2dHex | 1403 | | |
| Hex6HexNAc2 | 1419 | | |
| Hex4HexNAc3dHex | 1444 | + | |
| Hex5HexNAc3 | 1460 | ++ | − |
| Hex3HexNAc4dHex | 1485 | ++ | −−− |
| Hex4HexNAc4 | 1501 | −− | −−− |
| Hex8HexNAc | 1540 | | |
| Hex3HexNAc5 | 1542 | +++ | |
| Hex6HexNAc2dHex | 1565 | | |
| Hex7HexNAc2 | 1581 | | |
| Hex4HexNAc3dHex2 | 1590 | +++ | +++ |
| Hex5HexNAc3dHex | 1606 | − | |
| Hex6HexNAc3 | 1622 | −− | − |
| Hex4HexNAc4dHex | 1647 | | −−− |
| Hex5HexNAc4 | 1663 | −−− | ++ |
| Hex3HexNAc5dHex | 1688 | ++ | −−− |
| Hex9HexNAc | 1702 | −−− | −−− |
| Hex4HexNAc5 | 1704 | +++ | −−− |
| Hex8HexNAc2 | 1743 | | |
| Hex5HexNAc3dHex2 | 1752 | +++ | |
| Hex6HexNAc3dHex | 1768 | − | |
| Hex7HexNAc3 | 1784 | −−− | −−− |
| Hex4HexNAc4dHex2 | 1793 | +++ | |
| Hex5HexNAc4dHex | 1809 | −−− | + |
| Hex4HexNAc5dHex | 1850 | | −−− |
| Hex3HexNAc6dHex | 1891 | ++ | −−− |
| Hex9HexNAc2 | 1905 | | |
| Hex5HexNAc4dHex2 | 1955 | −−− | |
| Hex4HexNAc5dHex2 | 1996 | | −−− |
| Hex5HexNAc5dHex | 2012 | −−− | −−− |
| Hex6HexNAc5 | 2028 | −−− | |
| Hex10HexNAc2 | 2067 | | |
| Hex5HexNAc4dHex3 | 2101 | | + |
| Hex6HexNAc5dHex | 2174 | −−− | |
| Hex7HexNAc6 | 2393 | −−− | −−− |
| Hex7HexNAc6dHex | 2539 | −−− | −−− |

TABLE 18

| Proposed composition | m/z | α-Man | β4-Gal | β-GlcNAc |
|---|---|---|---|---|
| Hex2HexNAc | 568 | −−− | − | −−− |
| HexHexNAc2 | 609 | +++ | − | −−− |
| Hex3HexNAc | 730 | −− | − | |
| HexHexNAc2dHex | 755 | +++ | | −−− |
| Hex2HexNAc2 | 771 | ++ | − | −− |
| Hex4HexNAc | 892 | −−− | − | −−− |
| Hex2HexNAc2dHex | 917 | −− | − | −− |
| Hex3HexNAc2 | 933 | − | − | −− |
| Hex2HexNAc3 | 974 | ++ | + | −−− |
| Hex5HexNAc | 1054 | −−− | | |
| Hex3HexNAc2dHex | 1079 | −−− | − | −− |
| Hex4HexNAc2 | 1095 | −− | − | − |
| Hex2HexNAc3dHex | 1120 | ++ | + | −−− |
| Hex3HexNAc3 | 1136 | + | ++ | −− |
| Hex6HexNAc | 1216 | −− | | |
| Hex4HexNAc2dHex | 1241 | −−− | | |
| Hex5HexNAc2 | 1257 | −−− | | |
| Hex3HexNAc3dHex | 1282 | | + | −− |
| Hex4HexNAc3 | 1298 | + | | |
| Hex3HexNAc4 | 1339 | | ++ | −−− |
| Hex7HexNAc | 1378 | −−− | | |
| Hex5HexNAc2dHex | 1403 | −−− | | |
| Hex6HexNAc2 | 1419 | −− | | |
| Hex3HexNAc3dHex2 | 1428 | +++ | | |
| Hex4HexNAc3dHex | 1444 | | | |
| Hex5HexNAc3 | 1460 | | + | |
| Hex3HexNAc4dHex | 1485 | | ++ | −−− |
| Hex4HexNAc4 | 1501 | | −− | −−− |
| Hex8HexNAc | 1540 | −−− | | −−− |
| Hex3HexNAc5 | 1542 | + | ++ | −−− |
| Hex6HexNAc2dHex | 1565 | −−− | | − |
| Hex7HexNAc2 | 1581 | −− | | |
| Hex4HexNAc3dHex2 | 1590 | −−− | | ++ |
| Hex5HexNAc3dHex | 1606 | − | −− | + |
| Hex6HexNAc3 | 1622 | −− | −− | ++ |
| Hex4HexNAc4dHex | 1647 | | −− | −−− |
| Hex5HexNAc4 | 1663 | | −−− | + |
| Hex3HexNAc5dHex | 1688 | | ++ | −−− |
| Hex4HexNAc5 | 1704 | | +++ | |
| Hex8HexNAc2 | 1743 | −− | | |
| Hex5HexNAc3dHex2 | 1752 | | | +++ |
| Hex6HexNAc3dHex | 1768 | − | −− | + |
| Hex7HexNAc3 | 1784 | −−− | −− | |
| Hex4HexNAc4dHex2 | 1793 | | + | −−− |
| Hex5HexNAc4dHex | 1809 | | −−− | |
| Hex6HexNAc4 | 1825 | −−− | − | + |
| Hex4HexNAc5dHex | 1850 | −−− | | −−− |
| Hex5HexNAc5 | 1866 | | −−− | −−− |
| Hex3HexNAc6dHex | 1891 | −−− | ++ | −−− |
| Hex9HexNAc2 | 1905 | −−− | | |
| Hex5HexNAc4dHex2 | 1955 | | | ++ |
| Hex6HexNAc4dHex | 1971 | | −−− | + |
| Hex7HexNAc4 | 1987 | | +++ | |
| Hex4HexNAc5dHex2 | 1996 | | | −−− |
| Hex5HexNAc5dHex | 2012 | | −−− | −−− |
| Hex6HexNAc5 | 2028 | | −−− | |
| Hex10HexNAc2 | 2067 | −−− | | |
| Hex5HexNAc4dHex3 | 2101 | | | + |
| Hex6HexNAc5dHex | 2174 | | −−− | |
| Hex6HexNAc6 | 2231 | | −−− | −−− |
| Hex5HexNAc5dHex3 | 2304 | | | −−− |
| Hex6HexNAc5dHex2 | 2320 | | −−− | −−− |
| Hex6HexNAc6dHex | 2377 | | −−− | −−− |
| Hex7HexNAc6 | 2393 | | −−− | −− |
| Hex6HexNAc5dHex3 | 2466 | | | |
| Hex7HexNAc6dHex | 2539 | | −−− | −−− |
| Hex8HexNAc6dHex4 | 3140 | | −−− | −−− |

TABLE 19
See also Example 14.

| | | FES | FES | | |
|---|---|---|---|---|---|
| Reagent | Target | 22 | 30 | mEF | % stain |
| FITC-PSA | α-Man | − | − | + | |
| FITC-RCA | β-Gal (Galβ4GlcNAc) | + | − | +/− | |
| FITC-PNA | β-Gal (Galβ3GalNAc) | + | + | − | |
| FITC-MAA | α2,3-sialyl-LN | + | + | − | |
| FITC-SNA | α2,6-sialyl-LN | + | n.d. | + | |
| FITC-PWA | I-antigen | + | + | n.d. | |
| FITC-STA | i-antigen | + | − | + | |
| FITC-WFA | β-GalNAc | + | + | − | |
| NeuGc-PAA- | NeuGc-lectin | + | + | + | |
| biotin | | | | | |
| anti-GM3(Gc) | NeuGcα3Galβ4Glc | + | + | + | |
| mAb | | | | | |
| FITC-LTA | α-Fuc | + | − | + | |
| FITC-UEA | α-Fuc | + | − | + | |
| mAb Lex | Lewis^{x} | + | n.d. | − | |
| mAb sLex | sialyl-Lewis^{x} | + | n.d. | − | |
| GF 279 Le c | Galβ3GlcNAc | | + | − | 95-100 |
| GF 283 Le b | | | + | − | 20-35 |
| GF 284 H Type 2 | | | + | − | 15-20 |
| GF 285 H Type 2 | | | − | + | 95-100 |
| GF 286 H Type 2 | | | + | − | 10-20 |
| GF 287 H Type 1 | | | + | − | 90-100 |
| GF 288 Globo-H | | | + | − | 20-35 |
| GF 289 Ley | | | − | + | 95-100 |
| GF 290 H Type 2 | | | + | − | 20-35 |

+, specific binding.
−, no specific binding.
n.d., not determined.
% of stain means approximate percentage of cell stained with a binder.

Summary of antibody stainings and FACS analysis of bone marrow derived mesenchymal stem cells and osteogenic cells derived from them.

| | | BM- | | | posit | |
|---|---|---|---|---|---|---|
| Code | Antigen | MSC | posit (%) | Osteog | (%) | Change |
| GF274 | PNAd (peripheral lymph node addressin; CD62L ligand) closely | − | 0% | − | 0% | |
| | associated with L-selectin (CD34, GlyCAM-1, MAdCAM-1), sulfomucin | | | | | |
| GF275 | CA15-3 (Cancer antigen 15-3; sialylated carbohydrate epitope of the | +* | ~50% | + | 100% | |
| | MUC-1 glycoprotein) | | | | | |
| GF276 | oncofetal antigen, tumor associated glycoprotein (TAG-72) or CA 72-4 | −* | 0% | + | ~90% | ↑↑ |
| GF277 | human sialosyl-Tn antigen (STn, sCD175) | (+)* | >50% | + | ~90% | ↑ |
| GF278 | human Tn antigen (Tn, CD175 B1.1) | (+)* | >50% | + | ~80% | ↑ |
| GF295 | Blood group antigen precursor (BG1), Lewis c Gb3GN (pLN) | − | 0% | − | 0% | |
| GF280 | TF-antigen isoform (Nemod TF2) | −* | 0% | − | 0% | |
| GF281 | TF-antigen isoform (A68-E/E3) | −* | 0% | − | 0% | |
| GF296 | asialoganglioside GM1 | − | 0% | − | 0%** | |
| GF297 | Globoside GL4 | + | 100% | + | ~75% | |
| GF298 | Human CD77 (=blood group substance pk), GB3 | + | 80-90% | + | ~50% | |
| GF299 | Forssman antigen, glycosphingolipid (FO GSL) differentiation ag | − | 0% | − | 0% | |
| GF300 | Asialo GM2 | − | 0% | − | 0%** | |
| GF301 | Lewis b blood group antigen | −* | 0% | − | 0% | |
| GF302 | H type 2 blood group antigen | +* | ~50% | + | <50% | |
| GF303 | Blood group H1(O) antigen (BG4) | −* | 0% | + | >50% | ↑↑ |
| GF288 | Globo-H | −* | 0% | NT | | |
| GF304 | Lewis a | − | 0% | − | ** | |
| GF305 | Lewis x, CD15, 3-FAL, SSEA-1,3-fucosyl-N-acetyllactosamine | (+/−) | <5% | − | 0% | ↓ |
| GF306 | Sialyl Lewis a | − | 0% | − | 0% | |
| GF307 | Sialyl Lewis x | + | ~20% | (+/−) | <10%** | ↓ |
| GF353 | SSEA-3 (stage-specific embryonic antigen-3) | + | ~50% | (+/−) | ~10% | ↓↓ |
| GF354 | SSEA-4 (stage-specific embryonic antigen-4) | +* | ~75% | − | <5% | ↓↓ |
| GF365 | Nemod TFI, DC176, GalB1-3GalNAc | − | 0% | − | 0% | |
| GF374 | Glycodelin A, GdA, PP14 (A87-D/F4) | (+/−) | <5% | − | 0% | |
| GF375 | Glycodelin A, GdA, PP14 (A87-D/C5) | − | 0% | − | 0% | |
| GF376 | Glycodelin A, GdA, PP14 (A87-B/D2) | − | 0% | − | 0% | |

+ = positive,
(+) = weak positive,
(+/−) = single positive cells,
− = negative;
NT = not tested;
*= result has been confirmed by FACS analysis,
**= in certain cell batches higher binding or binding cells were observed and in the invention is directed to these markers.

TABLE 20

| Lectins | Target | % of positive cells |
|---|---|---|
| FITC-GNA | α-Man | 27.8 |
| FITC-HHA | α-Man | 95.3 |
| FITC-PSA | α-Man | 95.5 |
| FITC-RCA | β-Gal (Galβ4GlcNAc) | 94.8 |
| FITC-PNA | β-Gal (Galβ3GalNAc) | 31.1 |
| FITC-MAA | α2,3-sialylation | 89.9 |
| FITC-SNA | α2,6-sialylation | 14.3 |
| FITC-PWA | I-antigen | 1.9 |
| FITC-STA | i-antigen | 11.9 |
| FITC-LTA | α-Fuc | 2.8 |
| FITC-UEA | α-Fuc | 8.0 |

TABLE 21

| | | BM MSC | | | | | | | |
|---|---|---|---|---|---|---|---|---|---|
| | | lectin concentration, μg/ml | | | | | | | |
| Lectin | Target | 0.25 | 0.5 | 1 | 2.5 | 5 | 10 | 20 | 40 |
| FITC-GNA | α-Man | −¹⁾ | − | ++ | ++ | ++ | ++ | ++ | ++ |
| FITC-HHA | α-Man | ++ | ++ | +++ | +++ | +++ | +++ | +++ | +++ |
| FITC-PSA | α-Man | ++ | ++ | ++ | +++ | +++ | +++ | +++ | +++ |
| FITC-RCA | β-Gal (Galβ4GlcNAc) | − | − | +/− | +/− | + | + | ++ | ++ |
| FITC-PNA | β-Gal (Galβ3GalNAc) | − | − | − | − | +/− | +/− | +/− | + |
| FITC-MAA | α2,3-sialylation | − | − | − | +/− | + | ++ | ++ | ++ |
| FITC-SNA | α2,6-sialylation | − | − | − | − | +/− | +/− | + | + |
| FITC-PWA | I-antigen | − | − | − | − | − | − | +/− | +/− |
| FITC-STA | i-antigen | − | − | − | − | − | +/− | +/− | +/− |
| FITC-LTA | α-Fuc | − | − | − | − | − | − | − | − |
| FITC-UEA | α-Fuc | − | − | − | +/− | +/− | + | ++ | ++ |
| FITC-MBL | α-Man/β-GlcNAc | − | − | − | − | − | − | +/− | + |

¹⁾Grading of staining/labelling: +++ very intense, ++ intense, + low, +/− barely detectable, − not labelled.

TABLE 22
N-glycan structural feature analysis based on proposed monosaccharide
compositions of four hESC lines FES 21, FES 22, FES 29, and FES 30. The numbers refer to
percentage from either neutral (A-E) or acidic (J-L) N-glycan pools, or from subfractions of
hybrid/monoantenary and complex-type N-glycans (N ≧ 3, F-I and M-P). EB 29 and EB 30:
embryoid bodies derived from hESC lines FES 29 and FES 30, respectively; st.3 29: stage 3
differentiated cells derived from hESC line FES 29. H: hexose; N: N-acetylhexosamine; F:
deoxyhexose.

| | | | | | FES 21* | FES 22 | FES 29 | FES 30 | EB | st. 3 |
|---|---|---|---|---|---|---|---|---|---|---|
| Neutral | A | N = 2 and 5 ≦ H ≦ 10 | | high-mannose type | 84^{#} | 73 | 79 | 79 | 73 | 72 |
| N-glycans | B | N = 2 and 1 ≦ H ≦ 4 | | low-mannose type | 5 | 11 | 7 | 8 | 12 | 12 |
| | C | N = 3 and H ≧ 2 | | hybrid/monoantennary | 3 | 7 | 3 | 3 | 5 | 6 |
| | D | N ≧ 4 and H ≧ 3 | | complex-type | 6 | 9 | 10 | 10 | 8 | 8 |
| | E | other types | | | 2 | 0 | 1 | 0 | 2 | 2 |
| | N ≧ 3 | F | F ≧ 1 | fucosylation | 8 | 11 | 10 | 10 | 14 | 15 |
| | | G | F ≧ 2 | complex fucosylation | 1 | 0 | 2 | 2 | 2 | 2 |
| | | H^{§} | N > H ≧ 2 | terminal N (N > H) | 1 | 2 | 1 | 1 | 3 | 3 |
| | | I | N = H ≧ 5 | terminal N (N = H) | 0 | 2 | 0 | 0 | 1 | 1 |
| Sialylated | J | N = 3 and H ≧ 3 | | hybrid/monoantennary | 8 | 2 | 5 | 9 | 13 | 14 |
| N-glycans | K | N ≧ 4 and H ≧ 3 | | complex-type | 91 | 98 | 94 | 90 | 83 | 77 |
| | L | other types | | | 1 | 0 | 1 | 1 | 4 | 9 |
| | N ≧ 3 | M | F ≧ 1 | fucosylation | 85 | 96 | 75 | 78 | 83 | 86 |
| | | N | F ≧ 2 | complex fucosylation | 24 | 34 | 23 | 19 | 12 | 11 |
| | | O | N > H ≧ 3 | terminal N (N > H) | 10 | 8 | 6 | 5 | 10 | 10 |
| | | P | N = H ≧ 5 | terminal N (N = H) | 3 | 4 | 4 | 2 | 14 | 20 |

TABLE 23
Comparison of lectin ligand profile in hESCs and MEFs

| Lectin | hESC | MEF |
|---|---|---|
| PSA | − | + |
| MAA | + | − |
| PNA | + | − |
| RCA | + | + |

+ present in cell surface
− not present in cell surface

TABLE 24
Summary of the results of BM MSC grown on different
immobilized lectin surfaces.

| | Proliferation | Effect vs. |
|---|---|---|
| Coating | factor | plastic |
| plastic | 3.8 | |
| RCA | 1.0 | n.g. |
| PSA | 3.9 | (+) |
| LTA | 4.0 | + |
| SNA | 3.7 | (−) |
| GS II | 4.9 | + |
| UEA | 2.1 | − |
| ECA | 4.4 | + |
| MAA | 3.7 | (−) |
| STA | 3.1 | − |
| PWA | 4.2 | + |
| WFA | 2.9 | − |
| NPA | 3.6 | (−) |

Proliferation factor = the number of cells on day 3/the number of cells on day 1.
Triplicates were used in calculations.
Effect vs. plastic: ‘n.g.’ = no growth; ‘−’ = slower growth rate; ‘+’ = faster growth rate than on plastic; ‘( )’ nearly equal to plastic.

TABLE 25

| | | Terminal | |
|---|---|---|---|
| m/z* | Preferred monosaccharide compositions | epitopes | Group^{#} |
| 989 | Hex3HexNAc2SP | | SP |
| 1030 | Hex2HexNAc3SP | | HY, SP, |
| | | | N > H |
| 1151 | Hex4HexNac2SP | | SP |
| 1192 | Hex3HexNAc3SP | | HY, SP |
| 1272 | NeuAc2Hex2HexNAcdHex | NeuAcα6/8/9 | F |
| | | Fucα3/4 | |
| 1297 | Hex4HexNAc2dHexSP | | F, SP |
| 1313 | NeuAc2HexHexNAc2dHex | Fucα2 | F |
| 1338 | Hex3HexNAc3dHexSP | Fucα3/4 | HY, F, |
| | | | SP |
| 1354 | Hex4HexNAc3SP | | HY, SP |
| 1395 | Hex3HexNac4SP | | CO, SP, |
| | | | N > H |
| 1403 | NeuAcHex3HexNAc3 | NeuAcα6/8/9 | HY |
| 1419 | NeuGcHex3HexNAc3 | | HY |
| 1475 | NeuAc2Hex2HexNAcdHex | | F |
| 1500 | Hex4HexNAc3dHexSP | | HY, F, |
| | | | SP |
| 1516 | Hex5HexNAc3dHexSP/NeuAc2HexHexNAc3dHex | | HY, F |
| | | | (SP) |
| 1541 | Hex3HexNAc4dHexSP | | CO, F, |
| | | | SP, |
| | | | N > H |
| 1549 | NeuAcHex3HexNAc3dHex | NeuAcα6/8/9 | HY, F |
| 1557 | Hex4HexNAc4SP | | CO, SP |
| 1565 | NeuAcHex4HexNAc3 | NeuAcα6/8/9 | HY |
| | | NeuAcα3 | |
| 1581 | NeuGcHex4HexNAc3 | | HY |
| 1637 | NeuAc2Hex3HexNAc2dHex | | F |
| 1662 | Hex5HexNAc3dHexSP | Fucα3/4 | HY, F, |
| | | | SP |
| 1678 | NeuAc2Hex2HexNAc3dHex | Fucα3/4 | HY, F, |
| | | | N > H |
| 1703 | Hex4HexNAc4dHexSP | | CO, F, |
| | | | SP |
| 1711 | NeuAcHex4HexNAc3dHex | NeuAcα6/8/9 | HY, F |
| 1719 | Hex5HexNAc4SP | | CO, SP |
| 1727 | NeuAcHex5HexNAc3 | NeuAcα6/8/9 | HY |
| | | NeuAcα3 | |
| | | Fucα3/4 | |
| 1743 | NeuGcHex5HexNAc3 | NeuGcα3 | HY |
| 1752 | NeuAcHex3HexNAc4dHex | NeuAcα6/8/9 | CO, F, |
| | | Fucα2 | N > H |
| 1760 | Hex4HexNAc5SP | | CO, SP, |
| | | | N > H |
| 1768 | NeuAcHex4HexNAc4 | NeuAcα6/8/9 | CO |
| 1783 | Hex7HexNAc2dHexSP | | F, SP |
| 1799 | Hex5HexNAc4SP2/NeuAc2Hex4HexNAc2dHex | | (CO) |
| | | | (F) (SP) |
| 1840 | NeuAc2Hex3HexNAc3dHex | | HY, F |
| 1865 | Hex5HexNAc4dHexSP | | CO, F, |
| | | | SP |
| 1873 | NeuAcHex5HexNAc3dHex | NeuAcα6/8/9 | HY, F |
| | | NeuAcα3 | |
| | | Fucα2 | |
| 1881 | Hex6HexNAc4SP | | CO, SP |
| 1889 | NeuAcHex6HexNAc3 | NeuAcα6/8/9 | HY |
| | | NeuAcα3 | |
| 1906 | Hex4HexNAc5dHexSP | | CO, F, |
| | | | SP, |
| | | | N > H |
| 1914 | NeuAcHex4HexNAc4dHex | NeuAcα6/8/9 | CO, F |
| | | NeuAcα3 | |
| 1930 | NeuAcHex5HexNAc4 | NeuAcα6/8/9 | CO |
| 1946 | NeuGcHex5HexNAc4 | | CO |
| 1955 | NeuAcHex3HexNAc5dHex | NeuAcα6/8/9 | CO, F, |
| | | Fucα2 | N > H |
| 1971 | NeuAcHex4HexNAc5 | | CO, |
| | | | N > H |
| 2002 | NeuAc2Hex4HexNAc3dHex/Hex8HexNAc3SP | Fucα2 | HY (F) |
| | | | (SP) |
| 2003 | NeuAcHex4HexNAc3dHex3 | NeuAcα3 | HY, FC |
| | | NeuAcα6/8/9 | |
| | | Fucα3/4 | |
| 2010 | NeuAcHex5HexNAc4SP | NeuAcα6/8/9 | CO, SP |
| | | Fucα3/4 | |
| 2011 | Hex5HexNAc4dHex2SP | NeuAcα3 | CO, FC, |
| | | Fucα2 | SP |
| 2027 | Hex6HexNAc4dHexSP | | CO, F, |
| | | | SP |
| 2035 | NeuAcHex6HexNAc3dHex | NeuAcα3 | HY, F |
| | | NeuAcα6/8/9 | |
| | | Fucα2 | |
| 2051 | NeuAcHex7HexNAc3 | NeuAcα6/8/9 | HY |
| | | Fucα3/4 | |
| 2052 | Hex4HexNAc5dHex2SP | NeuAcα3 | SP |
| | | Fucα2 | |
| 2076 | NeuAcHex5HexNAc4dHex | NeuAcα6/8/9 | CO, F |
| 2092 | NeuGcHex5HexNAc4dHex/NeuAcHex6HexNAc4 | NeuAcα3 | CO (F) |
| | | Fucα3/4 | |
| 2108 | NeuGcHex6HexNAc4 | NeuGcα3 | CO |
| 2117 | NeuAcHex4HexNAc5dHex | NeuAcα6/8/9 | CO, F |
| 2133 | NeuAcHex5HexNAc5 | | CO, |
| | | | N = H |
| 2156 | NeuAcHex5HexNAc4dHexSP/NeuAcHex8HexNAc2dHex | NeuAcα6/8/9 | (CO) F |
| | | | (SP) |
| 2164 | NeuAc2Hex5HexNAc3dHex | Fucα2 | HY, F |
| 2174 | NeuAcHex4HexNAc6 | NeuAcα3 | CO, |
| | | NeuAcα6/8/9 | N > H |
| | | Fucα3/4 | |
| 2189 | NeuAc2Hex3HexNAc4dHex2/Hex7HexNAc4dHexSP | Fucα2 | CO |
| | | | F(C) |
| | | | (SP) |
| | | | (N > H) |
| 2190 | NeuAcHex3HexNAc4dHex4 | NeuAcα3 | CO, FC, |
| | | Fucα3/4 | N > H |
| 2198 | Hex4HexNAc5dHexSP | NeuAcα3 | CO, F, |
| | | Fucα3/4 | SP, |
| | | | N > H |
| 2221 | NeuAc2Hex5HexNAc4 | NeuAcα3 | CO |
| | | NeuAcα6/8/9 | |
| 2222 | NeuAcHex5HexNAc4dHex2 | NeuAcα3 | CO, FC |
| | | NeuAcα6/8/9 | |
| | | Fucα3/4 | |
| | | Fucα2 | |
| 2230 | Hex6HexNAc5dHexSP | Fucα3/4 | CO, F, |
| | | | SP |
| 2238 | NeuGcHex5HexNAc4dHex2/NeuAcHex6HexNAc4dHex | NeuAcα3 | CO, |
| | | NeuAcα6/8/9 | F(C) |
| | | Fucα3/4 | |
| 2253 | NeuGc2Hex5HexNAc4 | NeuAcα6/8/9 | CO |
| | | Fucα2 | |
| 2254 | NeuAcHex7HexNAc4/NeuGcHex6HexNAc4dHex | Fucα3/4 | CO (F) |
| 2263 | NeuAcHex4HexNAc5dHex2 | NeuAcα6/8/9 | CO, FC, |
| | | Fucα3/4 | N > H |
| 2279 | NeuAcHex5HexNAc5dHex | NeuAcα6/8/9 | CO, F, |
| | | | N = H |
| 2295 | NeuAcHex6HexNAc5 | | CO |
| 2319 | Hex6HexNAc4dHex3SP | NeuAcα3 | CO, FC, |
| | | NeuAcα6/8/9 | SP |
| | | Fucα3/4 | |
| 2367 | NeuAc2Hex5HexNAc4dHex | NeuAcα6/8/9 | CO, F |
| | | NeuAcα3 | |
| | | Fucα2 | |
| 2368 | NeuAcHex5HexNAc4dHex3 | NeuAcα3 | CO, FC |
| | | NeuAcα6/8/9 | |
| | | Fucα2 | |
| | | Fucα3/4 | |
| 2383 | NeuGcNeuAcHex5HexNAc4dHex/NeuAc2Hex6HexNAc4 | NeuAcα6/8/9 | CO (F) |
| | | NeuAcα3 | |
| | | Fucα2 | |
| 2389 | NeuAc3Hex5HexNAc3SP | NeuAcα3 | HY, SP |
| | | NeuAcα6/8/9 | |
| 2399 | NeuGc2Hex5HexNAc4dHex | NeuAcα3 | CO, F |
| | | NeuAcα6/8/9 | |
| | | Fucα3/4 | |
| 2406 | NeuAc2Hex6HexNAc3dHexSP | NeuAcα3 | HY, F, |
| | | NeuAcα6/8/9 | SP |
| | | Fucα2 | |
| 2408 | NeuAc2Hex4HexNAc5dHex | NeuAcα3 | CO, F, |
| | | NeuAcα6/8/9 | N > H |
| | | Fucα3/4 | |
| 2441 | NeuAcHex6HexNAc5dHex | | CO, F |
| 2447 | NeuAc2Hex5HexNAc4dHexSP | NeuAcα3 | CO, F, |
| | | NeuAcα6/8/9 | SP |
| | | Fucα3/4 | |
| 2448 | NeuAcHex5HexNAc4dHex3SP | NeuAcα3 | CO, FC, |
| | | NeuAcα6/8/9 | SP |
| | | Fucα3/4 | |
| 2457 | NeuAcHex7HexNAc5 | | CO |
| 2512 | NeuAc3Hex5HexNAc4 | NeuAcα3 | CO |
| | | NeuAcα6/8/9 | |
| | | Fucα2 | |
| 2513 | NeuAc2Hex5HexNAc4dHex2 | NeuAcα3 | CO, FC |
| | | NeuAcα6/8/9 | |
| | | Fucα3/4 | |
| 2528 | NeuGcNeuAc2Hex5HexNAc4 | NeuAcα3 | CO |
| | | NeuAcα6/8/9 | |
| | | Fucα2 | |
| 2529 | NeuGcNeuAcHex5HexNAc4dHex2/NeuAc2Hex6HexNAc4dHex | NeuAcα3 | CO, |
| | | NeuAcα6/8/9 | F(C) |
| | | Fucα3/4 | |
| 2544 | NeuGc2NeuAcHex5HexNAc4 | NeuAcα3 | CO |
| | | NeuAcα6/8/9 | |
| | | Fucα3/4 | |
| 2586 | NeuAc2Hex6HexNAc5 | NeuAcα3 | CO |
| | | NeuAcα6/8/9 | |
| | | Fucα2 | |
| 2587 | NeuAcHex6HexNAc5dHex2 | NeuAcα3 | CO, FC |
| | | NeuAcα6/8/9 | |
| 2603 | NeuAcHex7HexNAc5dHex/NeuGcHex6HexNAc5dHex2 | | CO, |
| | | | F(C) |
| 2619 | NeuAcHex8HexNAc5/NeuGcHex7HexNAc5dHex | Fucα2 | CO (F) |
| 2660 | NeuAcHex7HexNAc6 | Fucα3/4 | CO |
| 2732 | NeuAc2Hex6HexNAc5dHex | NeuAcα6/8/9 | CO, F |
| | | NeuAcα3 | |
| 2733 | NeuAcHex6HexNAc5dHex3 | NeuAcα3 | CO, FC |
| | | NeuAcα6/8/9 | |
| | | Fucα2 | |
| 2765 | NeuAcHex8HexNAc5dHex | NeuAcα6/8/9 | CO, F |
| | | NeuAcα3 | |
| 2781 | NeuGcHex8HexNAc5dHex/NeuAcHex9HexNAc5 | Fucα3/4 | CO (F) |
| 2878 | NeuAc3Hex6HexNAc5 | NeuAcα3 | CO |
| | | NeuAcα6/8/9 | |
| | | Fucα3/4 | |
| 2894 | NeuGcNeuAc2Hex6HexNAc5 | NeuAcα3 | CO |
| | | NeuAcα6/8/9 | |
| | | Fucα3/4 | |
| 2952 | NeuAc2Hex7HexNAc6 | NeuAcα6/8/9 | CO |
| 3024 | NeuAc3Hex6HexNAc5dHex | NeuAcα3 | CO, F |
| | | NeuAcα6/8/9 | |
| | | Fucα2 | |
| 3098 | NeuAc2Hex7HexNAc6dHex | NeuAcα3 | CO, F |
| | | NeuAcα6/8/9 | |
| | | Fucα3/4 | |

*[M − H]⁻ ion, first isotope.
^{#}Preferred structure group based on monosaccharide compositions according to the present invention. HY, hybrid-type or monoantennary; CO, complex-type; F, fucosylation; FC, complex fucosylation; N = H, terminal HexNAc (HexNAc = Hex); N > H, terminal HexNAc (HexNAc > Hex); SP, sulphate and/or phosphate ester; “( )” indicates that the glycan signal includes also other structure types.

TABLE 26
Detected acidic O-glycan signals from hESC.
Acidic O-glycan reducing oligosaccharides, [M − H]⁻ ions

| | | exp. |
|---|---|---|
| Proposed structure | calc. m/z | m/z |
| NeuAc2HexHexNAc | 964.33 | 964.35 |
| SaHex2HexNAc2 | 1038.36 | 1038.49 |
| NeuAcHex2HexNAc2dHex | 1184.42 | 1184.5 |
| Hex3HexNAc3SP | 1192.36 | 1192.73 |
| SaHex3HexNAc2 | 1200.42 | 1200.43 |
| NeuAc2Hex2HexNAc2/ | 1329.46 | 1329.56 |
| NeuGcNeuAcHexHexNAc2dHex | | |
| Hex3HexNAc3dHexSP | 1338.41 | 1338.6 |
| SaHex3HexNAc3 | 1403.49 | 1403.62 |
| Sa2Hex2HexNAcdHex | 1475.52 | 1475.79 |
| NeuAcHex6HexNAc/NeuAcHex3HexNAc3SP | 1483.49 | 1483.71 |
| SaHex3HexNAc3dHex | 1549.55 | 1549.9 |
| Hex4HexNAc4SP | 1557.49 | 1557.72 |
| SaHex4HexNAc3 | 1565.55 | 1565.66 |
| NeuAc2Hex3HexNAc3 | 1694.59 | 1694.8 |
| Hex4HexNAc4dHexSP | 1703.55 | 1703.9 |
| SaHex4HexNAc3dHex | 1711.61 | 1711.78 |
| SaHex5HexNAc3 | 1727.60 | 1727.96 |
| SaHex4HexNAc4 | 1768.57 | 1768.75 |
| SaHex6HexNAc3 | 1889.65 | 1889.96 |
| SaHex4HexNAc4dHex | 1914.68 | 1915.04 |
| SaHex5HexNAc4 | 1930.68 | 1930.83 |
| SaHex5HexNAc4dHex | 2076.74 | 2076.91 |
| NeuGcHex5HexNAc4dHex/SaHex6HexNAc4 | 2092.73 | 2092.86 |
| Sa2Hex5HexNAc4 | 2221.78 | 2221.82 |
| SaHex5HexNAc4dHex2 | 2222.80 | 2222.93 |
| NeuGcHex5HexNAc4dHex2/SaHex6HexNAc4dHex | 2238.79 | 2238.9 |
| SaHex7HexNAc4/NeuGcHex6HexNAc4dHex | 2254.79 | 2254.88 |
| SaHex5HexNAc4dHex3 | 2368.85 | 2368.26 |
| SaHex6HexNAc5dHex | 2441.87 | 2442.23 |

TABLE 27

| | Preferred | | | |
|---|---|---|---|---|
| | monosaccharide | Terminal | Experimental structures included in the glycan | |
| m/z* | compositions | epitopes | signal according to the invention^{§} | Group^{#} |
| 1825 | Hex6HexNAc4 | Galβ4 | Galβ4GlcNAc→Hex₅HexNAc₃ | CO |
| | | Galα | Galα3Gal→Hex₄HexNAc₄ | |
| | | | Galβ4GlcNAc→[Galα3Gal→]Hex₃HexNAc₃ | |
| 1987 | Hex7HexNAc4 | Galα | Galα3Gal→Hex₅HexNAc₄ | CO |
| | | | (Galα3Gal→)₂Hex₃HexNAc₄ | |
| 2133 | Hex7HexNAc4dHex1 | Galα | Galα3Gal→Hex₅HexNAc₄dHex₁ | CO, F |
| | | | (Galα3Gal→)₂Hex₃HexNAc₄dHex₁ | |
| 2190 | Hex7HexNAc5 | Galα | Galα3Gal→Hex₅HexNAc₅ | CO |
| 2336 | Hex7HexNAc5dHex | Galβ4 | Galβ4GlcNAc→Hex₆HexNAc₄dHex₁ | CO, F |
| | | Galα | Galα3Gal→Hex₅HexNAc₅dHex₁ | |
| | | | Galβ4GlcNAc→[Galα3Gal→]Hex₄HexNAc₄dHex₁ | |
| 2352 | Hex8HexNAc5 | Galβ4 | Galβ4GlcNAc→Hex₇HexNAc₄ | CO |
| | | Galα | Galα3Gal→Hex₆HexNAc₅ | |
| | | | Galβ4GlcNAc→[Galα3Gal→]Hex₅HexNAc₄ | |
| | | | Galβ4GlcNAc→[Galα3Gal→]₂Hex₃HexNAc₄ | |
| 2498 | Hex8HexNAc5dHex | Galβ4 | Galβ4GlcNAc→Hex₇HexNAc₄dHex₁ | CO, F |
| | | Galα | Galα3Gal→Hex₆HexNAc₅dHex₁ | |
| | | | Galβ4GlcNAc→[Galα3Gal→]Hex₅HexNAc₄dHex₁ | |
| | | | Galβ4GlcNAc→[Galα3Gal→]₂Hex₃HexNAc₄dHex₁ | |
| 2514 | Hex9HexNAc5 | Galα | Galα3Gal→Hex₇HexNAc₅ | CO |
| | | | (Galα3Gal→)₂Hex₅HexNAc₅ | |
| | | | (Galα3Gal→)₃Hex₃HexNAc₅ | |
| 2660 | Hex9HexNAc5dHex | Galα | Galα3Gal→Hex₇HexNAc₅dHex₁ | CO, F |
| | | | (Galα3Gal→)₂Hex₅HexNAc₅dHex₁ | |
| | | | (Galα3Gal→)₃Hex₃HexNAc₅dHex₁ | |

*[M + Na]⁺ ion, first isotope.
^{§}“→” indicates linkage to a monosaccharide in the rest of the structure; “[ ]” indicates branch in the structure.
^{#}Preferred structure group based on monosaccharide compositions according to the present invention. HI, high-mannose; LO, low-mannose; S, soluble mannosylated; HF, fucosylated high-mannose; G, glucosylated high-mannose; HY, hybrid-type or monoantennary; CO, complex-type; F, fucosylation; FC, complex fucosylation; N = H, terminal HexNAc (HexNAc = Hex); N > H, terminal HexNAc (HexNAc > Hex).

TABLE 28

| | | | | | CB CD34+ | | BM & CB | |
|---|---|---|---|---|---|---|---|---|
| Trivial name | Terminal epitope | hESC 1) | EB | st.3 | & CD133+ | CB MNC | MSC | adipo/osteo |
| LN type 1, Le^{c} | Galβ3GlcNAc | N+ | 2) | +/− | q | N+/− | q | |
| | | O+ | +/− | | | O+/− | | |
| | | L++ | | | | L+ | | |
| Lea | Galβ3(Fucα4)GlcNAc | L+ | +/− | +/− | +/− | +/− | +/− | +/− |
| H type 1 | Fucα2Galβ3GlcNAc | L++ | +/− | +/− | +/− | +/− | +/− | +/− |
| Leb | Fucα2Galβ3(Fucα4)GlcNAc | + | +/− | +/− | +/− | +/− | +/− | +/− |
| sialyl Le^{a} | SAα3Galβ3(Fucα4)GlcNAc | | +/− | +/− | | | | |
| α3′-sialyl Le^{c} | SAα3Galβ3GlcNAc | | | | +/− | +/− | +/− | +/− |
| LN type 2 | Galβ4GlcNAc | N++ | + | + | N+ | N+ | N++ | N++ |
| | | O++ | | | O+ | O+ | O+ | |
| | | L+/− | | | | L+ | L++ | |
| Le^{x} | Galβ4(Fucα3)GlcNAc | N++ | +/− | +/− | N+ | N+/− | +/− | +/− |
| | | O+/− | | | O+ | O+ | | |
| | | L+/− | | | | L+/− | | |
| H type 2 | Fucα2Galβ4GlcNAc | N+ | +/− | +/− | N+ | +/− | +/− | +/− |
| | | O+/− | | | | | | |
| | | L+/− | | | | | | |
| Le^{y} | Fucα2Galβ4(Fucα3)GlcNAc | + | +/− | +/− | | | | |
| sialyl Le^{x} | SAα3Galβ4(Fucα3)GlcNAc | + | +/− | +/− | +/− | +/− | +/− | +/− |
| α3′-sialyl LN | SAα3Galβ4GlcNAc | N++ | N+ | N+ | N++ | N+ | N++ | N++ |
| | | O+ | | | O+ | O+ | O+ | |
| α6′-sialyl LN | SAα6Galβ4GlcNAc | N+ | N++ | N++ | N+ | N++ | +/− | |
| Core 1 | Galβ3GalNAcα | O+ | +/− | +/− | O+ | O+ | O+ | |
| H type 3 | Fucα2Galβ3GalNAcα | O+ | +/− | +/− | +/− | +/− | +/− | |
| sialyl Core 1 | SAα3Galβ3GalNAcα | O+ | | | O+ | O+ | O+ | |
| disialyl Core 1 | SAα3Galβ3Saα6GalNAcα | O+ | | | O+ | O+ | O+ | |
| type 4 chain | Galβ3GalNAcβ | L+ | +/− | +/− | +/− | L+ | L+ | |
| H type 4 | Fucα2Galβ3GalNAcβ | L+ | +/− | +/− | +/− | +/− | +/− | |
| α3′-sialyl type 4 | SAα3Galβ3GalNAcβ | L++ | +/− | +/− | +/− | +/− | +/− | |
| LacdiNAc | GalNAcβ4GlcNAc | N+ | +/− | +/− | +/− | +/− | +/− | +/− |
| Lac | Galβ4Glc | L+ | q | q | q | L+ | L+ | |
| GlcNAcβ | GlcNAcβ | N+/− | q | q | N+ | +/− | +/− | q |
| | | L+ | | | | | | |
| Tn | GalNAcα | q | | | q | q | O+ | |
| sialyl Tn | SAα6GalNAcα | | | | | | O+ | |
| GalNAcβ | GalNAcβ | L+ | q | q | +/− | +/− | N+/− | |
| | | N+ | | | | | L+ | |
| poly-LN, i | repeats of Galβ4GlcNAcβ3 | + | q | q | + | + | ++ | q |
| poly-LN, I | Galβ4GlcNAcβ3(Galβ4GlcNAcβ6)Gal | L+ | +/− | +/− | +/− | L+ | L+ | q |

1) Stem cell and differentiated cell types are abbreviated as in other parts of the present document; st.3 indicates stage 3 differentiated, preferentially neuronal-type differentiated cells; adipo/osteo indicates cells differentiated into adipocyte or osteoblast direction from MSC.
2) Occurrence of terminal epitopes in glycoconjugates and/or specifically in N-glycans (N), O-glycans (O), and/or glycosphingolipids (L). Code: q, qualitative data; +/−, low expression; +, common; ++, abu

TABLE 29

| | | Neutral | | | Sialylated | | |
|---|---|---|---|---|---|---|---|
| | | glycans | | | glycans | | |
| Class | Definition | hESC | MSC | CB MNC | hESC | MSC | CB MNC |
| Examples of glycosphingolipid | | | | | | | |
| glycan classification | | | | | | | |
| Lac | n_{Hex} = 2 | 1 | 1 | 2 | 1 | a) | |
| Ltri | n_{Hex} = 2 and n_{HexNAc} = 1 | 18 | 33 | 12 | 25 | | |
| L1 | n_{Hex} = 3 and n_{HexNAc} = 1 | 46 | 32 | 46 | 56 | | |
| L2 | 3 ≦ n_{Hex} ≦ 4 and n_{HexNAc} = 2 | 11 | 15 | 4 | <1 | | |
| L3+ | i + 1 ≦ n_{Hex} ≦ i + 2 and n_{HexNAc} = i ≧ 3 | 1 | 7 | 3 | 1 | | |
| Gb | n_{Hex} = 4 and n_{HexNAc} = 1 | 20 | 1 | 1 | 16 | | |
| O | other types | 23 | 11 | 34 | 1 | | |
| F | fucosylated, n_{dHex} ≧ 1 | 43 | 12 | 7 | 1 | | |
| T | non-reducing terminal HexNAc, | 27 | 47 | 12 | 26 | | |
| | n_{Hex} ≦ n_{HexNAc} + 1 | | | | | | |
| SA1 | monosialylated, n_{Neu5Ac} = 1 | | | | 86 | | |
| SA2 | disialylated, n_{Neu5Ac} = 2 | | | | 14 | | |
| SP | sulphated or phosphorylated, +80 Da | | | | <1 | | |
| Examples of O-linked | | | | | | | |
| glycan classification | | | | | | | |
| O1 | n_{Hex} = 1 and n_{HexNAc} = 1 | a) | a) | | 43 | a) | |
| O2 | n_{Hex} = 2 and n_{HexNAc} = 2 | 53 | | | 35 | | |
| O3+ | n_{Hex} = i and n_{HexNAc} = i ≧ 3 | 13 | | | 13 | | |
| O | other types | 34 | | | 9 | | |
| F | fucosylated, n_{dHex} ≧ 1 | 1 | 47 | 64 | 5 | 15 | 15 |

| T | non-reducing terminal HexNAc, | 12 | a) | <1 | a) |
|---|---|---|---|---|---|
| | n_{Hex} ≦ n_{HexNAc} + 1 | | | | |
| SA1 | monosialylated, n_{Neu5Ac} = 1 | | | 39 | |
| SA2 | disialylated, n_{Neu5Ac} = 2 | | | 52 | |
| SP | sulphated or phosphorylated, +80 Da | | | 8 | 21 |

a) not included in present quantitative analysis.

### REFERENCES

Altmann, F., et al. (1999) Glycoconj. J. 16:109-23
Harvey, D. J., et al. (1993) Rapid Commun. Mass Spectrom. 7(7):614-9
Hirabayashi, J., et al. (2002) Biochim. Biophys. Acta. 1572:232-54.
Jaatinen, T., et al. (2006) Stem cells. 24:631-41.
Karlsson, H., et al. (2000) Glycobiology 10(12):1291-309
Kretzchmar, E., et al. (1994) Biol. Chem. Hoppe Seyler 375(5):23-7
Kubelka, V., et al. (1994) Arch. Biochem. Biophys. 308(1):148-57
Leskelä, H., et al. (2003) Biochem. Biophys. Res. Commun. 311:1008-13
Miller-Podraza, H., et al. (2000) Glycobiology. 10:975-982
Moore (1999) Trends Cell Biol. 9:441-6
Naven, T. J. & Harvey, D. J. (1996) Rapid Commun. Mass Spectrom. 10(11):1361-6
Nyman, T. A., et al. (1998) Eur. J. Biochem. 253(2):485-93
Papac, D., et al. (1996) Anal. Chem. 68(18):3215-23
Saarinen, J., et al. (1999) Eur. J. Biochem. 259(3):829-40
Skottman, H. et al. (2005) Stem cells
Staudacher, E., et al. (1992) Eur. J. Biochem. 207(3):987-93
Thomson, J. A., et al. (1998) Science 282:1145-7
Venable et al. (2005) BMC Developmental biology.

1-113. (canceled)

114. Method for culturing of human embryonic stem cells in vitro comprising the step of contacting human embryonic stem cell culture with a lectin binding to a non-reducing end terminal (Fucα2)ₙGalβ4GlcNAc, wherein n is 0 or 1.

115. The method according to claim 114, wherein human embryonal stem cells maintain undifferentiated state during culturing.

116. The method according to claim 115, wherein said lectin has specificity of from the group consisting of ECA, galectin-1, UEA-1 and DSA.

117. The method according to claim 115, wherein said lectin is immobilized to a surface by a covalent interaction.

118. The method according to claim 114, wherein said lectin is a recombinant molecule.

119. The method according to claim 118, wherein said lectin is recombinant ECA, wherein N-glycosylation site at position 113 has been mutated, preferably by a mutation of amino acid residue at position 113 N to Q changing the N-glycosylation site NNS to form QNS or mutating the serine residue to alanine or mutating a proline or bulky residue between 113 N and S in the NSS sequence.

120. Method according to claim 114 wherein said lectin is used as a media additive for culturing human embryonic stem cells.

121. A purified preparation of multipotent or pluripotent human embryonic stem cells obtained from the method according to claim 114.

122. Method for culturing of mesenchymal or embryonal stem cells comprising a step of contacting said cells with one or more covalently immobilized lectins or binders, which bind non-reducing end or reducing end terminal glycan structures and the lectins or binders recognize one or several terminal glycan structures selected from the group consisting of Gal 4GlcNAc, GalNAc 4GlcNAc, Neu5Ac 3Gal 4GlcNAc, Neu5Ac 6Gal 4GlcNAc, Fuc 2Gal 4GlcNAc, Gal 4(Fuc 3)GlcNAc, and GlcNAc 4(Fuc 6)GlcNAc.

123. The method according to claim 122, wherein the lectin or binder is conjugated from a glycan of the lectin or binder or includes chemical conjugation from specific amino acid residues from the surface of the lectin or binder protein/peptide.

124. The method according to claim 123, wherein the amino acid residue is cysteine cloned to the site of immobilization or to N-terminus of the binder.

125. The method according to claim 123, wherein conjugate structure is according to the Formula CONJ
B-(G-)ₘR1-R2-(S1-)ₙT-,
wherein B is the binder, G is glycan (when the binder is glycan conjugated),
R1 and R2 are chemoselective ligation groups, T is tag, preferably biotin, L is specifically binding ligand for the tag; S1 is an optional spacer group, preferably C₁-C₁₀ alkyls, m and n are integers being either 0 or 1, independently.

126. The method according to claim 122, wherein said lectin or binder binds to non-reducing end (Fucα2)ₙGalβ4GlcNAc, wherein n is 0 or 1.

127. The method according to claim 126, wherein the said binder or lectin is selected from the group consisting of: ECA, galectin, DSA and UEA-1, or a lectin or binder with a similar binding specificity.

128. The method according to claim 122, wherein the lectin or binder is a recombinant molecule.

129. The method according to claim 122, wherein the lectin is recombinant ECA, wherein N-glycosylation site at position 113 has been mutated, preferably by a mutation of amino acid residue at position 113 N to Q changing the N-glycosylation site NNS to form QNS or mutating the serine residue to alanine or mutating a proline or bulky residue between 113 N and S in the NSS sequence.

130. The method according to claim 122, wherein said cells are released from the immobilized lectin by a carbohydrate corresponding to the binding epitope of the lectin or parts thereof, preferably comprising Fucα2Galβ4GlcNAc, Galβ4GlcNAc, or Galβ4Glc removal of hESCs.

131. The method according to claim 122, wherein the lectin or binder is a remodelled lectin, wherein the remodeling is performed by removal of glycan or glycosylation site or inactivating the glycan.

132. The method according to claim 122, wherein the glycan of lectin or binder is inactivated by oxidation of the glycan and more preferably by periodate oxidation and derivation to inactive form or derivation and conjugation from to glycan to solid phase.

133. A preparation of human stem cells obtained using the method according to claim 122.

134. Method for modulating the status of human mesenchymal or embryonal stem cells comprising the steps of (i) contacting said cells with one or more lectins or binders, which bind non-reducing end or reducing end terminal glycan structures and the lectins or binders recognize one or several terminal glycan structures selected from the group consisting of Gal 4GlcNAc, GalNAc 4GlcNAc, Neu5Ac 3Gal 4GlcNAc, Neu5Ac 6Gal 4GlcNAc, Fuc 2Gal 4GlcNAc, Gal 4(Fuc 3)GlcNAc, and GlcNAc 4(Fuc 6)GlcNAc; (ii) incubating said cells for a period of time sufficient to achieve desired stimulation or a status change in the cells.

135. The method according to the claim 134, wherein the lectin is PSA or MAA and method is for increasing protease resistant adherence of the cells and/or morphologic status of the cells.

136. The method according to the claim 134, wherein the lectins with specificity of MAA for sialylated structures NeuNAc 3Gal 4GlcNAc, and galectin-1/ECA with N-acetyllactosamine Gal 4GlcNAc, are used for induction of some differentiation to adipocytic direction.

137. The method according to claim 134, wherein lectin specificity of HHA or ConA binding are used to support non-differentiated status of mesenchymal stem cells.

138. The method according to claim 134 wherein lectins PSA and LcHA/LCAshow differentiation of human mesenchymal stem cells by increased HLA-DR values, and ConA and MAA show low HLA-DR values.

139. A preparation of human stem cells obtained using the method according to claim 134.

140. A nucleic acid sequence, preferably in a host cell comprising the nucleic acid sequence encoding the recombinant protein as defined in claim 129, or a functional homolog or a functional fragment thereof.

141. A protein encoded by the nucleotide sequence as defined in claim 140.

142. A conjugate structure according to Formula CONJ
B-(G-)ₘR1-R2-(S1-)ₙT-,
wherein B is the binder, G is glycan (when the binder is glycan conjugated),
R1 and R2 are chemoselective ligation groups, T is tag, preferably biotin, L is specifically binding ligand for the tag; S1 is an optional spacer group, preferably C₁-C₁₀ alkyls, m and n are integers being either 0 or 1, independently.
